(19) United States
(12) Patent Application Publication
(10) Pub. No.: US 2025/0177574 A1
(43) Pub. Date: Jun. 5, 2025

(54) RETROVIRAL VECTORS

(71) Applicant: IP2IPO Innovations Limited, London (GB)

(72) Inventors: Deborah R. Gill, Oxford (GB); Stephen C. Hyde, Oxford (GB)

(21) Appl. No.: 18/927,553

(22) Filed: Oct. 25, 2024

Related U.S. Application Data
(63) Continuation of application No. 17/681,647, filed on Feb. 25, 2022.

(30) Foreign Application Priority Data
Feb. 26, 2021 (GB) 2102832.9

(51) Int. Cl. A61K 48/00; A61K 35/76; A61K 38/16; C12N 5/071; C12N 15/62; C12N 15/85

(52) U.S. Cl. CPC A61K 48/0075; A61K 35/76; A61K 38/162; C12N 5/0687; C12N 15/62; C12N 15/85; C12N 2740/15043; C12N 2760/18843; C12N 2800/107

(57) ABSTRACT

This invention relates to retroviral gene transfer vectors, particularly lentiviral vectors, pseudotyped with hemagglutinin-neuraminidase (HN) and fusion (F) proteins from a respiratory paramyxovirus, comprising a promoter and a transgene; and methods of making the same. The present invention also relates to the use of said vectors in gene therapy, particularly for the treatment of respiratory tract diseases such as Cystic Fibrosis (CF).

### CROSS-REFERENCE

This application is a continuation application of U.S. application Ser. No. 17/681,647, filed Feb. 25, 2022; which claims priority to UK Patent Application No. GB 2102832.9, filed on Feb. 26, 2021; both of which are incorporated herein by reference in their entirety.

### SEQUENCE LISTING

The instant application contains a Sequence Listing which has been submitted electronically in XML format and is hereby incorporated by reference it its entirety. Said XML copy, created on Oct. 25, 2024, is named 137266-5001-US01_SequenceListing.xml and is 184,243 bytes in size.

### BACKGROUND OF THE INVENTION

The present invention relates to retroviral gene transfer vectors, particularly lentiviral vectors, pseudotyped with hemagglutinin-neuraminidase (HN) and fusion (F) proteins from a respiratory paramyxovirus, comprising a promoter and a transgene; and methods of making the same.

Retroviruses are a family of RNA viruses (Retroviridae) that encode the enzyme reverse transcriptase. Lentiviruses are a genus of the Retroviridae family, and are characterised by a long incubation period. Retroviruses, and lentiviruses in particular, can deliver a significant amount of viral RNA into the DNA of the host cell and have the unique ability among retroviruses of being able to infect non-dividing cells, so they are one of the most efficient methods of a gene delivery vector.

Pseudotyping is the process of producing viruses or viral vectors in combination with foreign viral envelope proteins. As such, the foreign viral envelope proteins can be used to alter host tropism or an increased/decreased stability of the virus particles. For example, pseudotyping allows one to specify the character of the envelope proteins. A frequently used protein to pseudotype retroviral and lentiviral vectors is the glycoprotein G of the Vesicular stomatitis virus (VSV), short VSV-G.

Lentiviral vectors, especially those derived from HIV-1, are widely studied and frequently used vectors. The evolution of the lentiviral vectors backbone and the ability of viruses to deliver recombinant DNA molecules (transgenes) into target cells have led to their use in many applications. Two possible applications of viral vectors include restoration of functional genes in genetic therapy and in vitro recombinant protein production.

When designing retroviral/lentiviral vectors suitable for use as gene delivery vectors, one key driver is to make the vector as safe as possible for patients. A second key driver is the need to produce sufficient quantities of the vector not just to treat an individual patient, but to allow wider clinical access to the therapy for all patients who could benefit from the therapy. These two drivers can find themselves in conflict, as modifications which improve vector safety are often associated with decreased yield during vector production.

One example of a clinical setting which would benefit from gene transfer to the airway epithelium is treatment of Cystic Fibrosis (CF). CF is a fatal genetic disorder caused by mutations in the CF transmembrane conductance regulator (CFTR) gene, which acts as a chloride channel in airway epithelial cells. CF is characterised by recurrent chest infections, increased airway secretions, and eventually respiratory failure. In the UK, the current median age at death is ˜25 years. For most genotypes, there are no treatments targeting the basic defect; current treatments for symptomatic relief require hours of self-administered therapy daily. Gene therapy, unlike small molecule drugs, is independent of CFTR mutational class and is thus applicable to all affected CF individuals. However, to date there are no viral vectors approved for clinical use in the treatment of CF, and the same applies to other diseases, particularly many other respiratory tract diseases.

In addition to patient safety and yield issues, there are other difficulties conventionally associated with gene transfer to the airway epithelium.

Gene transfer efficiency to the airway epithelium is generally poor, at least in part because the respective receptors for many viral vectors appear to be predominantly localised to the basolateral surface of the airway epithelium. As such, prior to the inventors' research, the use of lentiviral pseudotypes required disruption of epithelial integrity to transduce the airways, for example by the use of detergents such as lysophosphatidylcholine or ethylene glycol bis(2-aminoethyl ether)-N,N,N′N′-tetraacetic acid, has been linked to an increased risk of sepsis. In addition, conventional gene transfer vectors struggle to penetrate the respiratory tract mucus layer, which also reduces gene transfer efficiency. The ability to administer conventional viral vectors repeatedly, mandatory for the life-long treatment of a self-renewing epithelium, is limited, because of patients' adaptive immune responses, which prevent successful repeat administration.

Administration of the vectors for clinical application is another pertinent factor. Therefore, viral stability through use of clinically relevant devices (e.g. bronchoscope and nebuliser) must be maintained for treatment efficacy.

There is accordingly a need for a gene therapy vector that is able to circumvent one or more of the problems described above. In particular, it is an object of the invention to provide a method for producing a pseudotyped retroviral or lentiviral (e.g. SIV) vector, and the means for carrying out said method, wherein the resulting vector is safe and adapted for improved gene transfer efficiency across the airway epithelium, and is produced at clinically relevant scale.

### SUMMARY OF THE INVENTION

The present inventors have previously developed a lentiviral vector, which has been pseudotyped with hemagglutinin-neuraminidase (HN) and fusion (F) proteins from a respiratory paramyxovirus, comprising a promoter and a transgene. Typically, the backbone of the vector is from a simian immunodeficiency virus (SIV), such as SIV1 or African green monkey SIV (SIV-AGM). Preferably the backbone of a viral vector of the invention is from SIV-AGM. The HN and F proteins function, respectively, to attach to sialic acids and mediate cell fusion for vector entry to target cells. The present inventors discovered that this specifically F/HN-pseudotyped lentiviral vector can efficiently transduce airway epithelium, resulting in transgene expression sustained for periods beyond the proposed lifespan of airway epithelial cells. Importantly, the present inventors also found that re-administration does not result in a loss of efficacy. These features make the vectors of the present invention attractive candidates for treating diseases via their use in expressing therapeutic proteins; (i) within the cells of the respiratory tract; (ii) secreted into the lumen of the respiratory tract; and (iii) secreted into the circulatory system.

However, there were potential safety concerns with this lentiviral vector. In particular, there was a significant degree of sequence homology between the genome vector and the GagPol vector used in its production. This sequence homology creates a theoretical risk that a replication competent lentivirus (RCL) could be generated either during manufacture, or in clinical use following administration to a patient. This represents a safety risk to the patient. The risk of generating replication competent viral particles is an issue for other retroviral/lentiviral vectors as well.

Whilst it would be desirable to mitigate this risk, it is not straightforward to do so, or at least not without eliciting other unacceptable disadvantages. In particular, it is established in the art that modifications aimed at reducing the risk of RCL, such as codon-optimisation of the manufacturing gag-pol genes typically negatively impacting the titre or yield of the vector. Given the large titres of vector required to treat even a single patient, such a reduction in yield has the potential to render its production commercially unviable.

The present inventors have now demonstrated that for the first time that the use of codon-optimised gal-pol genes from SIV do not negatively impact the manufactured titre of a SIV vector pseudotyped with hemagglutinin-neuraminidase (HN) and fusion (F) proteins from a respiratory paramyxovirus, and can even result in an increased titre of the vector. This is surprising, given that under normal manufacturing conditions (when the vector genome plasmid, rather than the gag-pol genes, is limiting), codon-optimisation of the gag-pol genes typically decreases vector yield.

Therefore, the present inventors are the first to provide a method for the production of a retroviral, particularly a lentiviral vector, such as SIV, pseudotyped with hemagglutinin-neuraminidase (HN) and fusion (F) proteins from a respiratory paramyxovirus with a reduced risk of RCL, without negatively affecting, or even increasing vector titre. Thus, the methods of the invention provide for safer vectors produced at commercially desirable yields.

Accordingly, the present invention provides a method of producing a retroviral vector pseudotyped with hemagglutinin-neuraminidase (HN) and fusion (F) proteins from a respiratory paramyxovirus, and which comprises a promoter and a transgene, wherein said method comprises the use of codon-optimised gag-pol genes. Preferably, the retroviral vector is a lentiviral vector, and optionally the lentiviral vector is selected from the group consisting of a Simian immunodeficiency virus (SIV) vector, a Human immunodeficiency virus (HIV) vector, a Feline immunodeficiency virus (FIV) vector, an Equine infectious anaemia virus (EIAV) vector, and a Visna/maedi virus vector. Particularly preferred are methods of producing an SIV vector.

The codon-optimised gag-pol genes may be SIV gag-pol genes. The codon-optimised gag-pol genes may comprise or consist of a nucleic acid sequence having at least 80% sequence identity to SEQ ID NO:1. The codon-optimised gag-pol genes may comprise or consist of the nucleic acid sequence of SEQ ID NO:1. The codon-optimised gag-pol genes may be comprised in a plasmid that comprises or consists of a nucleic acid sequence having at least 80% sequence identity to SEQ ID NO:5. The codon-optimised gag-pol genes may be comprised in a plasmid that comprises or consists of the nucleic acid sequence of SEQ ID NO:5.

The respiratory paramyxovirus may be a Sendai virus.

The titre of retroviral vector produced by a method of the invention may be: (a) equivalent to the titre of retroviral vector produced by a corresponding method which does not use codon-optimised gal-pol genes; or (b) increased compared with the titre of retroviral vector produced by a corresponding method which does not use codon-optimised gal-pol genes. Optionally, the titre of retroviral vector may be at least 1.5-fold, at least 2-fold, or at least 2.5-fold greater than the titre of retroviral vector produced by a corresponding method which does not use codon-optimised gal-pol genes.

The promoter may be selected the group consisting of a cytomegalovirus (CMV) promoter, elongation factor 1a (EF1a) promoter, and a hybrid human CMV enhancer/EF1a (hCEF) promoter. Preferably the vector comprises a hybrid human CMV enhancer/EF1a (hCEF) promoter.

The transgene may be selected from: (a) a secreted therapeutic protein, optionally Alpha-1 Antitrypsin (A1AT), Factor VIII, Surfactant Protein B (SFTPB), Factor VII, Factor IX, Factor X, Factor XI, von Willebrand Factor, Granulocyte-Macrophage Colony-Stimulating Factor (GM-CSF) and a monoclonal antibody against an infectious agent; or (b) CFTR, ABCA3, DNAH5, DNAH11, DNAI1, and DNAI2. Preferably the transgene encodes: (i) CFTR; (ii) A1AT; or (iii) FVIII.

In particularly preferred embodiments, the method produces a retroviral/lentiviral (e.g. SIV) vector wherein: (a) the promoter is a hCEF promoter and the transgene encodes CFTR; (b) the promoter is a hCEF promoter and the transgene encodes A1AT; or (c) the promoter is a hCEF or CMV promoter and the transgene encodes FVIII.

The method of the invention may comprise or consist of the following steps: (a) growing cells in suspension; (b) transfecting the cells with one or more plasmids; (c) adding a nuclease; (d) harvesting the lentivirus; (e) adding trypsin; and (d) purification. The one or more plasmids may comprise or consist of: (a) a vector genome plasmid, preferably selected from selected from pGM830 and pGM326 or variants thereof as defined herein; (b) a co-galpol plasmid, preferably pGM691 or variant thereof as defined herein; (c) a Rev plasmid, preferably pGM299 or variant thereof as defined herein; (d) a fusion (F) protein plasmid, preferably pGM301 or a variant thereof as defined herein; and (e) a hemagglutinin-neuraminidase (HN) plasmid, preferably pGM303 or a variant thereof as defined herein. The ratio of vector genome plasmid; co-gagpol plasmid; Rev plasmid; F plasmid; HN plasmid may be 20:9:6:6:6.

Steps (a)-(f) of the method may be carried out sequentially. The cells may be HEK293 cells (such as HEK293F or HEK293T cells) or 293T/17 cells. The addition of the nuclease may be at the pre-harvest stage. The addition of trypsin may be at the post-harvest stage. The purification step may comprise one or more chromatography step.

The vector genome plasmid may be modified to reduce the number of retroviral ORFs.

The invention also provides a nucleic acid comprising codon-optimised gag-pol genes, said nucleic acid having at least 80% sequence identity to SEQ ID NO:1. Preferably the nucleic acid comprises or consists of the nucleic acid sequence of SEQ ID NO:1.

The invention further provides a plasmid comprising a nucleic acid of the invention, wherein optionally: (a) the plasmid comprises or consists of a nucleic acid sequence having at least 80% sequence identity to SEQ ID NO:5; or (b) the plasmid comprises or consists of the nucleic acid sequence of SEQ ID NO:5. Optionally within the plasmid the nucleic acid is operably linked to a promoter driving expression of the Gag and Pol proteins, preferably a CAG promoter.

The invention also provides a host cell comprising a nucleic acid of the invention, and/or a plasmid of the invention.

The invention further provides a retroviral vector pseudotyped with hemagglutinin-neuraminidase (HN) and fusion (F) proteins from a respiratory paramyxovirus which is obtainable by a method of the invention.

The invention also provides a method of treating a disease comprising administering a retroviral vector pseudotyped with hemagglutinin-neuraminidase (HN) and fusion (F) proteins from a respiratory paramyxovirus which is obtainable by a method of the invention to a subject in need thereof. The disease to be treated may be a lung disease, preferably cystic fibrosis.

### BRIEF DESCRIPTION OF THE DRAWINGS

FIG. 1 shows an alignment of the wild-type (non-codon-optimised) gag-pol genes from pGM297 with the exemplary codon-optimised gag-pol genes of the invention from pGM691, showing the changes to the wild-type sequence.

FIGS. 2A-2F show schematic drawings of exemplary plasmids used for production of the vectors of the invention.

FIG. 2G shows a non-codon-optimised gag-pol plasmid (pDNA2a, specifically pGM297) that can be codon-optimised according to the invention.

FIG. 3 shows a schematic drawing of an exemplary pDNA1 plasmid used for production of the A1AT vectors of the invention.

FIGS. 4A-4D show schematic drawings of exemplary pDNA1 plasmids used for production of the FVIII vectors of the invention.

FIG. 5A illustrates homology between the pDNA1 plasmid pGM326 and the non-codon-optimised pDNA2a plasmid pGM297.

FIG. 5B compares the non-codon-optimised pDNA2a plasmid pGM297 and the codon-optimised pDNA2a plasmid pGM691 of the invention, with differences between the two annotated.

FIG. 5C shows a DNA matrix homology plot illustrates homology between the DNA sequence present in pGM297 (horizontal axis) and pGM691 (vertical axis). The solid diagonal line represents sequence homology, broken line highlights areas of reduced sequence identity; note the reduced sequence identity in the areas of gag and pol gene codon optimisation in pGM691. Note also the additional sequence present in pGM297 (located approximately 6000 to 7000 bases on the numbering shown on the horizontal axis)—this is the RRE region present in pGM297 but absent in pGM691.

FIG. 5D shows a ClustalW DNA sequence alignment of the gag pol regions of pGM297 (lower row of DNA sequence) and pGM691 (upper row of DNA sequence); sequence homology is indicated by boxed shaded regions, a consensus DNA sequence is shown underneath the pGM691 and pGM297 sequence listings. Note the complete DNA homology between the pGM297 and pGM691 sequence in (i) the gag pol Slip region, the overlapping portion of the gag pol genes, and (ii) the rabbit beta globin poly adenylation sequence (RBG pA). Note also that pGM297 contains the SIV RRE sequence while this is absent in pGM691.

FIG. 5E shows a restriction map of the codon-optimised gag-pol genes within the pGM693 plasmid

FIG. 6A shows that under design of experiment (DOE) conditions, the use of a codon-optimised pDNA2a plasmid pGM691 resulted in an observable increase in the titre of rSIV.F/HN hCEF-CFTR vector.

FIG. 6B shows that the increase in rSIV.F/HN hCEF-CFTR vector titre obtained using the codon-optimised pDNA2a plasmid pGM691 is exhibited across two different sets of experimental conditions.

FIG. 7 shows that the titre of rSIV.F/HN CMV-EGFP vector obtained using the codon-optimised pDNA2a plasmid pGM691 is greater than that obtained using the non-codon-optmised gagpol in the pDNA2a plasmid pGM297. This suggests that the advantageous properties of codon-optimised gagpol in F/HN pseudotyped vectors is not limited to the rSIV.F/HN hCEF-CFTR, but is a general property of using codon-optimised gagpol in F/HN pseudotyped vectors.

FIG. 8 shows a linear plasmid map for the Partial Gag RRE cPPT hCEF region of the pGM326 vector genome plasmid.

FIG. 9 shows an annotated schematic of the pGM326 vector genome plasmid. with SIV ORFs identified. In particular, two large ORFs, one of 189 amino acids (aa), one of 250aa were identified upstream of the hCEF promoter and soCFTR2 transgene.

FIG. 10 shows that the pGM326 vector genome plasmid and modified pGM830 vector genome plasmid in otherwise identical conditions (including non-coGagPol) produce comparable vector titres in both HEK293T cells (left panel) and A549 cells (right panel).

FIG. 11 shows the vector titre produced using coGagPol and either pGM326 or pGM830 in otherwise identical conditions, with an observable trend to increased vector titre when coGagPol is combined with pGM830.

### DETAILED DESCRIPTION OF THE INVENTION

### Definitions

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure. The meaning and scope of the terms should be clear; however, in the event of any latent ambiguity, definitions provided herein take precedent over any dictionary or extrinsic definition. It should be understood that this invention is not limited to the particular methodology, protocols, and reagents, etc., described herein and as such can vary.

This disclosure is not limited by the exemplary methods and materials disclosed herein, and any methods and materials similar or equivalent to those described herein can be used in the practice or testing of embodiments of this disclosure. The terminology used herein is for the purpose of describing particular embodiments only, and is not intended to limit the scope of the present invention, which is defined solely by the claims.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Unless otherwise indicated, any nucleic acid sequences are written left to right in 5′ to 3′ orientation; amino acid sequences are written left to right in amino to carboxy orientation. respectively.

The headings provided herein are not limitations of the various aspects or embodiments of this disclosure.

As used herein, the term “capable of” when used with a verb, encompasses or means the action of the corresponding verb. For example, “capable of interacting” also means interacting, “capable of cleaving” also means cleaves, “capable of binding” also means binds and “capable of specifically targeting . . . ” also means specifically targets.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Numeric ranges are inclusive of the numbers defining the range. Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

As used herein, the articles “a” and “an” may refer to one or to more than one (e.g. to at least one) of the grammatical object of the article. Further, unless otherwise required by context, singular terms shall include pluralities and plural terms shall include the singular. In this application, the use of “or” means “and/or” unless stated otherwise. Furthermore, the use of the term “including”, as well as other forms, such as “includes” and “included”, is not limiting.

“About” may generally mean an acceptable degree of error for the quantity measured given the nature or precision of the measurements. Exemplary degrees of error are within 20 percent (%), typically, within 10%, and more typically, within 5% of a given value or range of values. Preferably, the term “about” shall be understood herein as plus or minus (±) 5%, preferably ±4%, ±3%, ±2%, ±1%, ±0.5%, ±0.1%, of the numerical value of the number with which it is being used.

The term “consisting of” refers to compositions, methods, and respective components thereof as described herein, which are exclusive of any element not recited in that description of the invention.

As used herein the term “consisting essentially of” refers to those elements required for a given invention. The term permits the presence of elements that do not materially affect the basic and novel or functional characteristic(s) of that invention (i.e. inactive or non-immunogenic ingredients).

Embodiments described herein as “comprising” one or more features may also be considered as disclosure of the corresponding embodiments “consisting of” and/or “consisting essentially of” such features.

Concentrations, amounts, volumes, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

As used herein, the terms “vector”, “retroviral vector” and “retroviral F/HN vector” are used interchangeably to mean a retroviral vector pseudotyped with hemagglutinin-neuraminidase (HN) and fusion (F) proteins from a respiratory paramyxovirus, unless otherwise stated. The terms “lentiviral vector” and “lentiviral F/HN vector” are used interchangeably to mean a lentiviral vector pseudotyped with hemagglutinin-neuraminidase (HN) and fusion (F) proteins from a respiratory paramyxovirus, unless otherwise stated. All disclosure herein in relation to retroviral vectors of the invention applies equally and without reservation to lentiviral vectors of the invention and to SIV vectors that are pseudotyped with hemagglutinin-neuraminidase (HN) and fusion (F) proteins from a respiratory paramyxovirus (also referred to herein as SIV F/HN or SIV-FHN).

As used herein, the terms “titre” and “yield” are used interchangeably to mean the amount of lentiviral (e.g. SIV) vector produced by a method of the invention. Titre is the primary benchmark characterising manufacturing efficiency, with higher titres generally indicating that more retroviral/lentiviral (e.g. SIV) vector is manufactured (e.g. using the same amount of reagents). Titre or yield may relate to the number of vector genomes that have integrated into the genome of a target cell (integration titre), which is a measure of “active” virus particles, i.e. the number of particles capable of transducing a cell. Transducing units (TU/mL also referred to as TTU/mL) is a biological readout of the number of host cells that get transduced under certain tissue culture/virus dilutions conditions, and is a measure of the number of “active” virus particles. The total number of (active+inactive) virus particles may also be determined using any appropriate means, such as by measuring either how much Gag is present in the test solution or how many copies of viral RNA are in the test solution. Assumptions are then made that a lentivirus particle contains either 2000 Gag molecules or 2 viral RNA molecules. Once total particle number and a transducing titre/TU have been measured, a particle:infectivity ratio calculated. Amino acids are referred to herein using the name of the amino acid, the three-letter abbreviation or the single letter abbreviation.

As used herein, the terms “protein” and “polypeptide” are used interchangeably herein to designate a series of amino acid residues, connected to each other by peptide bonds between the alpha-amino and carboxyl groups of adjacent residues. The terms “protein”, and “polypeptide” refer to a polymer of amino acids, including modified amino acids (e.g., phosphorylated, glycated, glycosylated, etc.) and amino acid analogues, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogues of the foregoing.

As used herein, the terms “polynucleotides”, “nucleic acid” and “nucleic acid sequence” refers to any molecule, preferably a polymeric molecule, incorporating units of ribonucleic acid, deoxyribonucleic acid or an analogue thereof. The nucleic acid can be either single-stranded or double-stranded. A single-stranded nucleic acid can be one nucleic acid strand of a denatured double-stranded DNA Alternatively, it can be a single-stranded nucleic acid not derived from any double-stranded DNA. In one aspect, the nucleic acid can be DNA. In another aspect, the nucleic acid can be RNA Suitable nucleic acid molecules are DNA, including genomic DNA or cDNA. Other suitable nucleic acid molecules are RNA, including siRNA, shRNA, and antisense oligonucleotides. The terms “transgene” and “gene” are also used interchangeably and both terms encompass fragments or variants thereof encoding the target protein.

The transgenes of the present invention include nucleic acid sequences that have been removed from their naturally occurring environment, recombinant or cloned DNA isolates, and chemically synthesized analogues or analogues biologically synthesized by heterologous systems.

Minor variations in the amino acid sequences of the invention are contemplated as being encompassed by the present invention, providing that the variations in the amino acid sequence(s) maintain at least 60%, at least 70%, more preferably at least 80%, at least 85%, at least 90%, at least 95%, and most preferably at least 97% or at least 99% sequence identity to the amino acid sequence of the invention or a fragment thereof as defined anywhere herein. The term homology is used herein to mean identity. As such, the sequence of a variant or analogue sequence of an amino acid sequence of the invention may differ on the basis of substitution (typically conservative substitution) deletion or insertion. Proteins comprising such variations are referred to herein as variants.

Proteins of the invention may include variants in which amino acid residues from one species are substituted for the corresponding residue in another species, either at the conserved or non-conserved positions. Variants of protein molecules disclosed herein may be produced and used in the present invention. Following the lead of computational chemistry in applying multivariate data analysis techniques to the structure/property-activity relationships [see for example, Wold, et al. Multivariate data analysis in chemistry. Chemometrics-Mathematics and Statistics in Chemistry (Ed.: B. Kowalski); D. Reidel Publishing Company, Dordrecht, Holland, 1984 (ISBN 90-277-1846-6] quantitative activity-property relationships of proteins can be derived using well-known mathematical techniques, such as statistical regression, pattern recognition and classification [see for example Norman et al. Applied Regression Analysis. Wiley-Interscience; 3rd edition (April 1998) ISBN: 0471170828; Kandel, Abraham et al. Computer-Assisted Reasoning in Cluster Analysis. Prentice Hall PTR, (May 11, 1995), ISBN: 0133418847; Krzanowski, Wojtek. Principles of Multivariate Analysis: A User's Perspective (Oxford Statistical Science Series, No 22 (Paper)). Oxford University Press; (December 2000), ISBN: 0198507089; Witten, Ian H, et al Data Mining: Practical Machine Learning Tools and Techniques with Java Implementations. Morgan Kaufmann; (Oct. 11, 1999), ISBN:1558605525; Denison David G. T. (Editor) et al Bayesian Methods for Nonlinear Classification and Regression (Wiley Series in Probability and Statistics). John Wiley & Sons; (July 2002), ISBN: 0471490369; Ghose, Arup K. et al. Combinatorial Library Design and Evaluation Principles, Software, Tools, and Applications in Drug Discovery. ISBN: 0-8247-0487-8]. The properties of proteins can be derived from empirical and theoretical models (for example, analysis of likely contact residues or calculated physicochemical property) of proteins sequence, functional and three-dimensional structures and these properties can be considered individually and in combination.

Amino acids are referred to herein using the name of the amino acid, the three-letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Amino acid residues at non-conserved positions may be substituted with conservative or non-conservative residues. In particular, conservative amino acid replacements are contemplated.

A “conservative amino acid substitution” is one in which the amino acid residue is replaced with an amino acid residue having a similar side chain. Families of amino acid residues having similar side chains have been defined in the art, including basic side chains (e.g., lysine, arginine, or histidine), acidic side chains (e.g., aspartic acid or glutamic acid), uncharged polar side chains (e.g., glycine, asparagine, glutamine, serine, threonine, tyrosine, or cysteine), nonpolar side chains (e.g., alanine, valine, leucine, isoleucine, proline, phenylalanine, methionine, or tryptophan), beta-branched side chains (e.g., threonine, valine, isoleucine) and aromatic side chains (e.g., tyrosine, phenylalanine, tryptophan, or histidine). Thus, if an amino acid in a polypeptide is replaced with another amino acid from the same side chain family, the amino acid substitution is considered to be conservative. The inclusion of conservatively modified variants in a protein of the invention does not exclude other forms of variant, for example polymorphic variants, interspecies homologs, and alleles.

“Non-conservative amino acid substitutions” include those in which (i) a residue having an electropositive side chain (e.g., Arg, His or Lys) is substituted for, or by, an electronegative residue (e.g., Glu or Asp), (ii) a hydrophilic residue (e.g., Ser or Thr) is substituted for, or by, a hydrophobic residue (e.g., Ala, Leu, Ile, Phe or Val), (iii) a cysteine or proline is substituted for, or by, any other residue, or (iv) a residue having a bulky hydrophobic or aromatic side chain (e.g., Val, His, Ile or Trp) is substituted for, or by, one having a smaller side chain (e.g., Ala or Ser) or no side chain (e.g., Gly).

“Insertions” or “deletions” are typically in the range of about 1, 2, or 3 amino acids. The variation allowed may be experimentally determined by systematically introducing insertions or deletions of amino acids in a protein using recombinant DNA techniques and assaying the resulting recombinant variants for activity. This does not require more than routine experiments for a skilled person.

A “fragment” of a polypeptide comprises at least 50%, at least 60%, at least 70%, at least 80%, at least 90%, at least 95%, at least 97% or more of the original polypeptide.

The polynucleotides of the present invention may be prepared by any means known in the art. For example, large amounts of the polynucleotides may be produced by replication in a suitable host cell. The natural or synthetic DNA fragments coding for a desired fragment will be incorporated into recombinant nucleic acid constructs, typically DNA constructs, capable of introduction into and replication in a prokaryotic or eukaryotic cell. Usually the DNA constructs will be suitable for autonomous replication in a unicellular host, such as yeast or bacteria, but may also be intended for introduction to and integration within the genome of a cultured insect, mammalian, plant or other eukaryotic cell lines.

The polynucleotides of the present invention may also be produced by chemical synthesis, e.g. by the phosphoramidite method or the tri-ester method, and may be performed on commercial automated oligonucleotide synthesizers. A double-stranded fragment may be obtained from the single stranded product of chemical synthesis either by synthesizing the complementary strand and annealing the strand together under appropriate conditions or by adding the complementary strand using DNA polymerase with an appropriate primer sequence.

When applied to a nucleic acid sequence, the term “isolated” in the context of the present invention denotes that the polynucleotide sequence has been removed from its natural genetic milieu and is thus free of other extraneous or unwanted coding sequences (but may include naturally occurring 5′ and 3′ untranslated regions such as promoters and terminators), and is in a form suitable for use within genetically engineered protein production systems. Such isolated molecules are those that are separated from their natural environment.

In view of the degeneracy of the genetic code, considerable sequence variation is possible among the polynucleotides of the present invention. Degenerate codons encompassing all possible codons for a given amino acid are set forth below:

| | | Degenerate |
|---|---|---|
| Amino Acid | Codons | Codon |
| Cys | TGC TGT | TGY |
| Ser | AGC AGT TCA TCC TCG TCT | WSN |
| Thr | ACA ACC ACG ACT | ACN |
| Pro | CCA CCC CCG CCT | CCN |
| Ala | GCA GCC GCG GCT | GCN |
| Gly | GGA GGC GGG GGT | GGN |
| Asn | AAC AAT | AAY |
| Asp | GAC GAT | GAY |
| Glu | GAA GAG | GAR |
| Gln | CAA CAG | CAR |
| His | CAC CAT | CAY |
| Arg | AGA AGG CGA CGC CGG CGT | MGN |
| Lys | AAA AAG | AAR |
| Met | ATG | ATG |
| Ile | ATA ATC ATT | ATH |
| Leu | CTA CTC CTG CTT TTA TTG | YTN |
| Val | GTA GTC GTG GTT | GTN |
| Phe | TTC TTT | TTY |
| Tyr | TAC TAT | TAY |
| Trp | TGG | TGG |
| Ter | TAA TAG TGA | TRR |
| Asn/Asp | | RAY |
| Glu/Gln | | SAR |
| Any | | NNN |

One of ordinary skill in the art will appreciate that flexibility exists when determining a degenerate codon, representative of all possible codons encoding each amino acid. For example, some polynucleotides encompassed by the degenerate sequence may encode variant amino acid sequences, but one of ordinary skill in the art can easily identify such variant sequences by reference to the amino acid sequences of the present invention.

A “variant” nucleic acid sequence has substantial homology or substantial similarity to a reference nucleic acid sequence (or a fragment thereof). A nucleic acid sequence or fragment thereof is “substantially homologous” (or “substantially identical”) to a reference sequence if, when optimally aligned (with appropriate nucleotide insertions or deletions) with the other nucleic acid (or its complementary strand), there is nucleotide sequence identity in at least about 70%, 75%, 80%, 85, 90, 91, 92, 93, 94, 95, 96, 97, 98, 99 or more % of the nucleotide bases. Methods for homology determination of nucleic acid sequences are known in the art.

Alternatively, a “variant” nucleic acid sequence is substantially homologous with (or substantially identical to) a reference sequence (or a fragment thereof) if the “variant” and the reference sequence they are capable of hybridizing under stringent (e.g. highly stringent) hybridization conditions. Nucleic acid sequence hybridization will be affected by such conditions as salt concentration (e.g. NaCl), temperature, or organic solvents, in addition to the base composition, length of the complementary strands, and the number of nucleotide base mismatches between the hybridizing nucleic acids, as will be readily appreciated by those skilled in the art. Stringent temperature conditions are preferably employed, and generally include temperatures in excess of 30° C., typically in excess of 37° C. and preferably in excess of 45° C. Stringent salt conditions will ordinarily be less than 1000 mM, typically less than 500 mM, and preferably less than 200 mM. The pH is typically between 7.0 and 8.3. The combination of parameters is much more important than any single parameter.

Methods of determining nucleic acid percentage sequence identity are known in the art. By way of example, when assessing nucleic acid sequence identity, a sequence having a defined number of contiguous nucleotides may be aligned with a nucleic acid sequence (having the same number of contiguous nucleotides) from the corresponding portion of a nucleic acid sequence of the present invention. Tools known in the art for determining nucleic acid percentage sequence identity include Nucleotide BLAST (as described below).

One of ordinary skill in the art appreciates that different species exhibit “preferential codon usage”. As used herein, the term “preferential codon usage” refers to codons that are most frequently used in cells of a certain species, thus favouring one or a few representatives of the possible codons encoding each amino acid. For example, the amino acid threonine (Thr) may be encoded by ACA, ACC, ACG, or ACT, but in mammalian host cells ACC is the most commonly used codon; in other species, different codons may be preferential. Preferential codons for a particular host cell species can be introduced into the polynucleotides of the present invention by a variety of methods known in the art. Introduction of preferential codon sequences into recombinant DNA can, for example, enhance production of the protein by making protein translation more efficient within a particular cell type or species. Thus, according to the invention, in addition to the gag-pol genes any nucleic acid sequence may be codon-optimised for expression in a host or target cell. In particular, the vector genome (or corresponding plasmid), the REV gene (or corresponding plasmid), the fusion protein (F) gene (or correspond plasmid) and/or the hemagglutinin-neuraminidase (HN) gene (or corresponding plasmid, or any combination thereof may be codon-optimised.

A “fragment” of a polynucleotide of interest comprises a series of consecutive nucleotides from the sequence of said full-length polynucleotide. By way of example, a “fragment” of a polynucleotide of interest may comprise (or consist of) at least 30 consecutive nucleotides from the sequence of said polynucleotide (e.g. at least 35, 50, 75, 100, 150, 200, 250, 300, 350, 400, 450, 500, 550, 600, 650, 700, 750, 800 850, 900, 950 or 1000 consecutive nucleic acid residues of said polynucleotide). A fragment may include at least one antigenic determinant and/or may encode at least one antigenic epitope of the corresponding polypeptide of interest. Typically, a fragment as defined herein retains the same function as the full-length polynucleotide.

The terms “decrease”, “reduced”, “reduction”, or “inhibit” are all used herein to mean a decrease by a statistically significant amount. The terms “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” encompasses a complete inhibition or reduction as compared to a reference level. “Complete inhibition” is a 100% inhibition (i.e. abrogation) as compared to a reference level.

The terms “increased”, “increase”, “enhance”, or “activate” are all used herein to mean an increase by a statically significant amount. The terms “increased”, “increase”, “enhance”, or “activate” can mean an increase of at least 25%, at least 50% as compared to a reference level, for example an increase of at least about 50%, or at least about 75%, or at least about 80%, or at least about 90%, or at least about 100%, or at least about 150%, or at least about 200%, or at least about 250% or more compared with a reference level, or at least about a 1.5-fold, or at least about a 2-fold, or at least about a 2.5-fold, or at least about a 3-fold, or at least about a 4-fold, or at least about a 5-fold or at least about a 10-fold increase, or any increase between 1.5-fold and 10-fold or greater as compared to a reference level. In the context of a yield or titre, an “increase” is an observable or statistically significant increase in such level.

The terms “individual”, “subject”, and “patient”, are used interchangeably herein to refer to a mammalian subject for whom diagnosis, prognosis, disease monitoring, treatment, therapy, and/or therapy optimisation is desired. The mammal can be (without limitation) a human, non-human primate, mouse, rat, dog, cat, horse, or cow. In a preferred embodiment, the individual, subject, or patient is a human. An “individual” may be an adult, juvenile or infant. An “individual” may be male or female.

A “subject in need” of treatment for a particular condition can be an individual having that condition, diagnosed as having that condition, or at risk of developing that condition.

A subject can be one who has been previously diagnosed with or identified as suffering from or having a condition in need of treatment or one or more complications or symptoms related to such a condition, and optionally, have already undergone treatment for a condition as defined herein or the one or more complications or symptoms related to said condition. Alternatively, a subject can also be one who has not been previously diagnosed as having a condition as defined herein or one or more or symptoms or complications related to said condition. For example, a subject can be one who exhibits one or more risk factors for a condition, or one or more or symptoms or complications related to said condition or a subject who does not exhibit risk factors.

As used herein, the term “healthy individual” refers to an individual or group of individuals who are in a healthy state, e.g. individuals who have not shown any symptoms of the disease, have not been diagnosed with the disease and/or are not likely to develop the disease e.g. cystic fibrosis (CF) or any other disease described herein). Preferably said healthy individual(s) is not on medication affecting CF and has not been diagnosed with any other disease. The one or more healthy individuals may have a similar sex, age, and/or body mass index (BMI) as compared with the test individual. Application of standard statistical methods used in medicine permits determination of normal levels of expression in healthy individuals, and significant deviations from such normal levels.

Herein the terms “control” and “reference population” are used interchangeably.

The term “pharmaceutically acceptable” as used herein means approved by a regulatory agency of the Federal or a state government, or listed in the U.S. Pharmacopeia, European Pharmacopeia or other generally recognized pharmacopeia

The publications discussed herein are provided solely for their disclosure prior to the filing date of the present application. Nothing herein is to be construed as an admission that such publications constitute prior art to the claims appended hereto.

Disclosure related to the various methods of the invention are intended to be applied equally to other methods, therapeutic uses or methods, the data storage medium or device, the computer program product, and vice versa.

### Retroviral and Lentiviral Vectors

The invention relates to the production of a retroviral/lentiviral (e.g. SIV) construct. The term “retrovirus” refers to any member of the Retroviridae family of RNA viruses that encode the enzyme reverse transcriptase. The term “lentivirus” refers to a family of retroviruses. Examples of retroviruses suitable for use in the present invention include gammaretroviruses such as murine leukaemia virus (MLV) and feline leukaemia virus (FLV). Examples of lentiviruses suitable for use in the present invention include Simian immunodeficiency virus (SIV), Human immunodeficiency virus (HIV), Feline immunodeficiency virus (FIV), Equine infectious anaemia virus (EIAV), and Visna/maedi virus. Preferably the invention relates to lentiviral vectorsand the production thereof. A particularly preferred lentiviral vector is an SIV vector (including all strains and subtypes), such as a SIV-AGM (originally isolated from African green monkeys, Cercopithecus aethiops). Alternatively the invention relates to HIV vectors.

The retroviral/lentiviral (e.g. SIV) vectors of the present invention are typically pseudotyped with hemagglutinin-neuraminidase (HN) and fusion (F) proteins from a respiratory paramyxovirus. Preferably the respiratory paramyxovirus is a Sendai virus (murine parainfluenza virus type 1). The retroviral/lentiviral (e.g. SIV) vectors of the present invention may be pseudotyped with proteins from another virus, provided that the use of codon-optimised gag-pol genes (e.g. from SIV) does not negatively impact the manufactured titre of the vector, or even results in an increased titre of the vector. Non-limiting examples of other proteins that may be used to pseudotype retroviral/lentiviral (e.g. SIV) vectors of the present invention include G glycoprotein from Vesicular Stomatitis Virus (G-VSV) and severe acute respiratory syndrome coronavirus 2 (SARS-CoV-2) spike protein or modified forms thereof; such as those described in UK Patent Application Nos. 2118685.3 and 2105278.2, each of which is herein incorporated by reference in its entirety. Thus, the invention may relate to the production of SIV pseudotyped with G-VSV or SIV pseudotyped with a SARS-CoV-2 spike protein, using codon-optimised gag-pol genes.

A retroviral/lentiviral (e.g. SIV) vector produced according to the invention may be integrase-competent (IC). Alternatively, the lentiviral (e.g. SIV) vector may be integrase-deficient (ID).

Retroviral/Lentiviral vectors, such as those produced according to the invention, can integrate into the genome of transduced cells and lead to long-lasting expression, making them suitable for transduction of stem/progenitor cells. In the lung, several cell types with regenerative capacity have been identified as responsible for maintaining specific cell lineages in the conducting airways and alveoli. These include basal cells and submucosal gland duct cells in the upper airways, club cells and neuroendocrine cells in the bronchiolar airways, bronchioalveolar stem cells in the terminal bronchioles and type II pneumocytes in the alveoli. Therefore, and without being bound by theory, it is believed that said retroviral/lentiviral (e.g. SIV) vectors bring about long term gene expression of the transgene of interest by introducing the transgene into one or more long-lived airway epithelial cells or cell types, such as basal cells and submucosal gland duct cells in the upper airways, club cells and neuroendocrine cells in the bronchiolar airways, bronchioalveolar stem cells in the terminal bronchioles and type II pneumocytes in the alveoli.

Accordingly, the retroviral/lentiviral (e.g. SIV) vectors produced according to the invention may transduce one or more cells or cell lines with regenerative potential within the lung (including the airways and respiratory tract) to achieve long term gene expression. For example, the retroviral/lentiviral (e.g. SIV) vectors may transduce basal cells, such as those in the upper airways/respiratory tract. Basal cells have a central role in processes of epithelial maintenance and repair following injury. In addition, basal cells are widely distributed along the human respiratory epithelium, with a relative distribution ranging from 30% (larger airways) to 6% (smaller airways).

The retroviral/lentiviral (e.g. SIV) vectors produced according to the invention may be used to transduce isolated and expanded stem/progenitor cells ex vivo prior administration to a patient. Preferably, the retroviral/lentiviral (e.g. SIV) vectors produced according to the invention are used to transduce cells within the lung (or airways/respiratory tract) in vivo.

The retroviral/lentiviral (e.g. SIV) vectors of the invention demonstrate remarkable resistance to shear forces with only modest reduction in transduction ability when passaged through clinically-relevant delivery devices such as bronchoscopes, spray bottles and nebulisers.

The retroviral/lentiviral (e.g. SIV) vectors of the present invention enable high levels of transgene expression, resulting in high levels (therapeutic levels) of expression of a therapeutic protein. The retroviral/lentiviral (e.g. SIV) vectors of the present invention typically provide high expression levels of a transgene when administered to a patient. The terms high expression and therapeutic expression are used interchangeably herein. Expression may be measured by any appropriate method (qualitative or quantitative, preferably quantitative), and concentrations given in any appropriate unit of measurement, for example ng/ml or nM.

Expression of a transgene of interest may be given relative to the expression of the corresponding endogenous (defective) gene in a patient. Expression may be measured in terms of mRNA or protein expression. The expression of the transgene of the invention, such as a functional CFTR gene, may be quantified relative to the endogenous gene, such as the endogenous (dysfunctional) CFTR genes in terms of mRNA copies per cell or any other appropriate unit.

Expression levels of a transgene and/or the encoded therapeutic protein of the invention may be measured in the lung tissue, epithelial lining fluid and/or serum/plasma as appropriate. A high and/or therapeutic expression level may therefore refer to the concentration in the lung, epithelial lining fluid and/or serum/plasma.

The transgene included in the vector of the invention may be modified to facilitate expression. For example, the transgene sequence may be in CpG-depleted (or CpG-fee) and/or codon-optimised form to facilitate gene expression. Standard techniques for modifying the transgene sequence in this way are known in the art.

The retroviral/lentiviral (e.g. SIV) vectors of the invention exhibit efficient airway cell uptake, enhanced transgene expression, and suffer no loss of efficacy upon repeated administration. Accordingly, the retroviral/lentiviral (e.g. SIV) vectors of the invention are capable of producing long-lasting, repeatable, high-level expression in airway cells without inducing an undue immune response.

The retroviral/lentiviral (e.g. SIV) vectors of the present invention enable long-term transgene expression, resulting in long-term expression of a therapeutic protein. As described herein, the phrases “long-term expression”, “sustained expression”, “long-lasting expression” and “persistent expression” are used interchangeably. Long-term expression according to the present invention means expression of a therapeutic gene and/or protein, preferably at therapeutic levels, for at least 45 days, at least 60 days, at least 90 days, at least 120 days, at least 180 days, at least 250 days, at least 360 days, at least 450 days, at least 730 days or more. Preferably long-term expression means expression for at least 90 days, at least 120 days, at least 180 days, at least 250 days, at least 360 days, at least 450 days, at least 720 days or more, more preferably at least 360 days, at least 450 days, at least 720 days or more. This long-term expression may be achieved by repeated doses or by a single dose.

Repeated doses may be administered twice-daily, daily, twice-weekly, weekly, monthly, every two months, every three months, every four months, every six months, yearly, every two years, or more. Dosing may be continued for as long as required, for example, for at least six months, at least one year, two years, three years, four years, five years, ten years, fifteen years, twenty years, or more, up to for the lifetime of the patient to be treated.

The retroviral/lentiviral (e.g. SIV) vector comprises a promoter operably linked to a transgene, enabling expression of the transgene. Typically the promoter is a hybrid human CMV enhancer/EF1a (hCEF) promoter. This hCEF promoter may lack the intron corresponding to nucleotides 570-709 and the exon corresponding to nucleotides 728-733 of the hCEF promoter. A preferred example of an hCEF promoter sequence of the invention is provided by SEQ ID NO:10. The promoter may be a CMV promoter. An example of a CMV promoter sequence is provided by SEQ ID NO:11. The promoter may be a human elongation factor 1a (EF1a) promoter. An example of a EF1a promoter is provided by SEQ ID NO:12. Other promoters for transgene expression are known in the art and their suitability for the retroviral/lentiviral (e.g. SIV) vectors of the invention determined using routine techniques known in the art. Non-limiting examples of other promoters include UbC and UCOE. As described herein, the promoter may be modified to further regulate expression of the transgene of the invention.

The promoter included in the retroviral/lentiviral (e.g. SIV) vector of the invention may be specifically selected and/or modified to further refine regulation of expression of the therapeutic gene. Again, suitable promoters and standard techniques for their modification are known in the art. As a non-limiting example, a number of suitable (CpG-free) promoters suitable for use in the present invention are described in Pringle et al. (J. Mol. Med. Berl. 2012, 90(12):1487-96), which is herein incorporated by reference in its entirety. Preferably, the retroviral/lentiviral vectors (particularly SIV F/HN vectors) of the invention comprise a hCEF promoter having low or no CpG dinucleotide content. The hCEF promoter may have all CG dinucleotides replaced with any one of AG, TG or GT. Thus, the hCEF promoter may be CpG-free. A preferred example of a CpG-free hCEF promoter sequence of the invention is provided by SEQ ID NO:10. The absence of CpG dinucleotides further improves the performance of retroviral/lentiviral (e.g. SIV) vectors of the invention and in particular in situations where it is not desired to induce an immune response against an expressed antigen or an inflammatory response against the delivered expression construct. The elimination of CpG dinucleotides reduces the occurrence of flu-like symptoms and inflammation which may result from administration of constructs, particularly when administered to the airways.

The retroviral/lentiviral (e.g. SIV) vector of the invention may be modified to allow shut down of gene expression. Standard techniques for modifying the vector in this way are known in the art. As a non-limiting example, Tet-responsive promoters are widely used.

Preferably, the invention relates to F/HN retroviral/lentiviral vectors comprising a promoter and a transgene, particularly SIV F/HN vectors. The F/HN pseudotyping is particularly efficient at targeting cells in the airway epithelium, and as such, for therapeutic applications it is typically delivered to cells of the respiratory tract, including the cells of the airway epithelium. Accordingly, the retroviral/lentiviral (e.g. SIV) vectors of the invention are particularly suited for treatment of diseases or disorders of the airways, respiratory tract, or lung. Typically, the retroviral/lentiviral (e.g. SIV) vectors may be used for the treatment of a genetic respiratory disease.

A retroviral/lentiviral (e.g. SIV) vector of the invention may comprise a transgene that encodes a polypeptide or protein that is therapeutic for the treatment of such diseases, particularly a disease or disorder of the airways, respiratory tract, or lung.

Accordingly, a retroviral/lentiviral (e.g. SIV) vector of the invention may comprise a transgene encoding a protein selected from: (i) a secreted therapeutic protein, optionally Alpha-1 Antitrypsin (A1AT), Factor VIII, Surfactant Protein B (SFTPB), Factor VII, Factor IX, Factor X, Factor XI, von Willebrand Factor, Granulocyte-Macrophage Colony-Stimulating Factor (GM-CSF) and a monoclonal antibody against an infectious agent; or (ii) CFTR, ABCA3, DNAH5, DNAH11, DNAI1, and DNAI2. Other examples of transgenes that may be comprised in a retroviral/lentiviral (e.g. SIV) vector of the invention include genes related to or associated with other surfactant deficiencies.

Preferably, the transgene encodes a CFTR. An example of a CFTR cDNA is provided by SEQ ID NO:13. Variants thereof (as described therein) are also included, particularly variants with at least 90% (such as at least 90, 92, 94, 95, 96, 97, 98, 99 or 100% to SEQ ID NO:13.

The transgene may encode an A1AT. An example of an A1AT transgene is provided by SEQ ID NO:14, or by the complementary sequence of SEQ ID NO:15. SEQ ID NO:14 is a codon-optimized CpG depleted A1AT transgene previously designed by the present inventors to enhance translation in human cells. Such optimisation has been shown to enhance gene expression by up to 15-fold. Variants of same sequence (as defined herein) which possess the same technical effect of enhancing translation compared with the unmodified (wild-type) A1AT gene sequence are also encompassed by the present invention. The polypeptide encoded by said A1AT transgene, may be exemplified by the polypeptide of SEQ ID NO:16. Variants thereof (as described therein) are also included, particularly variants with at least 90% (such as at least 90, 92, 94, 95, 96, 97, 98, 99 or 100% to SEQ ID NO:14, 15 or 16.

The transgene may encode a FVIII. Examples of a FVIII transgene are provided by SEQ ID NOs:17 and 18, or by the respective complementary sequences of SEQ ID NO:19 and 20. The polypeptide encoded by the FVIII transgene. may be exemplified by the polypeptide of SEQ ID NO:21 or 22. Variants thereof (as described therein) are also included, particularly variants with at least 90% (such as at least 90, 92, 94, 95, 96, 97, 98, 99 or 100% to any one of SEQ ID NOs:17 to 22.

The transgene of the invention may be any one or more of DNAH5, DNAH11, DNAI1, and DNAI2, or other known related gene.

When the respiratory tract epithelium is targeted for delivery of the retroviral/lentiviral (e.g. SIV) vector, the transgene may encode A1AT, SFTPB, or GM-CSF. The transgene may encode a monoclonal antibody (mAb) against an infectious agent. The transgene may encode anti-TNF alpha. The transgene may encode a therapeutic protein implicated in an inflammatory, immune or metabolic condition.

A retroviral/lentiviral (e.g. SIV) vector of the invention may be delivered to the cells of the respiratory tract to allow production of proteins to be secreted into circulatory system. In such embodiments, the transgene may encode for Factor VII, Factor VIII, Factor IX, Factor X, Factor XI and/or von Willebrand's factor. Such a vector may be used in the treatment of diseases, particularly cardiovascular diseases and blood disorders, preferably blood clotting deficiencies such as haemophilia. Again, the transgene may encode an mAb against an infectious agent or a protein implicated in an inflammatory, immune or metabolic condition, such as, lysosomal storage disease.

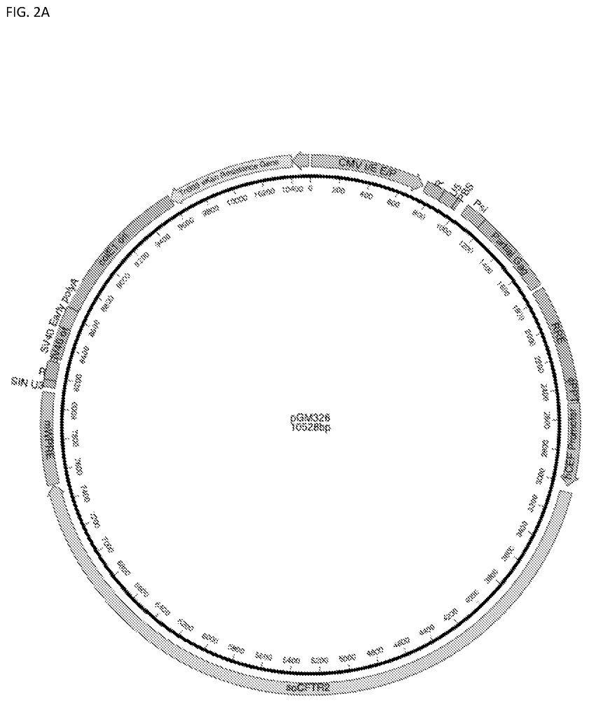

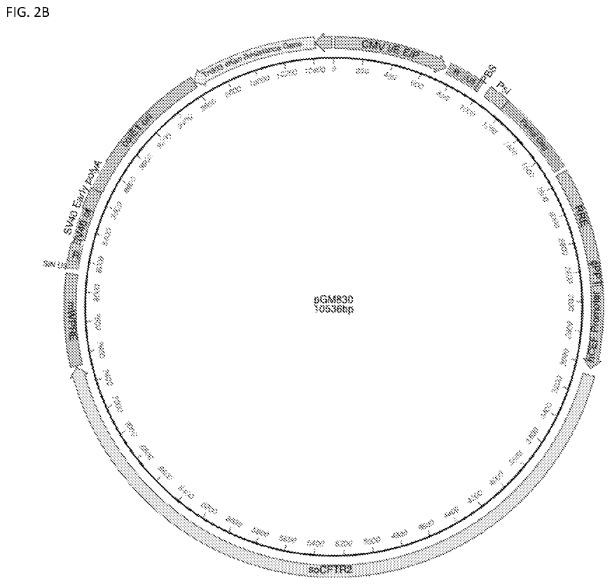

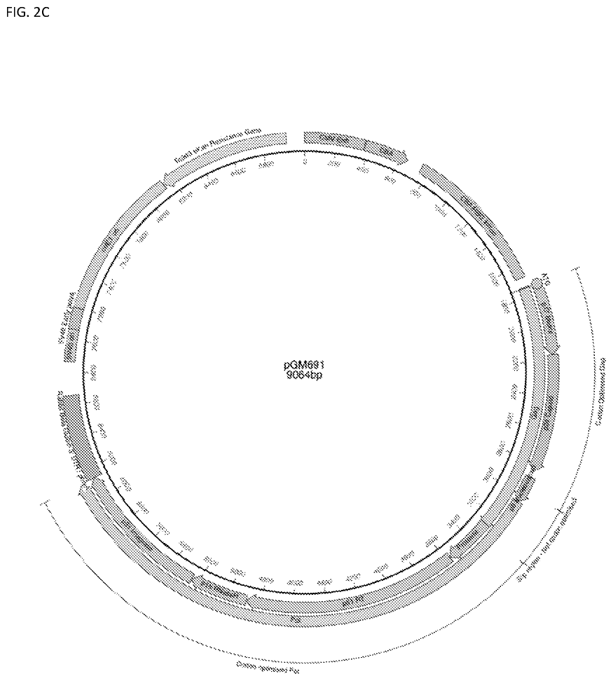

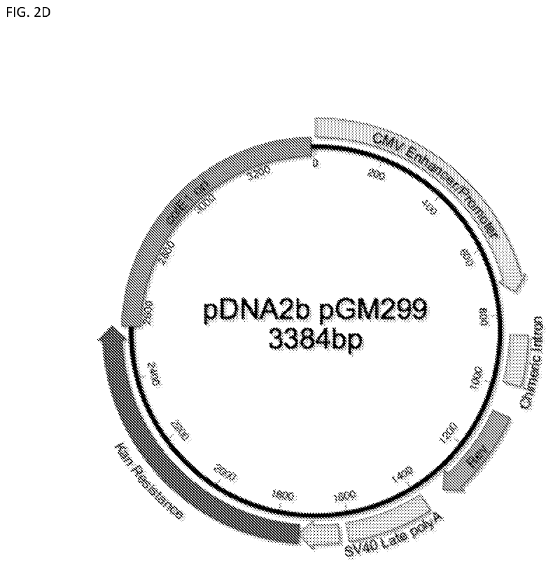

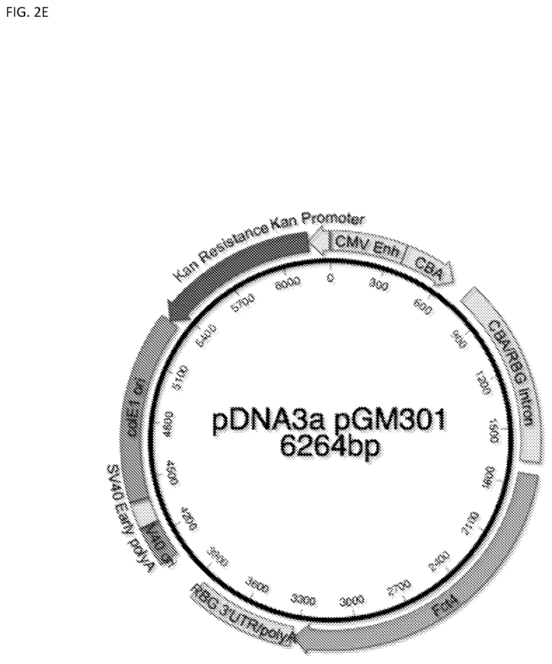

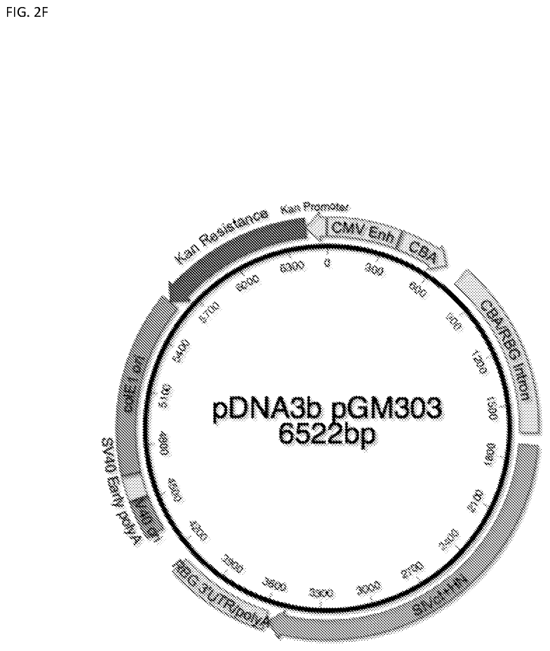

The retroviral/lentiviral (e.g. SIV) vector of the invention may have no intron positioned between the promoter and the transgene. Similarly, there may be no intron between the promoter and the transgene in the vector genome (pDNA1) plasmid (for example, pGM326 as described herein, illustrated in FIG. 2A and with the sequence of SEQ ID NO:3).

In some preferred embodiments, the retroviral/lentiviral (e.g. SIV) vector comprises a hCEF promoter and a CFTR transgene, including those described herein. Optionally said retroviral/lentiviral (e.g. SIV) vector may have no intron positioned between the promoter and the transgene. Such a retroviral/lentiviral (e.g. SIV) vector may be produced by the method described herein, using a genome plasmid carrying the CFTR transgene and a promoter.

In some preferred embodiments, the retroviral/lentiviral (e.g. SIV) vector comprises a hCEF promoter and an A1AT transgene, including those described herein. Optionally said retroviral/lentiviral (e.g. SIV) vector may have no intron positioned between the promoter and the transgene. Such a retroviral/lentiviral (e.g. SIV) vector may be produced by the method described herein, using a genome plasmid carrying the A1AT transgene and a promoter.

In some preferred embodiments, the retroviral/lentiviral (e.g. SIV) vector comprises a hCEF or CMW promoter and an FVIII transgene, including those described herein. Optionally said retroviral/lentiviral (e.g. SIV) vector may have no intron positioned between the promoter and the transgene. Such a retroviral/lentiviral (e.g. SIV) vector may be produced by the method described herein, using a genome plasmid carrying the FVIII transgene and a promoter.

The retroviral/lentiviral (e.g. SIV) vector as described herein comprises a transgene. The transgene comprises a nucleic acid sequence encoding a gene product, e.g., a protein, particularly a therapeutic protein.

For example, in one embodiment, the nucleic acid sequence encoding a CFTR, A1AT or FVIII comprises (or consists of) a nucleic acid sequence having at least 90% (such as at least 90, 92, 94, 95, 96, 97, 98, 99 or 100%) sequence identity to the CFTR, A1AT or FVIII nucleic acid sequence respectively, examples of which are described herein. In a further embodiment, the nucleic acid sequence encoding CFTR, A1AT or FVIII comprises (or consists of) a nucleic acid sequence having at least 95% (such as at least 95, 96, 97, 98, 99 or 100%) sequence identity to the CFTR, A1AT or FVIII nucleic acid sequence respectively, examples of which are described herein. In one embodiment, the nucleic acid sequence encoding CFTR is provided by SEQ ID NO:13, the nucleic acid sequence encoding A1AT is provided by SEQ ID NO:14, or by the complementary sequence of SEQ ID NO:15 and/or the nucleic acid sequence encoding FVIII is provided by SEQ ID NO:17 and 18, or by the respective complementary sequences of SEQ ID NO:19 and 20, or variants thereof.

The amino acid sequence of the CFTR, A1AT or FVIII transgene may comprise (or consist of) an amino acid sequence having at least 95% (such as at least 95, 96, 97, 98, 99 or 100%) sequence identity to the functional CFTR, A1AT or FVIII polypeptide sequence respectively.

The retroviral/lentiviral (e.g. SIV) vectors of the invention may comprise a central polypurine tract (cPPT) and/or the Woodchuck hepatitis virus posttranscriptional regulatory elements (WPRE). An exemplary WPRE sequence is provided by SEQ ID NO:23.

### Methods of Production

As described herein, the present inventors have demonstrated for the first time that the use of codon-optimised gal-pol genes from SIV does not negatively impact the manufactured titre of a SIV vector pseudotyped with hemagglutinin-neuraminidase (HN) and fusion (F) proteins from a respiratory paramyxovirus, and can even result in an increased titre of the vector. In addition, the inventors have further shown that the use of codon-optimised gag-pol genes can be further combined with the use of a modified vector genome plasmid as described herein whilst maintaining, or even increasing the vector titre.

Codon optimisation is a technique to maximise protein expression by increasing the translational efficiency of the encoding gene. Translational efficiency is increased by modification of the nucleic acid sequence. Codon optimisation is routine in the art, and it is within the routine practice of one of ordinary skill to devise a codon-optimised version of a given nucleic acid sequence. However, what is not straightforward is predicting the effect of codon optimisation on other parameters. For example, as described herein, conventional wisdom teaches that under normal manufacturing conditions (when the vector genome plasmid, rather than the gag-pol genes, is limiting), codon-optimisation of the gag-pol genes typically decreases vector yield.

Accordingly, the present invention provides a method of producing a retroviral/lentiviral (e.g. SIV) vector pseudotyped with hemagglutinin-neuraminidase (HN) and fusion (F) proteins from a respiratory paramyxovirus, and which comprises a promoter and a transgene, wherein said method comprises the use of codon-optimised gag-pol genes. Preferably said vector is a lentiviral vector, with Simian immunodeficiency virus (SIV) vectors being particularly preferred.

Typically the codon-optimised gag-pol genes used in the production methods of the invention are matched to the retroviral/lentiviral vector being produced. By way of non-limiting example, when the lentiviral vector is an HIV vector, the codon-optimised gag-pol genes used in the production methods of the invention are HIV gag-pol genes. By way of non-limiting example, when the lentiviral vector is an SIV vector, the codon-optimised gag-pol genes used in the production methods of the invention are SIV gag-pol genes.

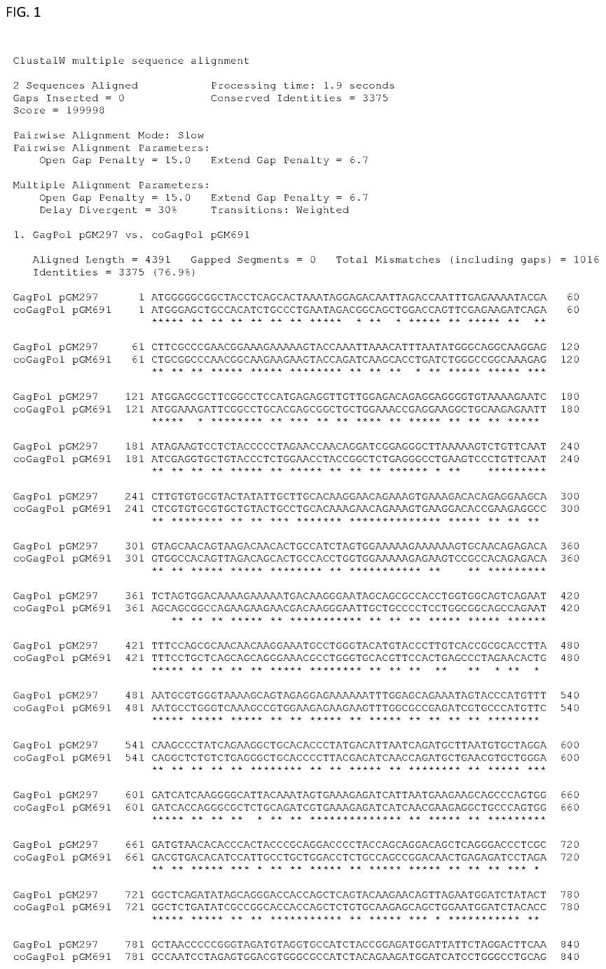

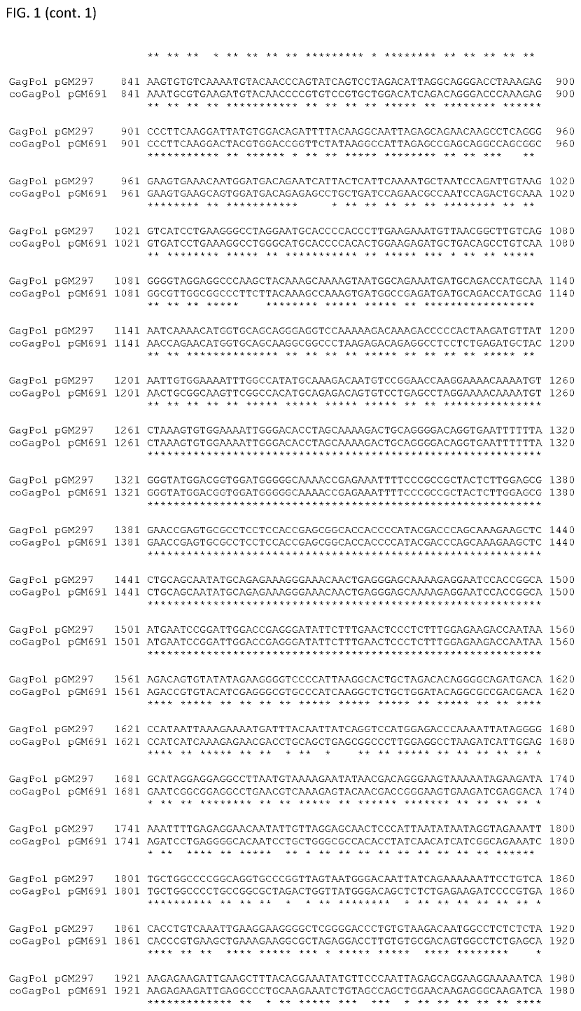

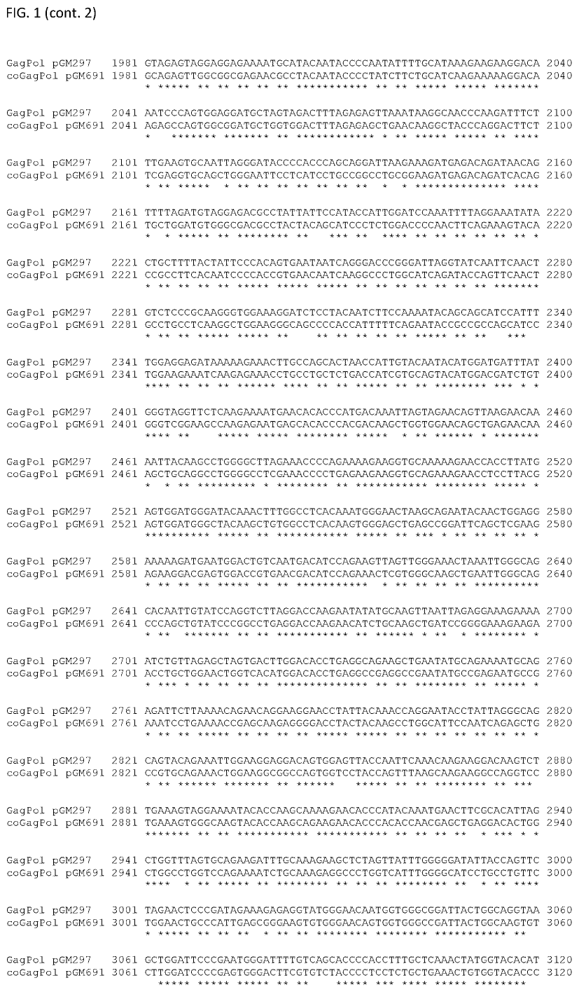

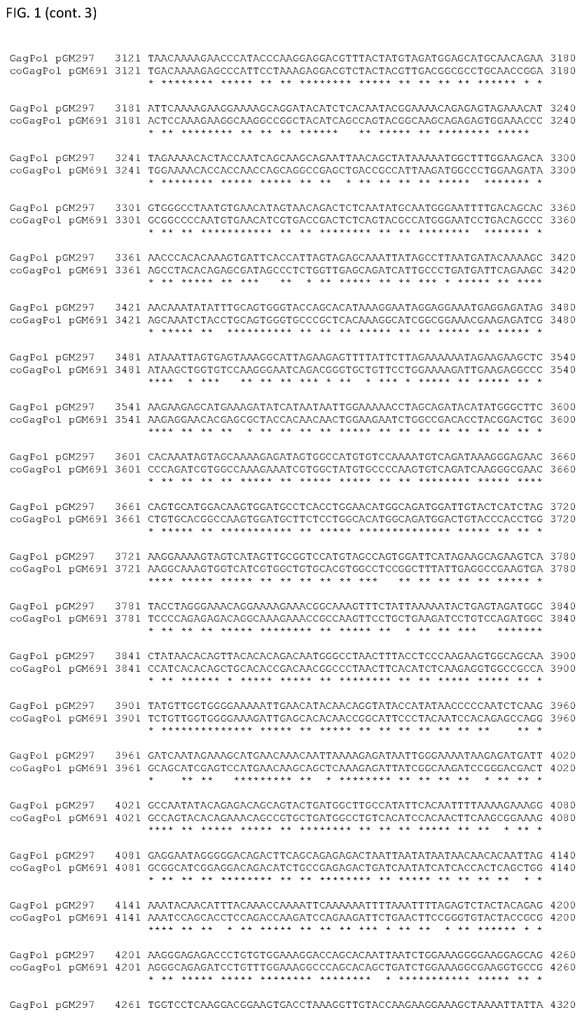

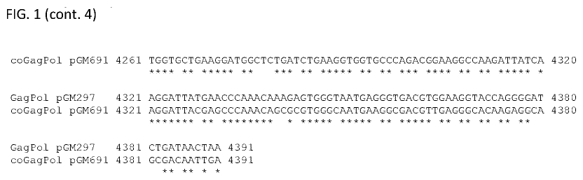

Preferably the codon-optimised gag-pol genes used in the production methods of the invention are SIV gag-pol genes. Exemplary wild-type SIV gag-pol genes that may be modified to produce codon-optimised gag-pol genes are given in SEQ ID NO:2. The modifications made to the wild-type gag-pol genes of SEQ ID NO:2 in order to arrive at an exemplary codon-optimised gag-pol genes of the invention (SEQ ID NO:1) are shown in the alignment in FIG. 1.

In addition to codon-optimisation, the codon-optimised gag-pol genes used in the production methods of the invention may comprise other modifications, such as a translational slip (which allows translation to slip from one region to another to allow the production of both Gag and Pol). Any suitable variation of codon usage may be used in the codon-optimised gag-pol genes of the invention, provided that (i) homology between the vector genome plasmid and GagPol plasmid is reduced to minimise the risk of RCL production and (ii) after codon optimisation there is production of sufficient GagPol without the inclusion of RRE (this further reduces homology and the risk of RCL production).

The codon-optimised gag-pol genes used in the production methods of the invention may be completely (100%) or partially codon-optimised. Partial codon-optimisation encompasses at least 70%, at least 80%, at least 95%, at least 90%, at least 95%, at least 96%, at least 97%, at least 98%, at least 99% or more codon optimisation.

Preferably, the gag-pol genes themselves are completely codon-optimised, but may comprise non-contain regions of non-codon-optimised sequence (e.g. between the gag and pol genes). By way of non-limiting example, to maintain the translational slip of reading frames between the gag and pol genes, the region around the translational slip sequence may not be codon-optimised (e.g. in case the precise translational slip sequence is important for this function). A non-codon-optimised translational slip sequence within codon-optimised gag-pol genes is exemplified in SEQ ID NO:1.

Preferably, the codon-optimised gag-pol genes used in a method of the invention comprise or consist of the nucleic acid sequence of SEQ ID NO:1, or a variant thereof (as defined herein). In particular, the codon-optimised gag-pol genes used in a method of the invention comprise or consist of a nucleic acid sequence having at least 80%, at least 85%, at least 90%, at least 95%, at least 96%, at least 97%, at least 98%, at least 99% or more sequence identity to SEQ ID NO:1. Preferably, the codon-optimised gag-pol genes used in a method of the invention comprise or consist of a nucleic acid sequence having at least 90%, more preferably at least 95%, even more preferably at least 98%, or more sequence identity to SEQ ID NO:1. The codon-optimised gag-pol genes of SEQ ID NO:1 comprise a translational slip, and so do not form a single conventional open reading frame.

The method of the invention may be a scalable GMP-compatible method. Thus, the method of the invention typically allows the generation of high titre purified F/HN retroviral/lentiviral (e.g. SIV) vectors. Typically a method of the invention produces a titre of retroviral/lentiviral (e.g. SIV) vector that is at least equivalent to the titre of retroviral/lentiviral (e.g. SIV) vector produced by a corresponding method which does not use codon-optimised gal-pol genes. As used herein, the term “equivalent” may be defined such that the use of the codon-optimised gag-pol genes does not significantly decrease the titre of retroviral/lentiviral (e.g. SIV) vector compared with the use of the corresponding non-codon-optimised gal-pol genes. By way of non-limiting example, a method of the invention produces a titre of retroviral/lentiviral (e.g. SIV) vector that is no more than 2-fold lower, no more than 1.5-fold lower, no more than 1.0-fold lower, no more than 0.5-fold lower, no more than 0.25-fold lower, or less than the titre of retroviral/lentiviral (e.g. SIV) vector compared with the use of the corresponding non-codon-optimised gal-pol genes. The term “equivalent” may be defined such that titre of retroviral/lentiviral (e.g. SIV) vector produced by a method using codon-optimised gag-pol genes is statistically unchanged (e.g. p<0.05, p<0.01) compared with the titre of retroviral/lentiviral (e.g. SIV) vector produced by a method using the corresponding non-codon-optimised gal-pol genes.

Preferably, a method of the invention produces a titre of retroviral/lentiviral (e.g. SIV) vector that is increased compared with the titre of retroviral/lentiviral (e.g. SIV) vector produced by a corresponding method which does not use codon-optimised gal-pol genes. The titre of retroviral/lentiviral (e.g. SIV) vector may be at least 1.5-fold, at least 2-fold, or at least 2.5-fold greater than the titre of retroviral/lentiviral (e.g. SIV) vector produced by a corresponding method which does not use codon-optimised gal-pol genes.

The production of retroviral/lentiviral (e.g. SIV) vectors typically employs one or more plasmids which provide the elements needed for the production of the vector: the genome for the retroviral/lentiviral vector, the Gag-Pol, Rev, F and HN. Multiple elements can be provided on a single plasmid. Preferably each element is provided on a separate plasmid, such that there five plasmids, one for each of the vector genome, the Gag-Pol, Rev, F and HN, respectively.

Alternatively, a single plasmid may provide the Gag-Pol and Rev elements, and may be referred to as a packaging plasmid (pDNA2). The remaining elements (genome, F and HN) may be provided by separate plasmids (pDNA1, pDNA3a, pDNA3b respectively), such that four plasmids are used for the production of a retroviral/lentiviral (e.g. SIV) vector according to the invention. In the four plasmid methods, pDNA1, pDNA3a and pDNA3b may be as described herein in the context of the five-plasmid method.

Preferably, the codon-optimised gag-pol genes used in a method of the invention are comprised in a plasmid that comprises or consists of a nucleic acid sequence of SEQ ID NO:5 (pGM691), or a variant thereof (as defined herein). In particular, the codon-optimised gag-pol genes used in a method of the invention are comprised in a plasmid that comprises or consists of a nucleic acid sequence having at least 80%, at least 85%, at least 90%, at least 95%, at least 96%, at least 97%, at least 98%, at least 99% or more sequence identity to SEQ ID NO:5. Preferably, the codon-optimised gag-pol genes used in a method of the invention are comprised in a plasmid that comprises or consists of a nucleic acid sequence having at least 90%, more preferably at least 95%, even more preferably at least 98%, or more sequence identity to SEQ ID NO:5. In the plasmid of SEQ ID NO:5 (or variants thereof): (i) the codon-optimised gag-pol genes of SEQ ID NO:1 comprise a translational slip, and so do not form a single conventional open reading frame; and (ii) the codon-optimised gag-pol genes of SEQ ID NO:1 are operably linked to a CAG promoter.

In the preferred five plasmid method of the invention, the vector genome plasmid encodes all the genetic material that is packaged into final retroviral/lentiviral vector, including the transgene. Typically only a portion of the genetic material found in the vector genome plasmid ends up in the virus. The vector genome plasmid may be designated herein as “pDNA1”, and typically comprises the transgene and the transgene promoter.

The other four plasmids are manufacturing plasmids encoding the Gag-Pol, Rev, F and HN proteins. These plasmids may be designated “pDNA2a”, “pDNA2b”, “pDNA3a” and “pDNA3b” respectively.

Modifications may be made to the vector genome plasmid (pDNA1), particularly to further improve the safety profile of the vector. As exemplified herein, such modifications may comprise or consist of modifying the pDNA1 sequence to remove viral, particularly retroviral/lentiviral (e.g. SIV), ORFs from the pDNA1 sequence. Thus, the methods of the invention may use a modified pDNA1 which comprises a reduced number of non-transgene ORFs. Said modified pDNA1 may comprise modifications within any region of the plasmid sequence. In particular, a modified pDNA1 may comprise modifications to remove: (i) 5′ to 3′ ORFs; (ii) ORFs of ≥100 amino acids; and/or (iii) ORFs upstream of the transgene and/or the promoter operably linked to the transgene. Whilst a modified pDNA1 may comprise no ORFs other than the transgene, this is not essential. Rather, a modified pDNA1 may still comprise ORFs other than the transgene, but may comprise a reduced number of non-transgene ORFs compared to the unmodified pDNA1 from which it is derived. By way of non-limiting example, a modified pDNA1 may comprise at least 1, at least 2, at least 3, at least 4, at least 5 or more fewer non-transgene ORFs compared with the corresponding unmodified pDNA1. As a specific example, pGM830 (which is derived from pGM326) comprises 2 fewer non-transgene ORFs compared with pGM326. A modified pDNA1 may comprise at least 1, at least 5, at least 6, at least 7, at least 8, at least 9, at least 10, at least 15, at least 20, or more modifications (e.g. 1, 2, 3, 4, 5, 6, 7, 8, 9, 10, 15 or 20 modifications) compared with the corresponding unmodified pDNA1. By way of non-limiting example, a modified pDNA1 may comprise between about 1 to about 20, such as between about 5 to about 15, or between about 5 to about 10 modifications compared with the corresponding unmodified pDNA1. As a specific example, pGM830 (which is derived from pGM326) comprises 7 modifications compared with pGM326.

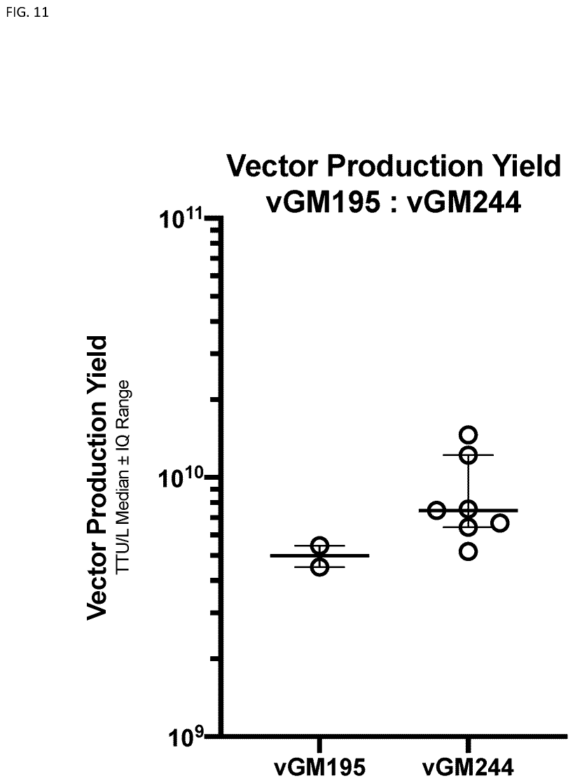

As exemplified herein, the use of the pGM380 as plasmid pDNA1 has the potential to produce an improved SIV titre compared with a production method in which the pDNA1 plasmid is pGM326 (FIG. 11), but in which all other plasmids and method parameters are kept constant. In other words, use of a modified pDNA1 such as pGM830 does not negatively impact the improved titre achieved using codon-optimised gal-pol genes, and can even potentially provide a further improvement in titre over and above the effect of using codon-optimised gal-pol genes, such as those provided by using pGM691 as pDNA2a. The term “increased titre” as defined herein applies equally to methods of the invention which use both codon-optimised gal-pol genes and a modified pDNA1.

Typically, the lentivirus is SIV, such as SIV1, preferably SIV-AGM. The F and HN proteins are derived from a respiratory paramyxovirus, preferably a Sendai virus.

In a specific embodiment relating to CFTR, the five plasmids are characterised by FIGS. 2A-2F, thus pDNA1 is the pGM326 plasmid of FIG. 2A or the pGM830 plasmid of FIG. 2B, pDNA2a is the pGM691 plasmid of FIG. 2C, pDNA2b is the pGM299 plasmid of FIG. 2D, pDNA3a is the pGM301 plasmid of FIG. 2E and pDNA3b is the pGM303 plasmid of FIG. 2F, or variants thereof any of these plasmids (as described herein). In this embodiment, the final CFTR containing retroviral/lentiviral vector may be referred to as vGM195 (see the Examples). The pGM691 plasmid and the vGM195 vector are preferred embodiments of the invention.

As exemplified herein, the use of the pGM691 as plasmid DNA2a has the potential to produce an improved SIV titre compared with a production method in which the pDNA2a plasmid is pGM297 (FIG. 2G), but in which all other plasmids and method parameters are kept constant.

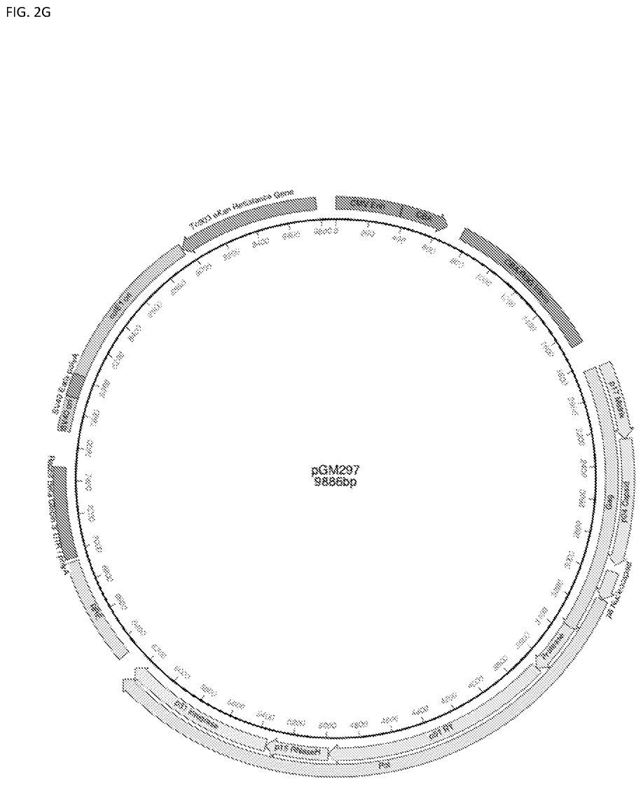

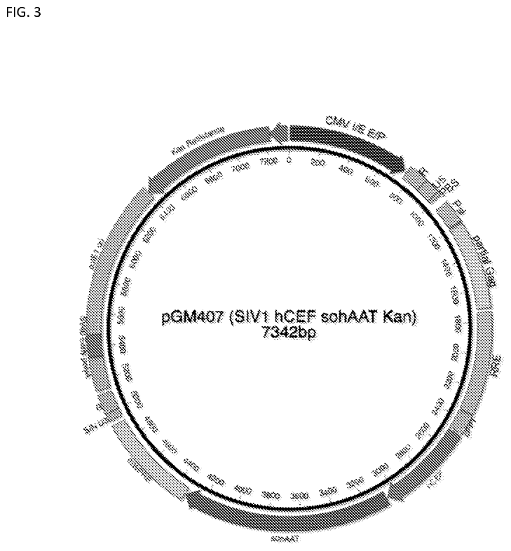

When a method of the invention is used to produce A1AT, the five plasmids may be characterised by FIG. 3 (thus plasmid pDNA1 may be pGM407) and all of FIGS. 2C-F (as above for the specific CFTR embodiment), or variants of any of these plasmids (as described herein).

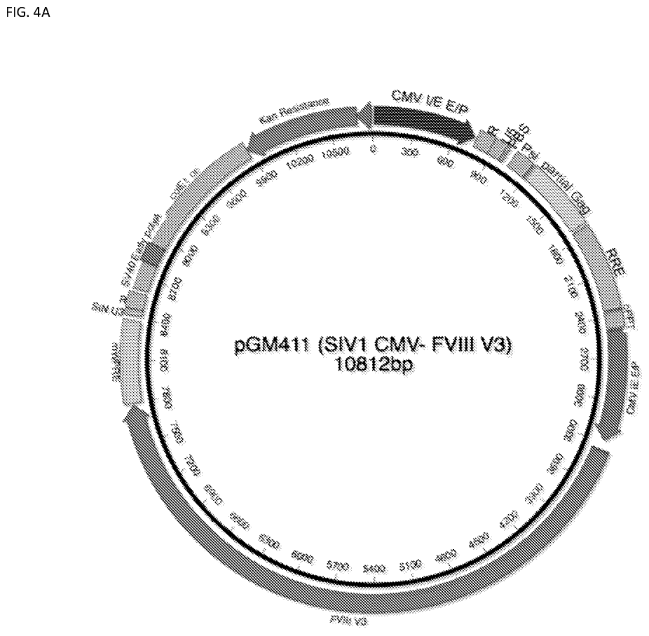

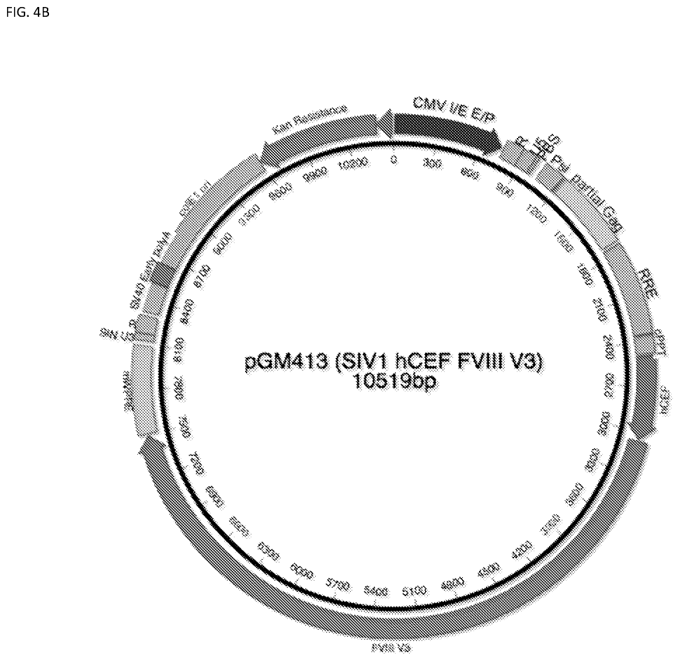

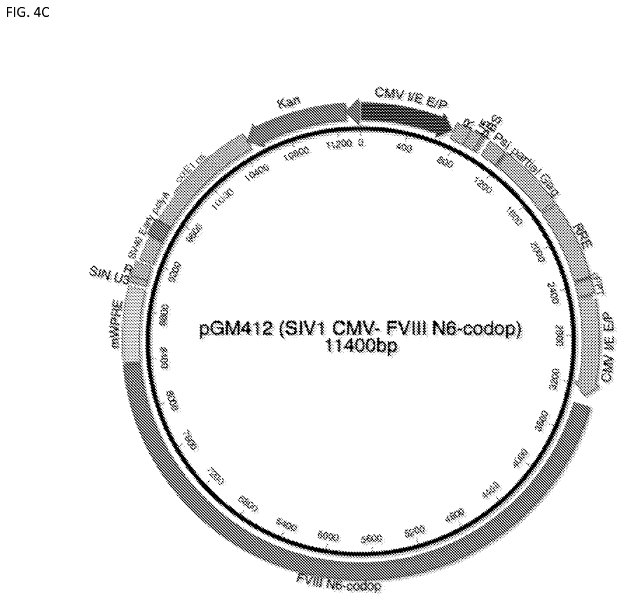

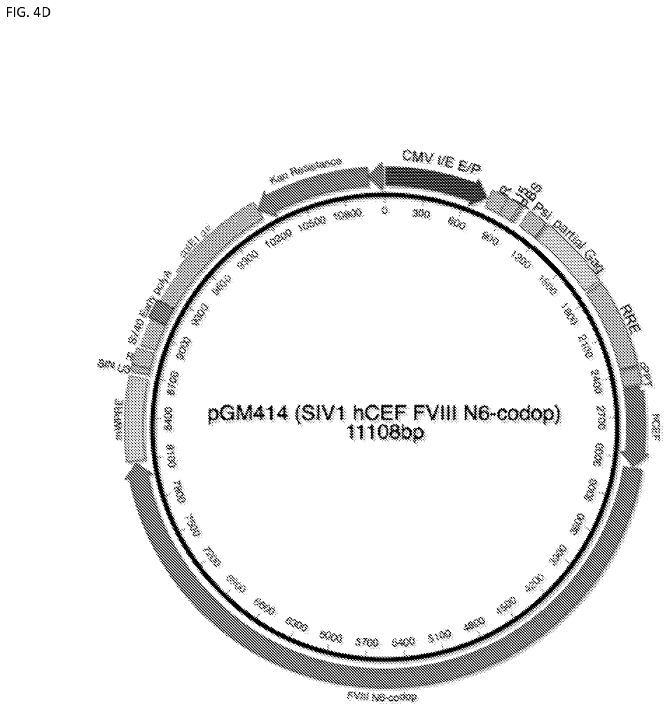

When a method of the invention is used to produce FVIII, the five plasmids may be characterised by one of FIGS. 4AD (thus plasmid pDNA1 may be pGM411, pGM412, pGM413 or pGM414) and all of FIGS. 2C-F, or variants of any of these plasmids (as described herein).

The plasmid as defined in FIG. 2A is represented by SEQ ID NO:3; the plasmid as defined in FIG. 2B is represented by SEQ ID NO:4; the plasmid as defined in FIG. 2C is represented by SEQ ID NO:5; the plasmid as defined in FIG. 2D is represented by SEQ ID NO:6; the plasmid as defined in FIG. 2E is represented by SEQ ID NO:7; the plasmid as defined in FIG. 2F is represented by SEQ ID NO:8; the plasmid as defined in FIG. 2G is represented by SEQ ID NO:9; the plasmid as defined in FIG. 3 is represented by SEQ ID NO:24 and the F/HN-SIV-CMV-HFVIII-V3, F/HN-SIV-hCEF-HFVIII-V3, F/HN-SIV-CMV-HFVIII-N6-co and/or F/HN-SIV-hCEF-HFVIII-N6-co plasmids as defined in FIGS. 4A to 4D are represented by SEQ ID NOs:25 to 28 respectively. Variants (as defined herein) of these plasmids are also encompassed by the present invention. In particular, variants having at least 90% (such as at least 90, 92, 94, 95, 96, 97, 98, 99, 99.5 or 100%) sequence identity to any one of SEQ ID NOs:3 to 9, 24 and 25 to 28 are encompassed.

In the five-plasmid method of the invention all five plasmids contribute to the formation of the final retroviral/lentiviral (e.g. SIV) vector. During manufacture of the retroviral/lentiviral (e.g. SIV) vector, the vector genome plasmid (pDNA1) provides the enhancer/promoter, Psi, RRE, cPPT, mWPRE, SIN LTR, SV40 polyA (see FIG. 2A or 2B), which are important for virus manufacture. Using pGM326 or pGM830 as non-limiting examples of a pDNA1, the CMV enhancer/promoter, SV40 polyA, colE1 Ori and KanR are involved in manufacture of the retroviral/lentiviral (e.g. SIV) vector of the invention (e.g. vGM195 or vGM244), but are not found in the final retroviral/lentiviral (e.g. SIV) vector. The RRE, cPPT (central polypurine tract), hCEF, soCFTR2 (transgene) and mWPRE from pGM326 or pGM830 are found in the final retroviral/lentiviral (e.g. SIV) vector. SIN LTR (long terminal repeats, SIN/IN self-inactivating) and Psi (packaging signal) may be found in the final retroviral/lentiviral (e.g. SIV) vector.

For other retroviral/lentiviral (e.g. SIV) vectors of the invention, corresponding elements from the other vector genome plasmids (pDNA1) are required for manufacture (but not found in the final vector), or are present in the final retroviral/lentiviral (e.g. SIV) vector.

The F and HN proteins from pDNA3a and pDNA3b (preferably Sendai F and HN proteins) are important for infection of target cells with the final retroviral/lentiviral (e.g. SIV) vector, i.e. for entry of a patient's epithelial cells (typically lung or nasal cells as described herein). The products of the pDNA2a and pDNA2b plasmids are important for virus transduction, i.e. for inserting the retroviral/lentiviral (e.g. SIV) DNA into the host's genome. The promoter, regulatory elements (such as WPRE) and transgene are important for transgene expression within the target cell(s).

A method of the invention may comprise or consist of the following steps: (a) growing cells in suspension; (b) transfecting the cells with one or more plasmids; (c) adding a nuclease; (d) harvesting the lentivirus (e.g. SIV); (e) adding trypsin; and (f) purification of the lentivirus (e.g. SIV).

This method may use the four- or five-plasmid system described herein. Thus, for the preferred five-plasmid method, the one or more plasmids may comprise or consist of: a vector genome plasmid pDNA1; a co-galpol plasmid, pDNA2a; a Rev plasmid, pDNA2b; a fusion (F) protein plasmid, pDNA3a; and a hemagglutinin-neuraminidase (HN) plasmid, pDNA3b. The pDNA1 may be selected from pGM326 and pGM830, preferably pGM830. The pDNA2a may be pGM691. The pDNA2b may be pGM299. The pDNA3a may be pGM301. The pDNA3b may be pGM303. Any combination of pDNA1, pDNA2a, pDNA2b, pDNA3a and pDNA3b may be used. Preferably, the pDNA1 is pGM326 or pGM830 (pGM830 being particularly preferred); the pDNA2a is pGM691; the pDNA2b is pGM299; the pDNA3a is pGM301; and the pDNA3b is pGM303. A SIV vector produced using pGM830, pGM691, pGM299, pGM301, and pGM303 is designated vGM244. A SIV vector produced using pGM326, pGM691, pGM299, pGM301, and pGM303 is designated vGM195.

Any appropriate ratio of vector genome plasmid:co-gagpol plasmid:Rev plasmid:F plasmid:HN plasmid may be used to further optimise (increase) the retroviral/lentiviral (e.g. SIV) titre produced. By way of non-limiting example, the ratio of vector genome plasmid:co-gagpol plasmid:Rev plasmid:F plasmid:HN plasmid may by in the range of 10-40:-4-20:3-12:3-12:3-12, typically 15-20:7-11:4-8:4-8:4-8, such as about 18-22:7-11:4-8:4-8:4-8, 19-21:8-10:5-7:5-7:5-7. Preferably the ratio of vector genome plasmid: co-gagpol plasmid: Rev plasmid: F plasmid: HN plasmid is about 20:9:6:6:6.

Steps (a)-(f) of the method are typically carried out sequentially, starting at step (a) and continuing through to step (f). The method may include one or more additional step, such as additional purification steps, buffer exchange, concentration of the retroviral/lentiviral (e.g. SIV) vector after purification, and/or formulation of the retroviral/lentiviral (e.g. SIV) vector after purification (or concentration). Each of the steps may comprise one or more sub-steps. For example, harvesting may involve one or more steps or sub-steps, and/or purification may involve one or more steps or sub-steps.

Any appropriate cell type may be transfected with the one or more plasmids (e.g. the five-plasmids described herein) to produce a retroviral/lentiviral (e.g. SIV) vector of the invention. Typically mammalian cells, particularly human cell lines are used. Non-limiting examples of cells suitable for use in the methods of the invention are HEK293 cells (such as HEK293F or HEK293T cells) and 293T/17 cells. Commercial cell lines suitable for the production of virus are also readily available (e.g. Gibco Viral Production Cells—Catalogue Number A35347 from ThermoFisher Scientific).

The cells may be grown in animal-component free media, including serum-free media. The cells may be grown in a media which contains human components. The cells may be grown in a defined media comprising or consisting of synthetically produced components.

Any appropriate transfection means may be used according to the invention. Selection of appropriate transfection means is within the routine practice of one of ordinary skill in the art. By way of non-limiting example, transfection may be carried out by the use of PEIPro™, Lipofectamine2000™ or Lipofectamine3000™.

Any appropriate nuclease may be used according to the invention. Selection of appropriate nuclease is within the routine practice of one of ordinary skill in the art. Typically the nuclease is an endonuclease. By way of non-limiting example, the nuclease may be Benzonase® or Denarase®. The addition of the nuclease may be at the pre-harvest stage or at the post-harvest stage, or between harvesting steps.

The trypsin activity may preferably be provided by an animal origin free, recombinant enzyme such as TrypLE Select™. The addition of trypsin may be at the pre-harvest stage or at the post-harvest stage, or between harvesting steps.

Any appropriate purification means may be used to purify the retroviral/lentiviral (e.g. SIV) vector. Non-limiting examples of suitable purification steps include depth/end filtration, tangential flow filtration (TFF) and chromatography. The purification step typically comprises at least on chromatography step. Non-limiting examples of chromatography steps that may be used in accordance with the invention include mixed-mode size exclusion chromatography (SEC) and/or anion exchange chromatography. Elution may be carried out with or without the use of a salt gradient, preferably without.

This method may be used to produce the retroviral/lentiviral (e.g. SIV) vectors of the invention, such as those comprising a CFTR, A1AT and or FVIII gene as described herein. Alternatively, the retroviral/lentiviral (e.g. SIV) vector of the invention comprises any of the above-mentioned genes, or the genes encoding the above-mentioned proteins.

The method of the invention. may use any combination of one or more of the specific plasmid constructs provided by FIGS. 2A-2F. FIG. 3 and/or FIG. 4A-4D is used to provide a retroviral/lentiviral (e.g. SIV) vector of the invention. Particularly the plasmid constructs of FIGS. 2C-2F are used, preferably in combination with the plasmid of FIG. 2B, FIG. 2A, FIG. 3 or FIG. 4A-4D, with the plasmid of FIG. 2B being particularly preferred.

The invention also provides codon-optimised SIV gag-pol genes. These codon-optimised SIV gag-pol genes are typically suitable for use in the methods of the invention. The codon-optimised gag-pol genes of the invention may comprise or consist of the nucleic acid sequence of SEQ ID NO:1, or a variant thereof (as defined herein). In particular, the codon-optimised gag-pol genes of the invention may comprise or consist of a nucleic acid sequence having at least 80%, at least 85%, at least 90%, at least 95%, at least 96%, at least 97%, at least 98%, at least 99% or more sequence identity to SEQ ID NO:1. Preferably, the codon-optimised gag-pol genes of the invention may comprise or consist of a nucleic acid sequence having at least 90%, more preferably at least 95%, even more preferably at least 98%, or more sequence identity to SEQ ID NO:1. Accordingly, the invention provides a nucleic acid comprising codon-optimised gag-pol genes, said nucleic acid having at least 80%, at least 85%, at least 90%, at least 95%, at least 96%, at least 97%, at least 98%, at least 99% or more sequence identity to SEQ ID NO:1, preferably at least 90%. more preferably at least 95%, even more preferably at least 98%, or more sequence identity to SEQ ID NO:1. In a particularly preferred embodiment, the invention provides a nucleic acid which comprises or consists of the nucleic acid sequence of SEQ ID NO:1. The codon-optimised gag-pol genes (e.g. SIV gag-pol genes) of the invention are typically operably linked to a promoter to facilitate expression of the gag-pol proteins. Any suitable promoter may be used, including those described herein in the context of promoters for the transgene. Preferably, the promoter is a CAG promoter, as used on the exemplified pGM691 plasmid. An exemplary CAG promoter is set out in SEQ ID NO:29. The codon-optimised gag-pol genes of SEQ ID NO:1 comprise a translational slip, and so do not form a single conventional open reading frame.

The invention also provides plasmids comprising the codon-optimised SIV gag-pol genes of the invention, i.e. pDNA2a comprising the codon-optimised SIV gag-pol genes of the invention. These plasmids are typically suitable for use in the methods of the invention. The (pDNA2a) plasmid of the invention may comprise or consist of a nucleic acid sequence of SEQ ID NO:5 (pGM691), or a variant thereof (as defined herein). In particular, the (pDNA2a) plasmid of the invention may comprise or consist of a nucleic acid sequence having at least 80%, at least 85%, at least 90%, at least 95%, at least 96%, at least 97%, at least 98%, at least 99% or more sequence identity to SEQ ID NO:5. Preferably, the (pDNA2a) plasmid of the invention may comprise or consist of a nucleic acid sequence having at least 90%, more preferably at least 95%, even more preferably at least 98%, or more sequence identity to SEQ ID NO:5. Accordingly, the invention provides a plasmid comprising codon-optimised SIV gag-pol genes of the invention (as defined herein), particularly, a nucleic acid sequence comprising or consisting of SEQ ID NO:1, or a variant thereof (as defined herein). Said plasmid may comprise or consist of a nucleic acid sequence having at least 80%, at least 85%, at least 90%, at least 95%, at least 96%, at least 97%, at least 98%, at least 99% or more sequence identity to SEQ ID NO:5, preferably at least 90%, more preferably at least 95%, even more preferably at least 98%, or more sequence identity to SEQ ID NO:5. In a particularly preferred embodiment, the invention provides a plasmid which comprises or consists of the nucleic acid sequence of SEQ ID NO:5. In the plasmid of SEQ ID NO:5 (or variants thereof): (i) the codon-optimised gag-pol genes of SEQ ID NO:1 comprise a translational slip, and so do not form a single conventional open reading frame; and (ii) the codon-optimised gag-pol genes of SEQ ID NO:1 are operably linked to a CAG promoter (e.g. as exemplified herein).

The codon-optimised gag-pol genes (or nucleic acids comprising or consisting thereof) and plasmids comprising said genes or nucleic acids are advantageous in the production of retroviral/lentiviral (e.g. SIV) vectors using methods of the invention, as they allow for the production of high titre F/HN retroviral/lentiviral (e.g. SIV) vectors. Typically said codon-optimised gag-pol genes (or nucleic acids comprising or consisting thereof) and plasmids comprising said genes or nucleic acids can be used to produces a titre of retroviral/lentiviral (e.g. SIV) vector that is at least equivalent to the titre of retroviral/lentiviral (e.g. SIV) vector produced by a corresponding method which does not use codon-optimised gal-pol genes, as described herein.

Preferably, the codon-optimised gag-pol genes (or nucleic acids comprising or consisting thereof) and plasmids comprising said genes or nucleic acids allow for the production of a titre of retroviral/lentiviral (e.g. SIV) vector that is increased compared with the titre of retroviral/lentiviral (e.g. SIV) vector produced by a corresponding method which does not use codon-optimised gal-pol genes, as described herein.

The invention also provides host cells comprising (i) a retroviral/lentiviral (e.g. SIV) vector of the invention, (ii) codon-optimised gag-pol genes (or a nucleic acid comprising or consisting thereof) of the invention; and/or (iii) a plasmid comprising said genes or nucleic acid; or any combination thereof. Typically a host cell is a mammalian cell, particularly a human cell or cell line. Non-limiting examples of host cells include HEK293 cells (such as HEK293F or HEK293T cells) and 293T/17 cells. Commercial cell lines suitable for the production of virus are also readily available (as described herein).

The invention also provides a retroviral/lentiviral (e.g. SIV) vector obtainable by a method of the invention, or using codon-optimised gag-pol genes (or nucleic acids comprising or consisting thereof), a plasmid comprising said genes or nucleic acids, or host cell of the invention.

Typically the retroviral/lentiviral (e.g. SIV) vector obtainable by a method of the invention, or using codon-optimised gag-pol genes (or nucleic acids comprising or consisting thereof), a plasmid comprising said genes or nucleic acids, or host cell of the invention is produced at a high-titre. Titre may be measured in terms of transducing units, as defined here. As described herein, the methods of the invention typically produce retroviral/lentiviral (e.g. SIV) vector at equivalent or higher titres than corresponding methods which do not use codon-optimised gag-pol genes. Accordingly, the retroviral/lentiviral (e.g. SIV) vector obtainable by a method of the invention, or using codon-optimised gag-pol genes (or nucleic acids comprising or consisting thereof), a plasmid comprising said genes or nucleic acids, or host cell of the invention may optionally be at a titre of at least about 2.5×10⁶ TU/mL, at least about 3.0×10⁶ TU/mL, at least about 3.1×10⁶ TU/mL, at least about 3.2×10⁶ TU/mL, at least about 3.3×10⁶ TU/mL, at least about 3.4×10⁶ TU/mL, at least about 3.5×10⁶ TU/mL, at least about 3.6×10⁶ TU/mL, at least about 3.7×10⁶ TU/mL, at least about 3.8×10⁶ TU/mL, at least about 3.9×10⁶ TU/mL, at least about 4.0×10⁶ TU/mL or more. Preferably the retroviral/lentiviral (e.g. SIV) vector is produced at a titre of at least about 3.0×10⁶ TU/mL, or at least about 3.5×10⁶ TU/mL.

The production of high-titre retroviral/lentiviral (e.g. SIV) vectors may impart other desirable properties on the resulting vector products. For example, without being bound by theory, it is believed that production at high titres without the need for intense concentration by methods such as TFF results in a higher quality vector product than retroviral/lentiviral (e.g. SIV) vectors produced by corresponding methods without the use of codon-optimised gag-pol genes (and optionally a modified vector genome plasmid), because the vectors are exposed to less shear forces which can damage the viral particles and their RNA cargo.

The invention also provides a method of increasing retroviral/lentiviral (e.g. SIV) vector titre comprising the use of codon-optimised gag-pol genes (or nucleic acids comprising or consisting thereof), a plasmid comprising said genes or nucleic acids, or host cell of the invention. Said method of increasing retroviral/lentiviral (e.g. SIV) vector titre according to the invention may increase titre by at least 1.5-fold, at least 2-fold, or at least 2.5-fold or more compared with a corresponding method which uses non-codon-optimised versions of the gag-pol genes (or nucleic acids comprising or consisting thereof), or plasmids or host cells comprising said non-codon optimised genes or nucleic acids. Alternatively, a method of increasing retroviral/lentiviral (e.g. SIV) titre according to the invention may increase titre by at least about 25%, at least about 50%, at least about 100%, at least about 150%, at least about 200% or more compared with a corresponding method which uses non-codon-optimised versions of the gag-pol genes (or nucleic acids comprising or consisting thereof), or plasmids or host cells comprising said non-codon optimised genes or nucleic acids. Preferably, a method of increasing retroviral/lentiviral (e.g. SIV) titre according to the invention may increase titre by (a) by at least 1.5-fold or at least 2-fold; and/or (b) by at least about 25%, more preferably at least about 50%, even more preferably at least about 100%. Typically the corresponding method is identical to the method of the invention except for the use of codon-optimised gag-pol genes (or nucleic acids comprising or consisting thereof), a plasmid comprising said genes or nucleic acids, or host cell of the invention. All the disclosure herein in relation to method of producing a retroviral/lentiviral (e.g. SIV) vector applies equally and without reservation to the methods of increasing retroviral/lentiviral (e.g. SIV) titre of the invention.

The invention also provides the use of codon-optimised gag-pol genes (or nucleic acids comprising or consisting thereof), a plasmid comprising said genes or nucleic acids, or host cell of the invention to increase the titre of a retroviral/lentiviral (e.g. SIV) vector. Said use may increase retroviral/lentiviral (e.g. SIV) vector titre by at least 1.5-fold, at least 2-fold, or at least 2.5-fold or more compared with the use of a corresponding non-codon-optimised version of the gag-pol genes (or nucleic acids comprising or consisting thereof), or plasmids or host cells comprising said non-codon optimised genes or nucleic acids. Alternatively, said use may increase retroviral/lentiviral (e.g. SIV) titre by at least about 25%, at least about 50%, at least about 100%, at least about 150%, at least about 200% or more compared with the use of a corresponding non-codon-optimised version of the gag-pol genes (or nucleic acids comprising or consisting thereof), or plasmids or host cells comprising said non-codon optimised genes or nucleic acids. Preferably, said use increases retroviral/lentiviral (e.g. SIV) titre by (a) by at least 1.5-fold or at least 2-fold; and/or (b) at least about 25%, more preferably at least about 50%, even more preferably at least about 100%. Typically the corresponding use is identical to the method of the invention except for the use of codon-optimised gag-pol genes (or nucleic acids comprising or consisting thereof), a plasmid comprising said genes or nucleic acids, or host cell of the invention. All the disclosure herein in relation to method of producing a retroviral/lentiviral (e.g. SIV) vector applies equally and without reservation to the use of codon-optimised gag-pol genes (or nucleic acids comprising or consisting thereof), a plasmid comprising said genes or nucleic acids, or host cell of the invention to increase the titre of a retroviral/lentiviral (e.g. SIV) vector according to the invention. The use of codon-optimised gal-pol genes in combination with a modified vector genome plasmid (with reduced viral ORFs) may provide a further advantage, in terms of safety and/or vector titre. Thus, the increased vector yields as described herein may be achieved using codon-optimised gag-pol genes alone, or in combination with a modified vector genome plasmid. Any and all disclosure herein in relation to increased vector titre in the context of method using codon-optimised gag-pol genes applies equally and without reservation to methods using codon-optimised gag-pol genes in combination with a modified vector genome plasmid of the invention, and to vectors produced by such methods.

### Therapeutic Indications

The retroviral/lentiviral (e.g. SIV) vectors of the present invention enable higher and sustained gene expression through efficient gene transfer. The F/HN-pseudotyped retroviral/lentiviral (e.g. SIV) vectors of the invention are capable of: (i) airway transduction without disruption of epithelial integrity; (ii) persistent gene expression; (iii) lack of chronic toxicity; and (iv) efficient repeat administration. Long term/persistent stable gene expression, preferably at a therapeutically-effective level, may be achieved using repeat doses of a vector of the present invention. Alternatively, a single dose may be used to achieve the desired long-term expression.

Thus, advantageously, the retroviral/lentiviral (e.g. SIV) vectors of the present invention can be used in gene therapy. By way of example, the efficient airway cell uptake properties of the retroviral/lentiviral (e.g. SIV) vectors of the invention make them highly suitable for treating respiratory tract diseases. The retroviral/lentiviral (e.g. SIV) vectors of the invention can also be used in methods of gene therapy to promote secretion of therapeutic proteins. By way of further example, the invention provides secretion of therapeutic proteins into the lumen of the respiratory tract or the circulatory system. Thus, administration of a retroviral/lentiviral (e.g. SIV) vector of the invention and its uptake by airway cells may enable the use of the lungs (or nose or airways) as a “factory” to produce a therapeutic protein that is then secreted and enters the general circulation at therapeutic levels, where it can travel to cells/tissues of interest to elicit a therapeutic effect. In contrast to intracellular or membrane proteins, the production of such secreted proteins does not rely on specific disease target cells being transduced, which is a significant advantage and achieves high levels of protein expression. Thus, other diseases which are not respiratory tract diseases, such as cardiovascular diseases and blood disorders, particularly blood clotting deficiencies, can also be treated by the retroviral/lentiviral (e.g. SIV) vectors of the present invention.

Retroviral/lentiviral (e.g. SIV) vectors of the invention can effectively treat a disease by providing a transgene for the correction of the disease. For example, inserting a functional copy of the CFTR gene to ameliorate or prevent lung disease in CF patients, independent of the underlying mutation. Accordingly, retroviral/lentiviral (e.g. SIV) vectors of the invention may be used to treat cystic fibrosis (CF), typically by gene therapy with a CFTR transgene as described herein.

As another example, retroviral/lentiviral (e.g. SIV) vectors of the invention may be used to treat Alpha-1 Antitrypsin (A1AT) deficiency, typically by gene therapy with a A1AT transgene as described herein. A1AT is a secreted anti-protease that is produced mainly in the liver and then trafficked to the lung, with smaller amounts also being produced in the lung itself. The main function of A1AT is to bind and neutralise/inhibit neutrophil elastase. Gene therapy with A1AT according to the present invention is relevant to A1AT deficient patient, as well as in other lung diseases such as CF or chronic obstructive pulmonary disease (COPD), and offers the opportunity to overcome some of the problems encountered by conventional enzyme replacement therapy (in which A1AT isolated from human blood and administered intravenously every week), providing stable, long-lasting expression in the target tissue (lung/nasal epithelium), ease of administration and unlimited availability.

Transduction with a retroviral/lentiviral (e.g. SIV) vector of the invention may lead to secretion of the recombinant protein into the lumen of the lung as well as into the circulation. One benefit of this is that the therapeutic protein reaches the interstitium. A1AT gene therapy may therefore also be beneficial in other disease indications, non-limiting examples of which include type 1 and type 2 diabetes, acute myocardial infarction, ischemic heart disease, rheumatoid arthritis, inflammatory bowel disease, transplant rejection, graft versus host (GvH) disease, multiple sclerosis, liver disease, cirrhosis, vasculitides and infections, such as bacterial and/or viral infections.

A1AT has numerous other anti-inflammatory and tissue-protective effects, for example in pre-clinical models of diabetes, graft versus host disease and inflammatory bowel disease. The production of A1AT in the lung and/or nose following transduction according to the present invention may, therefore, be more widely applicable, including to these indications.

Other examples of diseases that may be treated with gene therapy of a secreted protein according to the present invention include cardiovascular diseases and blood disorders, particularly blood clotting deficiencies such as haemophilia (A, B or C), von Willebrand disease and Factor VII deficiency.

Other examples of diseases or disorders to be treated include Primary Ciliary Dyskinesia (PCD), acute lung injury, Surfactant Protein B (SFTB) deficiency, Pulmonary Alveolar Proteinosis (PAP), Chronic Obstructive Pulmonary Disease (COPD) and/or inflammatory, infectious, immune or metabolic conditions, such as lysosomal storage diseases.

Accordingly, the invention provides a method of treating a disease, the method comprising administering a retroviral/lentiviral (e.g. SIV) vector of the invention to a subject. Typically the retroviral/lentiviral (e.g. SIV) vector is produced using a method of the present invention. Any disease described herein may be treated according to the invention. In particular, the invention provides a method of treating a lung disease using a retroviral/lentiviral (e.g. SIV) vector of the invention. The disease to be treated may be a chronic disease. Preferably, a method of treating CF is provided.

The invention also provides a retroviral/lentiviral (e.g. SIV) vector as described herein for use in a method of treating a disease. Typically the retroviral/lentiviral (e.g. SIV) vector is produced using a method of the present invention. Any disease described herein may be treated according to the invention. In particular, the invention provides a retroviral/lentiviral (e.g. SIV) vector of the invention for use in a method of treating a lung disease. The disease to be treated may be a chronic disease. Preferably, a retroviral/lentiviral (e.g. SIV) vector for use in treating CF is provided.

The invention also provides the use of a retroviral/lentiviral (e.g. SIV) vector as described herein in the manufacture of a medicament for use in a method of treating a disease. Typically the retroviral/lentiviral (e.g. SIV) vector is produced using a method of the present invention. Any disease described herein may be treated according to the invention. In particular, the invention provides the use of a retroviral/lentiviral (e.g. SIV) vector of the invention for the manufacture of a medicament for use in a method of treating a lung disease. The disease to be treated may be a chronic disease. Preferably, the use of a retroviral/lentiviral (e.g. SIV) vector in the manufacture of a medicament for use in a method of treating CF is provided.

### Formulation and Administration

The retroviral/lentiviral (e.g. SIV) vectors of the invention may be administered in any dosage appropriate for achieving the desired therapeutic effect. Appropriate dosages may be determined by a clinician or other medical practitioner using standard techniques and within the normal course of their work. Non-limiting examples of suitable dosages include 1×10⁸ transduction units (TU), 1×10⁹ TU, 1×10¹⁰ TU, 1×10¹¹ TU or more.

The invention also provides compositions comprising the retroviral/lentiviral (e.g. SIV) vectors described above, and a pharmaceutically-acceptable carrier. Non-limiting examples of pharmaceutically acceptable carriers include water, saline, and phosphate-buffered saline. In some embodiments, however, the composition is in lyophilized form, in which case it may include a stabilizer, such as bovine serum albumin (BSA). In some embodiments, it may be desirable to formulate the composition with a preservative, such as thiomersal or sodium azide, to facilitate long-term storage.

The retroviral/lentiviral (e.g. SIV) vectors of the invention may be administered by any appropriate route. It may be desired to direct the compositions of the present invention (as described above) to the respiratory system of a subject. Efficient transmission of a therapeutic/prophylactic composition or medicament to the site of infection in the respiratory tract may be achieved by oral or intra-nasal administration, for example, as aerosols (e.g. nasal sprays), or by catheters. Typically the retroviral/lentiviral (e.g. SIV) vectors of the invention are stable in clinically relevant nebulisers, inhalers (including metered dose inhalers), catheters and aerosols, etc.

In some embodiments the nose is a preferred production site for a therapeutic protein using a retroviral/lentiviral (e.g. SIV) vector of the invention for at least one of the following reasons: (i) extracellular barriers such as inflammatory cells and sputum are less pronounced in the nose; (ii) ease of vector administration; (iii) smaller quantities of vector required; and (iv) ethical considerations. Thus, transduction of nasal epithelial cells with a retroviral/lentiviral (e.g. SIV) vector of the invention may result in efficient (high-level) and long-lasting expression of the therapeutic transgene of interest. Accordingly, nasal administration of a retroviral/lentiviral (e.g. SIV) vector of the invention may be preferred.

Formulations for intra-nasal administration may be in the form of nasal droplets or a nasal spray. An intra-nasal formulation may comprise droplets having approximate diameters in the range of 100-5000 μm, such as 500-4000 μm, 1000-3000 μm or 100-1000 μm. Alternatively, in terms of volume, the droplets may be in the range of about 0.001-100 μl, such as 0.1-50 μl or 1.0-25 μl, or such as 0.001-1 μl.

The aerosol formulation may take the form of a powder, suspension or solution. The size of aerosol particles is relevant to the delivery capability of an aerosol. Smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli. In the case of aerosol delivery of the medicament, the particles may have diameters in the approximate range of 0.1-50 μm, preferably 1-25 μm, more preferably 1-5 μm.

Aerosol particles may be for delivery using a nebulizer (e.g. via the mouth) or nasal spray. An aerosol formulation may optionally contain a propellant and/or surfactant.

The formulation of pharmaceutical aerosols is routine to those skilled in the art, see for example, Sciarra, J, in Remington's Pharmaceutical Sciences (supra). The agents may be formulated as solution aerosols, dispersion or suspension aerosols of dry powders, emulsions or semisolid preparations. The aerosol may be delivered using any propellant system known to those skilled in the art. The aerosols may be applied to the upper respiratory tract, for example by nasal inhalation, or to the lower respiratory tract or to both. The part of the lung that the medicament is delivered to may be determined by the disorder. Compositions comprising a vector of the invention, in particular where intranasal delivery is to be used, may comprise a humectant. This may help reduce or prevent drying of the mucus membrane and to prevent irritation of the membranes. Suitable humectants include, for instance, sorbitol, mineral oil, vegetable oil and glycerol; soothing agents; membrane conditioners; sweeteners; and combinations thereof. The compositions may comprise a surfactant. Suitable surfactants include non-ionic, anionic and cationic surfactants. Examples of surfactants that may be used include, for example, polyoxyethylene derivatives of fatty acid partial esters of sorbitol anhydrides, such as for example, Tween 80, Polyoxyl 40 Stearate, Polyoxy ethylene 50 Stearate, fusieates, bile salts and Octoxynol.

In some cases after an initial administration a subsequent administration of a retroviral/lentiviral (e.g. SIV) vector may be performed. The administration may, for instance, be at least a week, two weeks, a month, two months, six months, a year or more after the initial administration. In some instances, retroviral/lentiviral (e.g. SIV) vector of the invention may be administered at least once a week, once a fortnight, once a month, every two months, every six months, annually or at longer intervals. Preferably, administration is every six months, more preferably annually. The retroviral/lentiviral (e.g. SIV) vectors may, for instance, be administered at intervals dictated by when the effects of the previous administration are decreasing.

Any two or more retroviral/lentiviral (e.g. SIV) vectors of the invention may be administered separately, sequentially or simultaneously. Thus two retroviral/lentiviral (e.g. SIV) vectors or more retroviral/lentiviral (e.g. SIV) vectors, where at least one retroviral/lentiviral (e.g. SIV) vectors is a retroviral/lentiviral (e.g. SIV) vector of the invention, may be administered separately, simultaneously or sequentially and in particular two or more retroviral/lentiviral (e.g. SIV) vectors of the invention may be administered in such a manner. The two may be administered in the same or different compositions. In a preferred instance, the two retroviral/lentiviral (e.g. SIV) vectors may be delivered in the same composition.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van WaIIe et al., Align-M—A New Algorithm for Multiple Alignment of Highly Divergent Sequences, 20(9) Bioinformatics: 1428-1435 (2004).

Thus, percent sequence identity is determined by conventional methods. See, for example, Altschul et al., Bull. Math. Bio. 48:603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides/amino acids divided by the total number of nucleotides/amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### Alignment Scores for Determining Sequence Identity

A R N D C Q E G H I L K M F P S T W Y V
A 4
R −1 5
N −2 0 6
D −2 −2 1 6
C 0 −3 −3 −3 9
Q −1 1 0 0 −3 5
E −1 0 0 2 −4 2 5
G 0 −2 0 −1 −3 −2 −2 6
H −2 0 1 −1 −3 0 0 −2 8
I −1 −3 −3 −3 −1 −3 −3 −4 −3 4
L −1 −2 −3 −4 −1 −2 −3 −4 −3 2 4
K −1 2 0 −1 −3 1 1 −2 −1 −3 −2 5
M −1 −1 −2 −3 −1 0 −2 −3 −2 1 2 −1 5

F −2 −3 −3 −3 −2 −3 −3 −3 −1 0 0 −3 0 6
P −1 −2 −2 −1 −3 −1 −1 −2 −2 −3 −3 −1 −2 −4 7
S 1 −1 1 0 −1 0 0 0 −1 −2 −2 0 −1 −2 −1 4
T 0 −1 0 −1 −1 −1 −1 −2 −2 −1 −1 −1 −1 −2 −1 1 5
W −3 −3 −4 −4 −2 −2 −3 −2 −2 −3 −2 −3 −1 1 −4 −3 −2 1 1
Y −2 −2 −2 −3 −2 −1 −2 −3 2 −1 −1 −2 −1 3 −3 −2 −2 2 7
V 0 −3 −3 −3 −1 −2 −2 −3 −3 3 1 −2 1 −1 −2 −2 0 −3 −1 4

The percent identity is then calculated as;

$$\frac{Total number of identical matches}{\begin{matrix}[ length of the longer sequence plus the number of gaps \\ introduced into the longer sequence in order to align the \\ two sequences ]\end{matrix}} \times 100$$

Substantially homologous polypeptides are characterized as having one or more amino acid substitutions, deletions or additions. These changes are preferably of a minor nature, that is conservative amino acid substitutions (as described herein) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

In addition to the 20 standard amino acids, non-standard amino acids (such as 4- hydroxyproline, 6-N-methyl lysine, 2-aminoisobutyric acid, isovaline and α-methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8. 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244:1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

### EXAMPLES

The invention is now described with reference to the Examples below. These are not limiting on the scope of the invention, and a person skilled in the art would be appreciate that suitable equivalents could be used within the scope of the present invention. Thus, the Examples may be considered component parts of the invention, and the individual aspects described therein may be considered as disclosed independently, or in any combination.

### Example 1—Plasmid pGM691 Construction

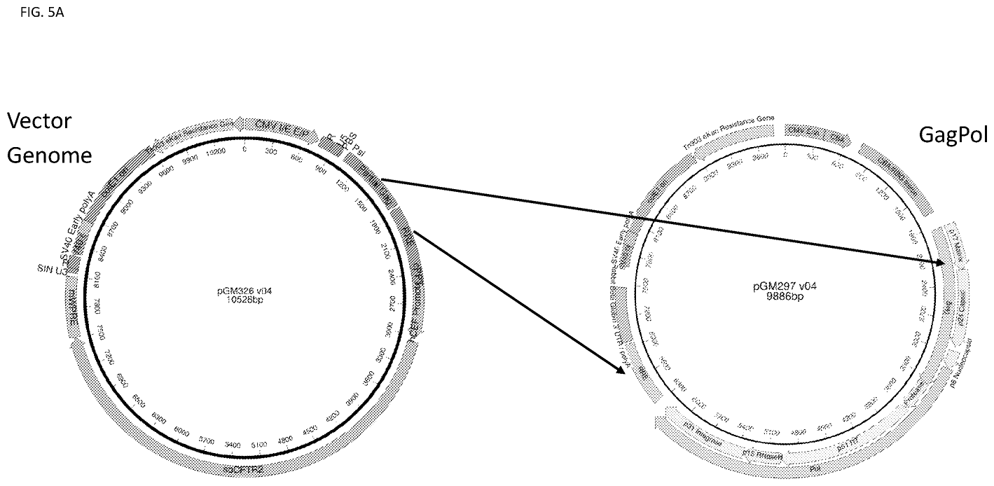

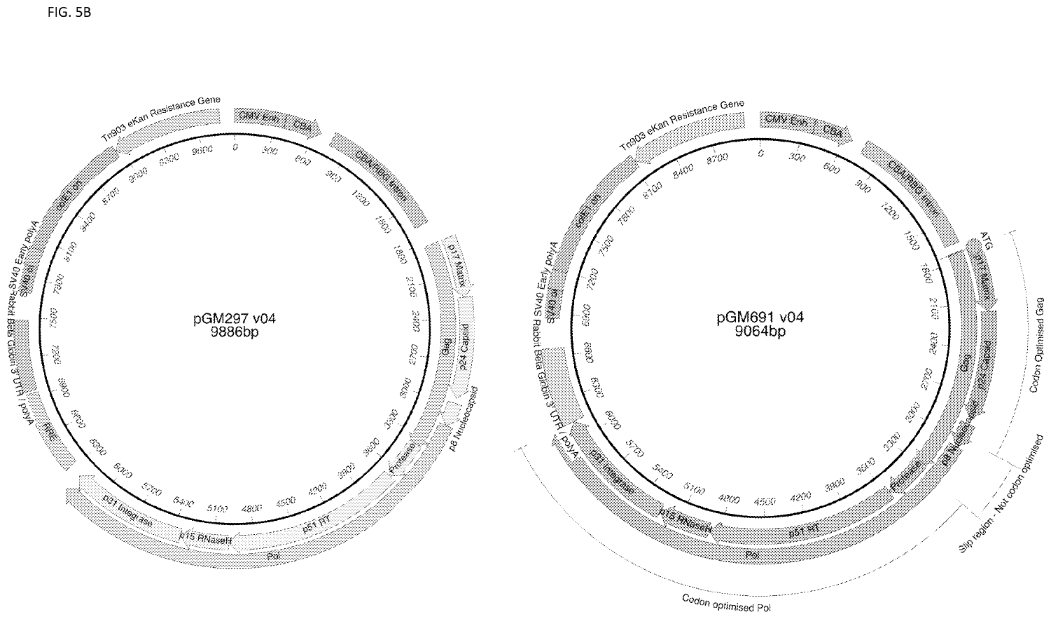

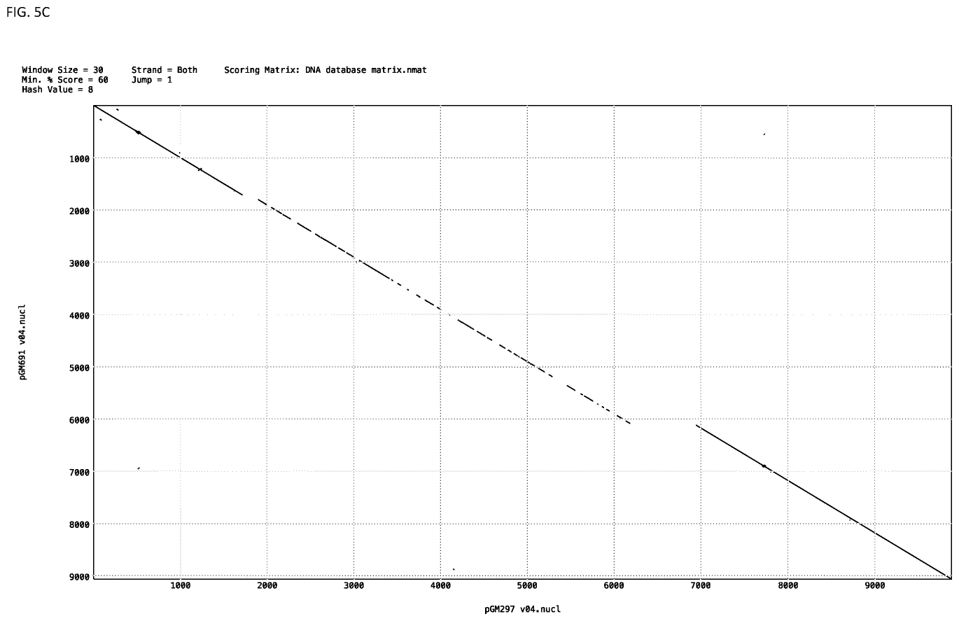

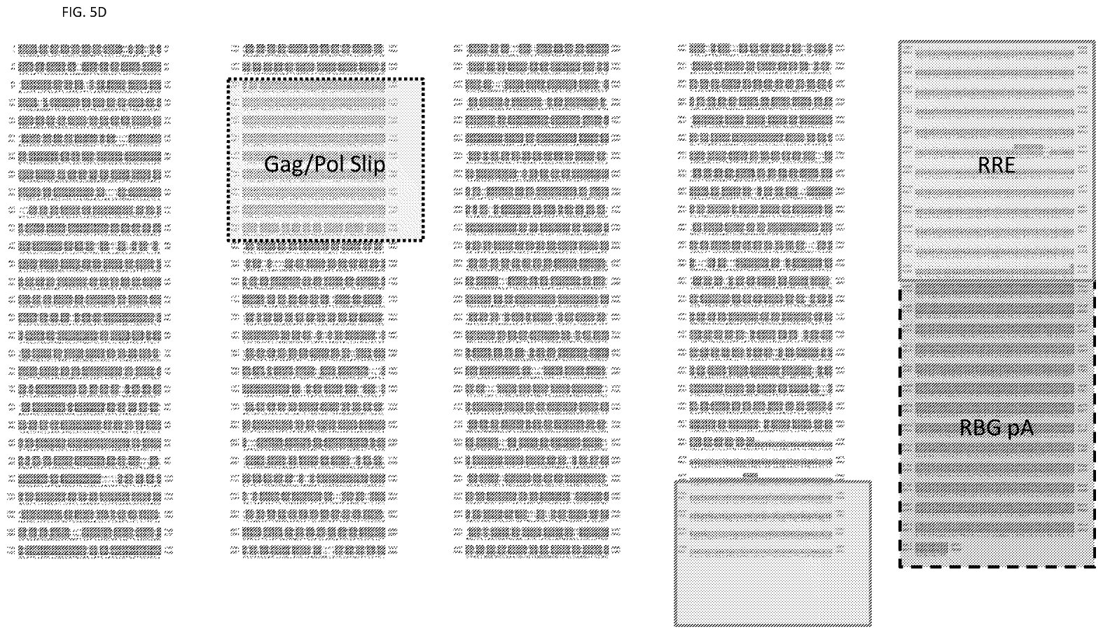

A comparison of the vector genome plasmid (pDNA1) of pGM326 with the GagPol plasmid (pDNA2a) of pGM297 was carried out. As shown in FIG. 5A, there is significant homology between the partial gagpol nucleotide sequence in pGM326 and the non-codon optimised gagpol sequence of pGM297.

A modified pDNA2a plasmid was designed to (i) reduce the homology between the partial gagpol nucleotide sequence in pGM326 and the non-codon optimised gagpol sequence of pGM297; (ii) to codon-optimise the gagpol genes for increased gagpol protein expression; (iii) to reduce the theoretical risk of generating replication-competent lentivirus (RCL) during manufacture or clinical use; and (iv) to eliminate gagpol expression dependency on Rev. A comparison of pGM297 with the modified pDNA2a (pGM691) is shown in FIGS. 5B-5D, with the changes annotated.

pGM691 was created by digesting pGM297 with the restriction enzymes XhoI, EcoRV and BglII to yield DNA fragments of 4583 bp, 3662 bp and 1641 bp. The 4583 bp fragment, containing the plasmid origin of replication and CBA promoter intron was purified and retained. The plasmid pGM693 was manufactured by GeneArt/LifeTechnologies via DNA synthesis. pGM693 was designed by the inventors to include a 4481 bp XhoI to BglII DNA fragment that included the codon optimised GagPol sequence ultimately found in pGM691, pGM693 was digested with XhoI and BglII to yield DNA fragments of 4481 bp, 1236 bp and 1048 bp. The 4481 bp fragment, containing the codon optimised GagPol sequence was purified and retained (see FIG. 5E). The two retained DNA fragments were ligated with DNA ligase and the resulting mixture of ligated DNA was transformed into E. coli Stbl3 cells; cells containing plasmids capable of replication were selected by resistance to kanamycin. Well-isolated individual colonies of kanamycin resistant, transformed Stbl3 cells were selected and expanded. DNA restriction analysis of the resultant clones identified a number of clones with the expected DNA structure; one was reserved and termed pGM691.

### Example 2—Production of rSIV.F/HN Vector hCEF-CFTR

The vector genome pGM326, which incorporates a CFTR transgene under the transcriptional control of the hCEF promoter was used in two design of experiments (DoE) studies to evaluate the production yields provided by using either pGM297 GagPol or pGM691 coGagPol.

In each DoE study a wide range of conditions was employed that included low, centre and high concentrations of each of the components used:

| Function | Code | Low | Centre | High |
|---|---|---|---|---|
| Genome | pGM326 | 0.2 | 1.1 | 2 |
| (co)GagPol | pGM297 or | 0.1 | 0.55 | 1 |
| | GM691 | | | |
| Rev | pGM299 | 0.1 | 0.55 | 1 |
| F | pGM301 | 0.1 | 0.55 | 1 |
| HN | pGM303 | 0.1 | 0.55 | 1 |
| Transfection | Lipofectamine | 4 | 7 | 10 |
| Reagent | 2000 | | | |

The units for transfection reagent was μL/mL, for all other reagents it was μg/mL.

A 3-level fractional factorial design was employed with duplicate vector stocks prepared for the majority of conditions and six replicate centre points. Overall, 31 vector stocks were prepared using otherwise identical conditions for pGM297 GagPol and pGM691 coGagPol.

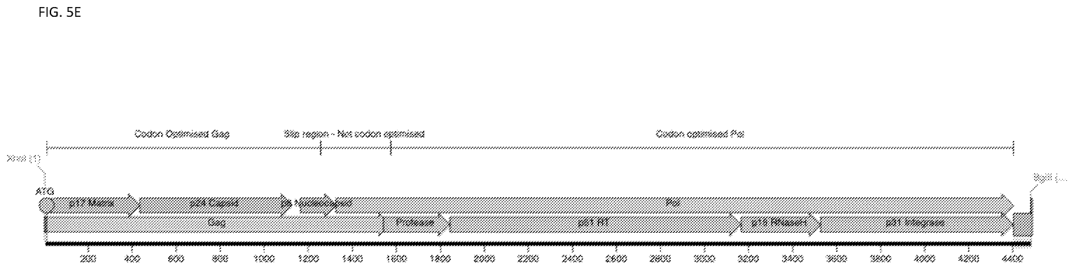

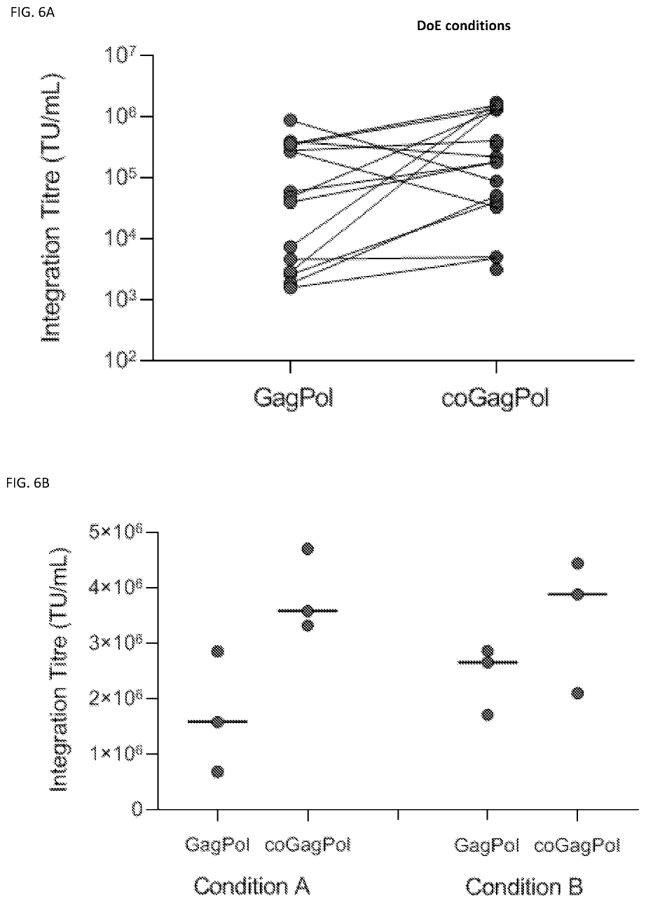

The integrating transducing unit titre (TU/mL), as determined by the detection of the ratio of vector specific and genome specific DNA sequences in transduced cells via quantitative PCR following transduction of 293T cells with dilutions of the vector stocks was plotted in FIG. 6A (replicate vector stocks represented as dots, the line indicates otherwise identical conditions).

Following on from the DOE experiments, vector genome pGM326, which incorporates a CFTR transgene under the transcriptional control of the hCEF promoter was used to prepare rSIV.F/HN vector stocks in triplicate using either pGM297 GagPol or pGM691 coGagPol as indicated.

For all preparations, Rev, F and HN were provided by pGM299, pGM301 and pGM303 respectively. The DNA mass ratio of vector genome:GagPol:Rev:F:HN used was 20:9:6:6:6 in all cases. For conditions A and B, the total DNA levels used were 2.2 μg/mL and 1.8 μg/mL respectively. For conditions A and B, the total Lipofectamine 2000 levels used were 7 μL/mL and 8 μL/mL respectively.

The integrating transducing unit titre (TU/mL), as determined by the ratio of vector specific to genome specific DNA sequences in transduced cells via quantitative PCR following transduction of 293T cells with dilutions of the vector stocks, is plotted (individual vector stocks represented as dots, the line indicates the group median).

Vector yields with the coGagPol as provided by pGM691 was observed to be ˜2.3-fold higher under Condition A and ˜1.5-fold higher under Condition B (FIG. 6B). Thus, use of pGM691 as pDNA2a observably increased SIV viral titre, independent of other culture conditions used. This is surprising, because there are multiple independent published studies which report that codon-optimisation of the gagpol genes is associated with a decrease in lentiviral titre.

### Example 3—Production of rSIV.F/HN CMV-EGFP

To investigate whether or not the ability of codon-optimised gagpol to maintain or increase vector titre was limited to the specific rSIV.F/HN construct (rSIV.F/HN hCEF-CFTR), experiments were conducted using plasmids to produce a different transgene operably linked to a different promoter.

HEK293T, Freestyle 293F (Life Technologies, Paisley, UK) and 293T/17 cells (CRL-11268; ATCC, Manassas, VA) were maintained in Dulbecco's minimal Eagle's medium (Invitrogen, Carlsbad, CA) containing 10% fetal bovine serum and supplemented with penicillin (100) U/ml) and streptomycin (100 μg/ml) or Freestyle™ 293 Expression Medium (Life Technologies).

SeV-F/HN-pseudotyped SIV vector was produced by transfecting HEK293T or 293T/17 cells cultured in FreeStyle™ 293 Expression Medium with a mixture of five plasmids with the following characteristics: pDNA1 (pGM311; which incorporates an EGFP transgene under the transcriptional control of the CMV promoter) encodes the lentiviral vector mRNA; pDNA2a (pGM691; FIG. 2C) encodes SIV Gag and Pol proteins; pDNA2b (pGM299: FIG. 2D) encodes SIV Rev proteins; pDNA3a (pGM301; FIG. 2E) encodes the Sendai virus-derived Fct4 protein [Kobayashi et al., 2003 J. Virol. 77:2607]; and pDNA3b (pGM303; FIG. 2F) encodes the Sendai virus-derived SIVct+HN [Kobayashi et al., 2003 J. Virol. 77:2607] complexed with PEIpro (Polyplus, Illkirch, France). Cell culture media was supplemented at 12-24 post-transfection with sodium butyrate. Sodium butyrate stimulates vector production via inhibiting histone deacetylase resulting in increasing expression of the SIV and Sendai virus fusion protein components encoded by the five plasmids. Cell culture media was supplemented at 44-52 hours and/or 68-76 hours post-transfection with 5 units/mL Benzonase Nuclease (Merck Millipore, Nottingham, UK). The culture supernatant containing the SIV vector was harvested 68-76.5 hours after transfection, and clarified by filtration through a 0.45 μm membrane. The SIV vector is treated by digestion with TrypLE Select™. Subsequently, SIV vector was further purified and concentrated by anion-exchange chromatography and tangential flow filtration.

rSIV.F/HN vector stocks in triplicate using either pGM297 GagPol or pGM691 coGagPol as indicated. The DNA mass ratio of vector genome:GagPol:Rev:F:HN used was 20:9:6:6:6 in all cases.

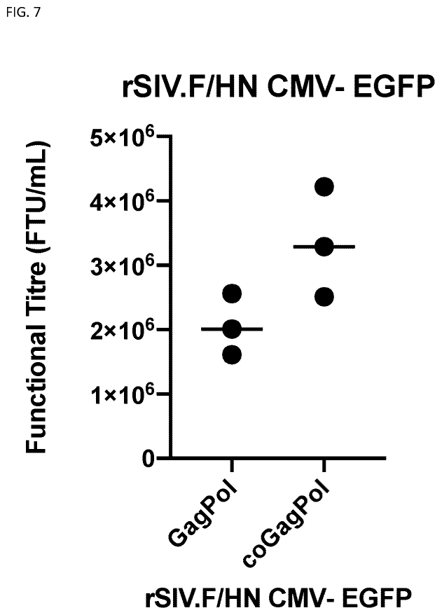

The functional transducing unit titre (FTU/mL), as determined by the detection of EGFP positive cells via flow cytometry following transduction of 293T cells with dilutions of the vector stocks was plotted in FIG. 7 (individual vector stocks represented as dots, the line indicates the group median). As for the rSIV.F/HN hCEF-CFTR constructs in Example 2, rSIV.F/HN CMV-EGFP vector yields with the coGagPol as provided by pGM691 were observed to be ˜1.6-fold higher than when the non-codon-optimised gagpol of pGM297 was used. This suggests that the ability of codon-optimised gagpol to maintain or increase vector titre was not limited to the specific rSIV.F/HN hCEF-CFTR construct, but rather is a function generally associated with the use of coGagPol.

### Example 3—Reducing the Number of Intact SIV ORFs within the Vector Genome Plasmid

Additional modifications to one or more of the construction plasmids can further improve the safety of the final vector product, providing a further clinical advantage.

The inventors reviewed sequences of the construction plasmids and identified several regions of concern within the vector genome plasmid pGM326. In particular, the pGM326 partial Gag RRE cPPT hCEF region contains;
77 start codons (ATGs);
32 ORFs ≥10 amino acids in length
2 large ORFs in the 5′ to 3′ direction
   189 amino acids from the most 5′ ATG in vector genome (Gag/RRE fusion), encoding p17 Matrix and part of p24 capsid
   250 amino acids from ATG internal to RRE (RRE/cPPT/hCEF fusion)

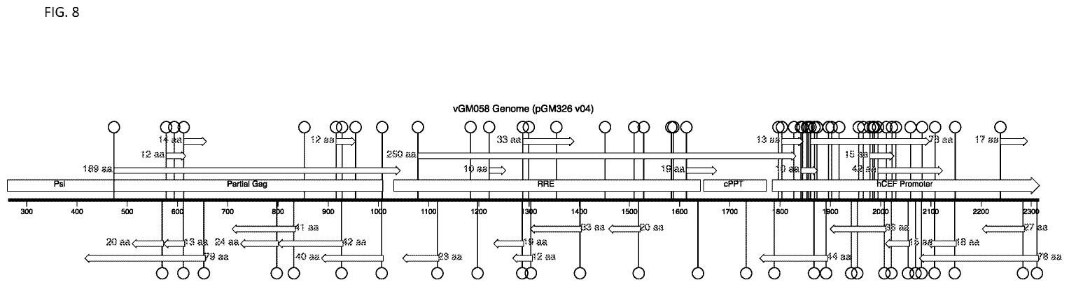

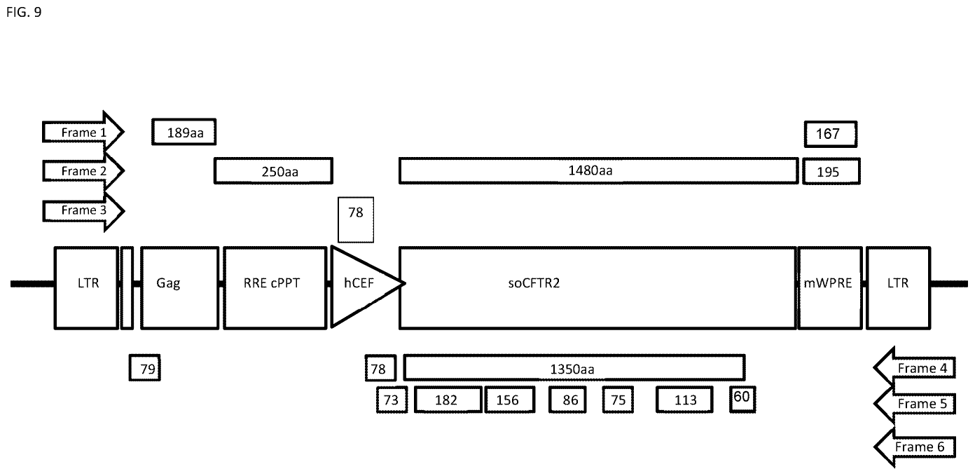

These are illustrated in FIG. 8. The 2 large ORFs (shown in FIG. 9) were of particular concern.

As such, the inventors designed a modified version of the pGM326 plasmid with a combination of additional modifications intended to reduce the number of intact SIV ORFs (and in particular to remove these 2 large ORFs) for improved safety. The modifications are made to the 2 large ORFs upstream of the hCEF promoter and CFTR transgene (soCFTR2). The changes made were as follows:
6 ATGs Eliminated (3×ATG-ATTG, 1×ATG-TTG, 2×ATG-AAG)
1 Stop inserted (TCC-TAAA)
1 Restriction site between partial Gag and RRE altered (EcoRI GAATTC-GCCTGCAGG SbfI)

The resulting vector genome plasmid is pGM830 as shown in FIG. 2B, with the sequence of SEQ ID NO:4.

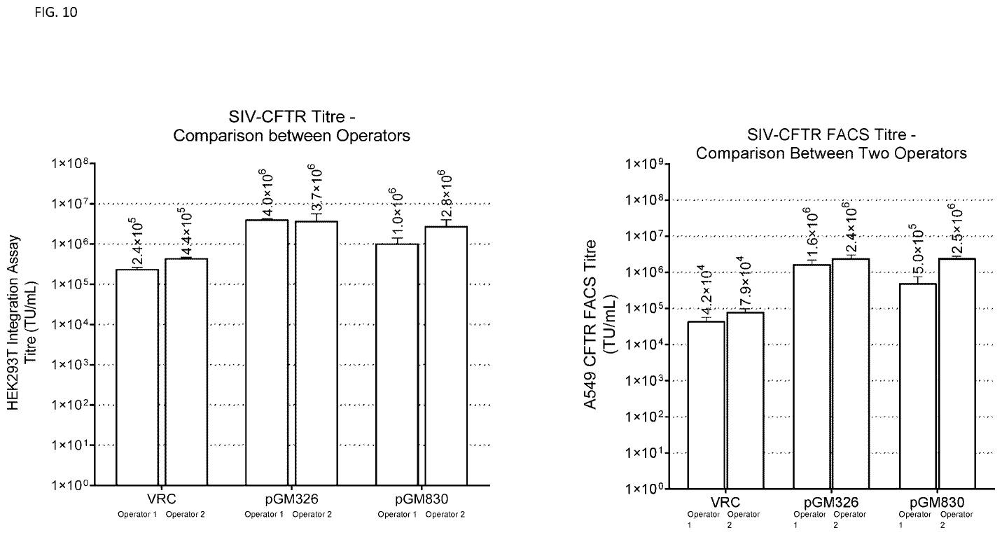

Comparisons of vector titre using either the pGM326 or pGM830 vector genome plasmids in an otherwise identical production protocol demonstrated that the use of pGM830 gave a comparable titre to pGM326 using both HEK293T and A549 cells (see FIG. 10), indicating that an improved safety profile could be achieved without adversely affecting titre.

### Example 4—Combination of coGagPol and a Modified Vector Genome Plasmid Maintains, or Even Increases Vector Titre

The experiments reported in Example 2 surprisingly demonstrated that, rather than the expected decrease in yield, generation of SIV.F/HN hCEF-CFTR using coGagPol trended to maintain or even increase vector titre. The experiments reported in Example 3 demonstrated that a further improvement to the safety profile of the vector could be achieved by modifying the vector genome plasmid, without adversely affecting the vector titre.

Following on from this, additional experiments were carried out in which the use of coGagPol was combined with the use of the pGM830 vector genome plasmid, to investigate whether these two safety-related modifications could be combined and vector titre maintained.

As illustrated in FIG. 11, the inventors surprisingly found that not only could the use of coGagPol be combined with the use of a modified vector genome plasmid (pGM830), but that this combination gave an observable trend to increase vector titre.

This suggests not only can vectors with further improved safety profiles be obtained by combining the use of coGagPol with a modified vector genome plasmid, but that surprisingly this can be achieved whilst maintaining or even increasing rSIV.F/HN hCEF-transgene titre.

### Sequence Information

### Key to Sequences

SEQ ID NO:1 codon-optimised SIV gal-pol nucleic acid sequence
SEQ ID NO:2 wild-type SIV gag-pol nucleic acid sequence
SEQ ID NO:3 Plasmid as defined in FIG. 2A (pDNA1 pGM326)
SEQ ID NO:4 Plasmid as defined in FIG. 2B (pDNA1 pGM830)
SEQ ID NO:5 Plasmid as defined in FIG. 2C (pDNA2a pGM691)
SEQ ID NO:6 Plasmid as defined in FIG. 2D (pDNA2b pGM299)
SEQ ID NO:7 Plasmid as defined in FIG. 2E (pDNA3a pGM301)
SEQ ID NO:8 Plasmid as defined in FIG. 2F (pDNA3b pGM303)
SEQ ID NO:9 Plasmid as defined in FIG. 2G (pDNA2a pGM297)
SEQ ID NO:10 Exemplified hCEF promoter
SEQ ID NO:11 Exemplified CMV promoter
SEQ ID NO:12 Exemplified EF1a promoter
SEQ ID NO:13 Exemplified CFTR transgene (soCFTR2)
SEQ ID NO:14 Exemplified A1AT transgene
SEQ ID NO:15 Complementary strand to the exemplified A1AT transgene
SEQ ID NO:16 Exemplified A1A1 polypeptide
SEQ ID NO:17 Exemplified FVIII transgene (N6)
SEQ ID NO:18 Exemplified FVIII transgene (V3)
SEQ ID NO:19 Complementary strand to the exemplified FVIII transgene (N6)
SEQ ID NO:20 Complementary strand to the exemplified FVIII transgene (V3)
SEQ ID NO:21 Exemplified FVIII polypeptide (N6)
SEQ ID NO:22 Exemplified FVIII polypeptide (V3)
SEQ ID NO:23 Exemplified WPRE component (mWPRE)
SEQ ID NO:24 F/HN-SIV-hCEF-soA1AT plasmid as defined in FIG. 3 (pDNA1 pGM407)
SEQ ID NO:25 F/HN-SIV-CMV-HFVIII-V3 plasmid as defined in FIG. 4A (pDNA1 pGM411)
SEQ ID NO:26 F/HN-SIV-hCEF-HFVIII-V3 plasmid as defined in FIG. 4B (pDNA1 pGM413)
SEQ ID NO:27 F/HN-SIV-CMV-HFVIII-N6-co plasmid as defined in FIG. 4C (pDNA1 pGM412)
SEQ ID NO:28 F/HN-SIV-hCEF-HFVIII-N6-co plasmid as defined in FIG. 4D (pDNA1 pGM414)
SEQ ID NO:29 Exemplary CAG promoter

| Sequences | |
|---|---|
| codon-optimised SIV gag-pol nucleic acid sequence (from pGM691) | |
| Length: 4391; Molecule Type: DNA; Features Location/Qualifiers: source, | |
| 1..4391; mol_type, other DNA; note, codon-optimised SIV gag-pol nucleic | |
| acid sequence (from pGM691); organism, synthetic construct | |
| SEQ ID NO: 1 | |
| ATGGGAGCTGCCACATCTGCCCTGAATAGACGGCAGCTGGACCAGTTCGAGAAGATCAGACTGCGGCCCAACGGC | |
| AAGAAGAAGTACCAGATCAAGCACCTGATCTGGGCCGGCAAAGAGATGGAAAGATTCGGCCTGCACGAGCGGCTG | |
| CTGGAAACCGAGGAAGGCTGCAAGAGAATTATCGAGGTGCTGTACCCTCTGGAACCTACCGGCTCTGAGGGCCTG | |
| AAGTCCCTGTTCAATCTCGTGTGCGTGCTGTACTGCCTGCACAAAGAACAGAAAGTGAAGGACACCGAAGAGGCC | |
| GTGGCCACAGTTAGACAGCACTGCCACCTGGTGGAAAAAGAGAAGTCCGCCACAGAGACAAGCAGCGGCCAGAAG | |
| AAGAACGACAAGGGAATTGCTGCCCCTCCTGGCGGCAGCCAGAATTTTCCTGCTCAGCAGCAGGGAAACGCCTGG | |
| GTGCACGTTCCACTGAGCCCTAGAACACTGAATGCCTGGGTCAAAGCCGTGGAAGAGAAGAAGTTTGGCGCCGAG | |
| ATCGTGCCCATGTTCCAGGCTCTGTCTGAGGGCTGCACCCCTTACGACATCAACCAGATGCTGAACGTGCTGGGA | |
| GATCACCAGGGCGCTCTGCAGATCGTGAAAGAGATCATCAACGAAGAGGCTGCCCAGTGGGACGTGACACATCCA | |
| TTGCCTGCTGGACCTCTGCCAGCCGGACAACTGAGAGATCCTAGAGGCTCTGATATCGCCGGCACCACCAGCTCT | |
| GTGCAAGAGCAGCTGGAATGGATCTACACCGCCAATCCTAGAGTGGACGTGGGCGCCATCTACAGAAGATGGATC | |
| ATCCTGGGCCTGCAGAAATGCGTGAAGATGTACAACCCCGTGTCCGTGCTGGACATCAGACAGGGACCCAAAGAG | |
| CCCTTCAAGGACTACGTGGACCGGTTCTATAAGGCCATTAGAGCCGAGCAGGCCAGCGGCGAAGTGAAGCAGTGG | |
| ATGACAGAGAGCCTGCTGATCCAGAACGCCAATCCAGACTGCAAAGTGATCCTGAAAGGCCTGGGCATGCACCCC | |
| ACACTGGAAGAGATGCTGACAGCCTGTCAAGGCGTTGGCGGCCCTTCTTACAAAGCCAAAGTGATGGCCGAGATG | |
| ATGCAGACCATGCAGAACCAGAACATGGTGCAGCAAGGCGGCCCTAAGAGACAGAGGCCTCCTCTGAGATGCTAC | |
| AACTGCGGCAAGTTCGGCCACATGCAGAGACAGTGTCCTGAGCCTAGGAAAACAAAATGTCTAAAGTGTGGAAAA | |
| TTGGGACACCTAGCAAAAGACTGCAGGGGACAGGTGAATTTTTTAGGGTATGGACGGTGGATGGGGGCAAAACCG | |
| AGAAATTTTCCCGCCGCTACTCTTGGAGCGGAACCGAGTGCGCCTCCTCCACCGAGCGGCACCACCCCATACGAC | |
| CCAGCAAAGAAGCTCCTGCAGCAATATGCAGAGAAAGGGAAACAACTGAGGGAGCAAAAGAGGAATCCACCGGCA | |
| ATGAATCCGGATTGGACCGAGGGATATTCTTTGAACTCCCTCTTTGGAGAAGACCAATAAAGACCGTGTACATCG | |
| AGGGCGTGCCCATCAAGGCTCTGCTGGATACAGGCGCCGACGACACCATCATCAAAGAGAACGACCTGCAGCTGA | |
| GCGGCCCTTGGAGGCCTAAGATCATTGGAGGAATCGGCGGAGGCCTGAACGTCAAAGAGTACAACGACCGGGAAG | |
| TGAAGATCGAGGACAAGATCCTGAGGGGCACAATCCTGCTGGGCGCCACACCTATCAACATCATCGGCAGAAATC | |
| TGCTGGCCCCTGCCGGCGCTAGACTGGTTATGGGACAGCTCTCTGAGAAGATCCCCGTGACACCCGTGAAGCTGA | |
| AAGAAGGCGCTAGAGGACCTTGTGTGCGACAGTGGCCTCTGAGCAAAGAGAAGATTGAGGCCCTGCAAGAAATCT | |
| GTAGCCAGCTGGAACAAGAGGGCAAGATCAGCAGAGTTGGCGGCGAGAACGCCTACAATACCCCTATCTTCTGCA | |
| TCAAGAAAAAGGACAAGAGCCAGTGGCGGATGCTGGTGGACTTTAGAGAGCTGAACAAGGCTACCCAGGACTTCT | |
| TCGAGGTGCAGCTGGGAATTCCTCATCCTGCCGGCCTGCGGAAGATGAGACAGATCACAGTGCTGGATGTGGGCG | |
| ACGCCTACTACAGCATCCCTCTGGACCCCAACTTCAGAAAGTACACCGCCTTCACAATCCCCACCGTGAACAATC | |
| AAGGCCCTGGCATCAGATACCAGTTCAACTGCCTGCCTCAAGGCTGGAAGGGCAGCCCCACCATTTTTCAGAATA | |
| CCGCCGCCAGCATCCTGGAAGAAATCAAGAGAAACCTGCCTGCTCTGACCATCGTGCAGTACATGGACGATCTGT | |
| GGGTCGGAAGCCAAGAGAATGAGCACACCCACGACAAGCTGGTGGAACAGCTGAGAACAAAGCTGCAGGCCTGGG | |
| GCCTCGAAACCCCTGAGAAGAAGGTGCAGAAAGAACCTCCTTACGAGTGGATGGGCTACAAGCTGTGGCCTCACA | |
| AGTGGGAGCTGAGCCGGATTCAGCTCGAAGAGAAGGACGAGTGGACCGTGAACGACATCCAGAAACTCGTGGGCA | |
| AGCTGAATTGGGCAGCCCAGCTGTATCCCGGCCTGAGGACCAAGAACATCTGCAAGCTGATCCGGGGAAAGAAGA | |
| ACCTGCTGGAACTGGTCACATGGACACCTGAGGCCGAGGCCGAATATGCCGAGAATGCCGAAATCCTGAAAACCG | |
| AGCAAGAGGGGACCTACTACAAGCCTGGCATTCCAATCAGAGCTGCCGTGCAGAAACTGGAAGGCGGCCAGTGGT | |
| CCTACCAGTTTAAGCAAGAAGGCCAGGTCCTGAAAGTGGGCAAGTACACCAAGCAGAAGAACACCCACACCAACG | |
| AGCTGAGGACACTGGCTGGCCTGGTCCAGAAAATCTGCAAAGAGGCCCTGGTCATTTGGGGCATCCTGCCTGTTC | |
| TGGAACTGCCCATTGAGCGGGAAGTGTGGGAACAGTGGTGGGCCGATTACTGGCAAGTGTCTTGGATCCCCGAGT | |
| GGGACTTCGTGTCTACCCCTCCTCTGCTGAAACTGTGGTACACCCTGACAAAAGAGCCCATTCCTAAAGAGGACG | |
| TCTACTACGTTGACGGCGCCTGCAACCGGAACTCCAAAGAAGGCAAGGCCGGCTACATCAGCCAGTACGGCAAGC | |
| AGAGAGTGGAAACCCTGGAAAACACCACCAACCAGCAGGCCGAGCTGACCGCCATTAAGATGGCCCTGGAAGATA | |
| GCGGCCCCAATGTGAACATCGTGACCGACTCTCAGTACGCCATGGGAATCCTGACAGCCCAGCCTACACAGAGCG | |
| ATAGCCCTCTGGTTGAGCAGATCATTGCCCTGATGATTCAGAAGCAGCAAATCTACCTGCAGTGGGTGCCCGCTC | |
| ACAAAGGCATCGGCGGAAACGAAGAGATCGATAAGCTGGTGTCCAAGGGAATCAGACGGGTGCTGTTCCTGGAAA | |
| AGATTGAAGAGGCCCAAGAGGAACACGAGCGCTACCACAACAACTGGAAGAATCTGGCCGACACCTACGGACTGC | |
| CCCAGATCGTGGCCAAAGAAATCGTGGCTATGTGCCCCAAGTGTCAGATCAAGGGCGAACCTGTGCACGGCCAAG | |
| TGGATGCTTCTCCTGGCACATGGCAGATGGACTGTACCCACCTGGAAGGCAAAGTGGTCATCGTGGCTGTGCACG | |
| TGGCCTCCGGCTTTATTGAGGCCGAAGTGATCCCCAGAGAGACAGGCAAAGAAACCGCCAAGTTCCTGCTGAAGA | |
| TCCTGTCCAGATGGCCCATCACACAGCTGCACACCGACAACGGCCCTAACTTCACATCTCAAGAGGTGGCCGCCA | |
| TCTGTTGGTGGGGAAAGATTGAGCACACAACCGGCATTCCCTACAATCCACAGAGCCAGGGCAGCATCGAGTCCA | |
| TGAACAAGCAGCTCAAAGAGATTATCGGCAAGATCCGGGACGACTGCCAGTACACAGAAACAGCCGTGCTGATGG | |
| CCTGTCACATCCACAACTTCAAGCGGAAAGGCGGCATCGGAGGACAGACATCTGCCGAGAGACTGATCAATATCA | |
| TCACCACTCAGCTGGAAATCCAGCACCTCCAGACCAAGATCCAGAAGATTCTGAACTTCCGGGTGTACTACCGCG | |
| AGGGCAGAGATCCTGTTTGGAAAGGCCCAGCACAGCTGATCTGGAAAGGCGAAGGTGCCGTGGTGCTGAAGGATG | |
| GCTCTGATCTGAAGGTGGTGCCCAGACGGAAGGCCAAGATTATCAAGGATTACGAGCCCAAACAGCGCGTGGGCA | |
| ATGAAGGCGACGTTGAGGGCACAAGAGGCAGCGACAATTGA | |
| wild-type SIV gag-pol nucleic acid sequence (from pGM297) | |
| Length: 4391; Molecule Type: DNA; Features Location/Qualifiers: source, | |
| 1..4391; mol_type, unassigned DNA; organism, Simian immunodeficiency virus | |
| SEQ ID NO: 2 | |
| ATGGGGGCGGCTACCTCAGCACTAAATAGGAGACAATTAGACCAATTTGAGAAAATACGACTTCGCCCGAACGGA | |
| AAGAAAAAGTACCAAATTAAACATTTAATATGGGCAGGCAAGGAGATGGAGCGCTTCGGCCTCCATGAGAGGTTG | |
| TTGGAGACAGAGGAGGGGTGTAAAAGAATCATAGAAGTCCTCTACCCCCTAGAACCAACAGGATCGGAGGGCTTA | |
| AAAAGTCTGTTCAATCTTGTGTGCGTACTATATTGCTTGCACAAGGAACAGAAAGTGAAAGACACAGAGGAAGCA | |
| GTAGCAACAGTAAGACAACACTGCCATCTAGTGGAAAAAGAAAAAAGTGCAACAGAGACATCTAGTGGACAAAAG | |
| AAAAATGACAAGGGAATAGCAGCGCCACCTGGTGGCAGTCAGAATTTTCCAGCGCAACAACAAGGAAATGCCTGG | |
| GTACATGTACCCTTGTCACCGCGCACCTTAAATGCGTGGGTAAAAGCAGTAGAGGAGAAAAAATTTGGAGCAGAA | |
| ATAGTACCCATGTTTCAAGCCCTATCAGAAGGCTGCACACCCTATGACATTAATCAGATGCTTAATGTGCTAGGA | |
| GATCATCAAGGGGCATTACAAATAGTGAAAGAGATCATTAATGAAGAAGCAGCCCAGTGGGATGTAACACACCCA | |
| CTACCCGCAGGACCCCTACCAGCAGGACAGCTCAGGGACCCTCGCGGCTCAGATATAGCAGGGACCACCAGCTCA | |
| GTACAAGAACAGTTAGAATGGATCTATACTGCTAACCCCCGGGTAGATGTAGGTGCCATCTACCGGAGATGGATT | |
| ATTCTAGGACTTCAAAAGTGTGTCAAAATGTACAACCCAGTATCAGTCCTAGACATTAGGCAGGGACCTAAAGAG | |
| CCCTTCAAGGATTATGTGGACAGATTTTACAAGGCAATTAGAGCAGAACAAGCCTCAGGGGAAGTGAAACAATGG | |
| ATGACAGAATCATTACTCATTCAAAATGCTAATCCAGATTGTAAGGTCATCCTGAAGGGCCTAGGAATGCACCCC | |
| ACCCTTGAAGAAATGTTAACGGCTTGTCAGGGGGTAGGAGGCCCAAGCTACAAAGCAAAAGTAATGGCAGAAATG | |
| ATGCAGACCATGCAAAATCAAAACATGGTGCAGCAGGGAGGTCCAAAAAGACAAAGACCCCCACTAAGATGTTAT | |
| AATTGTGGAAAATTTGGCCATATGCAAAGACAATGTCCGGAACCAAGGAAAACAAAATGTCTAAAGTGTGGAAAA | |
| TTGGGACACCTAGCAAAAGACTGCAGGGGACAGGTGAATTTTTTAGGGTATGGACGGTGGATGGGGGCAAAACCG | |
| AGAAATTTTCCCGCCGCTACTCTTGGAGCGGAACCGAGTGCGCCTCCTCCACCGAGCGGCACCACCCCATACGAC | |
| CCAGCAAAGAAGCTCCTGCAGCAATATGCAGAGAAAGGGAAACAACTGAGGGAGCAAAAGAGGAATCCACCGGCA | |
| ATGAATCCGGATTGGACCGAGGGATATTCTTTGAACTCCCTCTTTGGAGAAGACCAATAAAGACAGTGTATATAG | |
| AAGGGGTCCCCATTAAGGCACTGCTAGACACAGGGGCAGATGACACCATAATTAAAGAAAATGATTTACAATTAT | |
| CAGGTCCATGGAGACCCAAAATTATAGGGGGCATAGGAGGAGGCCTTAATGTAAAAGAATATAACGACAGGGAAG | |
| TAAAAATAGAAGATAAAATTTTGAGAGGAACAATATTGTTAGGAGCAACTCCCATTAATATAATAGGTAGAAATT | |
| TGCTGGCCCCGGCAGGTGCCCGGTTAGTAATGGGACAATTATCAGAAAAAATTCCTGTCACACCTGTCAAATTGA | |
| AGGAAGGGGCTCGGGGACCCTGTGTAAGACAATGGCCTCTCTCTAAAGAGAAGATTGAAGCTTTACAGGAAATAT | |
| GTTCCCAATTAGAGCAGGAAGGAAAAATCAGTAGAGTAGGAGGAGAAAATGCATACAATACCCCAATATTTTGCA | |
| TAAAGAAGAAGGACAAATCCCAGTGGAGGATGCTAGTAGACTTTAGAGAGTTAAATAAGGCAACCCAAGATTTCT | |
| TTGAAGTGCAATTAGGGATACCCCACCCAGCAGGATTAAGAAAGATGAGACAGATAACAGTTTTAGATGTAGGAG | |
| ACGCCTATTATTCCATACCATTGGATCCAAATTTTAGGAAATATACTGCTTTTACTATTCCCACAGTGAATAATC | |
| AGGGACCCGGGATTAGGTATCAATTCAACTGTCTCCCGCAAGGGTGGAAAGGATCTCCTACAATCTTCCAAAATA | |
| CAGCAGCATCCATTTTGGAGGAGATAAAAAGAAACTTGCCAGCACTAACCATTGTACAATACATGGATGATTTAT | |
| GGGTAGGTTCTCAAGAAAATGAACACACCCATGACAAATTAGTAGAACAGTTAAGAACAAAATTACAAGCCTGGG | |
| GCTTAGAAACCCCAGAAAAGAAGGTGCAAAAAGAACCACCTTATGAGTGGATGGGATACAAACTTTGGCCTCACA | |
| AATGGGAACTAAGCAGAATACAACTGGAGGAAAAAGATGAATGGACTGTCAATGACATCCAGAAGTTAGTTGGGA | |
| AACTAAATTGGGCAGCACAATTGTATCCAGGTCTTAGGACCAAGAATATATGCAAGTTAATTAGAGGAAAGAAAA | |
| ATCTGTTAGAGCTAGTGACTTGGACACCTGAGGCAGAAGCTGAATATGCAGAAAATGCAGAGATTCTTAAAACAG | |
| AACAGGAAGGAACCTATTACAAACCAGGAATACCTATTAGGGCAGCAGTACAGAAATTGGAAGGAGGACAGTGGA | |
| GTTACCAATTCAAACAAGAAGGACAAGTCTTGAPAGTAGGAAAATACACCAAGCAAAAGAACACCCATACAAATG | |
| AACTTCGCACATTAGCTGGTTTAGTGCAGAAGATTTGCAAAGAAGCTCTAGTTATTTGGGGGATATTACCAGTTC | |
| TAGAACTCCCGATAGAAAGAGAGGTATGGGAACAATGGTGGGCGGATTACTGGCAGGTAAGCTGGATTCCCGAAT | |
| GGGATTTTGTCAGCACCCCACCTTTGCTCAAACTATGGTACACATTAACAAAAGAACCCATACCCAAGGAGGACG | |
| TTTACTATGTAGATGGAGCATGCAACAGAAATTCAAAAGAAGGAAAAGCAGGATACATCTCACAATACGGAAAAC | |
| AGAGAGTAGAAACATTAGAAAACACTACCAATCAGCAAGCAGAATTAACAGCTATAAAAATGGCTTTGGAAGACA | |
| GTGGGCCTAATGTGAACATAGTAACAGACTCTCAATATGCAATGGGAATTTTGACAGCACAACCCACACAAAGTG | |
| ATTCACCATTAGTAGAGCAAATTATAGCCTTAATGATACAAAAGCAACAAATATATTTGCAGTGGGTACCAGCAC | |
| ATAAAGGAATAGGAGGAAATGAGGAGATAGATAAATTAGTGAGTAAAGGCATTAGAAGAGTTTTATTCTTAGAAA | |
| AAATAGAAGAAGCTCAAGAAGAGCATGAAAGATATCATAATAATTGGAAAAACCTAGCAGATACATATGGGCTTC | |
| CACAAATAGTAGCAAAAGAGATAGTGGCCATGTGTCCAAAATGTCAGATAAAGGGAGAACCAGTGCATGGACAAG | |
| TGGATGCCTCACCTGGAACATGGCAGATGGATTGTACTCATCTAGAAGGAAAAGTAGTCATAGTTGCGGTCCATG | |
| TAGCCAGTGGATTCATAGAAGCAGAAGTCATACCTAGGGAAACAGGAAAAGAAACGGCAAAGTTTCTATTAAAAA | |
| TACTGAGTAGATGGCCTATAACACAGTTACACACAGACAATGGGCCTAACTTTACCTCCCAAGAAGTGGCAGCAA | |
| TATGTTGGTGGGGAAAAATTGAACATACAACAGGTATACCATATAACCCCCAATCTCAAGGATCAATAGAAAGCA | |
| TGAACAAACAATTAAAAGAGATAATTGGGPAAATAAGAGATGATTGCCAATATACAGAGACAGCAGTACTGATGG | |
| CTTGCCATATTCACAATTTTAAAAGAAAGGGAGGAATAGGGGGACAGACTTCAGCAGAGAGACTAATTAATATAA | |
| TAACAACACAATTAGAAATACAACATTTACAAACCAAAATTCAAAAAATTTTAAATTTTAGAGTCTACTACAGAG | |
| AAGGGAGAGACCCTGTGTGGAAAGGACCAGCACAATTAATCTGGAAAGGGGAAGGAGCAGTGGTCCTCAAGGACG | |
| GAAGTGACCTAAAGGTTGTACCAAGAAGGAAAGCTAAAATTATTAAGGATTATGAACCCAAACAAAGAGTGGGTA | |
| ATGAGGGTGACGTGGAAGGTACCAGGGGATCTGATAACTAA | |
| Plasmid as defined in FIG. 2A (pDNA1 pGM326) | |
| Length: 10528; Molecule Type: DNA; Features Location/Qualifiers: source, | |
| 1..10528; mol_type, other DNA; note, pGM326; organism, synthetic construct | |
| SEQ ID NO: 3 | |
| GGTACCTCAATATTGGCCATTAGCCATATTATTCATTGGTTATATAGCATAAATCAATATTGGCTATTGGCCATT | |
| GCATACGTTGTATCTATATCATAATATGTACATTTATATTGGCTCATGTCCAATATGACCGCCATGTTGGCATTG | |
| ATTATTGACTAGTTATTAATAGTAATCAATTACGGGGTCATTAGTTCATAGCCCATATATGGAGTTCCGCGTTAC | |
| ATAACTTACGGTAAATGGCCCGCCTGGCTGACCGCCCAACGACCCCCGCCCATTGACGTCAATAATGACGTATGT | |
| TCCCATAGTAACGCCAATAGGGACTTTCCATTGACGTCAATGGGTGGAGTATTTACGGTAAACTGCCCACTTGGC | |
| AGTACATCAAGTGTATCATATGCCAAGTCCGCCCCCTATTGACGTCAATGACGGTAAATGGCCCGCCTGGCATTA | |
| TGCCCAGTACATGACCTTACGGGACTTTCCTACTTGGCAGTACATCTACGTATTAGTCATCGCTATTACCATGGT | |
| GATGCGGTTTTGGCAGTACACCAATGGGCGTGGATAGCGGTTTGACTCACGGGGATTTCCAAGTCTCCACCCCAT | |
| TGACGTCAATGGGAGTTTGTTTTGGCACCAAAATCAACGGGACTTTCCAAAATGTCGTAACAACTGCGATCGCCC | |
| GCCCCGTTGACGCAAATGGGCGGTAGGCGTGTACGGTGGGAGGTCTATATAAGCAGAGCTCGCTGGCTTGTAACT | |
| CAGTCTCTTACTAGGAGACCAGCTTGAGCCTGGGTGTTCGCTGGTTAGCCTAACCTGGTTGGCCACCAGGGGTAA | |
| GGACTCCTTGGCTTAGAAAGCTAATAAACTTGCCTGCATTAGAGCTTATCTGAGTCAAGTGTCCTCATTGACGCC | |
| TCACTCTCTTGAACGGGAATCTTCCTTACTGGGTTCTCTCTCTGACCCAGGCGAGAGAAACTCCAGCAGTGGCGC | |
| CCGAACAGGGACTTGAGTGAGAGTGTAGGCACGTACAGCTGAGAAGGCGTCGGACGCGAAGGAAGCGCGGGGTGC | |
| GACGCGACCAAGAAGGAGACTTGGTGAGTAGGCTTCTCGAGTGCCGGGAAAAAGCTCGAGCCTAGTTAGAGGACT | |
| AGGAGAGGCCGTAGCCGTAACTACTCTGGGCAAGTAGGGCAGGCGGTGGGTACGCAATGGGGGCGGCTACCTCAG | |
| CACTAAATAGGAGACAATTAGACCAATTTGAGAAAATACGACTTCGCCCGAACGGAPAGAAAAAGTACCAAATTA | |
| AACATTTAATATGGGCAGGCAAGGAGATGGAGCGCTTCGGCCTCCATGAGAGGTTGTTGGAGACAGAGGAGGGGT | |
| GTAAAAGAATCATAGAAGTCCTCTACCCCCTAGAACCAACAGGATCGGAGGGCTTAAAAAGTCTGTTCAATCTTG | |
| TGTGCGTGCTATATTGCTTGCACAAGGAACAGAAAGTGAAAGACACAGAGGAAGCAGTAGCAACAGTAAGACAAC | |
| ACTGCCATCTAGTGGAAAAAGAAAAAAGTGCAACAGAGACATCTAGTGGACAAAAGAAAAATGACAAGGGAATAG | |
| CAGCGCCACCTGGTGGCAGTCAGAATTTTCCAGCGCAACAACAAGGAAATGCCTGGGTACATGTACCCTTGTCAC | |
| CGCGCACCTTAAATGCGTGGGTAAAAGCAGTAGAGGAGAAAAAATTTGGAGCAGAAATAGTACCCATGTTTCAAG | |
| CCCTATCGAATTCCCGTTTGTGCTAGGGTTCTTAGGCTTCTTGGGGGCTGCTGGAACTGCAATGGGAGCAGCGGC | |
| GACAGCCCTGACGGTCCAGTCTCAGCATTTGCTTGCTGGGATACTGCAGCAGCAGAAGAATCTGCTGGCGGCTGT | |
| GGAGGCTCAACAGCAGATGTTGAAGCTGACCATTTGGGGTGTTAAAAACCTCAATGCCCGCGTCACAGCCCTTGA | |
| GAAGTACCTAGAGGATCAGGCACGACTAAACTCCTGGGGGTGCGCATGGAAACAAGTATGTCATACCACAGTGGA | |
| GTGGCCCTGGACAAATCGGACTCCGGATTGGCAAAATATGACTTGGTTGGAGTGGGAAAGACAAATAGCTGATTT | |
| GGAAAGCAACATTACGAGACAATTAGTGAAGGCTAGAGAACAAGAGGAAAAGAATCTAGATGCCTATCAGAAGTT | |
| AACTAGTTGGTCAGATTTCTGGTCTTGGTTCGATTTCTCAAAATGGCTTAACATTTTAAAAATGGGATTTTTAGT | |
| AATAGTAGGAATAATAGGGTTAAGATTACTTTACACAGTATATGGATGTATAGTGAGGGTTAGGCAGGGATATGT | |
| TCCTCTATCTCCACAGATCCATATCCGCGGCAATTTTAAAAGAAAGGGAGGAATAGGGGGACAGACTTCAGCAGA | |
| GAGACTAATTAATATAATAACAACACAATTAGAAATACAACATTTACAAACCAAAATTCAAAAAATTTTAAATTT | |
| TAGAGCCGCGGAGATCTGTTACATAACTTATGGTAAATGGCCTGCCTGGCTGACTGCCCAATGACCCCTGCCCAA | |
| TGATGTCAATAATGATGTATGTTCCCATGTAATGCCAATAGGGACTTTCCATTGATGTCAATGGGTGGAGTATTT | |
| ATGGTAACTGCCCACTTGGCAGTACATCAAGTGTATCATATGCCAAGTATGCCCCCTATTGATGTCAATGATGGT | |
| AAATGGCCTGCCTGGCATTATGCCCAGTACATGACCTTATGGGACTTTCCTACTTGGCAGTACATCTATGTATTA | |
| GTCATTGCTATTACCATGGGAATTCACTAGTGGAGAAGAGCATGCTTGAGGGCTGAGTGCCCCTCAGTGGGCAGA | |
| GAGCACATGGCCCACAGTCCCTGAGAAGTTGGGGGGAGGGGTGGGCAATTGAACTGGTGCCTAGAGAAGGTGGGG | |
| CTTGGGTAAACTGGGAAAGTGATGTGGTGTACTGGCTCCACCTTTTTCCCCAGGGTGGGGGAGAACCATATATAA | |
| GTGCAGTAGTCTCTGTGAACATTCAAGCTTCTGCCTTCTCCCTCCTGTGAGTTTGCTAGCCACCATGCAGAGAAG | |
| CCCTCTGGAGAAGGCCTCTGTGGTGAGCAAGCTGTTCTTCAGCTGGACCAGGCCCATCCTGAGGAAGGGCTACAG | |
| GCAGAGACTGGAGCTGTCTGACATCTACCAGATCCCCTCTGTGGACTCTGCTGACAACCTGTCTGAGAAGCTGGA | |
| GAGGGAGTGGGATAGAGAGCTGGCCAGCAAGAAGAACCCCAAGCTGATCAATGCCCTGAGGAGATGCTTCTTCTG | |
| GAGATTCATGTTCTATGGCATCTTCCTGTACCTGGGGGAAGTGACCAAGGCTGTGCAGCCTCTGCTGCTGGGCAG | |
| AATCATTGCCAGCTATGACCCTGACAACAAGGAGGAGAGGAGCATTGCCATCTACCTGGGCATTGGCCTGTGCCT | |
| GCTGTTCATTGTGAGGACCCTGCTGCTGCACCCTGCCATCTTTGGCCTGCACCACATTGGCATGCAGATGAGGAT | |
| TGCCATGTTCAGCCTGATCTACAAGAAAACCCTGAAGCTGTCCAGCAGAGTGCTGGACAAGATCAGCATTGGCCA | |
| GCTGGTGAGCCTGCTGAGCAACAACCTGAACAAGTTTGATGAGGGCCTGGCCCTGGCCCACTTTGTGTGGATTGC | |
| CCCTCTGCAGGTGGCCCTGCTGATGGGCCTGATTTGGGAGCTGCTGCAGGCCTCTGCCTTTTGTGGCCTGGGCTT | |
| CCTGATTGTGCTGGCCCTGTTTCAGGCTGGCCTGGGCAGGATGATGATGAAGTACAGGGACCAGAGGGCAGGCAA | |
| GATCAGTGAGAGGCTGGTGATCACCTCTGAGATGATTGAGAACATCCAGTCTGTGAAGGCCTACTGTTGGGAGGA | |
| AGCTATGGAGAAGATGATTGAAAACCTGAGGCAGACAGAGCTGAAGCTGACCAGGAAGGCTGCCTATGTGAGATA | |
| CTTCAACAGCTCTGCCTTCTTCTTCTCTGGCTTCTTTGTGGTGTTCCTGTCTGTGCTGCCCTATGCCCTGATCAA | |
| GGGGATCATCCTGAGAAAGATTTTCACCACCATCAGCTTCTGCATTGTGCTGAGGATGGCTGTGACCAGACAGTT | |
| CCCCTGGGCTGTGCAGACCTGGTATGACAGCCTGGGGGCCATCAACAAGATCCAGGACTTCCTGCAGAAGCAGGA | |
| GTACAAGACCCTGGAGTACAACCTGACCACCACAGAAGTGGTGATGGAGAATGTGACAGCCTTCTGGGAGGAGGG | |
| CTTTGGGGAGCTGTTTGAGAAGGCCAAGCAGAACAACAACAACAGAAAGACCAGCAATGGGGATGACTCCCTGTT | |
| CTTCTCCAACTTCTCCCTGCTGGGCACACCTGTGCTGAAGGACATCAACTTCAAGATTGAGAGGGGGCAGCTGCT | |
| GGCTGTGGCTGGATCTACAGGGGCTGGCAAGACCAGCCTGCTGATGATGATCATGGGGGAGCTGGAGCCTTCTGA | |
| GGGCAAGATCAAGCACTCTGGCAGGATCAGCTTTTGCAGCCAGTTCAGCTGGATCATGCCTGGCACCATCAAGGA | |
| GAACATCATCTTTGGAGTGAGCTATGATGAGTACAGATACAGGAGTGTGATCAAGGCCTGCCAGCTGGAGGAGGA | |
| CATCAGCAAGTTTGCTGAGAAGGACAACATTGTGCTGGGGGAGGGAGGCATTACACTGTCTGGGGGCCAGAGAGC | |
| CAGAATCAGCCTGGCCAGGGCTGTGTACAAGGATGCTGACCTGTACCTGCTGGACTCCCCCTTTGGCTACCTGGA | |
| TGTGCTGACAGAGAAGGAGATTTTTGAGAGCTGTGTGTGCAAGCTGATGGCCAACAAGACCAGAATCCTGGTGAC | |
| CAGCAAGATGGAGCACCTGAAGAAGGCTGACAAGATCCTGATCCTGCATGAGGGCAGCAGCTACTTCTATGGGAC | |
| CTTCTCTGAGCTGCAGAACCTGCAGCCTGACTTCAGCTCTAAGCTGATGGGCTGTGACAGCTTTGACCAGTTCTC | |
| TGCTGAGAGGAGGAACAGCATCCTGACAGAGACCCTGCACAGATTCAGCCTGGAGGGAGATGCCCCTGTGAGCTG | |
| GACAGAGACCAAGAAGCAGAGCTTCAAGCAGACAGGGGAGTTTGGGGAGAAGAGGAAGAACTCCATCCTGAACCC | |
| CATCAACAGCATCAGGAAGTTCAGCATTGTGCAGAAAACCCCCCTGCAGATGAATGGCATTGAGGAAGATTCTGA | |
| TGAGCCCCTGGAGAGGAGACTGAGCCTGGTGCCTGATTCTGAGCAGGGAGAGGCCATCCTGCCTAGGATCTCTGT | |
| GATCAGCACAGGCCCTACACTGCAGGCCAGAAGGAGGCAGTCTGTGCTGAACCTGATGACCCACTCTGTGAACCA | |
| GGGCCAGAACATCCACAGGAAAACCACAGCCTCCACCAGGAAAGTGAGCCTGGCCCCTCAGGCCAATCTGACAGA | |
| GCTGGACATCTACAGCAGGAGGCTGTCTCAGGAGACAGGCCTGGAGATTTCTGAGGAGATCAATGAGGAGGACCT | |
| GAAAGAGTGCTTCTTTGATGACATGGAGAGCATCCCTGCTGTGACCACCTGGAACACCTACCTGAGATACATCAC | |
| AGTGCACAAGAGCCTGATCTTTGTGCTGATCTGGTGCCTGGTGATCTTCCTGGCTGAAGTGGCTGCCTCTCTGGT | |
| GGTGCTGTGGCTGCTGGGAAACACCCCACTGCAGGACAAGGGCAACAGCACCCACAGCAGGAACAACAGCTATGC | |
| TGTGATCATCACCTCCACCTCCAGCTACTATGTGTTCTACATCTATGTGGGAGTGGCTGATACCCTGCTGGCTAT | |
| GGGCTTCTTTAGAGGCCTGCCCCTGGTGCACACACTGATCACAGTGAGCAAGATCCTCCACCACAAGATGCTGCA | |
| CTCTGTGCTGCAGGCTCCTATGAGCACCCTGAATACCCTGAAGGCTGGGGGCATCCTGAACAGATTCTCCAAGGA | |
| TATTGCCATCCTGGATGACCTGCTGCCTCTCACCATCTTTGACTTCATCCAGCTGCTGCTGATTGTGATTGGGGC | |
| CATTGCTGTGGTGGCAGTGCTGCAGCCCTACATCTTTGTGGCCACAGTGCCTGTGATTGTGGCCTTCATCATGCT | |
| GAGGGCCTACTTTCTGCAGACCTCCCAGCAGCTGAAGCAGCTGGAGTCTGAGGGCAGAAGCCCCATCTTCACCCA | |
| CCTGGTGACAAGCCTGAAGGGCCTGTGGACCCTGAGAGCCTTTGGCAGGCAGCCCTACTTTGAGACCCTGTTCCA | |
| CAAGGCCCTGAACCTGCACACAGCCAACTGGTTCCTCTACCTGTCCACCCTGAGATGGTTCCAGATGAGAATTGA | |
| GATGATCTTTGTCATCTTCTTCATTGCTGTGACCTTCATCAGCATTCTGACCACAGGAGAGGGAGAGGGCAGAGT | |
| GGGCATTATCCTGACCCTGGCCATGAACATCATGAGCACACTGCAGTGGGCAGTGAACAGCAGCATTGATGTGGA | |
| CAGCCTGATGAGGAGTGTGAGCAGAGTGTTCAAGTTCATTGATATGCCCACAGAGGGCAAGCCTACCAAGAGCAC | |
| CAAGCCCTACAAGAATGGCCAGCTGAGCAAAGTGATGATCATTGAGAACAGCCATGTGAAGAAGGATGATATCTG | |
| GCCCAGTGGAGGCCAGATGACAGTGAAGGACCTGACAGCCAAGTACACAGAGGGGGGCAATGCTATCCTGGAGAA | |
| CATCTCCTTCAGCATCTCCCCTGGCCAGAGAGTGGGACTGCTGGGAAGAACAGGCTCTGGCAAGTCTACCCTGCT | |
| GTCTGCCTTCCTGAGGCTGCTGAACACAGAGGGAGAGATCCAGATTGATGGAGTGTCCTGGGACAGCATCACACT | |
| GCAGCAGTGGAGGAAGGCCTTTGGTGTGATCCCCCAGAAAGTGTTCATCTTCAGTGGCACCTTCAGGAAGAACCT | |
| GGACCCCTATGAGCAGTGGTCTGACCAGGAGATTTGGAAAGTGGCTGATGAAGTGGGCCTGAGAAGTGTGATTGA | |
| GCAGTTCCCTGGCAAGCTGGACTTTGTCCTGGTGGATGGGGGCTGTGTGCTGAGCCATGGCCACAAGCAGCTGAT | |
| GTGCCTGGCCAGATCAGTGCTGAGCAAGGCCAAGATCCTGCTGCTGGATGAGCCTTCTGCCCACCTGGATCCTGT | |
| GACCTACCAGATCATCAGGAGGACCCTCAAGCAGGCCTTTGCTGACTGCACAGTCATCCTGTGTGAGCACAGGAT | |
| TGAGGCCATGCTGGAGTGCCAGCAGTTCCTGGTGATTGAGGAGAACAAAGTGAGGCAGTATGACAGCATCCAGAA | |
| GCTGCTGAATGAGAGGAGCCTGTTCAGGCAGGCCATCAGCCCCTCTGATAGAGTGAAGCTGTTCCCCCACAGGAA | |
| CAGCTCCAAGTGCAAGAGCAAGCCCCAGATTGCTGCCCTGAAGGAGGAGACAGAGGAGGAAGTGCAGGACACCAG | |
| GCTGTGAGGGCCCAATCAACCTCTGGATTACAAAATTTGTGAAAGATTGACTGGTATTCTTAACTATGTTGCTCC | |
| TTTTACGCTATGTGGATACGCTGCTTTAATGCCTTTGTATCATGCTATTGCTTCCCGTATGGCTTTCATTTTCTC | |
| CTCCTTGTATAAATCCTGGTTGCTGTCTCTTTATGAGGAGTTGTGGCCCGTTGTCAGGCAACGTGGCGTGGTGTG | |
| CACTGTGTTTGCTGACGCAACCCCCACTGGTTGGGGCATTGCCACCACCTGTCAGCTCCTTTCCGGGACTTTCGC | |
| TTTCCCCCTCCCTATTGCCACGGCGGAACTCATCGCCGCCTGCCTTGCCCGCTGCTGGACAGGGGCTCGGCTGTT | |
| GGGCACTGACAATTCCGTGGTGTTGTCGGGGAAATCATCGTCCTTTCCTTGGCTGCTCGCCTGTGTTGCCACCTG | |
| GATTCTGCGCGGGACGTCCTTCTGCTACGTCCCTTCGGCCCTCAATCCAGCGGACCTTCCTTCCCGCGGCCTGCT | |
| GCCGGCTCTGCGGCCTCTTCCGCGTCTTCGCCTTCGCCCTCAGACGAGTCGGATCTCCCTTTGGGCCGCCTCCCC | |
| GCAAGCTTCGCACTTTTTAAAAGAAAAGGGAGGACTGGATGGGATTTATTACTCCGATAGGACGCTGGCTTGTAA | |
| CTCAGTCTCTTACTAGGAGACCAGCTTGAGCCTGGGTGTTCGCTGGTTAGCCTAACCTGGTTGGCCACCAGGGGT | |
| AAGGACTCCTTGGCTTAGAAAGCTAATAAACTTGCCTGCATTAGAGCTCTTACGCGTCCCGGGCTCGAGATCCGC | |
| ATCTCAATTAGTCAGCAACCATAGTCCCGCCCCTAACTCCGCCCATCCCGCCCCTAACTCCGCCCAGTTCCGCCC | |
| ATTCTCCGCCCCATGGCTGACTAATTTTTTTTATTTATGCAGAGGCCGAGGCCGCCTCGGCCTCTGAGCTATTCC | |
| AGAAGTAGTGAGGAGGCTTTTTTGGAGGCCTAGGCTTTTGCAAAAAGCTAACTTGTTTATTGCAGCTTATAATGG | |
| TTACAAATAAAGCAATAGCATCACAAATTTCACAAATAAAGCATTTTTTTCACTGCATTCTAGTTGTGGTTTGTC | |
| CAAACTCATCAATGTATCTTATCATGTCTGTCCGCTTCCTCGCTCACTGACTCGCTGCGCTCGGTCGTTCGGCTG | |
| CGGCGAGCGGTATCAGCTCACTCAAAGGCGGTAATACGGTTATCCACAGAATCAGGGGATAACGCAGGAAAGAAC | |
| ATGTGAGCAAAAGGCCAGCAAAAGGCCAGGAACCGTAAAAAGGCCGCGTTGCTGGCGTTTTTCCATAGGCTCCGC | |
| CCCCCTGACGAGCATCACAAAAATCGACGCTCAAGTCAGAGGTGGCGAAACCCGACAGGACTATAAAGATACCAG | |
| GCGTTTCCCCCTGGAAGCTCCCTCGTGCGCTCTCCTGTTCCGACCCTGCCGCTTACCGGATACCTGTCCGCCTTT | |
| CTCCCTTCGGGAAGCGTGGCGCTTTCTCATAGCTCACGCTGTAGGTATCTCAGTTCGGTGTAGGTCGTTCGCTCC | |
| AAGCTGGGCTGTGTGCACGAACCCCCCGTTCAGCCCGACCGCTGCGCCTTATCCGGTAACTATCGTCTTGAGTCC | |
| AACCCGGTAAGACACGACTTATCGCCACTGGCAGCAGCCACTGGTAACAGGATTAGCAGAGCGAGGTATGTAGGC | |
| GGTGCTACAGAGTTCTTGAAGTGGTGGCCTAACTACGGCTACACTAGAAGAACAGTATTTGGTATCTGCGCTCTG | |
| CTGAAGCCAGTTACCTTCGGAAAAAGAGTTGGTAGCTCTTGATCCGGCAAACAAACCACCGCTGGTAGCGGTGGT | |
| TTTTTTGTTTGCAAGCAGCAGATTACGCGCAGAAAAAAAGGATCTCAAGAAGATCCTTTGATCTTTTCTACGGGG | |
| TCTGACGCTCAGTGGAACGAAAACTCACGTTAAGGGATTTTGGTCATGAGATTATCAAAAAGGATCTTCACCTAG | |
| ATCCTTTTAAATTAAAAATGAAGTTTTAAATCAATCTAAAGTATATATGAGTAAACTTGGTCTGACAGTTAGAAA | |
| AACTCATCGAGCATCAAATGAAACTGCAATTTATTCATATCAGGATTATCAATACCATATTTTTGAAAAAGCCGT | |
| TTCTGTAATGAAGGAGAAAACTCACCGAGGCAGTTCCATAGGATGGCAAGATCCTGGTATCGGTCTGCGATTCCG | |
| ACTCGTCCAACATCAATACAACCTATTAATTTCCCCTCGTCAAAAATAAGGTTATCAAGTGAGAAATCACCATGA | |
| GTGACGACTGAATCCGGTGAGAATGGCAACAGCTTATGCATTTCTTTCCAGACTTGTTCAACAGGCCAGCCATTA | |
| CGCTCGTCATCAAAATCACTCGCATCAACCAAACCGTTATTCATTCGTGATTGCGCCTGAGCGAGACGAAATACG | |
| CGATCGCTGTTAAAAGGACAATTACAAACAGGAATCGAATGCAACCGGCGCAGGAACACTGCCAGCGCATCAACA | |
| ATATTTTCACCTGAATCAGGATATTCTTCTAATACCTGGAATGCTGTTTTTCCGGGGATCGCAGTGGTGAGTAAC | |
| CATGCATCATCAGGAGTACGGATAAAATGCTTGATGGTCGGAAGAGGCATAAATTCCGTCAGCCAGTTTAGTCTG | |
| ACCATCTCATCTGTAACATCATTGGCAACGCTACCTTTGCCATGTTTCAGAAACAACTCTGGCGCATCGGGCTTC | |
| CCATACAATCGATAGATTGTCGCACCTGATTGCCCGACATTATCGCGAGCCCATTTATACCCATATAAATCAGCA | |
| TCCATGTTGGAATTTAATCGCGGCCTAGAGCAAGACGTTTCCCGTTGAATATGGCTCATAACACCCCTTGTATTA | |
| CTGTTTATGTAAGCAGACAGTTTTATTGTTCATGATGATATATTTTTATCTTGTGCAATGTAACATCAGAGATTT | |
| TGAGACACAACAATTGGTCGACGGATCC | |
| Plasmid as defined in FIG. 2B (pDNA1 pGM830) | |
| Length: 10536; Molecule Type: DNA; Features Location/Qualifiers: source, | |
| 1..10536; mol_type, other DNA; note, pGM830; organism, synthetic construct | |
| SEQ ID NO: 4 | |
| GGTACCTCAATATTGGCCATTAGCCATATTATTCATTGGTTATATAGCATAAATCAATATTGGCTATTGGCCATT | |
| GCATACGTTGTATCTATATCATAATATGTACATTTATATTGGCTCATGTCCAATATGACCGCCATGTTGGCATTG | |
| ATTATTGACTAGTTATTAATAGTAATCAATTACGGGGTCATTAGTTCATAGCCCATATATGGAGTTCCGCGTTAC | |
| ATAACTTACGGTAAATGGCCCGCCTGGCTGACCGCCCAACGACCCCCGCCCATTGACGTCAATAATGACGTATGT | |
| TCCCATAGTAACGCCAATAGGGACTTTCCATTGACGTCAATGGGTGGAGTATTTACGGTAAACTGCCCACTTGGC | |
| AGTACATCAAGTGTATCATATGCCAAGTCCGCCCCCTATTGACGTCAATGACGGTAAATGGCCCGCCTGGCATTA | |
| TGCCCAGTACATGACCTTACGGGACTTTCCTACTTGGCAGTACATCTACGTATTAGTCATCGCTATTACCATGGT | |
| GATGCGGTTTTGGCAGTACACCAATGGGCGTGGATAGCGGTTTGACTCACGGGGATTTCCAAGTCTCCACCCCAT | |
| TGACGTCAATGGGAGTTTGTTTTGGCACCAAAATCAACGGGACTTTCCAAAATGTCGTAACAACTGCGATCGCCC | |
| GCCCCGTTGACGCAAATGGGCGGTAGGCGTGTACGGTGGGAGGTCTATATAAGCAGAGCTCGCTGGCTTGTAACT | |
| CAGTCTCTTACTAGGAGACCAGCTTGAGCCTGGGTGTTCGCTGGTTAGCCTAACCTGGTTGGCCACCAGGGGTAA | |
| GGACTCCTTGGCTTAGAAAGCTAATAAACTTGCCTGCATTAGAGCTTATCTGAGTCAAGTGTCCTCATTGACGCC | |
| TCACTCTCTTGAACGGGAATCTTCCTTACTGGGTTCTCTCTCTGACCCAGGCGAGAGAAACTCCAGCAGTGGCGC | |
| CCGAACAGGGACTTGAGTGAGAGTGTAGGCACGTACAGCTGAGAAGGCGTCGGACGCGAAGGAAGCGCGGGGTGC | |
| GACGCGACCAAGAAGGAGACTTGGTGAGTAGGCTTCTCGAGTGCCGGGAAAAAGCTCGAGCCTAGTTAGAGGACT | |
| AGGAGAGGCCGTAGCCGTAACTACTCTGGGCAAGTAGGGCAGGCGGTGGGTACGCAATTGGGGGCGGCTACCTCA | |
| GCACTAAATAGGAGACAATTAGACCAATTTGAGAAAATACGACTTCGCCCGAACGGAAAGAAAAAGTACCAAATT | |
| AAACATTTAATATTGGGCAGGCAAGGAGATTGGAGCGCTTCGGCCTCCATGAGAGGTTGTTGGAGACAGAGGAGG | |
| GGTGTAAAAGAATCATAGAAGTCCTCTACCCCCTAGAACCAACAGGATCGGAGGGCTTAAAAAGTCTGTTCAATC | |
| TTGTGTGCGTGCTATATTGCTTGCACAAGGAACAGAAAGTGAAAGACACAGAGGAAGCAGTAGCAACAGTAAGAC | |
| AACACTGCCATCTAGTGGAAAAAGAAAAAAGTGCAACAGAGACATCTAGTGGACAAAAGAAAAATGACAAGGGAA | |
| TAGCAGCGCCACCTGGTGGCAGTCAGAATTTTCCAGCGCAACAACAAGGAAATTGCCTGGGTACATGTACCCTTG | |
| TCACCGCGCACCTTAAATGCGTGGGTAAAAGCAGTAGAGGAGAAAAAATTTGGAGCAGAAATAGTACCCATGTTT | |
| CAAGCCCTATCGCCTGCAGGCCGTTTGTGCTAGGGTTCTTAGGCTTCTTGGGGGCTGCTGGAACTGCATTGGGAG | |
| CAGCGGCGACAGCCCTGACGGTCCAGTCTCAGCATTTGCTTGCTGGGATACTGCAGCAGCAGAAGAATCTGCTGG | |
| CGGCTGTGGAGGCTCAACAGCAGATGTTGAAGCTGACCATTTGGGGTGTTAAAAACCTCAATGCCCGCGTCACAG | |
| CCCTTGAGAAGTACCTAGAGGATCAGGCACGACTAAACTCCTGGGGGTGCGCATGGAAACAAGTATGTCATACCA | |
| CAGTGGAGTGGCCCTGGACAAATCGGACTCCGGATTGGCAAAATAAGACTTGGTTGGAGTGGGAAAGACAAATAG | |
| CTGATTTGGAAAGCAACATTACGAGACAATTAGTGAAGGCTAGAGAACAAGAGGAAAAGAATCTAGATGCCTATC | |
| AGAAGTTAACTAGTTGGTCAGATTTCTGGTCTTGGTTCGATTTCTCAAAATGGCTTAACATTTTAAAAAAGGGAT | |
| TTTTAGTAATAGTAGGAATAATAGGGTTAAGATTACTTTACACAGTATATGGATGTATAGTGAGGGTTAGGCAGG | |
| GATATGTTCCTCTATCTCCACAGATCCATATAAAGCGGCAATTTTAAAAGAAAGGGAGGAATAGGGGGACAGACT | |
| TCAGCAGAGAGACTAATTAATATAATAACAACACAATTAGAAATACAACATTTACAAACCAAAATTCAAAAAATT | |
| TTAAATTTTAGAGCCGCGGAGATCTGTTACATAACTTATGGTAAATGGCCTGCCTGGCTGACTGCCCAATGACCC | |
| CTGCCCAATGATGTCAATAATGATGTATGTTCCCATGTAATGCCAATAGGGACTTTCCATTGATGTCAATGGGTG | |
| GAGTATTTATGGTAACTGCCCACTTGGCAGTACATCAAGTGTATCATATGCCAAGTATGCCCCCTATTGATGTCA | |
| ATGATGGTAAATGGCCTGCCTGGCATTATGCCCAGTACATGACCTTATGGGACTTTCCTACTTGGCAGTACATCT | |
| ATGTATTAGTCATTGCTATTACCATGGGAATTCACTAGTGGAGAAGAGCATGCTTGAGGGCTGAGTGCCCCTCAG | |
| TGGGCAGAGAGCACATGGCCCACAGTCCCTGAGAAGTTGGGGGGAGGGGTGGGCAATTGAACTGGTGCCTAGAGA | |
| AGGTGGGGCTTGGGTAAACTGGGAAAGTGATGTGGTGTACTGGCTCCACCTTTTTCCCCAGGGTGGGGGAGAACC | |
| ATATATAAGTGCAGTAGTCTCTGTGAACATTCAAGCTTCTGCCTTCTCCCTCCTGTGAGTTTGCTAGCCACCATG | |
| CAGAGAAGCCCTCTGGAGAAGGCCTCTGTGGTGAGCAAGCTGTTCTTCAGCTGGACCAGGCCCATCCTGAGGAAG | |
| GGCTACAGGCAGAGACTGGAGCTGTCTGACATCTACCAGATCCCCTCTGTGGACTCTGCTGACAACCTGTCTGAG | |
| AAGCTGGAGAGGGAGTGGGATAGAGAGCTGGCCAGCAAGAAGAACCCCAAGCTGATCAATGCCCTGAGGAGATGC | |
| TTCTTCTGGAGATTCATGTTCTATGGCATCTTCCTGTACCTGGGGGAAGTGACCAAGGCTGTGCAGCCTCTGCTG | |
| CTGGGCAGAATCATTGCCAGCTATGACCCTGACAACAAGGAGGAGAGGAGCATTGCCATCTACCTGGGCATTGGC | |
| CTGTGCCTGCTGTTCATTGTGAGGACCCTGCTGCTGCACCCTGCCATCTTTGGCCTGCACCACATTGGCATGCAG | |
| ATGAGGATTGCCATGTTCAGCCTGATCTACAAGAAAACCCTGAAGCTGTCCAGCAGAGTGCTGGACAAGATCAGC | |
| ATTGGCCAGCTGGTGAGCCTGCTGAGCAACAACCTGAACAAGTTTGATGAGGGCCTGGCCCTGGCCCACTTTGTG | |
| TGGATTGCCCCTCTGCAGGTGGCCCTGCTGATGGGCCTGATTTGGGAGCTGCTGCAGGCCTCTGCCTTTTGTGGC | |
| CTGGGCTTCCTGATTGTGCTGGCCCTGTTTCAGGCTGGCCTGGGCAGGATGATGATGAAGTACAGGGACCAGAGG | |
| GCAGGCAAGATCAGTGAGAGGCTGGTGATCACCTCTGAGATGATTGAGAACATCCAGTCTGTGAAGGCCTACTGT | |
| TGGGAGGAAGCTATGGAGAAGATGATTGAAAACCTGAGGCAGACAGAGCTGAAGCTGACCAGGAAGGCTGCCTAT | |
| GTGAGATACTTCAACAGCTCTGCCTTCTTCTTCTCTGGCTTCTTTGTGGTGTTCCTGTCTGTGCTGCCCTATGCC | |
| CTGATCAAGGGGATCATCCTGAGAAAGATTTTCACCACCATCAGCTTCTGCATTGTGCTGAGGATGGCTGTGACC | |
| AGACAGTTCCCCTGGGCTGTGCAGACCTGGTATGACAGCCTGGGGGCCATCAACAAGATCCAGGACTTCCTGCAG | |
| AAGCAGGAGTACAAGACCCTGGAGTACAACCTGACCACCACAGAAGTGGTGATGGAGAATGTGACAGCCTTCTGG | |
| GAGGAGGGCTTTGGGGAGCTGTTTGAGAAGGCCAAGCAGAACAACAACAACAGAAAGACCAGCAATGGGGATGAC | |
| TCCCTGTTCTTCTCCAACTTCTCCCTGCTGGGCACACCTGTGCTGAAGGACATCAACTTCAAGATTGAGAGGGGG | |
| CAGCTGCTGGCTGTGGCTGGATCTACAGGGGCTGGCAAGACCAGCCTGCTGATGATGATCATGGGGGAGCTGGAG | |
| CCTTCTGAGGGCAAGATCAAGCACTCTGGCAGGATCAGCTTTTGCAGCCAGTTCAGCTGGATCATGCCTGGCACC | |
| ATCAAGGAGAACATCATCTTTGGAGTGAGCTATGATGAGTACAGATACAGGAGTGTGATCAAGGCCTGCCAGCTG | |
| GAGGAGGACATCAGCAAGTTTGCTGAGAAGGACAACATTGTGCTGGGGGAGGGAGGCATTACACTGTCTGGGGGC | |
| CAGAGAGCCAGAATCAGCCTGGCCAGGGCTGTGTACAAGGATGCTGACCTGTACCTGCTGGACTCCCCCTTTGGC | |
| TACCTGGATGTGCTGACAGAGAAGGAGATTTTTGAGAGCTGTGTGTGCAAGCTGATGGCCAACAAGACCAGAATC | |
| CTGGTGACCAGCAAGATGGAGCACCTGAAGAAGGCTGACAAGATCCTGATCCTGCATGAGGGCAGCAGCTACTTC | |
| TATGGGACCTTCTCTGAGCTGCAGAACCTGCAGCCTGACTTCAGCTCTAAGCTGATGGGCTGTGACAGCTTTGAC | |
| CAGTTCTCTGCTGAGAGGAGGAACAGCATCCTGACAGAGACCCTGCACAGATTCAGCCTGGAGGGAGATGCCCCT | |
| GTGAGCTGGACAGAGACCAAGAAGCAGAGCTTCAAGCAGACAGGGGAGTTTGGGGAGAAGAGGAAGAACTCCATC | |
| CTGAACCCCATCAACAGCATCAGGAAGTTCAGCATTGTGCAGAAAACCCCCCTGCAGATGAATGGCATTGAGGAA | |
| GATTCTGATGAGCCCCTGGAGAGGAGACTGAGCCTGGTGCCTGATTCTGAGCAGGGAGAGGCCATCCTGCCTAGG | |
| ATCTCTGTGATCAGCACAGGCCCTACACTGCAGGCCAGAAGGAGGCAGTCTGTGCTGAACCTGATGACCCACTCT | |
| GTGAACCAGGGCCAGAACATCCACAGGAAAACCACAGCCTCCACCAGGAAAGTGAGCCTGGCCCCTCAGGCCAAT | |
| CTGACAGAGCTGGACATCTACAGCAGGAGGCTGTCTCAGGAGACAGGCCTGGAGATTTCTGAGGAGATCAATGAG | |
| GAGGACCTGAAAGAGTGCTTCTTTGATGACATGGAGAGCATCCCTGCTGTGACCACCTGGAACACCTACCTGAGA | |
| TACATCACAGTGCACAAGAGCCTGATCTTTGTGCTGATCTGGTGCCTGGTGATCTTCCTGGCTGAAGTGGCTGCC | |
| TCTCTGGTGGTGCTGTGGCTGCTGGGAAACACCCCACTGCAGGACAAGGGCAACAGCACCCACAGCAGGAACAAC | |
| AGCTATGCTGTGATCATCACCTCCACCTCCAGCTACTATGTGTTCTACATCTATGTGGGAGTGGCTGATACCCTG | |
| CTGGCTATGGGCTTCTTTAGAGGCCTGCCCCTGGTGCACACACTGATCACAGTGAGCAAGATCCTCCACCACAAG | |
| ATGCTGCACTCTGTGCTGCAGGCTCCTATGAGCACCCTGAATACCCTGAAGGCTGGGGGCATCCTGAACAGATTC | |
| TCCAAGGATATTGCCATCCTGGATGACCTGCTGCCTCTCACCATCTTTGACTTCATCCAGCTGCTGCTGATTGTG | |
| ATTGGGGCCATTGCTGTGGTGGCAGTGCTGCAGCCCTACATCTTTGTGGCCACAGTGCCTGTGATTGTGGCCTTC | |
| ATCATGCTGAGGGCCTACTTTCTGCAGACCTCCCAGCAGCTGAAGCAGCTGGAGTCTGAGGGCAGAAGCCCCATC | |
| TTCACCCACCTGGTGACAAGCCTGAAGGGCCTGTGGACCCTGAGAGCCTTTGGCAGGCAGCCCTACTTTGAGACC | |
| CTGTTCCACAAGGCCCTGAACCTGCACACAGCCAACTGGTTCCTCTACCTGTCCACCCTGAGATGGTTCCAGATG | |
| AGAATTGAGATGATCTTTGTCATCTTCTTCATTGCTGTGACCTTCATCAGCATTCTGACCACAGGAGAGGGAGAG | |
| GGCAGAGTGGGCATTATCCTGACCCTGGCCATGAACATCATGAGCACACTGCAGTGGGCAGTGAACAGCAGCATT | |
| GATGTGGACAGCCTGATGAGGAGTGTGAGCAGAGTGTTCAAGTTCATTGATATGCCCACAGAGGGCAAGCCTACC | |
| AAGAGCACCAAGCCCTACAAGAATGGCCAGCTGAGCAAAGTGATGATCATTGAGAACAGCCATGTGAAGAAGGAT | |
| GATATCTGGCCCAGTGGAGGCCAGATGACAGTGAAGGACCTGACAGCCAAGTACACAGAGGGGGGCAATGCTATC | |
| CTGGAGAACATCTCCTTCAGCATCTCCCCTGGCCAGAGAGTGGGACTGCTGGGAAGAACAGGCTCTGGCAAGTCT | |
| ACCCTGCTGTCTGCCTTCCTGAGGCTGCTGAACACAGAGGGAGAGATCCAGATTGATGGAGTGTCCTGGGACAGC | |
| ATCACACTGCAGCAGTGGAGGAAGGCCTTTGGTGTGATCCCCCAGAAAGTGTTCATCTTCAGTGGCACCTTCAGG | |
| AAGAACCTGGACCCCTATGAGCAGTGGTCTGACCAGGAGATTTGGAAAGTGGCTGATGAAGTGGGCCTGAGAAGT | |
| GTGATTGAGCAGTTCCCTGGCAAGCTGGACTTTGTCCTGGTGGATGGGGGCTGTGTGCTGAGCCATGGCCACAAG | |
| CAGCTGATGTGCCTGGCCAGATCAGTGCTGAGCAAGGCCAAGATCCTGCTGCTGGATGAGCCTTCTGCCCACCTG | |
| GATCCTGTGACCTACCAGATCATCAGGAGGACCCTCAAGCAGGCCTTTGCTGACTGCACAGTCATCCTGTGTGAG | |
| CACAGGATTGAGGCCATGCTGGAGTGCCAGCAGTTCCTGGTGATTGAGGAGAACAAAGTGAGGCAGTATGACAGC | |
| ATCCAGAAGCTGCTGAATGAGAGGAGCCTGTTCAGGCAGGCCATCAGCCCCTCTGATAGAGTGAAGCTGTTCCCC | |
| CACAGGAACAGCTCCAAGTGCAAGAGCAAGCCCCAGATTGCTGCCCTGAAGGAGGAGACAGAGGAGGAAGTGCAG | |
| GACACCAGGCTGTGAGGGCCCAATCAACCTCTGGATTACAAAATTTGTGAAAGATTGACTGGTATTCTTAACTAT | |
| GTTGCTCCTTTTACGCTATGTGGATACGCTGCTTTAATGCCTTTGTATCATGCTATTGCTTCCCGTATGGCTTTC | |
| ATTTTCTCCTCCTTGTATAAATCCTGGTTGCTGTCTCTTTATGAGGAGTTGTGGCCCGTTGTCAGGCAACGTGGC | |
| GTGGTGTGCACTGTGTTTGCTGACGCAACCCCCACTGGTTGGGGCATTGCCACCACCTGTCAGCTCCTTTCCGGG | |
| ACTTTCGCTTTCCCCCTCCCTATTGCCACGGCGGAACTCATCGCCGCCTGCCTTGCCCGCTGCTGGACAGGGGCT | |
| CGGCTGTTGGGCACTGACAATTCCGTGGTGTTGTCGGGGAAATCATCGTCCTTTCCTTGGCTGCTCGCCTGTGTT | |
| GCCACCTGGATTCTGCGCGGGACGTCCTTCTGCTACGTCCCTTCGGCCCTCAATCCAGCGGACCTTCCTTCCCGC | |
| GGCCTGCTGCCGGCTCTGCGGCCTCTTCCGCGTCTTCGCCTTCGCCCTCAGACGAGTCGGATCTCCCTTTGGGCC | |
| GCCTCCCCGCAAGCTTCGCACTTTTTAAAAGAAAAGGGAGGACTGGATGGGATTTATTACTCCGATAGGACGCTG | |
| GCTTGTAACTCAGTCTCTTACTAGGAGACCAGCTTGAGCCTGGGTGTTCGCTGGTTAGCCTAACCTGGTTGGCCA | |
| CCAGGGGTAAGGACTCCTTGGCTTAGAAAGCTAATAAACTTGCCTGCATTAGAGCTCTTACGCGTCCCGGGCTCG | |
| AGATCCGCATCTCAATTAGTCAGCAACCATAGTCCCGCCCCTAACTCCGCCCATCCCGCCCCTAACTCCGCCCAG | |
| TTCCGCCCATTCTCCGCCCCATGGCTGACTAATTTTTTTTATTTATGCAGAGGCCGAGGCCGCCTCGGCCTCTGA | |
| GCTATTCCAGAAGTAGTGAGGAGGCTTTTTTGGAGGCCTAGGCTTTTGCAAAAAGCTAACTTGTTTATTGCAGCT | |
| TATAATGGTTACAAATAAAGCAATAGCATCACAAATTTCACAAATAAAGCATTTTTTTCACTGCATTCTAGTTGT | |
| GGTTTGTCCAAACTCATCAATGTATCTTATCATGTCTGTCCGCTTCCTCGCTCACTGACTCGCTGCGCTCGGTCG | |
| TTCGGCTGCGGCGAGCGGTATCAGCTCACTCAAAGGCGGTAATACGGTTATCCACAGAATCAGGGGATAACGCAG | |
| GAAAGAACATGTGAGCAAAAGGCCAGCAAAAGGCCAGGAACCGTAAAAAGGCCGCGTTGCTGGCGTTTTTCCATA | |
| GGCTCCGCCCCCCTGACGAGCATCACAAAAATCGACGCTCAAGTCAGAGGTGGCGAAACCCGACAGGACTATAAA | |
| GATACCAGGCGTTTCCCCCTGGAAGCTCCCTCGTGCGCTCTCCTGTTCCGACCCTGCCGCTTACCGGATACCTGT | |
| CCGCCTTTCTCCCTTCGGGAAGCGTGGCGCTTTCTCATAGCTCACGCTGTAGGTATCTCAGTTCGGTGTAGGTCG | |
| TTCGCTCCAAGCTGGGCTGTGTGCACGAACCCCCCGTTCAGCCCGACCGCTGCGCCTTATCCGGTAACTATCGTC | |
| TTGAGTCCAACCCGGTAAGACACGACTTATCGCCACTGGCAGCAGCCACTGGTAACAGGATTAGCAGAGCGAGGT | |
| ATGTAGGCGGTGCTACAGAGTTCTTGAAGTGGTGGCCTAACTACGGCTACACTAGAAGAACAGTATTTGGTATCT | |
| GCGCTCTGCTGAAGCCAGTTACCTTCGGAAAAAGAGTTGGTAGCTCTTGATCCGGCAAACAAACCACCGCTGGTA | |
| GCGGTGGTTTTTTTGTTTGCAAGCAGCAGATTACGCGCAGAAAAAAAGGATCTCAAGAAGATCCTTTGATCTTTT | |
| CTACGGGGTCTGACGCTCAGTGGAACGAAAACTCACGTTAAGGGATTTTGGTCATGAGATTATCAAAAAGGATCT | |
| TCACCTAGATCCTTTTAAATTAAAAATGAAGTTTTAAATCAATCTAAAGTATATATGAGTAAACTTGGTCTGACA | |
| GTTAGAAAAACTCATCGAGCATCAAATGAAACTGCAATTTATTCATATCAGGATTATCAATACCATATTTTTGAA | |
| AAAGCCGTTTCTGTAATGAAGGAGAAAACTCACCGAGGCAGTTCCATAGGATGGCAAGATCCTGGTATCGGTCTG | |
| CGATTCCGACTCGTCCAACATCAATACAACCTATTAATTTCCCCTCGTCAAAAATAAGGTTATCAAGTGAGAAAT | |
| CACCATGAGTGACGACTGAATCCGGTGAGAATGGCAACAGCTTATGCATTTCTTTCCAGACTTGTTCAACAGGCC | |
| AGCCATTACGCTCGTCATCAAAATCACTCGCATCAACCAAACCGTTATTCATTCGTGATTGCGCCTGAGCGAGAC | |
| GAAATACGCGATCGCTGTTAAAAGGACAATTACAAACAGGAATCGAATGCAACCGGCGCAGGAACACTGCCAGCG | |
| CATCAACAATATTTTCACCTGAATCAGGATATTCTTCTAATACCTGGAATGCTGTTTTTCCGGGGATCGCAGTGG | |
| TGAGTAACCATGCATCATCAGGAGTACGGATAAAATGCTTGATGGTCGGAAGAGGCATAAATTCCGTCAGCCAGT | |
| TTAGTCTGACCATCTCATCTGTAACATCATTGGCAACGCTACCTTTGCCATGTTTCAGAAACAACTCTGGCGCAT | |
| CGGGCTTCCCATACAATCGATAGATTGTCGCACCTGATTGCCCGACATTATCGCGAGCCCATTTATACCCATATA | |
| AATCAGCATCCATGTTGGAATTTAATCGCGGCCTAGAGCAAGACGTTTCCCGTTGAATATGGCTCATAACACCCC | |
| TTGTATTACTGTTTATGTAAGCAGACAGTTTTATTGTTCATGATGATATATTTTTATCTTGTGCAATGTAACATC | |
| AGAGATTTTGAGACACAACAATTGGTCGACGGATCC | |
| Plasmid as defined in FIG. 2C (pDNA2a pGM691) | |
| Length: 9064; Molecule Type: DNA; Features Location/Qualifiers: source, | |
| 1..9064; mol_type, other DNA; note, pGM691; organism, synthetic construct | |
| SEQ ID NO: 5 | |
| ATTGATTATTGACTAGTTATTAATAGTAATCAATTACGGGGTCATTAGTTCATAGCCCATATATGGAGTTCCGCG | |
| TTACATAACTTACGGTAAATGGCCCGCCTGGCTGACCGCCCAACGACCCCCGCCCATTGACGTCAATAATGACGT | |
| ATGTTCCCATAGTAACGCCAATAGGGACTTTCCATTGACGTCAATGGGTGGAGTATTTACGGTAAACTGCCCACT | |
| TGGCAGTACATCAAGTGTATCATATGCCAAGTACGCCCCCTATTGACGTCAATGACGGTAAATGGCCCGCCTGGC | |
| ATTATGCCCAGTACATGACCTTATGGGACTTTCCTACTTGGCAGTACATCTACGTATTAGTCATCGCTATTACCA | |
| TGGTCGAGGTGAGCCCCACGTTCTGCTTCACTCTCCCCATCTCCCCCCCCTCCCCACCCCCAATTTTGTATTTAT | |
| TTATTTTTTAATTATTTTGTGCAGCGATGGGGGCGGGGGGGGGGGGGGGGCGCGCGCCAGGCGGGGCGGGGCGGG | |
| GCGAGGGGCGGGGCGGGGCGAGGCGGAGAGGTGCGGCGGCAGCCAATCAGAGCGGCGCGCTCCGAAAGTTTCCTT | |
| TTATGGCGAGGCGGCGGCGGCGGCGGCCCTATAAAAAGCGAAGCGCGCGGCGGGCGGGAGTCGCTGCGCGCTGCC | |
| TTCGCCCCGTGCCCCGCTCCGCCGCCGCCTCGCGCCGCCCGCCCCGGCTCTGACTGACCGCGTTACTCCCACAGG | |
| TGAGCGGGCGGGACGGCCCTTCTCCTCCGGGCTGTAATTAGCGCTTGGTTTAATGACGGCTTGTTTCTTTTCTGT | |
| GGCTGCGTGAAAGCCTTGAGGGGCTCCGGGAGGGCCCTTTGTGCGGGGGGAGCGGCTCGGGGGGTGCGTGCGTGT | |
| GTGTGTGCGTGGGGAGCGCCGCGTGCGGCTCCGCGCTGCCCGGCGGCTGTGAGCGCTGCGGGCGCGGCGCGGGGC | |
| TTTGTGCGCTCCGCAGTGTGCGCGAGGGGAGCGCGGCCGGGGGCGGTGCCCCGCGGTGCGGGGGGGGCTGCGAGG | |
| GGAACAAAGGCTGCGTGCGGGGTGTGTGCGTGGGGGGGTGAGCAGGGGGTGTGGGCGCGTCGGTCGGGCTGCAAC | |
| CCCCCCTGCACCCCCCTCCCCGAGTTGCTGAGCACGGCCCGGCTTCGGGTGCGGGGCTCCGTACGGGGCGTGGCG | |
| CGGGGCTCGCCGTGCCGGGCGGGGGGTGGCGGCAGGTGGGGGTGCCGGGCGGGGCGGGGCCGCCTCGGGCCGGGG | |
| AGGGCTCGGGGGAGGGGCGCGGCGGCCCCCGGAGCGCCGGCGGCTGTCGAGGCGCGGCGAGCCGCAGCCATTGCC | |
| TTTTATGGTAATCGTGCGAGAGGGCGCAGGGACTTCCTTTGTCCCAAATCTGTGCGGAGCCGAAATCTGGGAGGC | |
| GCCGCCGCACCCCCTCTAGCGGGCGCGGGGCGAAGCGGTGCGGCGCCGGCAGGAAGGAAATGGGCGGGGAGGGCC | |
| TTCGTGCGTCGCCGCGCCGCCGTCCCCTTCTCCCTCTCCAGCCTCGGGGCTGTCCGCGGGGGGACGGCTGCCTTC | |
| GGGGGGGACGGGGCAGGGCGGGGTTCGGCTTCTGGCGTGTGACCGGCGGCTCTAGAGCCTCTGCTAACCATGTTC | |
| ATGCCTTCTTCTTTTTCCTACAGCTCCTGGGCAACGTGCTGGTTATTGTGCTGTCTCATCATTTTGGCAAAGAAT | |
| TGCTCGAGCCACCATGGGAGCTGCCACATCTGCCCTGAATAGACGGCAGCTGGACCAGTTCGAGAAGATCAGACT | |
| GCGGCCCAACGGCAAGAAGAAGTACCAGATCAAGCACCTGATCTGGGCCGGCAAAGAGATGGAAAGATTCGGCCT | |
| GCACGAGCGGCTGCTGGAAACCGAGGAAGGCTGCAAGAGAATTATCGAGGTGCTGTACCCTCTGGAACCTACCGG | |
| CTCTGAGGGCCTGAAGTCCCTGTTCAATCTCGTGTGCGTGCTGTACTGCCTGCACAAAGAACAGAAAGTGAAGGA | |
| CACCGAAGAGGCCGTGGCCACAGTTAGACAGCACTGCCACCTGGTGGAAAAAGAGAAGTCCGCCACAGAGACAAG | |
| CAGCGGCCAGAAGAAGAACGACAAGGGAATTGCTGCCCCTCCTGGCGGCAGCCAGAATTTTCCTGCTCAGCAGCA | |
| GGGAAACGCCTGGGTGCACGTTCCACTGAGCCCTAGAACACTGAATGCCTGGGTCAAAGCCGTGGAAGAGAAGAA | |
| GTTTGGCGCCGAGATCGTGCCCATGTTCCAGGCTCTGTCTGAGGGCTGCACCCCTTACGACATCAACCAGATGCT | |
| GAACGTGCTGGGAGATCACCAGGGCGCTCTGCAGATCGTGAAAGAGATCATCAACGAAGAGGCTGCCCAGTGGGA | |
| CGTGACACATCCATTGCCTGCTGGACCTCTGCCAGCCGGACAACTGAGAGATCCTAGAGGCTCTGATATCGCCGG | |
| CACCACCAGCTCTGTGCAAGAGCAGCTGGAATGGATCTACACCGCCAATCCTAGAGTGGACGTGGGCGCCATCTA | |
| CAGAAGATGGATCATCCTGGGCCTGCAGAAATGCGTGAAGATGTACAACCCCGTGTCCGTGCTGGACATCAGACA | |
| GGGACCCAAAGAGCCCTTCAAGGACTACGTGGACCGGTTCTATAAGGCCATTAGAGCCGAGCAGGCCAGCGGCGA | |
| AGTGAAGCAGTGGATGACAGAGAGCCTGCTGATCCAGAACGCCAATCCAGACTGCAAAGTGATCCTGAAAGGCCT | |
| GGGCATGCACCCCACACTGGAAGAGATGCTGACAGCCTGTCAAGGCGTTGGCGGCCCTTCTTACAAAGCCAAAGT | |
| GATGGCCGAGATGATGCAGACCATGCAGAACCAGAACATGGTGCAGCAAGGCGGCCCTAAGAGACAGAGGCCTCC | |
| TCTGAGATGCTACAACTGCGGCAAGTTCGGCCACATGCAGAGACAGTGTCCTGAGCCTAGGAAAACAAAATGTCT | |
| AAAGTGTGGAAAATTGGGACACCTAGCAAAAGACTGCAGGGGACAGGTGAATTTTTTAGGGTATGGACGGTGGAT | |
| GGGGGCAAAACCGAGAAATTTTCCCGCCGCTACTCTTGGAGCGGAACCGAGTGCGCCTCCTCCACCGAGCGGCAC | |
| CACCCCATACGACCCAGCAAAGAAGCTCCTGCAGCAATATGCAGAGAAAGGGAAACAACTGAGGGAGCAAAAGAG | |
| GAATCCACCGGCAATGAATCCGGATTGGACCGAGGGATATTCTTTGAACTCCCTCTTTGGAGAAGACCAATAAAG | |
| ACCGTGTACATCGAGGGCGTGCCCATCAAGGCTCTGCTGGATACAGGCGCCGACGACACCATCATCAAAGAGAAC | |
| GACCTGCAGCTGAGCGGCCCTTGGAGGCCTAAGATCATTGGAGGAATCGGCGGAGGCCTGAACGTCAAAGAGTAC | |
| AACGACCGGGAAGTGAAGATCGAGGACAAGATCCTGAGGGGCACAATCCTGCTGGGCGCCACACCTATCAACATC | |
| ATCGGCAGAAATCTGCTGGCCCCTGCCGGCGCTAGACTGGTTATGGGACAGCTCTCTGAGAAGATCCCCGTGACA | |
| CCCGTGAAGCTGAAAGAAGGCGCTAGAGGACCTTGTGTGCGACAGTGGCCTCTGAGCAAAGAGAAGATTGAGGCC | |
| CTGCAAGAAATCTGTAGCCAGCTGGAACAAGAGGGCAAGATCAGCAGAGTTGGCGGCGAGAACGCCTACAATACC | |
| CCTATCTTCTGCATCAAGAAAAAGGACAAGAGCCAGTGGCGGATGCTGGTGGACTTTAGAGAGCTGAACAAGGCT | |
| ACCCAGGACTTCTTCGAGGTGCAGCTGGGAATTCCTCATCCTGCCGGCCTGCGGAAGATGAGACAGATCACAGTG | |
| CTGGATGTGGGCGACGCCTACTACAGCATCCCTCTGGACCCCAACTTCAGAAAGTACACCGCCTTCACAATCCCC | |
| ACCGTGAACAATCAAGGCCCTGGCATCAGATACCAGTTCAACTGCCTGCCTCAAGGCTGGAAGGGCAGCCCCACC | |
| ATTTTTCAGAATACCGCCGCCAGCATCCTGGAAGAAATCAAGAGAAACCTGCCTGCTCTGACCATCGTGCAGTAC | |
| ATGGACGATCTGTGGGTCGGAAGCCAAGAGAATGAGCACACCCACGACAAGCTGGTGGAACAGCTGAGAACAAAG | |
| CTGCAGGCCTGGGGCCTCGAAACCCCTGAGAAGAAGGTGCAGAAAGAACCTCCTTACGAGTGGATGGGCTACAAG | |
| CTGTGGCCTCACAAGTGGGAGCTGAGCCGGATTCAGCTCGAAGAGAAGGACGAGTGGACCGTGAACGACATCCAG | |
| AAACTCGTGGGCAAGCTGAATTGGGCAGCCCAGCTGTATCCCGGCCTGAGGACCAAGAACATCTGCAAGCTGATC | |
| CGGGGAAAGAAGAACCTGCTGGAACTGGTCACATGGACACCTGAGGCCGAGGCCGAATATGCCGAGAATGCCGAA | |
| ATCCTGAAAACCGAGCAAGAGGGGACCTACTACAAGCCTGGCATTCCAATCAGAGCTGCCGTGCAGAAACTGGAA | |
| GGCGGCCAGTGGTCCTACCAGTTTAAGCAAGAAGGCCAGGTCCTGAAAGTGGGCAAGTACACCAAGCAGAAGAAC | |
| ACCCACACCAACGAGCTGAGGACACTGGCTGGCCTGGTCCAGAAAATCTGCAAAGAGGCCCTGGTCATTTGGGGC | |
| ATCCTGCCTGTTCTGGAACTGCCCATTGAGCGGGAAGTGTGGGAACAGTGGTGGGCCGATTACTGGCAAGTGTCT | |
| TGGATCCCCGAGTGGGACTTCGTGTCTACCCCTCCTCTGCTGAAACTGTGGTACACCCTGACAAAAGAGCCCATT | |
| CCTAAAGAGGACGTCTACTACGTTGACGGCGCCTGCAACCGGAACTCCAAAGAAGGCAAGGCCGGCTACATCAGC | |
| CAGTACGGCAAGCAGAGAGTGGAAACCCTGGAAAACACCACCAACCAGCAGGCCGAGCTGACCGCCATTAAGATG | |
| GCCCTGGAAGATAGCGGCCCCAATGTGAACATCGTGACCGACTCTCAGTACGCCATGGGAATCCTGACAGCCCAG | |
| CCTACACAGAGCGATAGCCCTCTGGTTGAGCAGATCATTGCCCTGATGATTCAGAAGCAGCAAATCTACCTGCAG | |
| TGGGTGCCCGCTCACAAAGGCATCGGCGGAAACGAAGAGATCGATAAGCTGGTGTCCAAGGGAATCAGACGGGTG | |
| CTGTTCCTGGAAAAGATTGAAGAGGCCCAAGAGGAACACGAGCGCTACCACAACAACTGGAAGAATCTGGCCGAC | |
| ACCTACGGACTGCCCCAGATCGTGGCCAAAGAAATCGTGGCTATGTGCCCCAAGTGTCAGATCAAGGGCGAACCT | |
| GTGCACGGCCAAGTGGATGCTTCTCCTGGCACATGGCAGATGGACTGTACCCACCTGGAAGGCAAAGTGGTCATC | |
| GTGGCTGTGCACGTGGCCTCCGGCTTTATTGAGGCCGAAGTGATCCCCAGAGAGACAGGCAAAGAAACCGCCAAG | |
| TTCCTGCTGAAGATCCTGTCCAGATGGCCCATCACACAGCTGCACACCGACAACGGCCCTAACTTCACATCTCAA | |
| GAGGTGGCCGCCATCTGTTGGTGGGGAAAGATTGAGCACACAACCGGCATTCCCTACAATCCACAGAGCCAGGGC | |
| AGCATCGAGTCCATGAACAAGCAGCTCAAAGAGATTATCGGCAAGATCCGGGACGACTGCCAGTACACAGAAACA | |
| GCCGTGCTGATGGCCTGTCACATCCACAACTTCAAGCGGAAAGGCGGCATCGGAGGACAGACATCTGCCGAGAGA | |
| CTGATCAATATCATCACCACTCAGCTGGAAATCCAGCACCTCCAGACCAAGATCCAGAAGATTCTGAACTTCCGG | |
| GTGTACTACCGCGAGGGCAGAGATCCTGTTTGGAAAGGCCCAGCACAGCTGATCTGGAAAGGCGAAGGTGCCGTG | |
| GTGCTGAAGGATGGCTCTGATCTGAAGGTGGTGCCCAGACGGAAGGCCAAGATTATCAAGGATTACGAGCCCAAA | |
| CAGCGCGTGGGCAATGAAGGCGACGTTGAGGGCACAAGAGGCAGCGACAATTGAAATTCACTCCTCAGGTGCAGG | |
| CTGCCTATCAGAAGGTGGTGGCTGGTGTGGCCAATGCCCTGGCTCACAAATACCACTGAGATCTTTTTCCCTCTG | |
| CCAAAAATTATGGGGACATCATGAAGCCCCTTGAGCATCTGACTTCTGGCTAATAAAGGAAATTTATTTTCATTG | |
| CAATAGTGTGTTGGAATTTTTTGTGTCTCTCACTCGGAAGGACATATGGGAGGGCAAATCATTTAAAACATCAGA | |
| ATGAGTATTTGGTTTAGAGTTTGGCAACATATGCCCATATGCTGGCTGCCATGAACAAAGGTTGGCTATAAAGAG | |
| GTCATCAGTATATGAAACAGCCCCCTGCTGTCCATTCCTTATTCCATAGAAAAGCCTTGACTTGAGGTTAGATTT | |
| TTTTTATATTTTGTTTTGTGTTATTTTTTTCTTTAACATCCCTAAAATTTTCCTTACATGTTTTACTAGCCAGAT | |
| TTTTCCTCCTCTCCTGACTACTCCCAGTCATAGCTGTCCCTCTTCTCTTATGGAGATCCCTCGACCTGCAGCCCA | |
| AGCTTGGCGTAATCATGGTCATAGCTGTTTCCTGTGTGAAATTGTTATCCGCTCACAATTCCACACAACATACGA | |
| GCCGGAAGCATAAAGTGTAAAGCCTGGGGTGCCTAATGAGTGAGCTAACTCACATTAATTGCGTTGCGCTCACTG | |
| CCCGCTTTCCAGTCGGGAAACCTGTCGTGCCAGCGGATCCGCATCTCAATTAGTCAGCAACCATAGTCCCGCCCC | |
| TAACTCCGCCCATCCCGCCCCTAACTCCGCCCAGTTCCGCCCATTCTCCGCCCCATGGCTGACTAATTTTTTTTA | |
| TTTATGCAGAGGCCGAGGCCGCCTCGGCCTCTGAGCTATTCCAGAAGTAGTGAGGAGGCTTTTTTGGAGGCCTAG | |
| GCTTTTGCAAAAAGCTAACTTGTTTATTGCAGCTTATAATGGTTACAAATAAAGCAATAGCATCACAAATTTCAC | |
| AAATAAAGCATTTTTTTCACTGCATTCTAGTTGTGGTTTGTCCAAACTCATCAATGTATCTTATCATGTCTGTCC | |
| GCTTCCTCGCTCACTGACTCGCTGCGCTCGGTCGTTCGGCTGCGGCGAGCGGTATCAGCTCACTCAAAGGCGGTA | |
| ATACGGTTATCCACAGAATCAGGGGATAACGCAGGAAAGAACATGTGAGCAAAAGGCCAGCAAAAGGCCAGGAAC | |
| CGTAAAAAGGCCGCGTTGCTGGCGTTTTTCCATAGGCTCCGCCCCCCTGACGAGCATCACAAAAATCGACGCTCA | |
| AGTCAGAGGTGGCGAAACCCGACAGGACTATAAAGATACCAGGCGTTTCCCCCTGGAAGCTCCCTCGTGCGCTCT | |
| CCTGTTCCGACCCTGCCGCTTACCGGATACCTGTCCGCCTTTCTCCCTTCGGGAAGCGTGGCGCTTTCTCATAGC | |
| TCACGCTGTAGGTATCTCAGTTCGGTGTAGGTCGTTCGCTCCAAGCTGGGCTGTGTGCACGAACCCCCCGTTCAG | |
| CCCGACCGCTGCGCCTTATCCGGTAACTATCGTCTTGAGTCCAACCCGGTAAGACACGACTTATCGCCACTGGCA | |
| GCAGCCACTGGTAACAGGATTAGCAGAGCGAGGTATGTAGGCGGTGCTACAGAGTTCTTGAAGTGGTGGCCTAAC | |
| TACGGCTACACTAGAAGAACAGTATTTGGTATCTGCGCTCTGCTGAAGCCAGTTACCTTCGGAAAAAGAGTTGGT | |
| AGCTCTTGATCCGGCAAACAAACCACCGCTGGTAGCGGTGGTTTTTTTGTTTGCAAGCAGCAGATTACGCGCAGA | |
| AAAAAAGGATCTCAAGAAGATCCTTTGATCTTTTCTACGGGGTCTGACGCTCAGTGGAACGAAAACTCACGTTAA | |
| GGGATTTTGGTCATGAGATTATCAAAAAGGATCTTCACCTAGATCCTTTTAAATTAAAAATGAAGTTTTAAATCA | |
| ATCTAAAGTATATATGAGTAAACTTGGTCTGACAGTTAGAAAAACTCATCGAGCATCAAATGAAACTGCAATTTA | |
| TTCATATCAGGATTATCAATACCATATTTTTGAAAAAGCCGTTTCTGTAATGAAGGAGAAAACTCACCGAGGCAG | |
| TTCCATAGGATGGCAAGATCCTGGTATCGGTCTGCGATTCCGACTCGTCCAACATCAATACAACCTATTAATTTC | |
| CCCTCGTCAAAAATAAGGTTATCAAGTGAGAAATCACCATGAGTGACGACTGAATCCGGTGAGAATGGCAACAGC | |
| TTATGCATTTCTTTCCAGACTTGTTCAACAGGCCAGCCATTACGCTCGTCATCAAAATCACTCGCATCAACCAAA | |
| CCGTTATTCATTCGTGATTGCGCCTGAGCGAGACGAAATACGCGATCGCTGTTAAAAGGACAATTACAAACAGGA | |
| ATCGAATGCAACCGGCGCAGGAACACTGCCAGCGCATCAACAATATTTTCACCTGAATCAGGATATTCTTCTAAT | |
| ACCTGGAATGCTGTTTTTCCGGGGATCGCAGTGGTGAGTAACCATGCATCATCAGGAGTACGGATAAAATGCTTG | |
| ATGGTCGGAAGAGGCATAAATTCCGTCAGCCAGTTTAGTCTGACCATCTCATCTGTAACATCATTGGCAACGCTA | |
| CCTTTGCCATGTTTCAGAAACAACTCTGGCGCATCGGGCTTCCCATACAATCGATAGATTGTCGCACCTGATTGC | |
| CCGACATTATCGCGAGCCCATTTATACCCATATAAATCAGCATCCATGTTGGAATTTAATCGCGGCCTAGAGCAA | |
| GACGTTTCCCGTTGAATATGGCTCATAACACCCCTTGTATTACTGTTTATGTAAGCAGACAGTTTTATTGTTCAT | |
| GATGATATATTTTTATCTTGTGCAATGTAACATCAGAGATTTTGAGACACAACAATTGGTCGAC | |
| Plasmid as defined in FIG. 2D (pDNA2b pGM299) | |
| Length: 3384; Molecule Type: DNA; Features Location/Qualifiers: source, | |
| 1..3384; mol_type, other DNA; note, pGM299; organism, synthetic construct | |
| SEQ ID NO: 6 | |
| TCAATATTGGCCATTAGCCATATTATTCATTGGTTATATAGCATAAATCAATATTGGCTATTGGCCATTGCATAC | |
| GTTGTATCTATATCATAATATGTACATTTATATTGGCTCATGTCCAATATGACCGCCATGTTGGCATTGATTATT | |
| GACTAGTTATTAATAGTAATCAATTACGGGGTCATTAGTTCATAGCCCATATATGGAGTTCCGCGTTACATAACT | |
| TACGGTAAATGGCCCGCCTGGCTGACCGCCCAACGACCCCCGCCCATTGACGTCAATAATGACGTATGTTCCCAT | |
| AGTAACGCCAATAGGGACTTTCCATTGACGTCAATGGGTGGAGTATTTACGGTAAACTGCCCACTTGGCAGTACA | |
| TCAAGTGTATCATATGCCAAGTCCGCCCCCTATTGACGTCAATGACGGTAAATGGCCCGCCTGGCATTATGCCCA | |
| GTACATGACCTTACGGGACTTTCCTACTTGGCAGTACATCTACGTATTAGTCATCGCTATTACCATGGTGATGCG | |
| GTTTTGGCAGTACACCAATGGGCGTGGATAGCGGTTTGACTCACGGGGATTTCCAAGTCTCCACCCCATTGACGT | |
| CAATGGGAGTTTGTTTTGGCACCAAAATCAACGGGACTTTCCAAAATGTCGTAATAACCCCGCCCCGTTGACGCA | |
| AATGGGCGGTAGGCGTGTACGGTGGGAGGTCTATATAAGCAGAGCTCGTTTAGTGAACCGTCAGATCACTAGAAG | |
| CTTTATTGCGGTAGTTTATCACAGTTAAATTGCTAACGCAGTCAGTGCTTCTGACACAACAGTCTCGAACTTAAG | |
| CTGCAGAAGTTGGTCGTGAGGCACTGGGCAGGTAAGTATCAAGGTTACAAGACAGGTTTAAGGAGACCAATAGAA | |
| ACTGGGCTTGTCGAGACAGAGAAGACTCTTGCGTTTCTGATAGGCACCTATTGGTCTTACTGACATCCACTTTGC | |
| CTTTCTCTCCACAGGTGTCCACTCCCAGTTCAATTACAGCTCTTAAGGCTAGAGTACTTAATACGACTCACTATA | |
| GGCTAGCCTCGAGAATTCGATTATGCCCCTAGGACCAGAAGAAAGAAGATTGCTTCGCTTGATTTGGCTCCTTTA | |
| CAGCACCAATCCATATCCACCAAGTGGGGAAGGGACGGCCAGACAACGCCGACGAGCCAGGAGAAGGTGGAGACA | |
| ACAGCAGGATCAAATTAGAGTCTTGGTAGAAAGACTCCAAGAGCAGGTGTATGCAGTTGACCGCCTGGCTGACGA | |
| GGCTCAACACTTGGCTATACAACAGTTGCCTGACCCTCCTCATTCAGCTTAGAATCACTAGTGAATTCACGCGTG | |
| GTACCTCTAGAGTCGACCCGGGCGGCCGCTTCGAGCAGACATGATAAGATACATTGATGAGTTTGGACAAACCAC | |
| AACTAGAATGCAGTGAAAAAAATGCTTTATTTGTGAAATTTGTGATGCTATTGCTTTATTTGTAACCATTATAAG | |
| CTGCAATAAACAAGTTAACAACAACAATTGCATTCATTTTATGTTTCAGGTTCAGGGGGAGATGTGGGAGGTTTT | |
| TTAAAGCAAGTAAAACCTCTACAAATGTGGTAAAATCGATAAGGATCCGTCGACCAATTGTTGTGTCTCAAAATC | |
| TCTGATGTTACATTGCACAAGATAAAAATATATCATCATGAACAATAAAACTGTCTGCTTACATAAACAGTAATA | |
| CAAGGGGTGTTATGAGCCATATTCAACGGGAAACGTCTTGCTCTAGGCCGCGATTAAATTCCAACATGGATGCTG | |
| ATTTATATGGGTATAAATGGGCTCGCGATAATGTCGGGCAATCAGGTGCGACAATCTATCGATTGTATGGGAAGC | |
| CCGATGCGCCAGAGTTGTTTCTGAAACATGGCAAAGGTAGCGTTGCCAATGATGTTACAGATGAGATGGTCAGAC | |
| TAAACTGGCTGACGGAATTTATGCCTCTTCCGACCATCAAGCATTTTATCCGTACTCCTGATGATGCATGGTTAC | |
| TCACCACTGCGATCCCCGGAAAAACAGCATTCCAGGTATTAGAAGAATATCCTGATTCAGGTGAAAATATTGTTG | |
| ATGCGCTGGCAGTGTTCCTGCGCCGGTTGCATTCGATTCCTGTTTGTAATTGTCCTTTTAACAGCGATCGCGTAT | |
| TTCGTCTCGCTCAGGCGCAATCACGAATGAATAACGGTTTGGTTGATGCGAGTGATTTTGATGACGAGCGTAATG | |
| GCTGGCCTGTTGAACAAGTCTGGAAAGAAATGCATAAGCTGTTGCCATTCTCACCGGATTCAGTCGTCACTCATG | |
| GTGATTTCTCACTTGATAACCTTATTTTTGACGAGGGGAAATTAATAGGTTGTATTGATGTTGGACGAGTCGGAA | |
| TCGCAGACCGATACCAGGATCTTGCCATCCTATGGAACTGCCTCGGTGAGTTTTCTCCTTCATTACAGAAACGGC | |
| TTTTTCAAAAATATGGTATTGATAATCCTGATATGAATAAATTGCAGTTTCATTTGATGCTCGATGAGTTTTTCT | |
| AACTGTCAGACCAAGTTTACTCATATATACTTTAGATTGATTTAAAACTTCATTTTTAATTTAAAAGGATCTAGG | |
| TGAAGATCCTTTTTGATAATCTCATGACCAAAATCCCTTAACGTGAGTTTTCGTTCCACTGAGCGTCAGACCCCG | |
| TAGAAAAGATCAAAGGATCTTCTTGAGATCCTTTTTTTCTGCGCGTAATCTGCTGCTTGCAAACAAAAAAACCAC | |
| CGCTACCAGCGGTGGTTTGTTTGCCGGATCAAGAGCTACCAACTCTTTTTCCGAAGGTAACTGGCTTCAGCAGAG | |
| CGCAGATACCAAATACTGTTCTTCTAGTGTAGCCGTAGTTAGGCCACCACTTCAAGAACTCTGTAGCACCGCCTA | |
| CATACCTCGCTCTGCTAATCCTGTTACCAGTGGCTGCTGCCAGTGGCGATAAGTCGTGTCTTACCGGGTTGGACT | |
| CAAGACGATAGTTACCGGATAAGGCGCAGCGGTCGGGCTGAACGGGGGGTTCGTGCACACAGCCCAGCTTGGAGC | |
| GAACGACCTACACCGAACTGAGATACCTACAGCGTGAGCTATGAGAAAGCGCCACGCTTCCCGAAGGGAGAAAGG | |
| CGGACAGGTATCCGGTAAGCGGCAGGGTCGGAACAGGAGAGCGCACGAGGGAGCTTCCAGGGGGAAACGCCTGGT | |
| ATCTTTATAGTCCTGTCGGGTTTCGCCACCTCTGACTTGAGCGTCGATTTTTGTGATGCTCGTCAGGGGGGCGGA | |
| GCCTATGGAAAAACGCCAGCAACGCGGCCTTTTTACGGTTCCTGGCCTTTTGCTGGCCTTTTGCTCACATGGCTC | |
| GACAGATCT | |
| Plasmid as defined in FIG. 2E (pDNA3a pGM301) | |
| Length: 6264; Molecule Type: DNA; Features Location/Qualifiers: source, | |
| 1..6264; mol_type, other DNA; note, pGM301; organism, synthetic construct | |
| SEQ ID NO: 7 | |
| ATTGATTATTGACTAGTTATTAATAGTAATCAATTACGGGGTCATTAGTTCATAGCCCATATATGGAGTTCCGCG | |
| TTACATAACTTACGGTAAATGGCCCGCCTGGCTGACCGCCCAACGACCCCCGCCCATTGACGTCAATAATGACGT | |
| ATGTTCCCATAGTAACGCCAATAGGGACTTTCCATTGACGTCAATGGGTGGAGTATTTACGGTAAACTGCCCACT | |
| TGGCAGTACATCAAGTGTATCATATGCCAAGTACGCCCCCTATTGACGTCAATGACGGTAAATGGCCCGCCTGGC | |
| ATTATGCCCAGTACATGACCTTATGGGACTTTCCTACTTGGCAGTACATCTACGTATTAGTCATCGCTATTACCA | |
| TGGTCGAGGTGAGCCCCACGTTCTGCTTCACTCTCCCCATCTCCCCCCCCTCCCCACCCCCAATTTTGTATTTAT | |
| TTATTTTTTAATTATTTTGTGCAGCGATGGGGGCGGGGGGGGGGGGGGGGCGCGCGCCAGGCGGGGCGGGGCGGG | |
| GCGAGGGGCGGGGCGGGGCGAGGCGGAGAGGTGCGGCGGCAGCCAATCAGAGCGGCGCGCTCCGAAAGTTTCCTT | |
| TTATGGCGAGGCGGCGGCGGCGGCGGCCCTATAAAAAGCGAAGCGCGCGGCGGGCGGGAGTCGCTGCGCGCTGCC | |
| TTCGCCCCGTGCCCCGCTCCGCCGCCGCCTCGCGCCGCCCGCCCCGGCTCTGACTGACCGCGTTACTCCCACAGG | |
| TGAGCGGGCGGGACGGCCCTTCTCCTCCGGGCTGTAATTAGCGCTTGGTTTAATGACGGCTTGTTTCTTTTCTGT | |
| GGCTGCGTGAAAGCCTTGAGGGGCTCCGGGAGGGCCCTTTGTGCGGGGGGAGCGGCTCGGGGGGTGCGTGCGTGT | |
| GTGTGTGCGTGGGGAGCGCCGCGTGCGGCTCCGCGCTGCCCGGCGGCTGTGAGCGCTGCGGGCGCGGCGCGGGGC | |
| TTTGTGCGCTCCGCAGTGTGCGCGAGGGGAGCGCGGCCGGGGGCGGTGCCCCGCGGTGCGGGGGGGGCTGCGAGG | |
| GGAACAAAGGCTGCGTGCGGGGTGTGTGCGTGGGGGGGTGAGCAGGGGGTGTGGGCGCGTCGGTCGGGCTGCAAC | |
| CCCCCCTGCACCCCCCTCCCCGAGTTGCTGAGCACGGCCCGGCTTCGGGTGCGGGGCTCCGTACGGGGCGTGGCG | |
| CGGGGCTCGCCGTGCCGGGCGGGGGGTGGCGGCAGGTGGGGGTGCCGGGCGGGGCGGGGCCGCCTCGGGCCGGGG | |
| AGGGCTCGGGGGAGGGGCGCGGCGGCCCCCGGAGCGCCGGCGGCTGTCGAGGCGCGGCGAGCCGCAGCCATTGCC | |
| TTTTATGGTAATCGTGCGAGAGGGCGCAGGGACTTCCTTTGTCCCAAATCTGTGCGGAGCCGAAATCTGGGAGGC | |
| GCCGCCGCACCCCCTCTAGCGGGCGCGGGGCGAAGCGGTGCGGCGCCGGCAGGAAGGAAATGGGCGGGGAGGGCC | |
| TTCGTGCGTCGCCGCGCCGCCGTCCCCTTCTCCCTCTCCAGCCTCGGGGCTGTCCGCGGGGGGACGGCTGCCTTC | |
| GGGGGGGACGGGGCAGGGCGGGGTTCGGCTTCTGGCGTGTGACCGGCGGCTCTAGAGCCTCTGCTAACCATGTTC | |
| ATGCCTTCTTCTTTTTCCTACAGCTCCTGGGCAACGTGCTGGTTATTGTGCTGTCTCATCATTTTGGCAAAGAAT | |
| TCGATTGCCATGGCAACATATATCCAGAGAGTACAGTGCATCTCAACATCACTACTGGTTGTTCTCACCACATTG | |
| GTCTCGTGTCAGATTCCCAGGGATAGGCTCTCTAACATAGGGGTCATAGTCGATGAAGGGAAATCACTGAAGATA | |
| GCTGGATCCCACGAATCGAGGTACATAGTACTGAGTCTAGTTCCGGGGGTAGACTTTGAGAATGGGTGCGGAACA | |
| GCCCAGGTTATCCAGTACAAGAGCCTACTGAACAGGCTGTTAATCCCATTGAGGGATGCCTTAGATCTTCAGGAG | |
| GCTCTGATAACTGTCACCAATGATACGACACAAAATGCCGGTGCTCCCCAGTCGAGATTCTTCGGTGCTGTGATT | |
| GGTACTATCGCACTTGGAGTGGCGACATCAGCACAAATCACCGCAGGGATTGCACTAGCCGAAGCGAGGGAGGCC | |
| AAAAGAGACATAGCGCTCATCAAAGAATCGATGACAAAAACACACAAGTCTATAGAACTGCTGCAAAACGCTGTG | |
| GGGGAACAAATTCTTGCTCTAAAGACACTCCAGGATTTCGTGAATGATGAGATCAAACCCGCAATAAGCGAATTA | |
| GGCTGTGAGACTGCTGCCTTAAGACTGGGTATAAAATTGACACAGCATTACTCCGAGCTGTTAACTGCGTTCGGC | |
| TCGAATTTCGGAACCATCGGAGAGAAGAGCCTCACGCTGCAGGCGCTGTCTTCACTTTACTCTGCTAACATTACT | |
| GAGATTATGACCACAATCAGGACAGGGCAGTCTAACATCTATGATGTCATTTATACAGAACAGATCAAAGGAACG | |
| GTGATAGATGTGGATCTAGAGAGATACATGGTCACCCTGTCTGTGAAGATCCCTATTCTTTCTGAAGTCCCAGGT | |
| GTGCTCATACACAAGGCATCATCTATTTCTTACAACATAGACGGGGAGGAATGGTATGTGACTGTCCCCAGCCAT | |
| ATACTCAGTCGTGCTTCTTTCTTAGGGGGTGCAGACATAACCGATTGTGTTGAGTCCAGATTGACCTATATATGC | |
| CCCAGGGATCCCGCACAACTGATACCTGACAGCCAGCAAAAGTGTATCCTGGGGGACACAACAAGGTGTCCTGTC | |
| ACAAAAGTTGTGGACAGCCTTATCCCCAAGTTTGCTTTTGTGAATGGGGGCGTTGTTGCTAACTGCATAGCATCC | |
| ACATGTACCTGCGGGACAGGCCGAAGACCAATCAGTCAGGATCGCTCTAAAGGTGTAGTATTCCTAACCCATGAC | |
| AACTGTGGTCTTATAGGTGTCAATGGGGTAGAATTGTATGCTAACCGGAGAGGGCACGATGCCACTTGGGGGGTC | |
| CAGAACTTGACAGTCGGTCCTGCAATTGCTATCAGACCCGTTGATATTTCTCTCAACCTTGCTGATGCTACGAAT | |
| TTCTTGCAAGACTCTAAGGCTGAGCTTGAGAAAGCACGGAAAATCCTCTCGGAGGTAGGTAGATGGTACAACTCA | |
| AGAGAGACTGTGATTACGATCATAGTAGTTATGGTCGTAATATTGGTGGTCATTATAGTGATCATCATCGTGCTT | |
| TATAGACTCAGAAGGTGAAATCACTAGTGAATTCACTCCTCAGGTGCAGGCTGCCTATCAGAAGGTGGTGGCTGG | |
| TGTGGCCAATGCCCTGGCTCACAAATACCACTGAGATCTTTTTCCCTCTGCCAAAAATTATGGGGACATCATGAA | |
| GCCCCTTGAGCATCTGACTTCTGGCTAATAAAGGAAATTTATTTTCATTGCAATAGTGTGTTGGAATTTTTTGTG | |
| TCTCTCACTCGGAAGGACATATGGGAGGGCAAATCATTTAAAACATCAGAATGAGTATTTGGTTTAGAGTTTGGC | |
| AACATATGCCCATATGCTGGCTGCCATGAACAAAGGTTGGCTATAAAGAGGTCATCAGTATATGAAACAGCCCCC | |
| TGCTGTCCATTCCTTATTCCATAGAAAAGCCTTGACTTGAGGTTAGATTTTTTTTATATTTTGTTTTGTGTTATT | |
| TTTTTCTTTAACATCCCTAAAATTTTCCTTACATGTTTTACTAGCCAGATTTTTCCTCCTCTCCTGACTACTCCC | |
| AGTCATAGCTGTCCCTCTTCTCTTATGGAGATCCCTCGACCTGCAGCCCAAGCTTGGCGTAATCATGGTCATAGC | |
| TGTTTCCTGTGTGAAATTGTTATCCGCTCACAATTCCACACAACATACGAGCCGGAAGCATAAAGTGTAAAGCCT | |
| GGGGTGCCTAATGAGTGAGCTAACTCACATTAATTGCGTTGCGCTCACTGCCCGCTTTCCAGTCGGGAAACCTGT | |
| CGTGCCAGCGGATCCGCATCTCAATTAGTCAGCAACCATAGTCCCGCCCCTAACTCCGCCCATCCCGCCCCTAAC | |
| TCCGCCCAGTTCCGCCCATTCTCCGCCCCATGGCTGACTAATTTTTTTTATTTATGCAGAGGCCGAGGCCGCCTC | |
| GGCCTCTGAGCTATTCCAGAAGTAGTGAGGAGGCTTTTTTGGAGGCCTAGGCTTTTGCAAAAAGCTAACTTGTTT | |
| ATTGCAGCTTATAATGGTTACAAATAAAGCAATAGCATCACAAATTTCACAAATAAAGCATTTTTTTCACTGCAT | |
| TCTAGTTGTGGTTTGTCCAAACTCATCAATGTATCTTATCATGTCTGTCCGCTTCCTCGCTCACTGACTCGCTGC | |
| GCTCGGTCGTTCGGCTGCGGCGAGCGGTATCAGCTCACTCAAAGGCGGTAATACGGTTATCCACAGAATCAGGGG | |
| ATAACGCAGGAAAGAACATGTGAGCAAAAGGCCAGCAAAAGGCCAGGAACCGTAAAAAGGCCGCGTTGCTGGCGT | |
| TTTTCCATAGGCTCCGCCCCCCTGACGAGCATCACAAAAATCGACGCTCAAGTCAGAGGTGGCGAAACCCGACAG | |
| GACTATAAAGATACCAGGCGTTTCCCCCTGGAAGCTCCCTCGTGCGCTCTCCTGTTCCGACCCTGCCGCTTACCG | |
| GATACCTGTCCGCCTTTCTCCCTTCGGGAAGCGTGGCGCTTTCTCATAGCTCACGCTGTAGGTATCTCAGTTCGG | |
| TGTAGGTCGTTCGCTCCAAGCTGGGCTGTGTGCACGAACCCCCCGTTCAGCCCGACCGCTGCGCCTTATCCGGTA | |
| ACTATCGTCTTGAGTCCAACCCGGTAAGACACGACTTATCGCCACTGGCAGCAGCCACTGGTAACAGGATTAGCA | |
| GAGCGAGGTATGTAGGCGGTGCTACAGAGTTCTTGAAGTGGTGGCCTAACTACGGCTACACTAGAAGAACAGTAT | |
| TTGGTATCTGCGCTCTGCTGAAGCCAGTTACCTTCGGAAAAAGAGTTGGTAGCTCTTGATCCGGCAAACAAACCA | |
| CCGCTGGTAGCGGTGGTTTTTTTGTTTGCAAGCAGCAGATTACGCGCAGAAAAAAAGGATCTCAAGAAGATCCTT | |
| TGATCTTTTCTACGGGGTCTGACGCTCAGTGGAACGAAAACTCACGTTAAGGGATTTTGGTCATGAGATTATCAA | |
| AAAGGATCTTCACCTAGATCCTTTTAAATTAAAAATGAAGTTTTAAATCAATCTAAAGTATATATGAGTAAACTT | |
| GGTCTGACAGTTAGAAAAACTCATCGAGCATCAAATGAAACTGCAATTTATTCATATCAGGATTATCAATACCAT | |
| ATTTTTGAAAAAGCCGTTTCTGTAATGAAGGAGAAAACTCACCGAGGCAGTTCCATAGGATGGCAAGATCCTGGT | |
| ATCGGTCTGCGATTCCGACTCGTCCAACATCAATACAACCTATTAATTTCCCCTCGTCAAAAATAAGGTTATCAA | |
| GTGAGAAATCACCATGAGTGACGACTGAATCCGGTGAGAATGGCAACAGCTTATGCATTTCTTTCCAGACTTGTT | |
| CAACAGGCCAGCCATTACGCTCGTCATCAAAATCACTCGCATCAACCAAACCGTTATTCATTCGTGATTGCGCCT | |
| GAGCGAGACGAAATACGCGATCGCTGTTAAAAGGACAATTACAAACAGGAATCGAATGCAACCGGCGCAGGAACA | |
| CTGCCAGCGCATCAACAATATTTTCACCTGAATCAGGATATTCTTCTAATACCTGGAATGCTGTTTTTCCGGGGA | |
| TCGCAGTGGTGAGTAACCATGCATCATCAGGAGTACGGATAAAATGCTTGATGGTCGGAAGAGGCATAAATTCCG | |
| TCAGCCAGTTTAGTCTGACCATCTCATCTGTAACATCATTGGCAACGCTACCTTTGCCATGTTTCAGAAACAACT | |
| CTGGCGCATCGGGCTTCCCATACAATCGATAGATTGTCGCACCTGATTGCCCGACATTATCGCGAGCCCATTTAT | |
| ACCCATATAAATCAGCATCCATGTTGGAATTTAATCGCGGCCTAGAGCAAGACGTTTCCCGTTGAATATGGCTCA | |
| TAACACCCCTTGTATTACTGTTTATGTAAGCAGACAGTTTTATTGTTCATGATGATATATTTTTATCTTGTGCAA | |
| TGTAACATCAGAGATTTTGAGACACAACAATTGGTCGAC | |
| Plasmid as defined in FIG. 2F (pDNA3b pGM303) | |
| Length: 6522; Molecule Type: DNA; Features Location/Qualifiers: source, | |
| 1..6522; mol_type, other DNA; note, pGM303; organism, synthetic construct | |
| SEQ ID NO: 8 | |
| ATTGATTATTGACTAGTTATTAATAGTAATCAATTACGGGGTCATTAGTTCATAGCCCATATATGGAGTTCCGCG | |
| TTACATAACTTACGGTAAATGGCCCGCCTGGCTGACCGCCCAACGACCCCCGCCCATTGACGTCAATAATGACGT | |
| ATGTTCCCATAGTAACGCCAATAGGGACTTTCCATTGACGTCAATGGGTGGAGTATTTACGGTAAACTGCCCACT | |
| TGGCAGTACATCAAGTGTATCATATGCCAAGTACGCCCCCTATTGACGTCAATGACGGTAAATGGCCCGCCTGGC | |
| ATTATGCCCAGTACATGACCTTATGGGACTTTCCTACTTGGCAGTACATCTACGTATTAGTCATCGCTATTACCA | |
| TGGTCGAGGTGAGCCCCACGTTCTGCTTCACTCTCCCCATCTCCCCCCCCTCCCCACCCCCAATTTTGTATTTAT | |
| TTATTTTTTAATTATTTTGTGCAGCGATGGGGGCGGGGGGGGGGGGGGGGCGCGCGCCAGGCGGGGCGGGGCGGG | |
| GCGAGGGGCGGGGCGGGGCGAGGCGGAGAGGTGCGGCGGCAGCCAATCAGAGCGGCGCGCTCCGAAAGTTTCCTT | |
| TTATGGCGAGGCGGCGGCGGCGGCGGCCCTATAAAAAGCGAAGCGCGCGGCGGGCGGGAGTCGCTGCGCGCTGCC | |
| TTCGCCCCGTGCCCCGCTCCGCCGCCGCCTCGCGCCGCCCGCCCCGGCTCTGACTGACCGCGTTACTCCCACAGG | |
| TGAGCGGGCGGGACGGCCCTTCTCCTCCGGGCTGTAATTAGCGCTTGGTTTAATGACGGCTTGTTTCTTTTCTGT | |
| GGCTGCGTGAAAGCCTTGAGGGGCTCCGGGAGGGCCCTTTGTGCGGGGGGAGCGGCTCGGGGGGTGCGTGCGTGT | |
| GTGTGTGCGTGGGGAGCGCCGCGTGCGGCTCCGCGCTGCCCGGCGGCTGTGAGCGCTGCGGGCGCGGCGCGGGGC | |
| TTTGTGCGCTCCGCAGTGTGCGCGAGGGGAGCGCGGCCGGGGGCGGTGCCCCGCGGTGCGGGGGGGGCTGCGAGG | |
| GGAACAAAGGCTGCGTGCGGGGTGTGTGCGTGGGGGGGTGAGCAGGGGGTGTGGGCGCGTCGGTCGGGCTGCAAC | |
| CCCCCCTGCACCCCCCTCCCCGAGTTGCTGAGCACGGCCCGGCTTCGGGTGCGGGGCTCCGTACGGGGCGTGGCG | |
| CGGGGCTCGCCGTGCCGGGCGGGGGGTGGCGGCAGGTGGGGGTGCCGGGCGGGGCGGGGCCGCCTCGGGCCGGGG | |
| AGGGCTCGGGGGAGGGGCGCGGCGGCCCCCGGAGCGCCGGCGGCTGTCGAGGCGCGGCGAGCCGCAGCCATTGCC | |
| TTTTATGGTAATCGTGCGAGAGGGCGCAGGGACTTCCTTTGTCCCAAATCTGTGCGGAGCCGAAATCTGGGAGGC | |
| GCCGCCGCACCCCCTCTAGCGGGCGCGGGGCGAAGCGGTGCGGCGCCGGCAGGAAGGAAATGGGCGGGGAGGGCC | |
| TTCGTGCGTCGCCGCGCCGCCGTCCCCTTCTCCCTCTCCAGCCTCGGGGCTGTCCGCGGGGGGACGGGGCAGGGC | |
| GGGGTTCGGCTTCTGGCGTGTGACCGGCGGCTCTAGAGCCTCTGCTAACCATGTTCATGCCTTCTTCTTTTTCCT | |
| ACAGCTCCTGGGCAACGTGCTGGTTATTGTGCTGTCTCATCATTTTGGCAAAGAATTCCTCGAGCATGTGGTCTG | |
| AGTTAAAAATCAGGAGCAACGACGGAGGTGAAGGACCAGAGGACGCCAACGACCCCCGGGGAAAGGGGGTGCAAC | |
| ACATCCATATCCAGCCATCTCTACCTGTTTATGGACAGAGGGTTAGGGATGGTGATAGGGGCAAACGTGACTCGT | |
| ACTGGTCTACTTCTCCTAGTGGTAGCACCACAAAACCAGCATCAGGTTGGGAGAGGTCAAGTAAAGCCGACACAT | |
| GGTTGCTGATTCTCTCATTCACCCAGTGGGCTTTGTCAATTGCCACAGTGATCATCTGTATCATAATTTCTGCTA | |
| GACAAGGGTATAGTATGAAAGAGTACTCAATGACTGTAGAGGCATTGAACATGAGCAGCAGGGAGGTGAAAGAGT | |
| CACTTACCAGTCTAATAAGGCAAGAGGTTATAGCAAGGGCTGTCAACATTCAGAGCTCTGTGCAAACCGGAATCC | |
| CAGTCTTGTTGAACAAAAACAGCAGGGATGTCATCCAGATGATTGATAAGTCGTGCAGCAGACAAGAGCTCACTC | |
| AGCACTGTGAGAGTACGATCGCAGTCCACCATGCCGATGGAATTGCCCCACTTGAGCCACATAGTTTCTGGAGAT | |
| GCCCTGTCGGAGAACCGTATCTTAGCTCAGATCCTGAAATCTCATTGCTGCCTGGTCCGAGCTTGTTATCTGGTT | |
| CTACAACGATCTCTGGATGTGTTAGGCTCCCTTCACTCTCAATTGGCGAGGCAATCTATGCCTATTCATCAAATC | |
| TCATTACACAAGGTTGTGCTGACATAGGGAAATCATATCAGGTCCTGCAGCTAGGGTACATATCACTCAATTCAG | |
| ATATGTTCCCTGATCTTAACCCCGTAGTGTCCCACACTTATGACATCAACGACAATCGGAAATCATGCTCTGTGG | |
| TGGCAACCGGGACTAGGGGTTATCAGCTTTGCTCCATGCCGACTGTAGACGAAAGAACCGACTACTCTAGTGATG | |
| GTATTGAGGATCTGGTCCTTGATGTCCTGGATCTCAAAGGGAGAACTAAGTCTCACCGGTATCGCAACAGCGAGG | |
| TAGATCTTGATCACCCGTTCTCTGCACTATACCCCAGTGTAGGCAACGGCATTGCAACAGAAGGCTCATTGATAT | |
| TTCTTGGGTATGGTGGACTAACCACCCCTCTGCAGGGTGATACAAAATGTAGGACCCAAGGATGCCAACAGGTGT | |
| CGCAAGACACATGCAATGAGGCTCTGAAAATTACATGGCTAGGAGGGAAACAGGTGGTCAGCGTGATCATCCAGG | |
| TCAATGACTATCTCTCAGAGAGGCCAAAGATAAGAGTCACAACCATTCCAATCACTCAAAACTATCTCGGGGCGG | |
| AAGGTAGATTATTAAAATTGGGTGATCGGGTGTACATCTATACAAGATCATCAGGCTGGCACTCTCAACTGCAGA | |
| TAGGAGTACTTGATGTCAGCCACCCTTTGACTATCAACTGGACACCTCATGAAGCCTTGTCTAGACCAGGAAATA | |
| AAGAGTGCAATTGGTACAATAAGTGTCCGAAGGAATGCATATCAGGCGTATACACTGATGCTTATCCATTGTCCC | |
| CTGATGCAGCTAACGTCGCTACCGTCACGCTATATGCCAATACATCGCGTGTCAACCCAACAATCATGTATTCTA | |
| ACACTACTAACATTATAAATATGTTAAGGATAAAGGATGTTCAATTAGAGGCTGCATATACCACGACATCGTGTA | |
| TCACGCATTTTGGTAAAGGCTACTGCTTTCACATCATCGAGATCAATCAGAAGAGCCTGAATACCTTACAGCCGA | |
| TGCTCTTTAAGACTAGCATCCCTAAATTATGCAAGGCCGAGTCTTAAGCGGCCGCGCATGCGAATTCACTCCTCA | |
| GGTGCAGGCTGCCTATCAGAAGGTGGTGGCTGGTGTGGCCAATGCCCTGGCTCACAAATACCACTGAGATCTTTT | |
| TCCCTCTGCCAAAAATTATGGGGACATCATGAAGCCCCTTGAGCATCTGACTTCTGGCTAATAAAGGAAATTTAT | |
| TTTCATTGCAATAGTGTGTTGGAATTTTTTGTGTCTCTCACTCGGAAGGACATATGGGAGGGCAAATCATTTAAA | |
| ACATCAGAATGAGTATTTGGTTTAGAGTTTGGCAACATATGCCCATATGCTGGCTGCCATGAACAAAGGTTGGCT | |
| ATAAAGAGGTCATCAGTATATGAAACAGCCCCCTGCTGTCTATTCCTTATTCCATAGAAAAGCCTTGACTTGAGG | |
| TTAGATTTTTTTTATATTTTGTTTTGTGTTATTTTTTTCTTTAACATCCCTAAAATTTTCCTTACATGTTTTACT | |
| AGCCAGATTTTTCCTCCTCTCCTGACTACTCCCAGTCATAGCTGTCCCTCTTCTCTTATGGAGATCCCTCGACCT | |
| GCAGCCCAAGCTTGGCGTAATCATGGTCATAGCTGTTTCCTGTGTGAAATTGTTATCCGCTCACAATTCCACACA | |
| ACATACGAGCCGGAAGCATAAAGTGTAAAGCCTGGGGTGCCTAATGAGTGAGCTAACTCACATTAATTGCGTTGC | |
| GCTCACTGCCCGCTTTCCAGTCGGGAAACCTGTCGTGCCAGCGGATCCGCATCTCAATTAGTCAGCAACCATAGT | |
| CCCGCCCCTAACTCCGCCCATCCCGCCCCTAACTCCGCCCAGTTCCGCCCATTCTCCGCCCCATGGCTGACTAAT | |
| TTTTTTTATTTATGCAGAGGCCGAGGCCGCCTCGGCCTCTGAGCTATTCCAGAAGTAGTGAGGAGGCTTTTTTGG | |
| AGGCCTAGGCTTTTGCAAAAAGCTAACTTGTTTATTGCAGCTTATAATGGTTACAAATAAAGCAATAGCATCACA | |
| AATTTCACAAATAAAGCATTTTTTTCACTGCATTCTAGTTGTGGTTTGTCCAAACTCATCAATGTATCTTATCAT | |
| GTCTGTCCGCTTCCTCGCTCACTGACTCGCTGCGCTCGGTCGTTCGGCTGCGGCGAGCGGTATCAGCTCACTCAA | |
| AGGCGGTAATACGGTTATCCACAGAATCAGGGGATAACGCAGGAAAGAACATGTGAGCAAAAGGCCAGCAAAAGG | |
| CCAGGAACCGTAAAAAGGCCGCGTTGCTGGCGTTTTTCCATAGGCTCCGCCCCCCTGACGAGCATCACAAAAATC | |
| GACGCTCAAGTCAGAGGTGGCGAAACCCGACAGGACTATAAAGATACCAGGCGTTTCCCCCTGGAAGCTCCCTCG | |
| TGCGCTCTCCTGTTCCGACCCTGCCGCTTACCGGATACCTGTCCGCCTTTCTCCCTTCGGGAAGCGTGGCGCTTT | |
| CTCATAGCTCACGCTGTAGGTATCTCAGTTCGGTGTAGGTCGTTCGCTCCAAGCTGGGCTGTGTGCACGAACCCC | |
| CCGTTCAGCCCGACCGCTGCGCCTTATCCGGTAACTATCGTCTTGAGTCCAACCCGGTAAGACACGACTTATCGC | |
| CACTGGCAGCAGCCACTGGTAACAGGATTAGCAGAGCGAGGTATGTAGGCGGTGCTACAGAGTTCTTGAAGTGGT | |
| GGCCTAACTACGGCTACACTAGAAGAACAGTATTTGGTATCTGCGCTCTGCTGAAGCCAGTTACCTTCGGAAAAA | |
| GAGTTGGTAGCTCTTGATCCGGCAAACAAACCACCGCTGGTAGCGGTGGTTTTTTTGTTTGCAAGCAGCAGATTA | |
| CGCGCAGAAAAAAAGGATCTCAAGAAGATCCTTTGATCTTTTCTACGGGGTCTGACGCTCAGTGGAACGAAAACT | |
| CACGTTAAGGGATTTTGGTCATGAGATTATCAAAAAGGATCTTCACCTAGATCCTTTTAAATTAAAAATGAAGTT | |
| TTAAATCAATCTAAAGTATATATGAGTAAACTTGGTCTGACAGTTAGAAAAACTCATCGAGCATCAAATGAAACT | |
| GCAATTTATTCATATCAGGATTATCAATACCATATTTTTGAAAAAGCCGTTTCTGTAATGAAGGAGAAAACTCAC | |
| CGAGGCAGTTCCATAGGATGGCAAGATCCTGGTATCGGTCTGCGATTCCGACTCGTCCAACATCAATACAACCTA | |
| TTAATTTCCCCTCGTCAAAAATAAGGTTATCAAGTGAGAAATCACCATGAGTGACGACTGAATCCGGTGAGAATG | |
| GCAACAGCTTATGCATTTCTTTCCAGACTTGTTCAACAGGCCAGCCATTACGCTCGTCATCAAAATCACTCGCAT | |
| CAACCAAACCGTTATTCATTCGTGATTGCGCCTGAGCGAGACGAAATACGCGATCGCTGTTAAAAGGACAATTAC | |
| AAACAGGAATCGAATGCAACCGGCGCAGGAACACTGCCAGCGCATCAACAATATTTTCACCTGAATCAGGATATT | |
| CTTCTAATACCTGGAATGCTGTTTTTCCGGGGATCGCAGTGGTGAGTAACCATGCATCATCAGGAGTACGGATAA | |
| AATGCTTGATGGTCGGAAGAGGCATAAATTCCGTCAGCCAGTTTAGTCTGACCATCTCATCTGTAACATCATTGG | |
| CAACGCTACCTTTGCCATGTTTCAGAAACAACTCTGGCGCATCGGGCTTCCCATACAATCGATAGATTGTCGCAC | |
| CTGATTGCCCGACATTATCGCGAGCCCATTTATACCCATATAAATCAGCATCCATGTTGGAATTTAATCGCGGCC | |
| TAGAGCAAGACGTTTCCCGTTGAATATGGCTCATAACACCCCTTGTATTACTGTTTATGTAAGCAGACAGTTTTA | |
| TTGTTCATGATGATATATTTTTATCTTGTGCAATGTAACATCAGAGATTTTGAGACACAACAATTGGTCGAC | |
| Plasmid as defined in FIG. 2G (pDNA2a pGM297) | |
| Length: 9886; Molecule Type: DNA; Features Location/Qualifiers: source, | |
| 1..9886; mol_type, other DNA; note, pGM297; organism, synthetic construct | |
| SEQ ID NO: 9 | |
| ATTGATTATTGACTAGTTATTAATAGTAATCAATTACGGGGTCATTAGTTCATAGCCCATATATGGAGTTCCGCG | |
| TTACATAACTTACGGTAAATGGCCCGCCTGGCTGACCGCCCAACGACCCCCGCCCATTGACGTCAATAATGACGT | |
| ATGTTCCCATAGTAACGCCAATAGGGACTTTCCATTGACGTCAATGGGTGGAGTATTTACGGTAAACTGCCCACT | |
| TGGCAGTACATCAAGTGTATCATATGCCAAGTACGCCCCCTATTGACGTCAATGACGGTAAATGGCCCGCCTGGC | |
| ATTATGCCCAGTACATGACCTTATGGGACTTTCCTACTTGGCAGTACATCTACGTATTAGTCATCGCTATTACCA | |
| TGGTCGAGGTGAGCCCCACGTTCTGCTTCACTCTCCCCATCTCCCCCCCCTCCCCACCCCCAATTTTGTATTTAT | |
| TTATTTTTTAATTATTTTGTGCAGCGATGGGGGCGGGGGGGGGGGGGGGGCGCGCGCCAGGCGGGGCGGGGCGGG | |
| GCGAGGGGCGGGGCGGGGCGAGGCGGAGAGGTGCGGCGGCAGCCAATCAGAGCGGCGCGCTCCGAAAGTTTCCTT | |
| TTATGGCGAGGCGGCGGCGGCGGCGGCCCTATAAAAAGCGAAGCGCGCGGCGGGCGGGAGTCGCTGCGCGCTGCC | |
| TTCGCCCCGTGCCCCGCTCCGCCGCCGCCTCGCGCCGCCCGCCCCGGCTCTGACTGACCGCGTTACTCCCACAGG | |
| TGAGCGGGCGGGACGGCCCTTCTCCTCCGGGCTGTAATTAGCGCTTGGTTTAATGACGGCTTGTTTCTTTTCTGT | |
| GGCTGCGTGAAAGCCTTGAGGGGCTCCGGGAGGGCCCTTTGTGCGGGGGGAGCGGCTCGGGGGGTGCGTGCGTGT | |
| GTGTGTGCGTGGGGAGCGCCGCGTGCGGCTCCGCGCTGCCCGGCGGCTGTGAGCGCTGCGGGCGCGGCGCGGGGC | |
| TTTGTGCGCTCCGCAGTGTGCGCGAGGGGAGCGCGGCCGGGGGCGGTGCCCCGCGGTGCGGGGGGGGCTGCGAGG | |
| GGAACAAAGGCTGCGTGCGGGGTGTGTGCGTGGGGGGGTGAGCAGGGGGTGTGGGCGCGTCGGTCGGGCTGCAAC | |
| CCCCCCTGCACCCCCCTCCCCGAGTTGCTGAGCACGGCCCGGCTTCGGGTGCGGGGCTCCGTACGGGGCGTGGCG | |
| CGGGGCTCGCCGTGCCGGGCGGGGGGTGGCGGCAGGTGGGGGTGCCGGGCGGGGCGGGGCCGCCTCGGGCCGGGG | |
| AGGGCTCGGGGGAGGGGCGCGGCGGCCCCCGGAGCGCCGGCGGCTGTCGAGGCGCGGCGAGCCGCAGCCATTGCC | |
| TTTTATGGTAATCGTGCGAGAGGGCGCAGGGACTTCCTTTGTCCCAAATCTGTGCGGAGCCGAAATCTGGGAGGC | |
| GCCGCCGCACCCCCTCTAGCGGGCGCGGGGCGAAGCGGTGCGGCGCCGGCAGGAAGGAAATGGGCGGGGAGGGCC | |
| TTCGTGCGTCGCCGCGCCGCCGTCCCCTTCTCCCTCTCCAGCCTCGGGGCTGTCCGCGGGGGGACGGCTGCCTTC | |
| GGGGGGGACGGGGCAGGGCGGGGTTCGGCTTCTGGCGTGTGACCGGCGGCTCTAGAGCCTCTGCTAACCATGTTC | |
| ATGCCTTCTTCTTTTTCCTACAGCTCCTGGGCAACGTGCTGGTTATTGTGCTGTCTCATCATTTTGGCAAAGAAT | |
| TGCTCGAGACTAGTGACTTGGTGAGTAGGCTTCGAGCCTAGTTAGAGGACTAGGAGAGGCCGTAGCCGTAACTAC | |
| TCTGGGCAAGTAGGGCAGGCGGTGGGTACGCAATGGGGGCGGCTACCTCAGCACTAAATAGGAGACAATTAGACC | |
| AATTTGAGAAAATACGACTTCGCCCGAACGGAAAGAAAAAGTACCAAATTAAACATTTAATATGGGCAGGCAAGG | |
| AGATGGAGCGCTTCGGCCTCCATGAGAGGTTGTTGGAGACAGAGGAGGGGTGTAAAAGAATCATAGAAGTCCTCT | |
| ACCCCCTAGAACCAACAGGATCGGAGGGCTTAAAAAGTCTGTTCAATCTTGTGTGCGTACTATATTGCTTGCACA | |
| AGGAACAGAAAGTGAAAGACACAGAGGAAGCAGTAGCAACAGTAAGACAACACTGCCATCTAGTGGAAAAAGAAA | |
| AAAGTGCAACAGAGACATCTAGTGGACAAAAGAAAAATGACAAGGGAATAGCAGCGCCACCTGGTGGCAGTCAGA | |
| ATTTTCCAGCGCAACAACAAGGAAATGCCTGGGTACATGTACCCTTGTCACCGCGCACCTTAAATGCGTGGGTAA | |
| AAGCAGTAGAGGAGAAAAAATTTGGAGCAGAAATAGTACCCATGTTTCAAGCCCTATCAGAAGGCTGCACACCCT | |
| ATGACATTAATCAGATGCTTAATGTGCTAGGAGATCATCAAGGGGCATTACAAATAGTGAAAGAGATCATTAATG | |
| AAGAAGCAGCCCAGTGGGATGTAACACACCCACTACCCGCAGGACCCCTACCAGCAGGACAGCTCAGGGACCCTC | |
| GCGGCTCAGATATAGCAGGGACCACCAGCTCAGTACAAGAACAGTTAGAATGGATCTATACTGCTAACCCCCGGG | |
| TAGATGTAGGTGCCATCTACCGGAGATGGATTATTCTAGGACTTCAAAAGTGTGTCAAAATGTACAACCCAGTAT | |
| CAGTCCTAGACATTAGGCAGGGACCTAAAGAGCCCTTCAAGGATTATGTGGACAGATTTTACAAGGCAATTAGAG | |
| CAGAACAAGCCTCAGGGGAAGTGAAACAATGGATGACAGAATCATTACTCATTCAAAATGCTAATCCAGATTGTA | |
| AGGTCATCCTGAAGGGCCTAGGAATGCACCCCACCCTTGAAGAAATGTTAACGGCTTGTCAGGGGGTAGGAGGCC | |
| CAAGCTACAAAGCAAAAGTAATGGCAGAAATGATGCAGACCATGCAAAATCAAAACATGGTGCAGCAGGGAGGTC | |
| CAAAAAGACAAAGACCCCCACTAAGATGTTATAATTGTGGAAAATTTGGCCATATGCAAAGACAATGTCCGGAAC | |
| CAAGGAAAACAAAATGTCTAAAGTGTGGAAAATTGGGACACCTAGCAAAAGACTGCAGGGGACAGGTGAATTTTT | |
| TAGGGTATGGACGGTGGATGGGGGCAAAACCGAGAAATTTTCCCGCCGCTACTCTTGGAGCGGAACCGAGTGCGC | |
| CTCCTCCACCGAGCGGCACCACCCCATACGACCCAGCAAAGAAGCTCCTGCAGCAATATGCAGAGAAAGGGAAAC | |
| AACTGAGGGAGCAAAAGAGGAATCCACCGGCAATGAATCCGGATTGGACCGAGGGATATTCTTTGAACTCCCTCT | |
| TTGGAGAAGACCAATAAAGACAGTGTATATAGAAGGGGTCCCCATTAAGGCACTGCTAGACACAGGGGCAGATGA | |
| CACCATAATTAAAGAAAATGATTTACAATTATCAGGTCCATGGAGACCCAAAATTATAGGGGGCATAGGAGGAGG | |
| CCTTAATGTAAAAGAATATAACGACAGGGAAGTAAAAATAGAAGATAAAATTTTGAGAGGAACAATATTGTTAGG | |
| AGCAACTCCCATTAATATAATAGGTAGAAATTTGCTGGCCCCGGCAGGTGCCCGGTTAGTAATGGGACAATTATC | |
| AGAAAAAATTCCTGTCACACCTGTCAAATTGAAGGAAGGGGCTCGGGGACCCTGTGTAAGACAATGGCCTCTCTC | |
| TAAAGAGAAGATTGAAGCTTTACAGGAAATATGTTCCCAATTAGAGCAGGAAGGAAAAATCAGTAGAGTAGGAGG | |
| AGAAAATGCATACAATACCCCAATATTTTGCATAAAGAAGAAGGACAAATCCCAGTGGAGGATGCTAGTAGACTT | |
| TAGAGAGTTAAATAAGGCAACCCAAGATTTCTTTGAAGTGCAATTAGGGATACCCCACCCAGCAGGATTAAGAAA | |
| GATGAGACAGATAACAGTTTTAGATGTAGGAGACGCCTATTATTCCATACCATTGGATCCAAATTTTAGGAAATA | |
| TACTGCTTTTACTATTCCCACAGTGAATAATCAGGGACCCGGGATTAGGTATCAATTCAACTGTCTCCCGCAAGG | |
| GTGGAAAGGATCTCCTACAATCTTCCAAAATACAGCAGCATCCATTTTGGAGGAGATAAAAAGAAACTTGCCAGC | |
| ACTAACCATTGTACAATACATGGATGATTTATGGGTAGGTTCTCAAGAAAATGAACACACCCATGACAAATTAGT | |
| AGAACAGTTAAGAACAAAATTACAAGCCTGGGGCTTAGAAACCCCAGAAAAGAAGGTGCAAAAAGAACCACCTTA | |
| TGAGTGGATGGGATACAAACTTTGGCCTCACAAATGGGAACTAAGCAGAATACAACTGGAGGAAAAAGATGAATG | |
| GACTGTCAATGACATCCAGAAGTTAGTTGGGAAACTAAATTGGGCAGCACAATTGTATCCAGGTCTTAGGACCAA | |
| GAATATATGCAAGTTAATTAGAGGAAAGAAAAATCTGTTAGAGCTAGTGACTTGGACACCTGAGGCAGAAGCTGA | |
| ATATGCAGAAAATGCAGAGATTCTTAAAACAGAACAGGAAGGAACCTATTACAAACCAGGAATACCTATTAGGGC | |
| AGCAGTACAGAAATTGGAAGGAGGACAGTGGAGTTACCAATTCAAACAAGAAGGACAAGTCTTGAAAGTAGGAAA | |
| ATACACCAAGCAAAAGAACACCCATACAAATGAACTTCGCACATTAGCTGGTTTAGTGCAGAAGATTTGCAAAGA | |
| AGCTCTAGTTATTTGGGGGATATTACCAGTTCTAGAACTCCCGATAGAAAGAGAGGTATGGGAACAATGGTGGGC | |
| GGATTACTGGCAGGTAAGCTGGATTCCCGAATGGGATTTTGTCAGCACCCCACCTTTGCTCAAACTATGGTACAC | |
| ATTAACAAAAGAACCCATACCCAAGGAGGACGTTTACTATGTAGATGGAGCATGCAACAGAAATTCAAAAGAAGG | |
| AAAAGCAGGATACATCTCACAATACGGAAAACAGAGAGTAGAAACATTAGAAAACACTACCAATCAGCAAGCAGA | |
| ATTAACAGCTATAAAAATGGCTTTGGAAGACAGTGGGCCTAATGTGAACATAGTAACAGACTCTCAATATGCAAT | |
| GGGAATTTTGACAGCACAACCCACACAAAGTGATTCACCATTAGTAGAGCAAATTATAGCCTTAATGATACAAAA | |
| GCAACAAATATATTTGCAGTGGGTACCAGCACATAAAGGAATAGGAGGAAATGAGGAGATAGATAAATTAGTGAG | |
| TAAAGGCATTAGAAGAGTTTTATTCTTAGAAAAAATAGAAGAAGCTCAAGAAGAGCATGAAAGATATCATAATAA | |
| TTGGAAAAACCTAGCAGATACATATGGGCTTCCACAAATAGTAGCAAAAGAGATAGTGGCCATGTGTCCAAAATG | |
| TCAGATAAAGGGAGAACCAGTGCATGGACAAGTGGATGCCTCACCTGGAACATGGCAGATGGATTGTACTCATCT | |
| AGAAGGAAAAGTAGTCATAGTTGCGGTCCATGTAGCCAGTGGATTCATAGAAGCAGAAGTCATACCTAGGGAAAC | |
| AGGAAAAGAAACGGCAAAGTTTCTATTAAAAATACTGAGTAGATGGCCTATAACACAGTTACACACAGACAATGG | |
| GCCTAACTTTACCTCCCAAGAAGTGGCAGCAATATGTTGGTGGGGAAAAATTGAACATACAACAGGTATACCATA | |
| TAACCCCCAATCTCAAGGATCAATAGAAAGCATGAACAAACAATTAAAAGAGATAATTGGGAAAATAAGAGATGA | |
| TTGCCAATATACAGAGACAGCAGTACTGATGGCTTGCCATATTCACAATTTTAAAAGAAAGGGAGGAATAGGGGG | |
| ACAGACTTCAGCAGAGAGACTAATTAATATAATAACAACACAATTAGAAATACAACATTTACAAACCAAAATTCA | |
| AAAAATTTTAAATTTTAGAGTCTACTACAGAGAAGGGAGAGACCCTGTGTGGAAAGGACCAGCACAATTAATCTG | |
| GAAAGGGGAAGGAGCAGTGGTCCTCAAGGACGGAAGTGACCTAAAGGTTGTACCAAGAAGGAAAGCTAAAATTAT | |
| TAAGGATTATGAACCCAAACAAAGAGTGGGTAATGAGGGTGACGTGGAAGGTACCAGGGGATCTGATAACTAAAT | |
| GGCAGGGAATAGTCAGATATTGGATGAGACAAAGAAATTTGAAATGGAACTATTATATGCATCAGCTGGCGGCCG | |
| CGAATTCACTAGTGATTCCCGTTTGTGCTAGGGTTCTTAGGCTTCTTGGGGGCTGCTGGAACTGCAATGGGAGCA | |
| GCGGCGACAGCCCTGACGGTCCAGTCTCAGCATTTGCTTGCTGGGATACTGCAGCAGCAGAAGAATCTGCTGGCG | |
| GCTGTGGAGGCTCAACAGCAGATGTTGAAGCTGACCATTTGGGGTGTTAAAAACCTCAATGCCCGCGTCACAGCC | |
| CTTGAGAAGTACCTAGAGGATCAGGCACGACTAAACTCCTGGGGGTGCGCATGGAAACAAGTATGTCATACCACA | |
| GTGGAGTGGCCCTGGACAAATCGGACTCCGGATTGGCAAAATATGACTTGGTTGGAGTGGGAAAGACAAATAGCT | |
| GATTTGGAAAGCAACATTACGAGACAATTAGTGAAGGCTAGAGAACAAGAGGAAAAGAATCTAGATGCCTATCAG | |
| AAGTTAACTAGTTGGTCAGATTTCTGGTCTTGGTTCGATTTCTCAAAATGGCTTAACATTTTAAAAATGGGATTT | |
| TTAGTAATAGTAGGAATAATAGGGTTAAGATTACTTTACACAGTATATGGATGTATAGTGAGGGTTAGGCAGGGA | |
| TATGTTCCTCTATCTCCACAGATCCATATCCAATCGAATTCCCGCGGCCGCAATTCACTCCTCAGGTGCAGGCTG | |
| CCTATCAGAAGGTGGTGGCTGGTGTGGCCAATGCCCTGGCTCACAAATACCACTGAGATCTTTTTCCCTCTGCCA | |
| AAAATTATGGGGACATCATGAAGCCCCTTGAGCATCTGACTTCTGGCTAATAAAGGAAATTTATTTTCATTGCAA | |
| TAGTGTGTTGGAATTTTTTGTGTCTCTCACTCGGAAGGACATATGGGAGGGCAAATCATTTAAAACATCAGAATG | |
| AGTATTTGGTTTAGAGTTTGGCAACATATGCCCATATGCTGGCTGCCATGAACAAAGGTTGGCTATAAAGAGGTC | |
| ATCAGTATATGAAACAGCCCCCTGCTGTCCATTCCTTATTCCATAGAAAAGCCTTGACTTGAGGTTAGATTTTTT | |
| TTATATTTTGTTTTGTGTTATTTTTTTCTTTAACATCCCTAAAATTTTCCTTACATGTTTTACTAGCCAGATTTT | |
| TCCTCCTCTCCTGACTACTCCCAGTCATAGCTGTCCCTCTTCTCTTATGGAGATCCCTCGACCTGCAGCCCAAGC | |
| TTGGCGTAATCATGGTCATAGCTGTTTCCTGTGTGAAATTGTTATCCGCTCACAATTCCACACAACATACGAGCC | |
| GGAAGCATAAAGTGTAAAGCCTGGGGTGCCTAATGAGTGAGCTAACTCACATTAATTGCGTTGCGCTCACTGCCC | |
| GCTTTCCAGTCGGGAAACCTGTCGTGCCAGCGGATCCGCATCTCAATTAGTCAGCAACCATAGTCCCGCCCCTAA | |
| CTCCGCCCATCCCGCCCCTAACTCCGCCCAGTTCCGCCCATTCTCCGCCCCATGGCTGACTAATTTTTTTTATTT | |
| ATGCAGAGGCCGAGGCCGCCTCGGCCTCTGAGCTATTCCAGAAGTAGTGAGGAGGCTTTTTTGGAGGCCTAGGCT | |
| TTTGCAAAAAGCTAACTTGTTTATTGCAGCTTATAATGGTTACAAATAAAGCAATAGCATCACAAATTTCACAAA | |
| TAAAGCATTTTTTTCACTGCATTCTAGTTGTGGTTTGTCCAAACTCATCAATGTATCTTATCATGTCTGTCCGCT | |
| TCCTCGCTCACTGACTCGCTGCGCTCGGTCGTTCGGCTGCGGCGAGCGGTATCAGCTCACTCAAAGGCGGTAATA | |
| CGGTTATCCACAGAATCAGGGGATAACGCAGGAAAGAACATGTGAGCAAAAGGCCAGCAAAAGGCCAGGAACCGT | |
| AAAAAGGCCGCGTTGCTGGCGTTTTTCCATAGGCTCCGCCCCCCTGACGAGCATCACAAAAATCGACGCTCAAGT | |
| CAGAGGTGGCGAAACCCGACAGGACTATAAAGATACCAGGCGTTTCCCCCTGGAAGCTCCCTCGTGCGCTCTCCT | |
| GTTCCGACCCTGCCGCTTACCGGATACCTGTCCGCCTTTCTCCCTTCGGGAAGCGTGGCGCTTTCTCATAGCTCA | |
| CGCTGTAGGTATCTCAGTTCGGTGTAGGTCGTTCGCTCCAAGCTGGGCTGTGTGCACGAACCCCCCGTTCAGCCC | |
| GACCGCTGCGCCTTATCCGGTAACTATCGTCTTGAGTCCAACCCGGTAAGACACGACTTATCGCCACTGGCAGCA | |
| GCCACTGGTAACAGGATTAGCAGAGCGAGGTATGTAGGCGGTGCTACAGAGTTCTTGAAGTGGTGGCCTAACTAC | |
| GGCTACACTAGAAGAACAGTATTTGGTATCTGCGCTCTGCTGAAGCCAGTTACCTTCGGAAAAAGAGTTGGTAGC | |
| TCTTGATCCGGCAAACAAACCACCGCTGGTAGCGGTGGTTTTTTTGTTTGCAAGCAGCAGATTACGCGCAGAAAA | |
| AAAGGATCTCAAGAAGATCCTTTGATCTTTTCTACGGGGTCTGACGCTCAGTGGAACGAAAACTCACGTTAAGGG | |
| ATTTTGGTCATGAGATTATCAAAAAGGATCTTCACCTAGATCCTTTTAAATTAAAAATGAAGTTTTAAATCAATC | |
| TAAAGTATATATGAGTAAACTTGGTCTGACAGTTAGAAAAACTCATCGAGCATCAAATGAAACTGCAATTTATTC | |
| ATATCAGGATTATCAATACCATATTTTTGAAAAAGCCGTTTCTGTAATGAAGGAGAAAACTCACCGAGGCAGTTC | |
| CATAGGATGGCAAGATCCTGGTATCGGTCTGCGATTCCGACTCGTCCAACATCAATACAACCTATTAATTTCCCC | |
| TCGTCAAAAATAAGGTTATCAAGTGAGAAATCACCATGAGTGACGACTGAATCCGGTGAGAATGGCAACAGCTTA | |
| TGCATTTCTTTCCAGACTTGTTCAACAGGCCAGCCATTACGCTCGTCATCAAAATCACTCGCATCAACCAAACCG | |
| TTATTCATTCGTGATTGCGCCTGAGCGAGACGAAATACGCGATCGCTGTTAAAAGGACAATTACAAACAGGAATC | |
| GAATGCAACCGGCGCAGGAACACTGCCAGCGCATCAACAATATTTTCACCTGAATCAGGATATTCTTCTAATACC | |
| TGGAATGCTGTTTTTCCGGGGATCGCAGTGGTGAGTAACCATGCATCATCAGGAGTACGGATAAAATGCTTGATG | |
| GTCGGAAGAGGCATAAATTCCGTCAGCCAGTTTAGTCTGACCATCTCATCTGTAACATCATTGGCAACGCTACCT | |
| TTGCCATGTTTCAGAAACAACTCTGGCGCATCGGGCTTCCCATACAATCGATAGATTGTCGCACCTGATTGCCCG | |
| ACATTATCGCGAGCCCATTTATACCCATATAAATCAGCATCCATGTTGGAATTTAATCGCGGCCTAGAGCAAGAC | |
| GTTTCCCGTTGAATATGGCTCATAACACCCCTTGTATTACTGTTTATGTAAGCAGACAGTTTTATTGTTCATGAT | |
| GATATATTTTTATCTTGTGCAATGTAACATCAGAGATTTTGAGACACAACAATTGGTCGAC | |
| Exemplified hCEF promoter | |
| Length: 574; Molecule Type: DNA; Features Location/Qualifiers: source, | |
| 1..574; mol_type, other DNA; note, hCEF promoter; organism, synthetic | |
| construct | |
| SEQ ID NO: 10 | |
| 1 | AGATCTGTTA CATAACTTAT GGTAAATGGC CTGCCTGGCT GACTGCCCAA TGACCCCTGC |
| 61 | CCAATGATGT CAATAATGAT GTATGTTCCC ATGTAATGCC AATAGGGACT TTCCATTGAT |
| 121 | GTCAATGGGT GGAGTATTTA TGGTAACTGC CCACTTGGCA GTACATCAAG TGTATCATAT |
| 181 | GCCAAGTATG CCCCCTATTG ATGTCAATGA TGGTAAATGG CCTGCCTGGC ATTATGCCCA |
| 241 | GTACATGACC TTATGGGACT TTCCTACTTG GCAGTACATC TATGTATTAG TCATTGCTAT |
| 301 | TACCATGGGA ATTCACTAGT GGAGAAGAGC ATGCTTGAGG GCTGAGTGCC CCTCAGTGGG |
| 361 | CAGAGAGCAC ATGGCCCACA GTCCCTGAGA AGTTGGGGGG AGGGGTGGGC AATTGAACTG |
| 421 | GTGCCTAGAG AAGGTGGGGC TTGGGTAAAC TGGGAAAGTG ATGTGGTGTA CTGGCTCCAC |
| 481 | CTTTTTCCCC AGGGTGGGGG AGAACCATAT ATAAGTGCAG TAGTCTCTGT GAACATTCAA |
| 541 | GCTTCTGCCT TCTCCCTCCT GTGAGTTTGC TAGC |
| Exemplified CMV promoter | |
| Length: 873; Molecule Type: DNA; Features Location/Qualifiers: source, | |
| 1..873; mol_type, unassigned DNA; organism, Human cytomegalovirus | |
| SEQ ID NO: 11 | |
| CCGCGGAGATCTCAATATTGGCCATTAGCCATATTATTCATTGGTTATATAGCATAAATCAATATTGGCT | |
| ATTGGCCATTGCATACGTTGTATCTATATCATAATATGTACATTTATATTGGCTCATGTCCAATATGACC | |
| GCCATGTTGGCATTGATTATTGACTAGTTATTAATAGTAATCAATTACGGGGTCATTAGTTCATAGCCCA | |
| TATATGGAGTTCCGCGTTACATAACTTACGGTAAATGGCCCGCCTGGCTGACCGCCCAACGACCCCCGCC | |
| CATTGACGTCAATAATGACGTATGTTCCCATAGTAACGCCAATAGGGACTTTCCATTGACGTCAATGGGT | |
| GGAGTATTTACGGTAAACTGCCCACTTGGCAGTACATCAAGTGTATCATATGCCAAGTCCGCCCCCTATT | |
| GACGTCAATGACGGTAAATGGCCCGCCTGGCATTATGCCCAGTACATGACCTTACGGGACTTTCCTACTT | |
| GGCAGTACATCTACGTATTAGTCATCGCTATTACCATGGTGATGCGGTTTTGGCAGTACACCAATGGGCG | |
| TGGATAGCGGTTTGACTCACGGGGATTTCCAAGTCTCCACCCCATTGACGTCAATGGGAGTTTGTTTTGG | |
| CACCAAAATCAACGGGACTTTCCAAAATGTCGTAATAACCCCGCCCCGTTGACGCAAATGGGCGGTAGGC | |
| GTGTACGGTGGGAGGTCTATATAAGCAGAGCTCGTTTAGTGAACCGTCAGATCACTAGAAGCTTTATTGC | |
| GGTAGTTTATCACAGTTAAATTGCTAACGCAGTCAGTGCTTCTGACACAACAGTCTCGAACTTAAGCTGC | |
| AGAAGTTGGTCGTGAGGCACTGGGCAGGCTAGC | |
| Exemplified EF1a promoter | |
| Length: 395; Molecule Type: DNA; Features Location/Qualifiers: source, | |
| 1..395; mol_type, unassigned DNA; organism, Homo sapiens | |
| SEQ ID NO: 12 | |
| AGATCCATATCCGCGGCAATTTTAAAAGAAAGGGAGGAATAGGGGGACAGACTTCAGCAGAGAGACTAATTAATA | |
| TAATAACAACACAATTAGAAATACAACATTTACAAACCAAAATTCAAAAAATTTTAAATTTTAGAGCCGCGGAGA | |
| TCCCGTGAGGCTCCGGTGCCCGTCAGTGGGCAGAGCGCACATCGCCCACAGTCCCCGAGAAGTTGGGGGGAGGGG | |
| TCGGCAATTGAACCGGTGCCTAGAGAAGGTGGCGCGGGGTAAACTGGGAAAGTGATGTCGTGTACTGGCTCCGCC | |
| TTTTTCCCGAGGGTGGGGGAGAACCGTATATAAGTGCAGTAGTCGCCGTGAACGTTCTTTTTCGCAACGGGTTTG | |
| CCGCCAGAACACAGGCTAGC | |
| Exemplified CFTR transgene (soCFTR2) | |
| Length: 4459; Molecule Type: DNA; Features Location/Qualifiers: source, | |
| 1..4459; mol_type, other DNA; note, soCFTR2; organism, synthetic construct | |
| SEQ ID NO: 13 | |
| 1 | GCTAGCCACC ATGCAGAGAA GCCCTCTGGA GAAGGCCTCT GTGGTGAGCA AGCTGTTCTT |
| 61 | CAGCTGGACC AGGCCCATCC TGAGGAAGGG CTACAGGCAG AGACTGGAGC TGTCTGACAT |
| 121 | CTACCAGATC CCCTCTGTGG ACTCTGCTGA CAACCTGTCT GAGAAGCTGG AGAGGGAGTG |
| 181 | GGATAGAGAG CTGGCCAGCA AGAAGAACCC CAAGCTGATC AATGCCCTGA GGAGATGCTT |
| 241 | CTTCTGGAGA TTCATGTTCT ATGGCATCTT CCTGTACCTG GGGGAAGTGA CCAAGGCTGT |
| 301 | GCAGCCTCTG CTGCTGGGCA GAATCATTGC CAGCTATGAC CCTGACAACA AGGAGGAGAG |
| 361 | GAGCATTGCC ATCTACCTGG GCATTGGCCT GTGCCTGCTG TTCATTGTGA GGACCCTGCT |
| 421 | GCTGCACCCT GCCATCTTTG GCCTGCACCA CATTGGCATG CAGATGAGGA TTGCCATGTT |
| 481 | CAGCCTGATC TACAAGAAAA CCCTGAAGCT GTCCAGCAGA GTGCTGGACA AGATCAGCAT |
| 541 | TGGCCAGCTG GTGAGCCTGC TGAGCAACAA CCTGAACAAG TTTGATGAGG GCCTGGCCCT |
| 601 | GGCCCACTTT GTGTGGATTG CCCCTCTGCA GGTGGCCCTG CTGATGGGCC TGATTTGGGA |
| 661 | GCTGCTGCAG GCCTCTGCCT TTTGTGGCCT GGGCTTCCTG ATTGTGCTGG CCCTGTTTCA |
| 721 | GGCTGGCCTG GGCAGGATGA TGATGAAGTA CAGGGACCAG AGGGCAGGCA AGATCAGTGA |
| 781 | GAGGCTGGTG ATCACCTCTG AGATGATTGA GAACATCCAG TCTGTGAAGG CCTACTGTTG |
| 841 | GGAGGAAGCT ATGGAGAAGA TGATTGAAAA CCTGAGGCAG ACAGAGCTGA AGCTGACCAG |
| 901 | GAAGGCTGCC TATGTGAGAT ACTTCAACAG CTCTGCCTTC TTCTTCTCTG GCTTCTTTGT |
| 961 | GGTGTTCCTG TCTGTGCTGC CCTATGCCCT GATCAAGGGG ATCATCCTGA GAAAGATTTT |
| 1021 | CACCACCATC AGCTTCTGCA TTGTGCTGAG GATGGCTGTG ACCAGACAGT TCCCCTGGGC |
| 1081 | TGTGCAGACC TGGTATGACA GCCTGGGGGC CATCAACAAG ATCCAGGACT TCCTGCAGAA |
| 1141 | GCAGGAGTAC AAGACCCTGG AGTACAACCT GACCACCACA GAAGTGGTGA TGGAGAATGT |
| 1201 | GACAGCCTTC TGGGAGGAGG GCTTTGGGGA GCTGTTTGAG AAGGCCAAGC AGAACAACAA |
| 1261 | CAACAGAAAG ACCAGCAATG GGGATGACTC CCTGTTCTTC TCCAACTTCT CCCTGCTGGG |
| 1321 | CACACCTGTG CTGAAGGACA TCAACTTCAA GATTGAGAGG GGGCAGCTGC TGGCTGTGGC |
| 1381 | TGGATCTACA GGGGCTGGCA AGACCAGCCT GCTGATGATG ATCATGGGGG AGCTGGAGCC |
| 1441 | TTCTGAGGGC AAGATCAAGC ACTCTGGCAG GATCAGCTTT TGCAGCCAGT TCAGCTGGAT |
| 1501 | CATGCCTGGC ACCATCAAGG AGAACATCAT CTTTGGAGTG AGCTATGATG AGTACAGATA |
| 1561 | CAGGAGTGTG ATCAAGGCCT GCCAGCTGGA GGAGGACATC AGCAAGTTTG CTGAGAAGGA |
| 1621 | CAACATTGTG CTGGGGGAGG GAGGCATTAC ACTGTCTGGG GGCCAGAGAG CCAGAATCAG |
| 1681 | CCTGGCCAGG GCTGTGTACA AGGATGCTGA CCTGTACCTG CTGGACTCCC CCTTTGGCTA |
| 1741 | CCTGGATGTG CTGACAGAGA AGGAGATTTT TGAGAGCTGT GTGTGCAAGC TGATGGCCAA |
| 1801 | CAAGACCAGA ATCCTGGTGA CCAGCAAGAT GGAGCACCTG AAGAAGGCTG ACAAGATCCT |
| 1861 | GATCCTGCAT GAGGGCAGCA GCTACTTCTA TGGGACCTTC TCTGAGCTGC AGAACCTGCA |
| 1921 | GCCTGACTTC AGCTCTAAGC TGATGGGCTG TGACAGCTTT GACCAGTTCT CTGCTGAGAG |
| 1981 | GAGGAACAGC ATCCTGACAG AGACCCTGCA CAGATTCAGC CTGGAGGGAG ATGCCCCTGT |
| 2041 | GAGCTGGACA GAGACCAAGA AGCAGAGCTT CAAGCAGACA GGGGAGTTTG GGGAGAAGAG |
| 2101 | GAAGAACTCC ATCCTGAACC CCATCAACAG CATCAGGAAG TTCAGCATTG TGCAGAAAAC |
| 2161 | CCCCCTGCAG ATGAATGGCA TTGAGGAAGA TTCTGATGAG CCCCTGGAGA GGAGACTGAG |
| 2221 | CCTGGTGCCT GATTCTGAGC AGGGAGAGGC CATCCTGCCT AGGATCTCTG TGATCAGCAC |
| 2281 | AGGCCCTACA CTGCAGGCCA GAAGGAGGCA GTCTGTGCTG AACCTGATGA CCCACTCTGT |
| 2341 | GAACCAGGGC CAGAACATCC ACAGGAAAAC CACAGCCTCC ACCAGGAAAG TGAGCCTGGC |
| 2401 | CCCTCAGGCC AATCTGACAG AGCTGGACAT CTACAGCAGG AGGCTGTCTC AGGAGACAGG |
| 2461 | CCTGGAGATT TCTGAGGAGA TCAATGAGGA GGACCTGAAA GAGTGCTTCT TTGATGACAT |
| 2521 | GGAGAGCATC CCTGCTGTGA CCACCTGGAA CACCTACCTG AGATACATCA CAGTGCACAA |
| 2581 | GAGCCTGATC TTTGTGCTGA TCTGGTGCCT GGTGATCTTC CTGGCTGAAG TGGCTGCCTC |
| 2641 | TCTGGTGGTG CTGTGGCTGC TGGGAAACAC CCCACTGCAG GACAAGGGCA ACAGCACCCA |
| 2701 | CAGCAGGAAC AACAGCTATG CTGTGATCAT CACCTCCACC TCCAGCTACT ATGTGTTCTA |
| 2761 | CATCTATGTG GGAGTGGCTG ATACCCTGCT GGCTATGGGC TTCTTTAGAG GCCTGCCCCT |
| 2821 | GGTGCACACA CTGATCACAG TGAGCAAGAT CCTCCACCAC AAGATGCTGC ACTCTGTGCT |
| 2881 | GCAGGCTCCT ATGAGCACCC TGAATACCCT GAAGGCTGGG GGCATCCTGA ACAGATTCTC |
| 2941 | CAAGGATATT GCCATCCTGG ATGACCTGCT GCCTCTCACC ATCTTTGACT TCATCCAGCT |
| 3001 | GCTGCTGATT GTGATTGGGG CCATTGCTGT GGTGGCAGTG CTGCAGCCCT ACATCTTTGT |
| 3061 | GGCCACAGTG CCTGTGATTG TGGCCTTCAT CATGCTGAGG GCCTACTTTC TGCAGACCTC |
| 3121 | CCAGCAGCTG AAGCAGCTGG AGTCTGAGGG CAGAAGCCCC ATCTTCACCC ACCTGGTGAC |
| 3181 | AAGCCTGAAG GGCCTGTGGA CCCTGAGAGC CTTTGGCAGG CAGCCCTACT TTGAGACCCT |
| 3241 | GTTCCACAAG GCCCTGAACC TGCACACAGC CAACTGGTTC CTCTACCTGT CCACCCTGAG |
| 3301 | ATGGTTCCAG ATGAGAATTG AGATGATCTT TGTCATCTTC TTCATTGCTG TGACCTTCAT |
| 3361 | CAGCATTCTG ACCACAGGAG AGGGAGAGGG CAGAGTGGGC ATTATCCTGA CCCTGGCCAT |
| 3421 | GAACATCATG AGCACACTGC AGTGGGCAGT GAACAGCAGC ATTGATGTGG ACAGCCTGAT |
| 3481 | GAGGAGTGTG AGCAGAGTGT TCAAGTTCAT TGATATGCCC ACAGAGGGCA AGCCTACCAA |
| 3541 | GAGCACCAAG CCCTACAAGA ATGGCCAGCT GAGCAAAGTG ATGATCATTG AGAACAGCCA |
| 3601 | TGTGAAGAAG GATGATATCT GGCCCAGTGG AGGCCAGATG ACAGTGAAGG ACCTGACAGC |
| 3661 | CAAGTACACA GAGGGGGGCA ATGCTATCCT GGAGAACATC TCCTTCAGCA TCTCCCCTGG |
| 3721 | CCAGAGAGTG GGACTGCTGG GAAGAACAGG CTCTGGCAAG TCTACCCTGC TGTCTGCCTT |
| 3781 | CCTGAGGCTG CTGAACACAG AGGGAGAGAT CCAGATTGAT GGAGTGTCCT GGGACAGCAT |
| 3841 | CACACTGCAG CAGTGGAGGA AGGCCTTTGG TGTGATCCCC CAGAAAGTGT TCATCTTCAG |
| 3901 | TGGCACCTTC AGGAAGAACC TGGACCCCTA TGAGCAGTGG TCTGACCAGG AGATTTGGAA |
| 3961 | AGTGGCTGAT GAAGTGGGCC TGAGAAGTGT GATTGAGCAG TTCCCTGGCA AGCTGGACTT |
| 4021 | TGTCCTGGTG GATGGGGGCT GTGTGCTGAG CCATGGCCAC AAGCAGCTGA TGTGCCTGGC |
| 4081 | CAGATCAGTG CTGAGCAAGG CCAAGATCCT GCTGCTGGAT GAGCCTTCTG CCCACCTGGA |
| 4141 | TCCTGTGACC TACCAGATCA TCAGGAGGAC CCTCAAGCAG GCCTTTGCTG ACTGCACAGT |
| 4201 | CATCCTGTGT GAGCACAGGA TTGAGGCCAT GCTGGAGTGC CAGCAGTTCC TGGTGATTGA |
| 4261 | GGAGAACAAA GTGAGGCAGT ATGACAGCAT CCAGAAGCTG CTGAATGAGA GGAGCCTGTT |
| 4321 | CAGGCAGGCC ATCAGCCCCT CTGATAGAGT GAAGCTGTTC CCCCACAGGA ACAGCTCCAA |
| 4381 | GTGCAAGAGC AAGCCCCAGA TTGCTGCCCT GAAGGAGGAG ACAGAGGAGG AAGTGCAGGA |
| 4441 | CACCAGGCTG TGAGGGCCC |
| Exemplified A1AT transgene | |
| Length: 1257; Molecule Type: DNA; Features Location/Qualifiers: source, | |
| 1..1257; mol_type, other DNA; note, sohAAT organism, synthetic | |
| construct | |
| SEQ ID NO: 14 | |
| ATGCCCAGCTCTGTGTCCTGGGGCATTCTGCTGCTGGCTGGCCTGTGCTGTCTGGTGCCTGTGTCCCTGG | |
| CTGAGGACCCTCAGGGGGATGCTGCCCAGAAAACAGACACCTCCCACCATGACCAGGACCACCCCACCTT | |
| CAACAAGATCACCCCCAACCTGGCAGAGTTTGCCTTCAGCCTGTACAGACAGCTGGCCCACCAGAGCAAC | |
| AGCACCAACATCTTTTTCAGCCCTGTGTCCATTGCCACAGCCTTTGCCATGCTGAGCCTGGGCACCAAGG | |
| CTGACACCCATGATGAGATCCTGGAAGGCCTGAACTTCAACCTGACAGAGATCCCTGAGGCCCAGATCCA | |
| TGAGGGCTTCCAGGAACTGCTGAGAACCCTGAACCAGCCAGACAGCCAGCTGCAGCTGACAACAGGCAAT | |
| GGGCTGTTCCTGTCTGAGGGCCTGAAGCTGGTGGACAAGTTTCTGGAAGATGTGAAGAAGCTGTACCACT | |
| CTGAGGCCTTCACAGTGAACTTTGGGGACACAGAAGAGGCCAAGAAACAGATCAATGACTATGTGGAAAA | |
| GGGCACCCAGGGCAAGATTGTGGACCTTGTGAAAGAGCTGGACAGGGACACTGTGTTTGCCCTTGTGAAC | |
| TACATCTTCTTCAAGGGCAAGTGGGAGAGGCCCTTTGAAGTGAAGGACACTGAGGAAGAGGACTTCCATG | |
| TGGACCAAGTGACCACAGTGAAGGTGCCAATGATGAAGAGACTGGGGATGTTCAATATCCAGCACTGCAA | |
| GAAACTGAGCAGCTGGGTGCTGCTGATGAAGTACCTGGGCAATGCTACAGCCATATTCTTTCTGCCTGAT | |
| GAGGGCAAGCTGCAGCACCTGGAAAATGAGCTGACCCATGACATCATCACCAAATTTCTGGAAAATGAGG | |
| ACAGAAGATCTGCCAGCCTGCATCTGCCCAAGCTGAGCATCACAGGCACATATGACCTGAAGTCTGTGCT | |
| GGGACAGCTGGGAATCACCAAGGTGTTCAGCAATGGGGCAGACCTGAGTGGAGTGACAGAGGAAGCCCCT | |
| CTGAAGCTGTCCAAGGCTGTGCACAAGGCAGTGCTGACCATTGATGAGAAGGGCACAGAGGCTGCTGGGG | |
| CCATGTTTCTGGAAGCCATCCCCATGTCCATCCCCCCAGAAGTGAAGTTCAACAAGCCCTTTGTGTTCCT | |
| GATGATTGAGCAGAACACCAAGAGCCCCCTGTTCATGGGCAAGGTTGTGAACCCCACCCAGAAATGA | |
| Complementary strand to the exemplified A1AT transgene | |
| Length: 1257; Molecule Type: DNA; Features Location/Qualifiers: source, | |
| 1..1257; mol_type, other DNA; note, sohAAT completmentary strand; | |
| organism, synthetic construct | |
| SEQ ID NO: 15 | |
| TACGGGTCGAGACACAGGACCCCGTAAGACGACGACCGACCGGACACGACAGACCACGGACACAGGGACC | |
| GACTCCTGGGAGTCCCCCTACGACGGGTCTTTTGTCTGTGGAGGGTGGTACTGGTCCTGGTGGGGTGGAA | |
| GTTGTTCTAGTGGGGGTTGGACCGTCTCAAACGGAAGTCGGACATGTCTGTCGACCGGGTGGTCTCGTTG | |
| TCGTGGTTGTAGAAAAAGTCGGGACACAGGTAACGGTGTCGGAAACGGTACGACTCGGACCCGTGGTTCC | |
| GACTGTGGGTACTACTCTAGGACCTTCCGGACTTGAAGTTGGACTGTCTCTAGGGACTCCGGGTCTAGGT | |
| ACTCCCGAAGGTCCTTGACGACTCTTGGGACTTGGTCGGTCTGTCGGTCGACGTCGACTGTTGTCCGTTA | |
| CCCGACAAGGACAGACTCCCGGACTTCGACCACCTGTTCAAAGACCTTCTACACTTCTTCGACATGGTGA | |
| GACTCCGGAAGTGTCACTTGAAACCCCTGTGTCTTCTCCGGTTCTTTGTCTAGTTACTGATACACCTTTT | |
| CCCGTGGGTCCCGTTCTAACACCTGGAACACTTTCTCGACCTGTCCCTGTGACACAAACGGGAACACTTG | |
| ATGTAGAAGAAGTTCCCGTTCACCCTCTCCGGGAAACTTCACTTCCTGTGACTCCTTCTCCTGAAGGTAC | |
| ACCTGGTTCACTGGTGTCACTTCCACGGTTACTACTTCTCTGACCCCTACAAGTTATAGGTCGTGACGTT | |
| CTTTGACTCGTCGACCCACGACGACTACTTCATGGACCCGTTACGATGTCGGTATAAGAAAGACGGACTA | |
| CTCCCGTTCGACGTCGTGGACCTTTTACTCGACTGGGTACTGTAGTAGTGGTTTAAAGACCTTTTACTCC | |
| TGTCTTCTAGACGGTCGGACGTAGACGGGTTCGACTCGTAGTGTCCGTGTATACTGGACTTCAGACACGA | |
| CCCTGTCGACCCTTAGTGGTTCCACAAGTCGTTACCCCGTCTGGACTCACCTCACTGTCTCCTTCGGGGA | |
| GACTTCGACAGGTTCCGACACGTGTTCCGTCACGACTGGTAACTACTCTTCCCGTGTCTCCGACGACCCC | |
| GGTACAAAGACCTTCGGTAGGGGTACAGGTAGGGGGGTCTTCACTTCAAGTTGTTCGGGAAACACAAGGA | |
| CTACTAACTCGTCTTGTGGTTCTCGGGGGACAAGTACCCGTTCCAACACTTGGGGTGGGTCTTTACT | |
| Exemplified A1AT polypeptide | |
| Length: 419; Molecule Type: AA; Features Location/Qualifiers: SOURCE, | |
| 1..419; MOL_TYPE, protein; ORGANISM, Homo sapiens | |
| SEQ ID NO: 16 | |
| AEDPQGDAAQKTDTSHHDQDHPTFAEDPQGDAAQKTDTSHHDQDHPTFNKITPNLAEFAFSLYRQLAHQSN | |
| STNIFFSPVSIATAFAMLSLGTKADTHDEILEGLNFNLTEIPEAQIHEGFQELLRTLNQPDSQLQLTTGNG | |
| LFLSEGLKLVDKFLEDVKKLYHSEAFTVNFGDTEEAKKQINDYVEKGTQGKIVDLVKELDRDTVFALVNYI | |
| FFKGKWERPFEVKDTEEEDFHVDQVTTVKVPMMKRLGMFNIQHCKKLSSWVLLMKYLGNATAIFFLPDEGK | |
| LQHLENELTHDIITKFLENEDRRSASLHLPKLSITGTYDLKSVLGQLGITKVFSNGADLSGVTEEAPLKLS | |
| KAVHKAVLTIDEKGTEAAGAMFLEAIPMSIPPEVKFNKPFVFLMIEQNTKSPLFMGKVVNPTQK | |
| Exemplified FVIII transgene (N6) | |
| Length: 5013; Molecule Type: DNA; Features Location/Qualifiers: source, | |
| 1..5013; mol_type, other DNA; note, codon-optimised FVIII transgene | |
| (N6); organism, synthetic construct | |
| SEQ ID NO: 17 | |
| ATGCAGATTGAGCTGAGCACCTGCTTCTTCCTGTGCCTGCTGAGGTTCTGCTTCTCTGCCACCAGGAGAT | |
| ACTACCTGGGGGCTGTGGAGCTGAGCTGGGACTACATGCAGTCTGACCTGGGGGAGCTGCCTGTGGATGC | |
| CAGGTTCCCCCCCAGAGTGCCCAAGAGCTTCCCCTTCAACACCTCTGTGGTGTACAAGAAGACCCTGTTT | |
| GTGGAGTTCACTGACCACCTGTTCAACATTGCCAAGCCCAGGCCCCCCTGGATGGGCCTGCTGGGCCCCA | |
| CCATCCAGGCTGAGGTGTATGACACTGTGGTGATCACCCTGAAGAACATGGCCAGCCACCCTGTGAGCCT | |
| GCATGCTGTGGGGGTGAGCTACTGGAAGGCCTCTGAGGGGGCTGAGTATGATGACCAGACCAGCCAGAGG | |
| GAGAAGGAGGATGACAAGGTGTTCCCTGGGGGCAGCCACACCTATGTGTGGCAGGTGCTGAAGGAGAATG | |
| GCCCCATGGCCTCTGACCCCCTGTGCCTGACCTACAGCTACCTGAGCCATGTGGACCTGGTGAAGGACCT | |
| GAACTCTGGCCTGATTGGGGCCCTGCTGGTGTGCAGGGAGGGCAGCCTGGCCAAGGAGAAGACCCAGACC | |
| CTGCACAAGTTCATCCTGCTGTTTGCTGTGTTTGATGAGGGCAAGAGCTGGCACTCTGAAACCAAGAACA | |
| GCCTGATGCAGGACAGGGATGCTGCCTCTGCCAGGGCCTGGCCCAAGATGCACACTGTGAATGGCTATGT | |
| GAACAGGAGCCTGCCTGGCCTGATTGGCTGCCACAGGAAGTCTGTGTACTGGCATGTGATTGGCATGGGC | |
| ACCACCCCTGAGGTGCACAGCATCTTCCTGGAGGGCCACACCTTCCTGGTCAGGAACCACAGGCAGGCCA | |
| GCCTGGAGATCAGCCCCATCACCTTCCTGACTGCCCAGACCCTGCTGATGGACCTGGGCCAGTTCCTGCT | |
| GTTCTGCCACATCAGCAGCCACCAGCATGATGGCATGGAGGCCTATGTGAAGGTGGACAGCTGCCCTGAG | |
| GAGCCCCAGCTGAGGATGAAGAACAATGAGGAGGCTGAGGACTATGATGATGACCTGACTGACTCTGAGA | |
| TGGATGTGGTGAGGTTTGATGATGACAACAGCCCCAGCTTCATCCAGATCAGGTCTGTGGCCAAGAAGCA | |
| CCCCAAGACCTGGGTGCACTACATTGCTGCTGAGGAGGAGGACTGGGACTATGCCCCCCTGGTGCTGGCC | |
| CCTGATGACAGGAGCTACAAGAGCCAGTACCTGAACAATGGCCCCCAGAGGATTGGCAGGAAGTACAAGA | |
| AGGTCAGGTTCATGGCCTACACTGATGAAACCTTCAAGACCAGGGAGGCCATCCAGCATGAGTCTGGCAT | |
| CCTGGGCCCCCTGCTGTATGGGGAGGTGGGGGACACCCTGCTGATCATCTTCAAGAACCAGGCCAGCAGG | |
| CCCTACAACATCTACCCCCATGGCATCACTGATGTGAGGCCCCTGTACAGCAGGAGGCTGCCCAAGGGGG | |
| TGAAGCACCTGAAGGACTTCCCCATCCTGCCTGGGGAGATCTTCAAGTACAAGTGGACTGTGACTGTGGA | |
| GGATGGCCCCACCAAGTCTGACCCCAGGTGCCTGACCAGATACTACAGCAGCTTTGTGAACATGGAGAGG | |
| GACCTGGCCTCTGGCCTGATTGGCCCCCTGCTGATCTGCTACAAGGAGTCTGTGGACCAGAGGGGCAACC | |
| AGATCATGTCTGACAAGAGGAATGTGATCCTGTTCTCTGTGTTTGATGAGAACAGGAGCTGGTACCTGAC | |
| TGAGAACATCCAGAGGTTCCTGCCCAACCCTGCTGGGGTGCAGCTGGAGGACCCTGAGTTCCAGGCCAGC | |
| AACATCATGCACAGCATCAATGGCTATGTGTTTGACAGCCTGCAGCTGTCTGTGTGCCTGCATGAGGTGG | |
| CCTACTGGTACATCCTGAGCATTGGGGCCCAGACTGACTTCCTGTCTGTGTTCTTCTCTGGCTACACCTT | |
| CAAGCACAAGATGGTGTATGAGGACACCCTGACCCTGTTCCCCTTCTCTGGGGAGACTGTGTTCATGAGC | |
| ATGGAGAACCCTGGCCTGTGGATTCTGGGCTGCCACAACTCTGACTTCAGGAACAGGGGCATGACTGCCC | |
| TGCTGAAAGTCTCCAGCTGTGACAAGAACACTGGGGACTACTATGAGGACAGCTATGAGGACATCTCTGC | |
| CTACCTGCTGAGCAAGAACAATGCCATTGAGCCCAGGAGCTTCAGCCAGAACAGCAGGCACCCCAGCACC | |
| AGGCAGAAGCAGTTCAATGCCACCACCATCCCTGAGAATGACATAGAGAAGACAGACCCATGGTTTGCCC | |
| ACCGGACCCCCATGCCCAAGATCCAGAATGTGAGCAGCTCTGACCTGCTGATGCTGCTGAGGCAGAGCCC | |
| CACCCCCCATGGCCTGAGCCTGTCTGACCTGCAGGAGGCCAAGTATGAAACCTTCTCTGATGACCCCAGC | |
| CCTGGGGCCATTGACAGCAACAACAGCCTGTCTGAGATGACCCACTTCAGGCCCCAGCTGCACCACTCTG | |
| GGGACATGGTGTTCACCCCTGAGTCTGGCCTGCAGCTGAGGCTGAATGAGAAGCTGGGCACCACTGCTGC | |
| CACTGAGCTGAAGAAGCTGGACTTCAAAGTCTCCAGCACCAGCAACAACCTGATCAGCACCATCCCCTCT | |
| GACAACCTGGCTGCTGGCACTGACAACACCAGCAGCCTGGGCCCCCCCAGCATGCCTGTGCACTATGACA | |
| GCCAGCTGGACACCACCCTGTTTGGCAAGAAGAGCAGCCCCCTGACTGAGTCTGGGGGCCCCCTGAGCCT | |
| GTCTGAGGAGAACAATGACAGCAAGCTGCTGGAGTCTGGCCTGATGAACAGCCAGGAGAGCAGCTGGGGC | |
| AAGAATGTGAGCAGCAGGGAGATCACCAGGACCACCCTGCAGTCTGACCAGGAGGAGATTGACTATGATG | |
| ACACCATCTCTGTGGAGATGAAGAAGGAGGACTTTGACATCTACGACGAGGACGAGAACCAGAGCCCCAG | |
| GAGCTTCCAGAAGAAGACCAGGCACTACTTCATTGCTGCTGTGGAGAGGCTGTGGGACTATGGCATGAGC | |
| AGCAGCCCCCATGTGCTGAGGAACAGGGCCCAGTCTGGCTCTGTGCCCCAGTTCAAGAAGGTGGTGTTCC | |
| AGGAGTTCACTGATGGCAGCTTCACCCAGCCCCTGTACAGAGGGGAGCTGAATGAGCACCTGGGCCTGCT | |
| GGGCCCCTACATCAGGGCTGAGGTGGAGGACAACATCATGGTGACCTTCAGGAACCAGGCCAGCAGGCCC | |
| TACAGCTTCTACAGCAGCCTGATCAGCTATGAGGAGGACCAGAGGCAGGGGGCTGAGCCCAGGAAGAACT | |
| TTGTGAAGCCCAATGAAACCAAGACCTACTTCTGGAAGGTGCAGCACCACATGGCCCCCACCAAGGATGA | |
| GTTTGACTGCAAGGCCTGGGCCTACTTCTCTGATGTGGACCTGGAGAAGGATGTGCACTCTGGCCTGATT | |
| GGCCCCCTGCTGGTGTGCCACACCAACACCCTGAACCCTGCCCATGGCAGGCAGGTGACTGTGCAGGAGT | |
| TTGCCCTGTTCTTCACCATCTTTGATGAAACCAAGAGCTGGTACTTCACTGAGAACATGGAGAGGAACTG | |
| CAGGGCCCCCTGCAACATCCAGATGGAGGACCCCACCTTCAAGGAGAACTACAGGTTCCATGCCATCAAT | |
| GGCTACATCATGGACACCCTGCCTGGCCTGGTGATGGCCCAGGACCAGAGGATCAGGTGGTACCTGCTGA | |
| GCATGGGCAGCAATGAGAACATCCACAGCATCCACTTCTCTGGCCATGTGTTCACTGTGAGGAAGAAGGA | |
| GGAGTACAAGATGGCCCTGTACAACCTGTACCCTGGGGTGTTTGAGACTGTGGAGATGCTGCCCAGCAAG | |
| GCTGGCATCTGGAGGGTGGAGTGCCTGATTGGGGAGCACCTGCATGCTGGCATGAGCACCCTGTTCCTGG | |
| TGTACAGCAACAAGTGCCAGACCCCCCTGGGCATGGCCTCTGGCCACATCAGGGACTTCCAGATCACTGC | |
| CTCTGGCCAGTATGGCCAGTGGGCCCCCAAGCTGGCCAGGCTGCACTACTCTGGCAGCATCAATGCCTGG | |
| AGCACCAAGGAGCCCTTCAGCTGGATCAAGGTGGACCTGCTGGCCCCCATGATCATCCATGGCATCAAGA | |
| CCCAGGGGGCCAGGCAGAAGTTCAGCAGCCTGTACATCAGCCAGTTCATCATCATGTACAGCCTGGATGG | |
| CAAGAAGTGGCAGACCTACAGGGGCAACAGCACTGGCACCCTGATGGTGTTCTTTGGCAATGTGGACAGC | |
| TCTGGCATCAAGCACAACATCTTCAACCCCCCCATCATTGCCAGATACATCAGGCTGCACCCCACCCACT | |
| ACAGCATCAGGAGCACCCTGAGGATGGAGCTGATGGGCTGTGACCTGAACAGCTGCAGCATGCCCCTGGG | |
| CATGGAGAGCAAGGCCATCTCTGATGCCCAGATCACTGCCAGCAGCTACTTCACCAACATGTTTGCCACC | |
| TGGAGCCCCAGCAAGGCCAGGCTGCACCTGCAGGGCAGGAGCAATGCCTGGAGGCCCCAGGTCAACAACC | |
| CCAAGGAGTGGCTGCAGGTGGACTTCCAGAAGACCATGAAGGTGACTGGGGTGACCACCCAGGGGGTGAA | |
| GAGCCTGCTGACCAGCATGTATGTGAAGGAGTTCCTGATCAGCAGCAGCCAGGATGGCCACCAGTGGACC | |
| CTGTTCTTCCAGAATGGCAAGGTGAAGGTGTTCCAGGGCAACCAGGACAGCTTCACCCCTGTGGTGAACA | |
| GCCTGGACCCCCCCCTGCTGACCAGATACCTGAGGATTCACCCCCAGAGCTGGGTGCACCAGATTGCCCT | |
| GAGGATGGAGGTGCTGGGCTGTGAGGCCCAGGACCTGTACTGA | |
| Exemplified FVIII transgene (V3) | |
| Length: 4425; Molecule Type: DNA; Features Location/Qualifiers: source, | |
| 1..4425; mol_type, other DNA; note, codon-optimised FVIII transgene | |
| (V3); organism, synthetic construct | |
| SEQ ID NO: 18 | |
| ATGCAGATTGAGCTGAGCACCTGCTTCTTCCTGTGCCTGCTGAGGTTCTGCTTCTCTGCCACCAGGAGAT | |
| ACTACCTGGGGGCTGTGGAGCTGAGCTGGGACTACATGCAGTCTGACCTGGGGGAGCTGCCTGTGGATGC | |
| CAGGTTCCCCCCCAGAGTGCCCAAGAGCTTCCCCTTCAACACCTCTGTGGTGTACAAGAAGACCCTGTTT | |
| GTGGAGTTCACTGACCACCTGTTCAACATTGCCAAGCCCAGGCCCCCCTGGATGGGCCTGCTGGGCCCCA | |
| CCATCCAGGCTGAGGTGTATGACACTGTGGTGATCACCCTGAAGAACATGGCCAGCCACCCTGTGAGCCT | |
| GCATGCTGTGGGGGTGAGCTACTGGAAGGCCTCTGAGGGGGCTGAGTATGATGACCAGACCAGCCAGAGG | |
| GAGAAGGAGGATGACAAGGTGTTCCCTGGGGGCAGCCACACCTATGTGTGGCAGGTGCTGAAGGAGAATG | |
| GCCCCATGGCCTCTGACCCCCTGTGCCTGACCTACAGCTACCTGAGCCATGTGGACCTGGTGAAGGACCT | |
| GAACTCTGGCCTGATTGGGGCCCTGCTGGTGTGCAGGGAGGGCAGCCTGGCCAAGGAGAAGACCCAGACC | |
| CTGCACAAGTTCATCCTGCTGTTTGCTGTGTTTGATGAGGGCAAGAGCTGGCACTCTGAAACCAAGAACA | |
| GCCTGATGCAGGACAGGGATGCTGCCTCTGCCAGGGCCTGGCCCAAGATGCACACTGTGAATGGCTATGT | |
| GAACAGGAGCCTGCCTGGCCTGATTGGCTGCCACAGGAAGTCTGTGTACTGGCATGTGATTGGCATGGGC | |
| ACCACCCCTGAGGTGCACAGCATCTTCCTGGAGGGCCACACCTTCCTGGTCAGGAACCACAGGCAGGCCA | |
| GCCTGGAGATCAGCCCCATCACCTTCCTGACTGCCCAGACCCTGCTGATGGACCTGGGCCAGTTCCTGCT | |
| GTTCTGCCACATCAGCAGCCACCAGCATGATGGCATGGAGGCCTATGTGAAGGTGGACAGCTGCCCTGAG | |
| GAGCCCCAGCTGAGGATGAAGAACAATGAGGAGGCTGAGGACTATGATGATGACCTGACTGACTCTGAGA | |
| TGGATGTGGTGAGGTTTGATGATGACAACAGCCCCAGCTTCATCCAGATCAGGTCTGTGGCCAAGAAGCA | |
| CCCCAAGACCTGGGTGCACTACATTGCTGCTGAGGAGGAGGACTGGGACTATGCCCCCCTGGTGCTGGCC | |
| CCTGATGACAGGAGCTACAAGAGCCAGTACCTGAACAATGGCCCCCAGAGGATTGGCAGGAAGTACAAGA | |
| AGGTCAGGTTCATGGCCTACACTGATGAAACCTTCAAGACCAGGGAGGCCATCCAGCATGAGTCTGGCAT | |
| CCTGGGCCCCCTGCTGTATGGGGAGGTGGGGGACACCCTGCTGATCATCTTCAAGAACCAGGCCAGCAGG | |
| CCCTACAACATCTACCCCCATGGCATCACTGATGTGAGGCCCCTGTACAGCAGGAGGCTGCCCAAGGGGG | |
| TGAAGCACCTGAAGGACTTCCCCATCCTGCCTGGGGAGATCTTCAAGTACAAGTGGACTGTGACTGTGGA | |
| GGATGGCCCCACCAAGTCTGACCCCAGGTGCCTGACCAGATACTACAGCAGCTTTGTGAACATGGAGAGG | |
| GACCTGGCCTCTGGCCTGATTGGCCCCCTGCTGATCTGCTACAAGGAGTCTGTGGACCAGAGGGGCAACC | |
| AGATCATGTCTGACAAGAGGAATGTGATCCTGTTCTCTGTGTTTGATGAGAACAGGAGCTGGTACCTGAC | |
| TGAGAACATCCAGAGGTTCCTGCCCAACCCTGCTGGGGTGCAGCTGGAGGACCCTGAGTTCCAGGCCAGC | |
| AACATCATGCACAGCATCAATGGCTATGTGTTTGACAGCCTGCAGCTGTCTGTGTGCCTGCATGAGGTGG | |
| CCTACTGGTACATCCTGAGCATTGGGGCCCAGACTGACTTCCTGTCTGTGTTCTTCTCTGGCTACACCTT | |
| CAAGCACAAGATGGTGTATGAGGACACCCTGACCCTGTTCCCCTTCTCTGGGGAGACTGTGTTCATGAGC | |
| ATGGAGAACCCTGGCCTGTGGATTCTGGGCTGCCACAACTCTGACTTCAGGAACAGGGGCATGACTGCCC | |
| TGCTGAAAGTCTCCAGCTGTGACAAGAACACTGGGGACTACTATGAGGACAGCTATGAGGACATCTCTGC | |
| CTACCTGCTGAGCAAGAACAATGCCATTGAGCCCAGGAGCTTCAGCCAGAATGCCACTAATGTGTCTAAC | |
| AACAGCAACACCAGCAATGACAGCAATGTGTCTCCCCCAGTGCTGAAGAGGCACCAGAGGGAGATCACCA | |
| GGACCACCCTGCAGTCTGACCAGGAGGAGATTGACTATGATGACACCATCTCTGTGGAGATGAAGAAGGA | |
| GGACTTTGACATCTACGACGAGGACGAGAACCAGAGCCCCAGGAGCTTCCAGAAGAAGACCAGGCACTAC | |
| TTCATTGCTGCTGTGGAGAGGCTGTGGGACTATGGCATGAGCAGCAGCCCCCATGTGCTGAGGAACAGGG | |
| CCCAGTCTGGCTCTGTGCCCCAGTTCAAGAAGGTGGTGTTCCAGGAGTTCACTGATGGCAGCTTCACCCA | |
| GCCCCTGTACAGAGGGGAGCTGAATGAGCACCTGGGCCTGCTGGGCCCCTACATCAGGGCTGAGGTGGAG | |
| GACAACATCATGGTGACCTTCAGGAACCAGGCCAGCAGGCCCTACAGCTTCTACAGCAGCCTGATCAGCT | |
| ATGAGGAGGACCAGAGGCAGGGGGCTGAGCCCAGGAAGAACTTTGTGAAGCCCAATGAAACCAAGACCTA | |
| CTTCTGGAAGGTGCAGCACCACATGGCCCCCACCAAGGATGAGTTTGACTGCAAGGCCTGGGCCTACTTC | |
| TCTGATGTGGACCTGGAGAAGGATGTGCACTCTGGCCTGATTGGCCCCCTGCTGGTGTGCCACACCAACA | |
| CCCTGAACCCTGCCCATGGCAGGCAGGTGACTGTGCAGGAGTTTGCCCTGTTCTTCACCATCTTTGATGA | |
| AACCAAGAGCTGGTACTTCACTGAGAACATGGAGAGGAACTGCAGGGCCCCCTGCAACATCCAGATGGAG | |
| GACCCCACCTTCAAGGAGAACTACAGGTTCCATGCCATCAATGGCTACATCATGGACACCCTGCCTGGCC | |
| TGGTGATGGCCCAGGACCAGAGGATCAGGTGGTACCTGCTGAGCATGGGCAGCAATGAGAACATCCACAG | |
| CATCCACTTCTCTGGCCATGTGTTCACTGTGAGGAAGAAGGAGGAGTACAAGATGGCCCTGTACAACCTG | |
| TACCCTGGGGTGTTTGAGACTGTGGAGATGCTGCCCAGCAAGGCTGGCATCTGGAGGGTGGAGTGCCTGA | |
| TTGGGGAGCACCTGCATGCTGGCATGAGCACCCTGTTCCTGGTGTACAGCAACAAGTGCCAGACCCCCCT | |
| GGGCATGGCCTCTGGCCACATCAGGGACTTCCAGATCACTGCCTCTGGCCAGTATGGCCAGTGGGCCCCC | |
| AAGCTGGCCAGGCTGCACTACTCTGGCAGCATCAATGCCTGGAGCACCAAGGAGCCCTTCAGCTGGATCA | |
| AGGTGGACCTGCTGGCCCCCATGATCATCCATGGCATCAAGACCCAGGGGGCCAGGCAGAAGTTCAGCAG | |
| CCTGTACATCAGCCAGTTCATCATCATGTACAGCCTGGATGGCAAGAAGTGGCAGACCTACAGGGGCAAC | |
| AGCACTGGCACCCTGATGGTGTTCTTTGGCAATGTGGACAGCTCTGGCATCAAGCACAACATCTTCAACC | |
| CCCCCATCATTGCCAGATACATCAGGCTGCACCCCACCCACTACAGCATCAGGAGCACCCTGAGGATGGA | |
| GCTGATGGGCTGTGACCTGAACAGCTGCAGCATGCCCCTGGGCATGGAGAGCAAGGCCATCTCTGATGCC | |
| CAGATCACTGCCAGCAGCTACTTCACCAACATGTTTGCCACCTGGAGCCCCAGCAAGGCCAGGCTGCACC | |
| TGCAGGGCAGGAGCAATGCCTGGAGGCCCCAGGTCAACAACCCCAAGGAGTGGCTGCAGGTGGACTTCCA | |
| GAAGACCATGAAGGTGACTGGGGTGACCACCCAGGGGGTGAAGAGCCTGCTGACCAGCATGTATGTGAAG | |
| GAGTTCCTGATCAGCAGCAGCCAGGATGGCCACCAGTGGACCCTGTTCTTCCAGAATGGCAAGGTGAAGG | |
| TGTTCCAGGGCAACCAGGACAGCTTCACCCCTGTGGTGAACAGCCTGGACCCCCCCCTGCTGACCAGATA | |
| CCTGAGGATTCACCCCCAGAGCTGGGTGCACCAGATTGCCCTGAGGATGGAGGTGCTGGGCTGTGAGGCC | |
| CAGGACCTGTACTGA | |
| Complementary strand to the exemplified FVIII transgene (N6) | |
| Length: 5013; Molecule Type: DNA; Features Location/Qualifiers: source, | |
| 1..5013; mol_type, other DNA; note, codon-optimised FVIII transgene | |
| (N6) complementary strand; organism, synthetic construct | |
| SEQ ID NO: 19 | |
| TACGTCTAACTCGACTCGTGGACGAAGAAGGACACGGACGACTCCAAGACGAAGAGACGGTGGTCCTCTA | |
| TGATGGACCCCCGACACCTCGACTCGACCCTGATGTACGTCAGACTGGACCCCCTCGACGGACACCTACG | |
| GTCCAAGGGGGGGTCTCACGGGTTCTCGAAGGGGAAGTTGTGGAGACACCACATGTTCTTCTGGGACAAA | |
| CACCTCAAGTGACTGGTGGACAAGTTGTAACGGTTCGGGTCCGGGGGGACCTACCCGGACGACCCGGGGT | |
| GGTAGGTCCGACTCCACATACTGTGACACCACTAGTGGGACTTCTTGTACCGGTCGGTGGGACACTCGGA | |
| CGTACGACACCCCCACTCGATGACCTTCCGGAGACTCCCCCGACTCATACTACTGGTCTGGTCGGTCTCC | |
| CTCTTCCTCCTACTGTTCCACAAGGGACCCCCGTCGGTGTGGATACACACCGTCCACGACTTCCTCTTAC | |
| CGGGGTACCGGAGACTGGGGGACACGGACTGGATGTCGATGGACTCGGTACACCTGGACCACTTCCTGGA | |
| CTTGAGACCGGACTAACCCCGGGACGACCACACGTCCCTCCCGTCGGACCGGTTCCTCTTCTGGGTCTGG | |
| GACGTGTTCAAGTAGGACGACAAACGACACAAACTACTCCCGTTCTCGACCGTGAGACTTTGGTTCTTGT | |
| CGGACTACGTCCTGTCCCTACGACGGAGACGGTCCCGGACCGGGTTCTACGTGTGACACTTACCGATACA | |
| CTTGTCCTCGGACGGACCGGACTAACCGACGGTGTCCTTCAGACACATGACCGTACACTAACCGTACCCG | |
| TGGTGGGGACTCCACGTGTCGTAGAAGGACCTCCCGGTGTGGAAGGACCAGTCCTTGGTGTCCGTCCGGT | |
| CGGACCTCTAGTCGGGGTAGTGGAAGGACTGACGGGTCTGGGACGACTACCTGGACCCGGTCAAGGACGA | |
| CAAGACGGTGTAGTCGTCGGTGGTCGTACTACCGTACCTCCGGATACACTTCCACCTGTCGACGGGACTC | |
| CTCGGGGTCGACTCCTACTTCTTGTTACTCCTCCGACTCCTGATACTACTACTGGACTGACTGAGACTCT | |
| ACCTACACCACTCCAAACTACTACTGTTGTCGGGGTCGAAGTAGGTCTAGTCCAGACACCGGTTCTTCGT | |
| GGGGTTCTGGACCCACGTGATGTAACGACGACTCCTCCTCCTGACCCTGATACGGGGGGACCACGACCGG | |
| GGACTACTGTCCTCGATGTTCTCGGTCATGGACTTGTTACCGGGGGTCTCCTAACCGTCCTTCATGTTCT | |
| TCCAGTCCAAGTACCGGATGTGACTACTTTGGAAGTTCTGGTCCCTCCGGTAGGTCGTACTCAGACCGTA | |
| GGACCCGGGGGACGACATACCCCTCCACCCCCTGTGGGACGACTAGTAGAAGTTCTTGGTCCGGTCGTCC | |
| GGGATGTTGTAGATGGGGGTACCGTAGTGACTACACTCCGGGGACATGTCGTCCTCCGACGGGTTCCCCC | |
| ACTTCGTGGACTTCCTGAAGGGGTAGGACGGACCCCTCTAGAAGTTCATGTTCACCTGACACTGACACCT | |
| CCTACCGGGGTGGTTCAGACTGGGGTCCACGGACTGGTCTATGATGTCGTCGAAACACTTGTACCTCTCC | |
| CTGGACCGGAGACCGGACTAACCGGGGGACGACTAGACGATGTTCCTCAGACACCTGGTCTCCCCGTTGG | |
| TCTAGTACAGACTGTTCTCCTTACACTAGGACAAGAGACACAAACTACTCTTGTCCTCGACCATGGACTG | |
| ACTCTTGTAGGTCTCCAAGGACGGGTTGGGACGACCCCACGTCGACCTCCTGGGACTCAAGGTCCGGTCG | |
| TTGTAGTACGTGTCGTAGTTACCGATACACAAACTGTCGGACGTCGACAGACACACGGACGTACTCCACC | |
| GGATGACCATGTAGGACTCGTAACCCCGGGTCTGACTGAAGGACAGACACAAGAAGAGACCGATGTGGAA | |
| GTTCGTGTTCTACCACATACTCCTGTGGGACTGGGACAAGGGGAAGAGACCCCTCTGACACAAGTACTCG | |
| TACCTCTTGGGACCGGACACCTAAGACCCGACGGTGTTGAGACTGAAGTCCTTGTCCCCGTACTGACGGG | |
| ACGACTTTCAGAGGTCGACACTGTTCTTGTGACCCCTGATGATACTCCTGTCGATACTCCTGTAGAGACG | |
| GATGGACGACTCGTTCTTGTTACGGTAACTCGGGTCCTCGAAGTCGGTCTTGTCGTCCGTGGGGTCGTGG | |
| TCCGTCTTCGTCAAGTTACGGTGGTGGTAGGGACTCTTACTGTATCTCTTCTGTCTGGGTACCAAACGGG | |
| TGGCCTGGGGGTACGGGTTCTAGGTCTTACACTCGTCGAGACTGGACGACTACGACGACTCCGTCTCGGG | |
| GTGGGGGGTACCGGACTCGGACAGACTGGACGTCCTCCGGTTCATACTTTGGAAGAGACTACTGGGGTCG | |
| GGACCCCGGTAACTGTCGTTGTTGTCGGACAGACTCTACTGGGTGAAGTCCGGGGTCGACGTGGTGAGAC | |
| CCCTGTACCACAAGTGGGGACTCAGACCGGACGTCGACTCCGACTTACTCTTCGACCCGTGGTGACGACG | |
| GTGACTCGACTTCTTCGACCTGAAGTTTCAGAGGTCGTGGTCGTTGTTGGACTAGTCGTGGTAGGGGAGA | |
| CTGTTGGACCGACGACCGTGACTGTTGTGGTCGTCGGACCCGGGGGGGTCGTACGGACACGTGATACTGT | |
| CGGTCGACCTGTGGTGGGACAAACCGTTCTTCTCGTCGGGGGACTGACTCAGACCCCCGGGGGACTCGGA | |
| CAGACTCCTCTTGTTACTGTCGTTCGACGACCTCAGACCGGACTACTTGTCGGTCCTCTCGTCGACCCCG | |
| TTCTTACACTCGTCGTCCCTCTAGTGGTCCTGGTGGGACGTCAGACTGGTCCTCCTCTAACTGATACTAC | |
| TGTGGTAGAGACACCTCTACTTCTTCCTCCTGAAACTGTAGATGCTGCTCCTGCTCTTGGTCTCGGGGTC | |
| CTCGAAGGTCTTCTTCTGGTCCGTGATGAAGTAACGACGACACCTCTCCGACACCCTGATACCGTACTCG | |
| TCGTCGGGGGTACACGACTCCTTGTCCCGGGTCAGACCGAGACACGGGGTCAAGTTCTTCCACCACAAGG | |
| TCCTCAAGTGACTACCGTCGAAGTGGGTCGGGGACATGTCTCCCCTCGACTTACTCGTGGACCCGGACGA | |
| CCCGGGGATGTAGTCCCGACTCCACCTCCTGTTGTAGTACCACTGGAAGTCCTTGGTCCGGTCGTCCGGG | |
| ATGTCGAAGATGTCGTCGGACTAGTCGATACTCCTCCTGGTCTCCGTCCCCCGACTCGGGTCCTTCTTGA | |
| AACACTTCGGGTTACTTTGGTTCTGGATGAAGACCTTCCACGTCGTGGTGTACCGGGGGTGGTTCCTACT | |
| CAAACTGACGTTCCGGACCCGGATGAAGAGACTACACCTGGACCTCTTCCTACACGTGAGACCGGACTAA | |
| CCGGGGGACGACCACACGGTGTGGTTGTGGGACTTGGGACGGGTACCGTCCGTCCACTGACACGTCCTCA | |
| AACGGGACAAGAAGTGGTAGAAACTACTTTGGTTCTCGACCATGAAGTGACTCTTGTACCTCTCCTTGAC | |
| GTCCCGGGGGACGTTGTAGGTCTACCTCCTGGGGTGGAAGTTCCTCTTGATGTCCAAGGTACGGTAGTTA | |
| CCGATGTAGTACCTGTGGGACGGACCGGACCACTACCGGGTCCTGGTCTCCTAGTCCACCATGGACGACT | |
| CGTACCCGTCGTTACTCTTGTAGGTGTCGTAGGTGAAGAGACCGGTACACAAGTGACACTCCTTCTTCCT | |
| CCTCATGTTCTACCGGGACATGTTGGACATGGGACCCCACAAACTCTGACACCTCTACGACGGGTCGTTC | |
| CGACCGTAGACCTCCCACCTCACGGACTAACCCCTCGTGGACGTACGACCGTACTCGTGGGACAAGGACC | |
| ACATGTCGTTGTTCACGGTCTGGGGGGACCCGTACCGGAGACCGGTGTAGTCCCTGAAGGTCTAGTGACG | |
| GAGACCGGTCATACCGGTCACCCGGGGGTTCGACCGGTCCGACGTGATGAGACCGTCGTAGTTACGGACC | |
| TCGTGGTTCCTCGGGAAGTCGACCTAGTTCCACCTGGACGACCGGGGGTACTAGTAGGTACCGTAGTTCT | |
| GGGTCCCCCGGTCCGTCTTCAAGTCGTCGGACATGTAGTCGGTCAAGTAGTAGTACATGTCGGACCTACC | |
| GTTCTTCACCGTCTGGATGTCCCCGTTGTCGTGACCGTGGGACTACCACAAGAAACCGTTACACCTGTCG | |
| AGACCGTAGTTCGTGTTGTAGAAGTTGGGGGGGTAGTAACGGTCTATGTAGTCCGACGTGGGGTGGGTGA | |
| TGTCGTAGTCCTCGTGGGACTCCTACCTCGACTACCCGACACTGGACTTGTCGACGTCGTACGGGGACCC | |
| GTACCTCTCGTTCCGGTAGAGACTACGGGTCTAGTGACGGTCGTCGATGAAGTGGTTGTACAAACGGTGG | |
| ACCTCGGGGTCGTTCCGGTCCGACGTGGACGTCCCGTCCTCGTTACGGACCTCCGGGGTCCAGTTGTTGG | |
| GGTTCCTCACCGACGTCCACCTGAAGGTCTTCTGGTACTTCCACTGACCCCACTGGTGGGTCCCCCACTT | |
| CTCGGACGACTGGTCGTACATACACTTCCTCAAGGACTAGTCGTCGTCGGTCCTACCGGTGGTCACCTGG | |
| GACAAGAAGGTCTTACCGTTCCACTTCCACAAGGTCCCGTTGGTCCTGTCGAAGTGGGGACACCACTTGT | |
| CGGACCTGGGGGGGGACGACTGGTCTATGGACTCCTAAGTGGGGGTCTCGACCCACGTGGTCTAACGGGA | |
| CTCCTACCTCCACGACCCGACACTCCGGGTCCTGGACATGACT | |
| Complementary strand to the exemplified FVIII transgene (V3) | |
| Length: 4425; Molecule Type: DNA; Features Location/Qualifiers: source, | |
| 1..4425; mol_type, other DNA; note, codon-optimised FVIII transgene | |
| (V3) complementary strand; organism, synthetic construct | |
| SEQ ID NO: 20 | |
| TACGTCTAACTCGACTCGTGGACGAAGAAGGACACGGACGACTCCAAGACGAAGAGACGGTGGTCCTCTA | |
| TGATGGACCCCCGACACCTCGACTCGACCCTGATGTACGTCAGACTGGACCCCCTCGACGGACACCTACG | |
| GTCCAAGGGGGGGTCTCACGGGTTCTCGAAGGGGAAGTTGTGGAGACACCACATGTTCTTCTGGGACAAA | |
| CACCTCAAGTGACTGGTGGACAAGTTGTAACGGTTCGGGTCCGGGGGGACCTACCCGGACGACCCGGGGT | |
| GGTAGGTCCGACTCCACATACTGTGACACCACTAGTGGGACTTCTTGTACCGGTCGGTGGGACACTCGGA | |
| CGTACGACACCCCCACTCGATGACCTTCCGGAGACTCCCCCGACTCATACTACTGGTCTGGTCGGTCTCC | |
| CTCTTCCTCCTACTGTTCCACAAGGGACCCCCGTCGGTGTGGATACACACCGTCCACGACTTCCTCTTAC | |
| CGGGGTACCGGAGACTGGGGGACACGGACTGGATGTCGATGGACTCGGTACACCTGGACCACTTCCTGGA | |
| CTTGAGACCGGACTAACCCCGGGACGACCACACGTCCCTCCCGTCGGACCGGTTCCTCTTCTGGGTCTGG | |
| GACGTGTTCAAGTAGGACGACAAACGACACAAACTACTCCCGTTCTCGACCGTGAGACTTTGGTTCTTGT | |
| CGGACTACGTCCTGTCCCTACGACGGAGACGGTCCCGGACCGGGTTCTACGTGTGACACTTACCGATACA | |
| CTTGTCCTCGGACGGACCGGACTAACCGACGGTGTCCTTCAGACACATGACCGTACACTAACCGTACCCG | |
| TGGTGGGGACTCCACGTGTCGTAGAAGGACCTCCCGGTGTGGAAGGACCAGTCCTTGGTGTCCGTCCGGT | |
| CGGACCTCTAGTCGGGGTAGTGGAAGGACTGACGGGTCTGGGACGACTACCTGGACCCGGTCAAGGACGA | |
| CAAGACGGTGTAGTCGTCGGTGGTCGTACTACCGTACCTCCGGATACACTTCCACCTGTCGACGGGACTC | |
| CTCGGGGTCGACTCCTACTTCTTGTTACTCCTCCGACTCCTGATACTACTACTGGACTGACTGAGACTCT | |
| ACCTACACCACTCCAAACTACTACTGTTGTCGGGGTCGAAGTAGGTCTAGTCCAGACACCGGTTCTTCGT | |
| GGGGTTCTGGACCCACGTGATGTAACGACGACTCCTCCTCCTGACCCTGATACGGGGGGACCACGACCGG | |
| GGACTACTGTCCTCGATGTTCTCGGTCATGGACTTGTTACCGGGGGTCTCCTAACCGTCCTTCATGTTCT | |
| TCCAGTCCAAGTACCGGATGTGACTACTTTGGAAGTTCTGGTCCCTCCGGTAGGTCGTACTCAGACCGTA | |
| GGACCCGGGGGACGACATACCCCTCCACCCCCTGTGGGACGACTAGTAGAAGTTCTTGGTCCGGTCGTCC | |
| GGGATGTTGTAGATGGGGGTACCGTAGTGACTACACTCCGGGGACATGTCGTCCTCCGACGGGTTCCCCC | |
| ACTTCGTGGACTTCCTGAAGGGGTAGGACGGACCCCTCTAGAAGTTCATGTTCACCTGACACTGACACCT | |
| CCTACCGGGGTGGTTCAGACTGGGGTCCACGGACTGGTCTATGATGTCGTCGAAACACTTGTACCTCTCC | |
| CTGGACCGGAGACCGGACTAACCGGGGGACGACTAGACGATGTTCCTCAGACACCTGGTCTCCCCGTTGG | |
| TCTAGTACAGACTGTTCTCCTTACACTAGGACAAGAGACACAAACTACTCTTGTCCTCGACCATGGACTG | |
| ACTCTTGTAGGTCTCCAAGGACGGGTTGGGACGACCCCACGTCGACCTCCTGGGACTCAAGGTCCGGTCG | |
| TTGTAGTACGTGTCGTAGTTACCGATACACAAACTGTCGGACGTCGACAGACACACGGACGTACTCCACC | |
| GGATGACCATGTAGGACTCGTAACCCCGGGTCTGACTGAAGGACAGACACAAGAAGAGACCGATGTGGAA | |
| GTTCGTGTTCTACCACATACTCCTGTGGGACTGGGACAAGGGGAAGAGACCCCTCTGACACAAGTACTCG | |
| TACCTCTTGGGACCGGACACCTAAGACCCGACGGTGTTGAGACTGAAGTCCTTGTCCCCGTACTGACGGG | |
| ACGACTTTCAGAGGTCGACACTGTTCTTGTGACCCCTGATGATACTCCTGTCGATACTCCTGTAGAGACG | |
| GATGGACGACTCGTTCTTGTTACGGTAACTCGGGTCCTCGAAGTCGGTCTTACGGTGATTACACAGATTG | |
| TTGTCGTTGTGGTCGTTACTGTCGTTACACAGAGGGGGTCACGACTTCTCCGTGGTCTCCCTCTAGTGGT | |
| CCTGGTGGGACGTCAGACTGGTCCTCCTCTAACTGATACTACTGTGGTAGAGACACCTCTACTTCTTCCT | |
| CCTGAAACTGTAGATGCTGCTCCTGCTCTTGGTCTCGGGGTCCTCGAAGGTCTTCTTCTGGTCCGTGATG | |
| AAGTAACGACGACACCTCTCCGACACCCTGATACCGTACTCGTCGTCGGGGGTACACGACTCCTTGTCCC | |
| GGGTCAGACCGAGACACGGGGTCAAGTTCTTCCACCACAAGGTCCTCAAGTGACTACCGTCGAAGTGGGT | |
| CGGGGACATGTCTCCCCTCGACTTACTCGTGGACCCGGACGACCCGGGGATGTAGTCCCGACTCCACCTC | |
| CTGTTGTAGTACCACTGGAAGTCCTTGGTCCGGTCGTCCGGGATGTCGAAGATGTCGTCGGACTAGTCGA | |
| TACTCCTCCTGGTCTCCGTCCCCCGACTCGGGTCCTTCTTGAAACACTTCGGGTTACTTTGGTTCTGGAT | |
| GAAGACCTTCCACGTCGTGGTGTACCGGGGGTGGTTCCTACTCAAACTGACGTTCCGGACCCGGATGAAG | |
| AGACTACACCTGGACCTCTTCCTACACGTGAGACCGGACTAACCGGGGGACGACCACACGGTGTGGTTGT | |
| GGGACTTGGGACGGGTACCGTCCGTCCACTGACACGTCCTCAAACGGGACAAGAAGTGGTAGAAACTACT | |
| TTGGTTCTCGACCATGAAGTGACTCTTGTACCTCTCCTTGACGTCCCGGGGGACGTTGTAGGTCTACCTC | |
| CTGGGGTGGAAGTTCCTCTTGATGTCCAAGGTACGGTAGTTACCGATGTAGTACCTGTGGGACGGACCGG | |
| ACCACTACCGGGTCCTGGTCTCCTAGTCCACCATGGACGACTCGTACCCGTCGTTACTCTTGTAGGTGTC | |
| GTAGGTGAAGAGACCGGTACACAAGTGACACTCCTTCTTCCTCCTCATGTTCTACCGGGACATGTTGGAC | |
| ATGGGACCCCACAAACTCTGACACCTCTACGACGGGTCGTTCCGACCGTAGACCTCCCACCTCACGGACT | |
| AACCCCTCGTGGACGTACGACCGTACTCGTGGGACAAGGACCACATGTCGTTGTTCACGGTCTGGGGGGA | |
| CCCGTACCGGAGACCGGTGTAGTCCCTGAAGGTCTAGTGACGGAGACCGGTCATACCGGTCACCCGGGGG | |
| TTCGACCGGTCCGACGTGATGAGACCGTCGTAGTTACGGACCTCGTGGTTCCTCGGGAAGTCGACCTAGT | |
| TCCACCTGGACGACCGGGGGTACTAGTAGGTACCGTAGTTCTGGGTCCCCCGGTCCGTCTTCAAGTCGTC | |
| GGACATGTAGTCGGTCAAGTAGTAGTACATGTCGGACCTACCGTTCTTCACCGTCTGGATGTCCCCGTTG | |
| TCGTGACCGTGGGACTACCACAAGAAACCGTTACACCTGTCGAGACCGTAGTTCGTGTTGTAGAAGTTGG | |
| GGGGGTAGTAACGGTCTATGTAGTCCGACGTGGGGTGGGTGATGTCGTAGTCCTCGTGGGACTCCTACCT | |
| CGACTACCCGACACTGGACTTGTCGACGTCGTACGGGGACCCGTACCTCTCGTTCCGGTAGAGACTACGG | |
| GTCTAGTGACGGTCGTCGATGAAGTGGTTGTACAAACGGTGGACCTCGGGGTCGTTCCGGTCCGACGTGG | |
| ACGTCCCGTCCTCGTTACGGACCTCCGGGGTCCAGTTGTTGGGGTTCCTCACCGACGTCCACCTGAAGGT | |
| CTTCTGGTACTTCCACTGACCCCACTGGTGGGTCCCCCACTTCTCGGACGACTGGTCGTACATACACTTC | |
| CTCAAGGACTAGTCGTCGTCGGTCCTACCGGTGGTCACCTGGGACAAGAAGGTCTTACCGTTCCACTTCC | |
| ACAAGGTCCCGTTGGTCCTGTCGAAGTGGGGACACCACTTGTCGGACCTGGGGGGGGACGACTGGTCTAT | |
| GGACTCCTAAGTGGGGGTCTCGACCCACGTGGTCTAACGGGACTCCTACCTCCACGACCCGACACTCCGG | |
| GTCCTGGACATGACT | |
| Exemplified FVIII polypeptide (N6) | |
| Length: 1670; Molecule Type: AA; Features Location/Qualifiers: SOURCE, | |
| 1..1670; MOL_TYPE, protein; ORGANISM, Homo sapiens | |
| SEQ ID NO: 21 | |
| MQIELSTCFFLCLLRFCFSATRRYYLGAVELSWDYMQSDLGELPVDARFPPRVPKSFPFNTSVVYKKTLFV | |
| EFTDHLFNIAKPRPPWMGLLGPTIQAEVYDTVVITLKNMASHPVSLHAVGVSYWKASEGAEYDDQTSQREK | |
| EDDKVFPGGSHTYVWQVLKENGPMASDPLCLTYSYLSHVDLVKDLNSGLIGALLVCREGSLAKEKTQTLHK | |
| FILLFAVFDEGKSWHSETKNSLMQDRDAASARAWPKMHTVNGYVNRSLPGLIGCHRKSVYWHVIGMGTTPE | |
| VHSIFLEGHTFLVRNHRQASLEISPITFLTAQTLLMDLGQFLLFCHISSHQHDGMEAYVKVDSCPEEPQLR | |
| MKNNEEAEDYDDDLTDSEMDVVRFDDDNSPSFIQIRSVAKKHPKTWVHYIAAEEEDWDYAPLVLAPDDRSY | |
| KSQYLNNGPQRIGRKYKKVRFMAYTDETFKTREAIQHESGILGPLLYGEVGDTLLIIFKNQASRPYNIYPH | |
| GITDVRPLYSRRLPKGVKHLKDFPILPGEIFKYKWTVTVEDGPTKSDPRCLTRYYSSFVNMERDLASGLIG | |
| PLLICYKESVDQRGNQIMSDKRNVILFSVFDENRSWYLTENIQRFLPNPAGVQLEDPEFQASNIMHSINGY | |
| VFDSLQLSVCLHEVAYWYILSIGAQTDFLSVFFSGYTFKHKMVYEDTLTLFPFSGETVFMSMENPGLWILG | |
| CHNSDFRNRGMTALLKVSSCDKNTGDYYEDSYEDISAYLLSKNNAIEPRSFSQNSRHPSTRQKQFNATTIP | |
| ENDIEKTDPWFAHRTPMPKIQNVSSSDLLMLLRQSPTPHGLSLSDLQEAKYETFSDDPSPGAIDSNNSLSE | |
| MTHFRPQLHHSGDMVFTPESGLQLRLNEKLGTTAATELKKLDFKVSSTSNNLISTIPSDNLAAGTDNTSSL | |
| GPPSMPVHYDSQLDTTLFGKKSSPLTESGGPLSLSEENNDSKLLESGLMNSQESSWGKNVSSREITRTTLQ | |
| SDQEEIDYDDTISVEMKKEDFDIYDEDENQSPRSFQKKTRHYFIAAVERLWDYGMSSSPHVLRNRAQSGSV | |
| PQFKKVVFQEFTDGSFTQPLYRGELNEHLGLLGPYIRAEVEDNIMVTFRNQASRPYSFYSSLISYEEDQRQ | |
| GAEPRKNFVKPNETKTYFWKVQHHMAPTKDEFDCKAWAYFSDVDLEKDVHSGLIGPLLVCHTNTLNPAHGR | |
| QVTVQEFALFFTIFDETKSWYFTENMERNCRAPCNIQMEDPTFKENYRFHAINGYIMDTLPGLVMAQDQRI | |
| RWYLLSMGSNENIHSIHFSGHVFTVRKKEEYKMALYNLYPGVFETVEMLPSKAGIWRVECLIGEHLHAGMS | |
| TLFLVYSNKCQTPLGMASGHIRDFQITASGQYGQWAPKLARLHYSGSINAWSTKEPFSWIKVDLLAPMIIH | |
| GIKTQGARQKFSSLYISQFIIMYSLDGKKWQTYRGNSTGTLMVFFGNVDSSGIKHNIFNPPIIARYIRLHP | |
| THYSIRSTLRMELMGCDLNSCSMPLGMESKAISDAQITASSYFTNMFATWSPSKARLHLQGRSNAWRPQVN | |
| NPKEWLQVDFQKTMKVTGVTTQGVKSLLTSMYVKEFLISSSQDGHQWTLFFQNGKVKVFQGNQDSFTPVVN | |
| SLDPPLLTRYLRIHPQSWVHQIALRMEVLGCEAQDLY | |
| Exemplified FVIII polypeptide (V3) | |
| Length: 1474; Molecule Type: AA; Features Location/Qualifiers: SOURCE, | |
| 1..1474; MOL_TYPE, protein; ORGANISM, Homo sapiens | |
| SEQ ID NO: 22 | |
| MQIELSTCFFLCLLRFCFSATRRYYLGAVELSWDYMQSDLGELPVDARFPPRVPKSFPFNTSVVYKKTLF | |
| VEFTDHLFNIAKPRPPWMGLLGPTIQAEVYDTVVITLKNMASHPVSLHAVGVSYWKASEGAEYDDQTSQR | |
| EKEDDKVFPGGSHTYVWQVLKENGPMASDPLCLTYSYLSHVDLVKDLNSGLIGALLVCREGSLAKEKTQT | |
| LHKFILLFAVFDEGKSWHSETKNSLMQDRDAASARAWPKMHTVNGYVNRSLPGLIGCHRKSVYWHVIGMG | |
| TTPEVHSIFLEGHTFLVRNHRQASLEISPITFLTAQTLLMDLGQFLLFCHISSHQHDGMEAYVKVDSCPE | |
| EPQLRMKNNEEAEDYDDDLTDSEMDVVRFDDDNSPSFIQIRSVAKKHPKTWVHYIAAEEEDWDYAPLVLA | |
| PDDRSYKSQYLNNGPQRIGRKYKKVRFMAYTDETFKTREAIQHESGILGPLLYGEVGDTLLIIFKNQASR | |
| PYNIYPHGITDVRPLYSRRLPKGVKHLKDFPILPGEIFKYKWTVTVEDGPTKSDPRCLTRYYSSFVNMER | |
| DLASGLIGPLLICYKESVDQRGNQIMSDKRNVILFSVFDENRSWYLTENIQRFLPNPAGVQLEDPEFQAS | |
| NIMHSINGYVFDSLQLSVCLHEVAYWYILSIGAQTDFLSVFFSGYTFKHKMVYEDTLTLFPFSGETVFMS | |
| MENPGLWILGCHNSDFRNRGMTALLKVSSCDKNTGDYYEDSYEDISAYLLSKNNAIEPRSFSQNATNVSN | |
| NSNTSNDSNVSPPVLKRHQREITRTTLQSDQEEIDYDDTISVEMKKEDFDIYDEDENQSPRSFQKKTRHY | |
| FIAAVERLWDYGMSSSPHVLRNRAQSGSVPQFKKVVFQEFTDGSFTQPLYRGELNEHLGLLGPYIRAEVE | |
| DNIMVTFRNQASRPYSFYSSLISYEEDQRQGAEPRKNFVKPNETKTYFWKVQHHMAPTKDEFDCKAWAYF | |
| SDVDLEKDVHSGLIGPLLVCHTNTLNPAHGRQVTVQEFALFFTIFDETKSWYFTENMERNCRAPCNIQME | |
| DPTFKENYRFHAINGYIMDTLPGLVMAQDQRIRWYLLSMGSNENIHSIHFSGHVFTVRKKEEYKMALYNL | |
| YPGVFETVEMLPSKAGIWRVECLIGEHLHAGMSTLFLVYSNKCQTPLGMASGHIRDFQITASGQYGQWAP | |
| KLARLHYSGSINAWSTKEPFSWIKVDLLAPMIIHGIKTQGARQKFSSLYISQFIIMYSLDGKKWQTYRGN | |
| STGTLMVFFGNVDSSGIKHNIFNPPIIARYIRLHPTHYSIRSTLRMELMGCDLNSCSMPLGMESKAISDA | |
| QITASSYFTNMFATWSPSKARLHLQGRSNAWRPQVNNPKEWLQVDFQKTMKVTGVTTQGVKSLLTSMYVK | |
| EFLISSSQDGHQWTLFFQNGKVKVFQGNQDSFTPVVNSLDPPLLTRYLRIHPQSWVHQIALRMEVLGCEA | |
| QDLY | |
| Exemplified WPRE component (mWPRE) | |
| Length: 600; Molecule Type: DNA; Features Location/Qualifiers: source, | |
| 1..600; mol_type, unassigned DNA; organism, Woodchuck hepatitis virus | |
| SEQ ID NO: 23 | |
| 1 | GGGCCCAATC AACCTCTGGA TTACAAAATT TGTGAAAGAT TGACTGGTAT TCTTAACTAT |
| 61 | GTTGCTCCTT TTACGCTATG TGGATACGCT GCTTTAATGC CTTTGTATCA TGCTATTGCT |
| 121 | TCCCGTATGG CTTTCATTTT CTCCTCCTTG TATAAATCCT GGTTGCTGTC TCTTTATGAG |
| 181 | GAGTTGTGGC CCGTTGTCAG GCAACGTGGC GTGGTGTGCA CTGTGTTTGC TGACGCAACC |
| 241 | CCCACTGGTT GGGGCATTGC CACCACCTGT CAGCTCCTTT CCGGGACTTT CGCTTTCCCC |
| 301 | CTCCCTATTG CCACGGCGGA ACTCATCGCC GCCTGCCTTG CCCGCTGCTG GACAGGGGCT |
| 361 | CGGCTGTTGG GCACTGACAA TTCCGTGGTG TTGTCGGGGA AATCATCGTC CTTTCCTTGG |
| 421 | CTGCTCGCCT GTGTTGCCAC CTGGATTCTG CGCGGGACGT CCTTCTGCTA CGTCCCTTCG |
| 481 | GCCCTCAATC CAGCGGACCT TCCTTCCCGC GGCCTGCTGC CGGCTCTGCG GCCTCTTCCG |
| 541 | CGTCTTCGCC TTCGCCCTCA GACGAGTCGG ATCTCCCTTT GGGCCGCCTC CCCGCAAGCT |
| F/HN-SIV-hCEF-soA1AT plasmid as defined in FIG. 3 (pDNA1 pGM407) | |
| Length: 7349; Molecule Type: DNA; Features Location/Qualifiers: source, | |
| 1..7349; mol_type, other DNA; note, pGM407; organism, synthetic construct | |
| SEQ ID NO: 24 | |
| 1 | GGTACCTCAA TATTGGCCAT TAGCCATATT ATTCATTGGT TATATAGCAT AAATCAATAT |
| 61 | TGGCTATTGG CCATTGCATA CGTTGTATCT ATATCATAAT ATGTACATTT ATATTGGCTC |
| 121 | ATGTCCAATA TGACCGCCAT GTTGGCATTG ATTATTGACT AGTTATTAAT AGTAATCAAT |
| 181 | TACGGGGTCA TTAGTTCATA GCCCATATAT GGAGTTCCGC GTTACATAAC TTACGGTAAA |
| 241 | TGGCCCGCCT GGCTGACCGC CCAACGACCC CCGCCCATTG ACGTCAATAA TGACGTATGT |
| 301 | TCCCATAGTA ACGCCAATAG GGACTTTCCA TTGACGTCAA TGGGTGGAGT ATTTACGGTA |
| 361 | AACTGCCCAC TTGGCAGTAC ATCAAGTGTA TCATATGCCA AGTCCGCCCC CTATTGACGT |
| 421 | CAATGACGGT AAATGGCCCG CCTGGCATTA TGCCCAGTAC ATGACCTTAC GGGACTTTCC |
| 481 | TACTTGGCAG TACATCTACG TATTAGTCAT CGCTATTACC ATGGTGATGC GGTTTTGGCA |
| 541 | GTACACCAAT GGGCGTGGAT AGCGGTTTGA CTCACGGGGA TTTCCAAGTC TCCACCCCAT |
| 601 | TGACGTCAAT GGGAGTTTGT TTTGGCACCA AAATCAACGG GACTTTCCAA AATGTCGTAA |
| 661 | CAACTGCGAT CGCCCGCCCC GTTGACGCAA ATGGGCGGTA GGCGTGTACG GTGGGAGGTC |
| 721 | TATATAAGCA GAGCTCGCTG GCTTGTAACT CAGTCTCTTA CTAGGAGACC AGCTTGAGCC |
| 781 | TGGGTGTTCG CTGGTTAGCC TAACCTGGTT GGCCACCAGG GGTAAGGACT CCTTGGCTTA |
| 841 | GAAAGCTAAT AAACTTGCCT GCATTAGAGC TTATCTGAGT CAAGTGTCCT CATTGACGCC |
| 901 | TCACTCTCTT GAACGGGAAT CTTCCTTACT GGGTTCTCTC TCTGACCCAG GCGAGAGAAA |
| 961 | CTCCAGCAGT GGCGCCCGAA CAGGGACTTG AGTGAGAGTG TAGGCACGTA CAGCTGAGAA |
| 1021 | GGCGTCGGAC GCGAAGGAAG CGCGGGGTGC GACGCGACCA AGAAGGAGAC TTGGTGAGTA |
| 1081 | GGCTTCTCGA GTGCCGGGAA AAAGCTCGAG CCTAGTTAGA GGACTAGGAG AGGCCGTAGC |
| 1141 | CGTAACTACT CTTGGGCAAG TAGGGCAGGC GGTGGGTACG CAATGGGGGC GGCTACCTCA |
| 1201 | GCACTAAATA GGAGACAATT AGACCAATTT GAGAAAATAC GACTTCGCCC GAACGGAAAG |
| 1261 | AAAAAGTACC AAATTAAACA TTTAATATGG GCAGGCAAGG AGATGGAGCG CTTCGGCCTC |
| 1321 | CATGAGAGGT TGTTGGAGAC AGAGGAGGGG TGTAAAAGAA TCATAGAAGT CCTCTACCCC |
| 1381 | CTAGAACCAA CAGGATCGGA GGGCTTAAAA AGTCTGTTCA ATCTTGTGTG CGTGCTATAT |
| 1441 | TGCTTGCACA AGGAACAGAA AGTGAAAGAC ACAGAGGAAG CAGTAGCAAC AGTAAGACAA |
| 1501 | CACTGCCATC TAGTGGAAAA AGAAAAAAGT GCAACAGAGA CATCTAGTGG ACAAAAGAAA |
| 1561 | AATGACAAGG GAATAGCAGC GCCACCTGGT GGCAGTCAGA ATTTTCCAGC GCAACAACAA |
| 1621 | GGAAATGCCT GGGTACATGT ACCCTTGTCA CCGCGCACCT TAAATGCGTG GGTAAAAGCA |
| 1681 | GTAGAGGAGA AAAAATTTGG AGCAGAAATA GTACCCATTT TTTTGTTTCA AGCCCTATCG |
| 1741 | AATTCCCGTT TGTGCTAGGG TTCTTAGGCT TCTTGGGGGC TGCTGGAACT GCAATGGGAG |
| 1801 | CAGCGGCGAC AGCCCTGACG GTCCAGTCTC AGCATTTGCT TGCTGGGATA CTGCAGCAGC |
| 1861 | AGAAGAATCT GCTGGCGGCT GTGGAGGCTC AACAGCAGAT GTTGAAGCTG ACCATTTGGG |
| 1921 | GTGTTAAAAA CCTCAATGCC CGCGTCACAG CCCTTGAGAA GTACCTAGAG GATCAGGCAC |
| 1981 | GACTAAACTC CTGGGGGTGC GCATGGAAAC AAGTATGTCA TACCACAGTG GAGTGGCCCT |
| 2041 | GGACAAATCG GACTCCGGAT TGGCAAAATA TGACTTGGTT GGAGTGGGAA AGACAAATAG |
| 2101 | CTGATTTGGA AAGCAACATT ACGAGACAAT TAGTGAAGGC TAGAGAACAA GAGGAAAAGA |
| 2161 | ATCTAGATGC CTATCAGAAG TTAACTAGTT GGTCAGATTT CTGGTCTTGG TTCGATTTCT |
| 2221 | CAAAATGGCT TAACATTTTA AAAATGGGAT TTTTAGTAAT AGTAGGAATA ATAGGGTTAA |
| 2281 | GATTACTTTA CACAGTATAT GGATGTATAG TGAGGGTTAG GCAGGGATAT GTTCCTCTAT |
| 2341 | CTCCACAGAT CCATATCCGC GGCAATTTTA AAAGAAAGGG AGGAATAGGG GGACAGACTT |
| 2401 | CAGCAGAGAG ACTAATTAAT ATAATAACAA CACAATTAGA AATACAACAT TTACAAACCA |
| 2461 | AAATTCAAAA AATTTTAAAT TTTAGAGCCG CGGAGATCTG TTACATAACT TATGGTAAAT |
| 2521 | GGCCTGCCTG GCTGACTGCC CAATGACCCC TGCCCAATGA TGTCAATAAT GATGTATGTT |
| 2581 | CCCATGTAAT GCCAATAGGG ACTTTCCATT GATGTCAATG GGTGGAGTAT TTATGGTAAC |
| 2641 | TGCCCACTTG GCAGTACATC AAGTGTATCA TATGCCAAGT ATGCCCCCTA TTGATGTCAA |
| 2701 | TGATGGTAAA TGGCCTGCCT GGCATTATGC CCAGTACATG ACCTTATGGG ACTTTCCTAC |
| 2761 | TTGGCAGTAC ATCTATGTAT TAGTCATTGC TATTACCATG GGAATTCACT AGTGGAGAAG |
| 2821 | AGCATGCTTG AGGGCTGAGT GCCCCTCAGT GGGCAGAGAG CACATGGCCC ACAGTCCCTG |
| 2881 | AGAAGTTGGG GGGAGGGGTG GGCAATTGAA CTGGTGCCTA GAGAAGGTGG GGCTTGGGTA |
| 2941 | AACTGGGAAA GTGATGTGGT GTACTGGCTC CACCTTTTTC CCCAGGGTGG GGGAGAACCA |
| 3001 | TATATAAGTG CAGTAGTCTC TGTGAACATT CAAGCTTCTG CCTTCTCCCT CCTGTGAGTT |
| 3061 | TGCTAGCCAC CATGCCCAGC TCTGTGTCCT GGGGCATTCT GCTGCTGGCT GGCCTGTGCT |
| 3121 | GTCTGGTGCC TGTGTCCCTG GCTGAGGACC CTCAGGGGGA TGCTGCCCAG AAAACAGACA |
| 3181 | CCTCCCACCA TGACCAGGAC CACCCCACCT TCAACAAGAT CACCCCCAAC CTGGCAGAGT |
| 3241 | TTGCCTTCAG CCTGTACAGA CAGCTGGCCC ACCAGAGCAA CAGCACCAAC ATCTTTTTCA |
| 3301 | GCCCTGTGTC CATTGCCACA GCCTTTGCCA TGCTGAGCCT GGGCACCAAG GCTGACACCC |
| 3361 | ATGATGAGAT CCTGGAAGGC CTGAACTTCA ACCTGACAGA GATCCCTGAG GCCCAGATCC |
| 3421 | ATGAGGGCTT CCAGGAACTG CTGAGAACCC TGAACCAGCC AGACAGCCAG CTGCAGCTGA |
| 3481 | CAACAGGCAA TGGGCTGTTC CTGTCTGAGG GCCTGAAGCT GGTGGACAAG TTTCTGGAAG |
| 3541 | ATGTGAAGAA GCTGTACCAC TCTGAGGCCT TCACAGTGAA CTTTGGGGAC ACAGAAGAGG |
| 3601 | CCAAGAAACA GATCAATGAC TATGTGGAAA AGGGCACCCA GGGCAAGATT GTGGACCTTG |
| 3661 | TGAAAGAGCT GGACAGGGAC ACTGTGTTTG CCCTTGTGAA CTACATCTTC TTCAAGGGCA |
| 3721 | AGTGGGAGAG GCCCTTTGAA GTGAAGGACA CTGAGGAAGA GGACTTCCAT GTGGACCAAG |
| 3781 | TGACCACAGT GAAGGTGCCA ATGATGAAGA GACTGGGGAT GTTCAATATC CAGCACTGCA |
| 3841 | AGAAACTGAG CAGCTGGGTG CTGCTGATGA AGTACCTGGG CAATGCTACA GCCATATTCT |
| 3901 | TTCTGCCTGA TGAGGGCAAG CTGCAGCACC TGGAAAATGA GCTGACCCAT GACATCATCA |
| 3961 | CCAAATTTCT GGAAAATGAG GACAGAAGAT CTGCCAGCCT GCATCTGCCC AAGCTGAGCA |
| 4021 | TCACAGGCAC ATATGACCTG AAGTCTGTGC TGGGACAGCT GGGAATCACC AAGGTGTTCA |
| 4081 | GCAATGGGGC AGACCTGAGT GGAGTGACAG AGGAAGCCCC TCTGAAGCTG TCCAAGGCTG |
| 4141 | TGCACAAGGC AGTGCTGACC ATTGATGAGA AGGGCACAGA GGCTGCTGGG GCCATGTTTC |
| 4201 | TGGAAGCCAT CCCCATGTCC ATCCCCCCAG AAGTGAAGTT CAACAAGCCC TTTGTGTTCC |
| 4261 | TGATGATTGA GCAGAACACC AAGAGCCCCC TGTTCATGGG CAAGGTTGTG AACCCCACCC |
| 4321 | AGAAATGAGG GCCCAATCAA CCTCTGGATT ACAAAATTTG TGAAAGATTG ACTGGTATTC |
| 4381 | TTAACTATGT TGCTCCTTTT ACGCTATGTG GATACGCTGC TTTAATGCCT TTGTATCATG |
| 4441 | CTATTGCTTC CCGTATGGCT TTCATTTTCT CCTCCTTGTA TAAATCCTGG TTGCTGTCTC |
| 4501 | TTTATGAGGA GTTGTGGCCC GTTGTCAGGC AACGTGGCGT GGTGTGCACT GTGTTTGCTG |
| 4561 | ACGCAACCCC CACTGGTTGG GGCATTGCCA CCACCTGTCA GCTCCTTTCC GGGACTTTCG |
| 4621 | CTTTCCCCCT CCCTATTGCC ACGGCGGAAC TCATCGCCGC CTGCCTTGCC CGCTGCTGGA |
| 4681 | CAGGGGCTCG GCTGTTGGGC ACTGACAATT CCGTGGTGTT GTCGGGGAAA TCATCGTCCT |
| 4741 | TTCCTTGGCT GCTCGCCTGT GTTGCCACCT GGATTCTGCG CGGGACGTCC TTCTGCTACG |
| 4801 | TCCCTTCGGC CCTCAATCCA GCGGACCTTC CTTCCCGCGG CCTGCTGCCG GCTCTGCGGC |
| 4861 | CTCTTCCGCG TCTTCGCCTT CGCCCTCAGA CGAGTCGGAT CTCCCTTTGG GCCGCCTCCC |
| 4921 | CGCAAGCTTC GCACTTTTTA AAAGAAAAGG GAGGACTGGA TGGGATTTAT TACTCCGATA |
| 4981 | GGACGCTGGC TTGTAACTCA GTCTCTTACT AGGAGACCAG CTTGAGCCTG GGTGTTCGCT |
| 5041 | GGTTAGCCTA ACCTGGTTGG CCACCAGGGG TAAGGACTCC TTGGCTTAGA AAGCTAATAA |
| 5101 | ACTTGCCTGC ATTAGAGCTC TTACGCGTCC CGGGCTCGAG ATCCGCATCT CAATTAGTCA |
| 5161 | GCAACCATAG TCCCGCCCCT AACTCCGCCC ATCCCGCCCC TAACTCCGCC CAGTTCCGCC |
| 5221 | CATTCTCCGC CCCATGGCTG ACTAATTTTT TTTATTTATG CAGAGGCCGA GGCCGCCTCG |
| 5281 | GCCTCTGAGC TATTCCAGAA GTAGTGAGGA GGCTTTTTTG GAGGCCTAGG CTTTTGCAAA |
| 5341 | AAGCTAACTT GTTTATTGCA GCTTATAATG GTTACAAATA AAGCAATAGC ATCACAAATT |
| 5401 | TCACAAATAA AGCATTTTTT TCACTGCATT CTAGTTGTGG TTTGTCCAAA CTCATCAATG |
| 5461 | TATCTTATCA TGTCTGTCCG CTTCCTCGCT CACTGACTCG CTGCGCTCGG TCGTTCGGCT |
| 5521 | GCGGCGAGCG GTATCAGCTC ACTCAAAGGC GGTAATACGG TTATCCACAG AATCAGGGGA |
| 5581 | TAACGCAGGA AAGAACATGT GAGCAAAAGG CCAGCAAAAG GCCAGGAACC GTAAAAAGGC |
| 5641 | CGCGTTGCTG GCGTTTTTCC ATAGGCTCCG CCCCCCTGAC GAGCATCACA AAAATCGACG |
| 5701 | CTCAAGTCAG AGGTGGCGAA ACCCGACAGG ACTATAAAGA TACCAGGCGT TTCCCCCTGG |
| 5761 | AAGCTCCCTC GTGCGCTCTC CTGTTCCGAC CCTGCCGCTT ACCGGATACC TGTCCGCCTT |
| 5821 | TCTCCCTTCG GGAAGCGTGG CGCTTTCTCA TAGCTCACGC TGTAGGTATC TCAGTTCGGT |
| 5881 | GTAGGTCGTT CGCTCCAAGC TGGGCTGTGT GCACGAACCC CCCGTTCAGC CCGACCGCTG |
| 5941 | CGCCTTATCC GGTAACTATC GTCTTGAGTC CAACCCGGTA AGACACGACT TATCGCCACT |
| 6001 | GGCAGCAGCC ACTGGTAACA GGATTAGCAG AGCGAGGTAT GTAGGCGGTG CTACAGAGTT |
| 6061 | CTTGAAGTGG TGGCCTAACT ACGGCTACAC TAGAAGAACA GTATTTGGTA TCTGCGCTCT |
| 6121 | GCTGAAGCCA GTTACCTTCG GAAAAAGAGT TGGTAGCTCT TGATCCGGCA AACAAACCAC |
| 6181 | CGCTGGTAGC GGTGGTTTTT TTGTTTGCAA GCAGCAGATT ACGCGCAGAA AAAAAGGATC |
| 6241 | TCAAGAAGAT CCTTTGATCT TTTCTACGGG GTCTGACGCT CAGTGGAACG AAAACTCACG |
| 6301 | TTAAGGGATT TTGGTCATGA GATTATCAAA AAGGATCTTC ACCTAGATCC TTTTAAATTA |
| 6361 | AAAATGAAGT TTTAAATCAA TCTAAAGTAT ATATGAGTAA ACTTGGTCTG ACAGTTAGAA |
| 6421 | AAACTCATCG AGCATCAAAT GAAACTGCAA TTTATTCATA TCAGGATTAT CAATACCATA |
| 6481 | TTTTTGAAAA AGCCGTTTCT GTAATGAAGG AGAAAACTCA CCGAGGCAGT TCCATAGGAT |
| 6541 | GGCAAGATCC TGGTATCGGT CTGCGATTCC GACTCGTCCA ACATCAATAC AACCTATTAA |
| 6601 | TTTCCCCTCG TCAAAAATAA GGTTATCAAG TGAGAAATCA CCATGAGTGA CGACTGAATC |
| 6661 | CGGTGAGAAT GGCAACAGCT TATGCATTTC TTTCCAGACT TGTTCAACAG GCCAGCCATT |
| 6721 | ACGCTCGTCA TCAAAATCAC TCGCATCAAC CAAACCGTTA TTCATTCGTG ATTGCGCCTG |
| 6781 | AGCGAGACGA AATACGCGAT CGCTGTTAAA AGGACAATTA CAAACAGGAA TCGAATGCAA |
| 6841 | CCGGCGCAGG AACACTGCCA GCGCATCAAC AATATTTTCA CCTGAATCAG GATATTCTTC |
| 6901 | TAATACCTGG AATGCTGTTT TTCCGGGGAT CGCAGTGGTG AGTAACCATG CATCATCAGG |
| 6961 | AGTACGGATA AAATGCTTGA TGGTCGGAAG AGGCATAAAT TCCGTCAGCC AGTTTAGTCT |
| 7021 | GACCATCTCA TCTGTAACAT CATTGGCAAC GCTACCTTTG CCATGTTTCA GAAACAACTC |
| 7081 | TGGCGCATCG GGCTTCCCAT ACAATCGATA GATTGTCGCA CCTGATTGCC CGACATTATC |
| 7141 | GCGAGCCCAT TTATACCCAT ATAAATCAGC ATCCATGTTG GAATTTAATC GCGGCCTAGA |
| 7201 | GCAAGACGTT TCCCGTTGAA TATGGCTCAT AACACCCCTT GTATTACTGT TTATGTAAGC |
| 7261 | AGACAGTTTT ATTGTTCATG ATGATATATT TTTATCTTGT GCAATGTAAC ATCAGAGATT |
| 7321 | TTGAGACACA ACAATTGGTC GACGGATCC |
| F/HN-SIV-CMV-HFVIII-V3 plasmid as defined in FIG. 4A (pDNA1 pGM411) | |
| Length: 10812; Molecule Type: DNA; Features Location/Qualifiers: source, | |
| 1..10812; mol_type, other DNA; note, pGM411; organism, synthetic construct | |
| SEQ ID NO: 25 | |
| 1 | GGTACCTCAA TATTGGCCAT TAGCCATATT ATTCATTGGT TATATAGCAT AAATCAATAT |
| 61 | TGGCTATTGG CCATTGCATA CGTTGTATCT ATATCATAAT ATGTACATTT ATATTGGCTC |
| 121 | ATGTCCAATA TGACCGCCAT GTTGGCATTG ATTATTGACT AGTTATTAAT AGTAATCAAT |
| 181 | TACGGGGTCA TTAGTTCATA GCCCATATAT GGAGTTCCGC GTTACATAAC TTACGGTAAA |
| 241 | TGGCCCGCCT GGCTGACCGC CCAACGACCC CCGCCCATTG ACGTCAATAA TGACGTATGT |
| 301 | TCCCATAGTA ACGCCAATAG GGACTTTCCA TTGACGTCAA TGGGTGGAGT ATTTACGGTA |
| 361 | AACTGCCCAC TTGGCAGTAC ATCAAGTGTA TCATATGCCA AGTCCGCCCC CTATTGACGT |
| 421 | CAATGACGGT AAATGGCCCG CCTGGCATTA TGCCCAGTAC ATGACCTTAC GGGACTTTCC |
| 481 | TACTTGGCAG TACATCTACG TATTAGTCAT CGCTATTACC ATGGTGATGC GGTTTTGGCA |
| 541 | GTACACCAAT GGGCGTGGAT AGCGGTTTGA CTCACGGGGA TTTCCAAGTC TCCACCCCAT |
| 601 | TGACGTCAAT GGGAGTTTGT TTTGGCACCA AAATCAACGG GACTTTCCAA AATGTCGTAA |
| 661 | CAACTGCGAT CGCCCGCCCC GTTGACGCAA ATGGGCGGTA GGCGTGTACG GTGGGAGGTC |
| 721 | TATATAAGCA GAGCTCGCTG GCTTGTAACT CAGTCTCTTA CTAGGAGACC AGCTTGAGCC |
| 781 | TGGGTGTTCG CTGGTTAGCC TAACCTGGTT GGCCACCAGG GGTAAGGACT CCTTGGCTTA |
| 841 | GAAAGCTAAT AAACTTGCCT GCATTAGAGC TTATCTGAGT CAAGTGTCCT CATTGACGCC |
| 901 | TCACTCTCTT GAACGGGAAT CTTCCTTACT GGGTTCTCTC TCTGACCCAG GCGAGAGAAA |
| 961 | CTCCAGCAGT GGCGCCCGAA CAGGGACTTG AGTGAGAGTG TAGGCACGTA CAGCTGAGAA |
| 1021 | GGCGTCGGAC GCGAAGGAAG CGCGGGGTGC GACGCGACCA AGAAGGAGAC TTGGTGAGTA |
| 1081 | GGCTTCTCGA GTGCCGGGAA AAAGCTCGAG CCTAGTTAGA GGACTAGGAG AGGCCGTAGC |
| 1141 | CGTAACTACT CTGGGCAAGT AGGGCAGGCG GTGGGTACGC AATGGGGGCG GCTACCTCAG |
| 1201 | CACTAAATAG GAGACAATTA GACCAATTTG AGAAAATACG ACTTCGCCCG AACGGAAAGA |
| 1261 | AAAAGTACCA AATTAAACAT TTAATATGGG CAGGCAAGGA GATGGAGCGC TTCGGCCTCC |
| 1321 | ATGAGAGGTT GTTGGAGACA GAGGAGGGGT GTAAAAGAAT CATAGAAGTC CTCTACCCCC |
| 1381 | TAGAACCAAC AGGATCGGAG GGCTTAAAAA GTCTGTTCAA TCTTGTGTGC GTGCTATATT |
| 1441 | GCTTGCACAA GGAACAGAAA GTGAAAGACA CAGAGGAAGC AGTAGCAACA GTAAGACAAC |
| 1501 | ACTGCCATCT AGTGGAAAAA GAAAAAAGTG CAACAGAGAC ATCTAGTGGA CAAAAGAAAA |
| 1561 | ATGACAAGGG AATAGCAGCG CCACCTGGTG GCAGTCAGAA TTTTCCAGCG CAACAACAAG |
| 1621 | GAAATGCCTG GGTACATGTA CCCTTGTCAC CGCGCACCTT AAATGCGTGG GTAAAAGCAG |
| 1681 | TAGAGGAGAA AAAATTTGGA GCAGAAATAG TACCCATGTT TCAAGCCCTA TCGAATTCCC |
| 1741 | GTTTGTGCTA GGGTTCTTAG GCTTCTTGGG GGCTGCTGGA ACTGCAATGG GAGCAGCGGC |
| 1801 | GACAGCCCTG ACGGTCCAGT CTCAGCATTT GCTTGCTGGG ATACTGCAGC AGCAGAAGAA |
| 1861 | TCTGCTGGCG GCTGTGGAGG CTCAACAGCA GATGTTGAAG CTGACCATTT GGGGTGTTAA |
| 1921 | AAACCTCAAT GCCCGCGTCA CAGCCCTTGA GAAGTACCTA GAGGATCAGG CACGACTAAA |
| 1981 | CTCCTGGGGG TGCGCATGGA AACAAGTATG TCATACCACA GTGGAGTGGC CCTGGACAAA |
| 2041 | TCGGACTCCG GATTGGCAAA ATATGACTTG GTTGGAGTGG GAAAGACAAA TAGCTGATTT |
| 2101 | GGAAAGCAAC ATTACGAGAC AATTAGTGAA GGCTAGAGAA CAAGAGGAAA AGAATCTAGA |
| 2161 | TGCCTATCAG AAGTTAACTA GTTGGTCAGA TTTCTGGTCT TGGTTCGATT TCTCAAAATG |
| 2221 | GCTTAACATT TTAAAAATGG GATTTTTAGT AATAGTAGGA ATAATAGGGT TAAGATTACT |
| 2281 | TTACACAGTA TATGGATGTA TAGTGAGGGT TAGGCAGGGA TATGTTCCTC TATCTCCACA |
| 2341 | GATCCATATC CGCGGCAATT TTAAAAGAAA GGGAGGAATA GGGGGACAGA CTTCAGCAGA |
| 2401 | GAGACTAATT AATATAATAA CAACACAATT AGAAATACAA CATTTACAAA CCAAAATTCA |
| 2461 | AAAAATTTTA AATTTTAGAG CCGCGGAGAT CTCAATATTG GCCATTAGCC ATATTATTCA |
| 2521 | TTGGTTATAT AGCATAAATC AATATTGGCT ATTGGCCATT GCATACGTTG TATCTATATC |
| 2581 | ATAATATGTA CATTTATATT GGCTCATGTC CAATATGACC GCCATGTTGG CATTGATTAT |
| 2641 | TGACTAGTTA TTAATAGTAA TCAATTACGG GGTCATTAGT TCATAGCCCA TATATGGAGT |
| 2701 | TCCGCGTTAC ATAACTTACG GTAAATGGCC CGCCTGGCTG ACCGCCCAAC GACCCCCGCC |
| 2761 | CATTGACGTC AATAATGACG TATGTTCCCA TAGTAACGCC AATAGGGACT TTCCATTGAC |
| 2821 | GTCAATGGGT GGAGTATTTA CGGTAAACTG CCCACTTGGC AGTACATCAA GTGTATCATA |
| 2881 | TGCCAAGTCC GCCCCCTATT GACGTCAATG ACGGTAAATG GCCCGCCTGG CATTATGCCC |
| 2941 | AGTACATGAC CTTACGGGAC TTTCCTACTT GGCAGTACAT CTACGTATTA GTCATCGCTA |
| 3001 | TTACCATGGT GATGCGGTTT TGGCAGTACA CCAATGGGCG TGGATAGCGG TTTGACTCAC |
| 3061 | GGGGATTTCC AAGTCTCCAC CCCATTGACG TCAATGGGAG TTTGTTTTGG CACCAAAATC |
| 3121 | AACGGGACTT TCCAAAATGT CGTAATAACC CCGCCCCGTT GACGCAAATG GGCGGTAGGC |
| 3181 | GTGTACGGTG GGAGGTCTAT ATAAGCAGAG CTCGTTTAGT GAACCGTCAG ATCACTAGAA |
| 3241 | GCTTTATTGC GGTAGTTTAT CACAGTTAAA TTGCTAACGC AGTCAGTGCT TCTGACACAA |
| 3301 | CAGTCTCGAA CTTAAGCTGC AGAAGTTGGT CGTGAGGCAC TGGGCAGGCT AGCCACCAAT |
| 3361 | GCAGATTGAG CTGAGCACCT GCTTCTTCCT GTGCCTGCTG AGGTTCTGCT TCTCTGCCAC |
| 3421 | CAGGAGATAC TACCTGGGGG CTGTGGAGCT GAGCTGGGAC TACATGCAGT CTGACCTGGG |
| 3481 | GGAGCTGCCT GTGGATGCCA GGTTCCCCCC CAGAGTGCCC AAGAGCTTCC CCTTCAACAC |
| 3541 | CTCTGTGGTG TACAAGAAGA CCCTGTTTGT GGAGTTCACT GACCACCTGT TCAACATTGC |
| 3601 | CAAGCCCAGG CCCCCCTGGA TGGGCCTGCT GGGCCCCACC ATCCAGGCTG AGGTGTATGA |
| 3661 | CACTGTGGTG ATCACCCTGA AGAACATGGC CAGCCACCCT GTGAGCCTGC ATGCTGTGGG |
| 3721 | GGTGAGCTAC TGGAAGGCCT CTGAGGGGGC TGAGTATGAT GACCAGACCA GCCAGAGGGA |
| 3781 | GAAGGAGGAT GACAAGGTGT TCCCTGGGGG CAGCCACACC TATGTGTGGC AGGTGCTGAA |
| 3841 | GGAGAATGGC CCCATGGCCT CTGACCCCCT GTGCCTGACC TACAGCTACC TGAGCCATGT |
| 3901 | GGACCTGGTG AAGGACCTGA ACTCTGGCCT GATTGGGGCC CTGCTGGTGT GCAGGGAGGG |
| 3961 | CAGCCTGGCC AAGGAGAAGA CCCAGACCCT GCACAAGTTC ATCCTGCTGT TTGCTGTGTT |
| 4021 | TGATGAGGGC AAGAGCTGGC ACTCTGAAAC CAAGAACAGC CTGATGCAGG ACAGGGATGC |
| 4081 | TGCCTCTGCC AGGGCCTGGC CCAAGATGCA CACTGTGAAT GGCTATGTGA ACAGGAGCCT |
| 4141 | GCCTGGCCTG ATTGGCTGCC ACAGGAAGTC TGTGTACTGG CATGTGATTG GCATGGGCAC |
| 4201 | CACCCCTGAG GTGCACAGCA TCTTCCTGGA GGGCCACACC TTCCTGGTCA GGAACCACAG |
| 4261 | GCAGGCCAGC CTGGAGATCA GCCCCATCAC CTTCCTGACT GCCCAGACCC TGCTGATGGA |
| 4321 | CCTGGGCCAG TTCCTGCTGT TCTGCCACAT CAGCAGCCAC CAGCATGATG GCATGGAGGC |
| 4381 | CTATGTGAAG GTGGACAGCT GCCCTGAGGA GCCCCAGCTG AGGATGAAGA ACAATGAGGA |
| 4441 | GGCTGAGGAC TATGATGATG ACCTGACTGA CTCTGAGATG GATGTGGTGA GGTTTGATGA |
| 4501 | TGACAACAGC CCCAGCTTCA TCCAGATCAG GTCTGTGGCC AAGAAGCACC CCAAGACCTG |
| 4561 | GGTGCACTAC ATTGCTGCTG AGGAGGAGGA CTGGGACTAT GCCCCCCTGG TGCTGGCCCC |
| 4621 | TGATGACAGG AGCTACAAGA GCCAGTACCT GAACAATGGC CCCCAGAGGA TTGGCAGGAA |
| 4681 | GTACAAGAAG GTCAGGTTCA TGGCCTACAC TGATGAAACC TTCAAGACCA GGGAGGCCAT |
| 4741 | CCAGCATGAG TCTGGCATCC TGGGCCCCCT GCTGTATGGG GAGGTGGGGG ACACCCTGCT |
| 4801 | GATCATCTTC AAGAACCAGG CCAGCAGGCC CTACAACATC TACCCCCATG GCATCACTGA |
| 4861 | TGTGAGGCCC CTGTACAGCA GGAGGCTGCC CAAGGGGGTG AAGCACCTGA AGGACTTCCC |
| 4921 | CATCCTGCCT GGGGAGATCT TCAAGTACAA GTGGACTGTG ACTGTGGAGG ATGGCCCCAC |
| 4981 | CAAGTCTGAC CCCAGGTGCC TGACCAGATA CTACAGCAGC TTTGTGAACA TGGAGAGGGA |
| 5041 | CCTGGCCTCT GGCCTGATTG GCCCCCTGCT GATCTGCTAC AAGGAGTCTG TGGACCAGAG |
| 5101 | GGGCAACCAG ATCATGTCTG ACAAGAGGAA TGTGATCCTG TTCTCTGTGT TTGATGAGAA |
| 5161 | CAGGAGCTGG TACCTGACTG AGAACATCCA GAGGTTCCTG CCCAACCCTG CTGGGGTGCA |
| 5221 | GCTGGAGGAC CCTGAGTTCC AGGCCAGCAA CATCATGCAC AGCATCAATG GCTATGTGTT |
| 5281 | TGACAGCCTG CAGCTGTCTG TGTGCCTGCA TGAGGTGGCC TACTGGTACA TCCTGAGCAT |
| 5341 | TGGGGCCCAG ACTGACTTCC TGTCTGTGTT CTTCTCTGGC TACACCTTCA AGCACAAGAT |
| 5401 | GGTGTATGAG GACACCCTGA CCCTGTTCCC CTTCTCTGGG GAGACTGTGT TCATGAGCAT |
| 5461 | GGAGAACCCT GGCCTGTGGA TTCTGGGCTG CCACAACTCT GACTTCAGGA ACAGGGGCAT |
| 5521 | GACTGCCCTG CTGAAAGTCT CCAGCTGTGA CAAGAACACT GGGGACTACT ATGAGGACAG |
| 5581 | CTATGAGGAC ATCTCTGCCT ACCTGCTGAG CAAGAACAAT GCCATTGAGC CCAGGAGCTT |
| 5641 | CAGCCAGAAT GCCACTAATG TGTCTAACAA CAGCAACACC AGCAATGACA GCAATGTGTC |
| 5701 | TCCCCCAGTG CTGAAGAGGC ACCAGAGGGA GATCACCAGG ACCACCCTGC AGTCTGACCA |
| 5761 | GGAGGAGATT GACTATGATG ACACCATCTC TGTGGAGATG AAGAAGGAGG ACTTTGACAT |
| 5821 | CTACGACGAG GACGAGAACC AGAGCCCCAG GAGCTTCCAG AAGAAGACCA GGCACTACTT |
| 5881 | CATTGCTGCT GTGGAGAGGC TGTGGGACTA TGGCATGAGC AGCAGCCCCC ATGTGCTGAG |
| 5941 | GAACAGGGCC CAGTCTGGCT CTGTGCCCCA GTTCAAGAAG GTGGTGTTCC AGGAGTTCAC |
| 6001 | TGATGGCAGC TTCACCCAGC CCCTGTACAG AGGGGAGCTG AATGAGCACC TGGGCCTGCT |
| 6061 | GGGCCCCTAC ATCAGGGCTG AGGTGGAGGA CAACATCATG GTGACCTTCA GGAACCAGGC |
| 6121 | CAGCAGGCCC TACAGCTTCT ACAGCAGCCT GATCAGCTAT GAGGAGGACC AGAGGCAGGG |
| 6181 | GGCTGAGCCC AGGAAGAACT TTGTGAAGCC CAATGAAACC AAGACCTACT TCTGGAAGGT |
| 6241 | GCAGCACCAC ATGGCCCCCA CCAAGGATGA GTTTGACTGC AAGGCCTGGG CCTACTTCTC |
| 6301 | TGATGTGGAC CTGGAGAAGG ATGTGCACTC TGGCCTGATT GGCCCCCTGC TGGTGTGCCA |
| 6361 | CACCAACACC CTGAACCCTG CCCATGGCAG GCAGGTGACT GTGCAGGAGT TTGCCCTGTT |
| 6421 | CTTCACCATC TTTGATGAAA CCAAGAGCTG GTACTTCACT GAGAACATGG AGAGGAACTG |
| 6481 | CAGGGCCCCC TGCAACATCC AGATGGAGGA CCCCACCTTC AAGGAGAACT ACAGGTTCCA |
| 6541 | TGCCATCAAT GGCTACATCA TGGACACCCT GCCTGGCCTG GTGATGGCCC AGGACCAGAG |
| 6601 | GATCAGGTGG TACCTGCTGA GCATGGGCAG CAATGAGAAC ATCCACAGCA TCCACTTCTC |
| 6661 | TGGCCATGTG TTCACTGTGA GGAAGAAGGA GGAGTACAAG ATGGCCCTGT ACAACCTGTA |
| 6721 | CCCTGGGGTG TTTGAGACTG TGGAGATGCT GCCCAGCAAG GCTGGCATCT GGAGGGTGGA |
| 6781 | GTGCCTGATT GGGGAGCACC TGCATGCTGG CATGAGCACC CTGTTCCTGG TGTACAGCAA |
| 6841 | CAAGTGCCAG ACCCCCCTGG GCATGGCCTC TGGCCACATC AGGGACTTCC AGATCACTGC |
| 6901 | CTCTGGCCAG TATGGCCAGT GGGCCCCCAA GCTGGCCAGG CTGCACTACT CTGGCAGCAT |
| 6961 | CAATGCCTGG AGCACCAAGG AGCCCTTCAG CTGGATCAAG GTGGACCTGC TGGCCCCCAT |
| 7021 | GATCATCCAT GGCATCAAGA CCCAGGGGGC CAGGCAGAAG TTCAGCAGCC TGTACATCAG |
| 7081 | CCAGTTCATC ATCATGTACA GCCTGGATGG CAAGAAGTGG CAGACCTACA GGGGCAACAG |
| 7141 | CACTGGCACC CTGATGGTGT TCTTTGGCAA TGTGGACAGC TCTGGCATCA AGCACAACAT |
| 7201 | CTTCAACCCC CCCATCATTG CCAGATACAT CAGGCTGCAC CCCACCCACT ACAGCATCAG |
| 7261 | GAGCACCCTG AGGATGGAGC TGATGGGCTG TGACCTGAAC AGCTGCAGCA TGCCCCTGGG |
| 7321 | CATGGAGAGC AAGGCCATCT CTGATGCCCA GATCACTGCC AGCAGCTACT TCACCAACAT |
| 7381 | GTTTGCCACC TGGAGCCCCA GCAAGGCCAG GCTGCACCTG CAGGGCAGGA GCAATGCCTG |
| 7441 | GAGGCCCCAG GTCAACAACC CCAAGGAGTG GCTGCAGGTG GACTTCCAGA AGACCATGAA |
| 7501 | GGTGACTGGG GTGACCACCC AGGGGGTGAA GAGCCTGCTG ACCAGCATGT ATGTGAAGGA |
| 7561 | GTTCCTGATC AGCAGCAGCC AGGATGGCCA CCAGTGGACC CTGTTCTTCC AGAATGGCAA |
| 7621 | GGTGAAGGTG TTCCAGGGCA ACCAGGACAG CTTCACCCCT GTGGTGAACA GCCTGGACCC |
| 7681 | CCCCCTGCTG ACCAGATACC TGAGGATTCA CCCCCAGAGC TGGGTGCACC AGATTGCCCT |
| 7741 | GAGGATGGAG GTGCTGGGCT GTGAGGCCCA GGACCTGTAC TGAGCGGCCG CGGGCCCAAT |
| 7801 | CAACCTCTGG ATTACAAAAT TTGTGAAAGA TTGACTGGTA TTCTTAACTA TGTTGCTCCT |
| 7861 | TTTACGCTAT GTGGATACGC TGCTTTAATG CCTTTGTATC ATGCTATTGC TTCCCGTATG |
| 7921 | GCTTTCATTT TCTCCTCCTT GTATAAATCC TGGTTGCTGT CTCTTTATGA GGAGTTGTGG |
| 7981 | CCCGTTGTCA GGCAACGTGG CGTGGTGTGC ACTGTGTTTG CTGACGCAAC CCCCACTGGT |
| 8041 | TGGGGCATTG CCACCACCTG TCAGCTCCTT TCCGGGACTT TCGCTTTCCC CCTCCCTATT |
| 8101 | GCCACGGCGG AACTCATCGC CGCCTGCCTT GCCCGCTGCT GGACAGGGGC TCGGCTGTTG |
| 8161 | GGCACTGACA ATTCCGTGGT GTTGTCGGGG AAATCATCGT CCTTTCCTTG GCTGCTCGCC |
| 8221 | TGTGTTGCCA CCTGGATTCT GCGCGGGACG TCCTTCTGCT ACGTCCCTTC GGCCCTCAAT |
| 8281 | CCAGCGGACC TTCCTTCCCG CGGCCTGCTG CCGGCTCTGC GGCCTCTTCC GCGTCTTCGC |
| 8341 | CTTCGCCCTC AGACGAGTCG GATCTCCCTT TGGGCCGCCT CCCCGCAAGC TTCGCACTTT |
| 8401 | TTAAAAGAAA AGGGAGGACT GGATGGGATT TATTACTCCG ATAGGACGCT GGCTTGTAAC |
| 8461 | TCAGTCTCTT ACTAGGAGAC CAGCTTGAGC CTGGGTGTTC GCTGGTTAGC CTAACCTGGT |
| 8521 | TGGCCACCAG GGGTAAGGAC TCCTTGGCTT AGAAAGCTAA TAAACTTGCC TGCATTAGAG |
| 8581 | CTCTTACGCG TCCCGGGCTC GAGATCCGCA TCTCAATTAG TCAGCAACCA TAGTCCCGCC |
| 8641 | CCTAACTCCG CCCATCCCGC CCCTAACTCC GCCCAGTTCC GCCCATTCTC CGCCCCATGG |
| 8701 | CTGACTAATT TTTTTTATTT ATGCAGAGGC CGAGGCCGCC TCGGCCTCTG AGCTATTCCA |
| 8761 | GAAGTAGTGA GGAGGCTTTT TTGGAGGCCT AGGCTTTTGC AAAAAGCTAA CTTGTTTATT |
| 8821 | GCAGCTTATA ATGGTTACAA ATAAAGCAAT AGCATCACAA ATTTCACAAA TAAAGCATTT |
| 8881 | TTTTCACTGC ATTCTAGTTG TGGTTTGTCC AAACTCATCA ATGTATCTTA TCATGTCTGT |
| 8941 | CCGCTTCCTC GCTCACTGAC TCGCTGCGCT CGGTCGTTCG GCTGCGGCGA GCGGTATCAG |
| 9001 | CTCACTCAAA GGCGGTAATA CGGTTATCCA CAGAATCAGG GGATAACGCA GGAAAGAACA |
| 9061 | TGTGAGCAAA AGGCCAGCAA AAGGCCAGGA ACCGTAAAAA GGCCGCGTTG CTGGCGTTTT |
| 9121 | TCCATAGGCT CCGCCCCCCT GACGAGCATC ACAAAAATCG ACGCTCAAGT CAGAGGTGGC |
| 9181 | GAAACCCGAC AGGACTATAA AGATACCAGG CGTTTCCCCC TGGAAGCTCC CTCGTGCGCT |
| 9241 | CTCCTGTTCC GACCCTGCCG CTTACCGGAT ACCTGTCCGC CTTTCTCCCT TCGGGAAGCG |
| 9301 | TGGCGCTTTC TCATAGCTCA CGCTGTAGGT ATCTCAGTTC GGTGTAGGTC GTTCGCTCCA |
| 9361 | AGCTGGGCTG TGTGCACGAA CCCCCCGTTC AGCCCGACCG CTGCGCCTTA TCCGGTAACT |
| 9421 | ATCGTCTTGA GTCCAACCCG GTAAGACACG ACTTATCGCC ACTGGCAGCA GCCACTGGTA |
| 9481 | ACAGGATTAG CAGAGCGAGG TATGTAGGCG GTGCTACAGA GTTCTTGAAG TGGTGGCCTA |
| 9541 | ACTACGGCTA CACTAGAAGA ACAGTATTTG GTATCTGCGC TCTGCTGAAG CCAGTTACCT |
| 9601 | TCGGAAAAAG AGTTGGTAGC TCTTGATCCG GCAAACAAAC CACCGCTGGT AGCGGTGGTT |
| 9661 | TTTTTGTTTG CAAGCAGCAG ATTACGCGCA GAAAAAAAGG ATCTCAAGAA GATCCTTTGA |
| 9721 | TCTTTTCTAC GGGGTCTGAC GCTCAGTGGA ACGAAAACTC ACGTTAAGGG ATTTTGGTCA |
| 9781 | TGAGATTATC AAAAAGGATC TTCACCTAGA TCCTTTTAAA TTAAAAATGA AGTTTTAAAT |
| 9841 | CAATCTAAAG TATATATGAG TAAACTTGGT CTGACAGTTA GAAAAACTCA TCGAGCATCA |
| 9901 | AATGAAACTG CAATTTATTC ATATCAGGAT TATCAATACC ATATTTTTGA AAAAGCCGTT |
| 9961 | TCTGTAATGA AGGAGAAAAC TCACCGAGGC AGTTCCATAG GATGGCAAGA TCCTGGTATC |
| 10021 | GGTCTGCGAT TCCGACTCGT CCAACATCAA TACAACCTAT TAATTTCCCC TCGTCAAAAA |
| 10081 | TAAGGTTATC AAGTGAGAAA TCACCATGAG TGACGACTGA ATCCGGTGAG AATGGCAACA |
| 10141 | GCTTATGCAT TTCTTTCCAG ACTTGTTCAA CAGGCCAGCC ATTACGCTCG TCATCAAAAT |
| 10201 | CACTCGCATC AACCAAACCG TTATTCATTC GTGATTGCGC CTGAGCGAGA CGAAATACGC |
| 10261 | GATCGCTGTT AAAAGGACAA TTACAAACAG GAATCGAATG CAACCGGCGC AGGAACACTG |
| 10321 | CCAGCGCATC AACAATATTT TCACCTGAAT CAGGATATTC TTCTAATACC TGGAATGCTG |
| 10381 | TTTTTCCGGG GATCGCAGTG GTGAGTAACC ATGCATCATC AGGAGTACGG ATAAAATGCT |
| 10441 | TGATGGTCGG AAGAGGCATA AATTCCGTCA GCCAGTTTAG TCTGACCATC TCATCTGTAA |
| 10501 | CATCATTGGC AACGCTACCT TTGCCATGTT TCAGAAACAA CTCTGGCGCA TCGGGCTTCC |
| 10561 | CATACAATCG ATAGATTGTC GCACCTGATT GCCCGACATT ATCGCGAGCC CATTTATACC |
| 10621 | CATATAAATC AGCATCCATG TTGGAATTTA ATCGCGGCCT AGAGCAAGAC GTTTCCCGTT |
| 10681 | GAATATGGCT CATAACACCC CTTGTATTAC TGTTTATGTA AGCAGACAGT TTTATTGTTC |
| 10741 | ATGATGATAT ATTTTTATCT TGTGCAATGT AACATCAGAG ATTTTGAGAC ACAACAATTG |
| 10801 | GTCGACGGAT CC |
| F/HN-SIV-hCEF-HFVIII-V3 plasmid as defined in FIG. 4B (pDNA1 pGM413) | |
| Length: 10519; Molecule Type: DNA; Features Location/Qualifiers: source, | |
| 1..10519; mol_type, other DNA; note, pGM413; organism, synthetic construct | |
| SEQ ID NO: 26 | |
| 1 | GGTACCTCAA TATTGGCCAT TAGCCATATT ATTCATTGGT TATATAGCAT AAATCAATAT |
| 61 | TGGCTATTGG CCATTGCATA CGTTGTATCT ATATCATAAT ATGTACATTT ATATTGGCTC |
| 121 | ATGTCCAATA TGACCGCCAT GTTGGCATTG ATTATTGACT AGTTATTAAT AGTAATCAAT |
| 181 | TACGGGGTCA TTAGTTCATA GCCCATATAT GGAGTTCCGC GTTACATAAC TTACGGTAAA |
| 241 | TGGCCCGCCT GGCTGACCGC CCAACGACCC CCGCCCATTG ACGTCAATAA TGACGTATGT |
| 301 | TCCCATAGTA ACGCCAATAG GGACTTTCCA TTGACGTCAA TGGGTGGAGT ATTTACGGTA |
| 361 | AACTGCCCAC TTGGCAGTAC ATCAAGTGTA TCATATGCCA AGTCCGCCCC CTATTGACGT |
| 421 | CAATGACGGT AAATGGCCCG CCTGGCATTA TGCCCAGTAC ATGACCTTAC GGGACTTTCC |
| 481 | TACTTGGCAG TACATCTACG TATTAGTCAT CGCTATTACC ATGGTGATGC GGTTTTGGCA |
| 541 | GTACACCAAT GGGCGTGGAT AGCGGTTTGA CTCACGGGGA TTTCCAAGTC TCCACCCCAT |
| 601 | TGACGTCAAT GGGAGTTTGT TTTGGCACCA AAATCAACGG GACTTTCCAA AATGTCGTAA |
| 661 | CAACTGCGAT CGCCCGCCCC GTTGACGCAA ATGGGCGGTA GGCGTGTACG GTGGGAGGTC |
| 721 | TATATAAGCA GAGCTCGCTG GCTTGTAACT CAGTCTCTTA CTAGGAGACC AGCTTGAGCC |
| 781 | TGGGTGTTCG CTGGTTAGCC TAACCTGGTT GGCCACCAGG GGTAAGGACT CCTTGGCTTA |
| 841 | GAAAGCTAAT AAACTTGCCT GCATTAGAGC TTATCTGAGT CAAGTGTCCT CATTGACGCC |
| 901 | TCACTCTCTT GAACGGGAAT CTTCCTTACT GGGTTCTCTC TCTGACCCAG GCGAGAGAAA |
| 961 | CTCCAGCAGT GGCGCCCGAA CAGGGACTTG AGTGAGAGTG TAGGCACGTA CAGCTGAGAA |
| 1021 | GGCGTCGGAC GCGAAGGAAG CGCGGGGTGC GACGCGACCA AGAAGGAGAC TTGGTGAGTA |
| 1081 | GGCTTCTCGA GTGCCGGGAA AAAGCTCGAG CCTAGTTAGA GGACTAGGAG AGGCCGTAGC |
| 1141 | CGTAACTACT CTGGGCAAGT AGGGCAGGCG GTGGGTACGC AATGGGGGCG GCTACCTCAG |
| 1201 | CACTAAATAG GAGACAATTA GACCAATTTG AGAAAATACG ACTTCGCCCG AACGGAAAGA |
| 1261 | AAAAGTACCA AATTAAACAT TTAATATGGG CAGGCAAGGA GATGGAGCGC TTCGGCCTCC |
| 1321 | ATGAGAGGTT GTTGGAGACA GAGGAGGGGT GTAAAAGAAT CATAGAAGTC CTCTACCCCC |
| 1381 | TAGAACCAAC AGGATCGGAG GGCTTAAAAA GTCTGTTCAA TCTTGTGTGC GTGCTATATT |
| 1441 | GCTTGCACAA GGAACAGAAA GTGAAAGACA CAGAGGAAGC AGTAGCAACA GTAAGACAAC |
| 1501 | ACTGCCATCT AGTGGAAAAA GAAAAAAGTG CAACAGAGAC ATCTAGTGGA CAAAAGAAAA |
| 1561 | ATGACAAGGG AATAGCAGCG CCACCTGGTG GCAGTCAGAA TTTTCCAGCG CAACAACAAG |
| 1621 | GAAATGCCTG GGTACATGTA CCCTTGTCAC CGCGCACCTT AAATGCGTGG GTAAAAGCAG |
| 1681 | TAGAGGAGAA AAAATTTGGA GCAGAAATAG TACCCATGTT TCAAGCCCTA TCGAATTCCC |
| 1741 | GTTTGTGCTA GGGTTCTTAG GCTTCTTGGG GGCTGCTGGA ACTGCAATGG GAGCAGCGGC |
| 1801 | GACAGCCCTG ACGGTCCAGT CTCAGCATTT GCTTGCTGGG ATACTGCAGC AGCAGAAGAA |
| 1861 | TCTGCTGGCG GCTGTGGAGG CTCAACAGCA GATGTTGAAG CTGACCATTT GGGGTGTTAA |
| 1921 | AAACCTCAAT GCCCGCGTCA CAGCCCTTGA GAAGTACCTA GAGGATCAGG CACGACTAAA |
| 1981 | CTCCTGGGGG TGCGCATGGA AACAAGTATG TCATACCACA GTGGAGTGGC CCTGGACAAA |
| 2041 | TCGGACTCCG GATTGGCAAA ATATGACTTG GTTGGAGTGG GAAAGACAAA TAGCTGATTT |
| 2101 | GGAAAGCAAC ATTACGAGAC AATTAGTGAA GGCTAGAGAA CAAGAGGAAA AGAATCTAGA |
| 2161 | TGCCTATCAG AAGTTAACTA GTTGGTCAGA TTTCTGGTCT TGGTTCGATT TCTCAAAATG |
| 2221 | GCTTAACATT TTAAAAATGG GATTTTTAGT AATAGTAGGA ATAATAGGGT TAAGATTACT |
| 2281 | TTACACAGTA TATGGATGTA TAGTGAGGGT TAGGCAGGGA TATGTTCCTC TATCTCCACA |
| 2341 | GATCCATATC CGCGGCAATT TTAAAAGAAA GGGAGGAATA GGGGGACAGA CTTCAGCAGA |
| 2401 | GAGACTAATT AATATAATAA CAACACAATT AGAAATACAA CATTTACAAA CCAAAATTCA |
| 2461 | AAAAATTTTA AATTTTAGAG CCGCGGAGAT CTGTTACATA ACTTATGGTA AATGGCCTGC |
| 2521 | CTGGCTGACT GCCCAATGAC CCCTGCCCAA TGATGTCAAT AATGATGTAT GTTCCCATGT |
| 2581 | AATGCCAATA GGGACTTTCC ATTGATGTCA ATGGGTGGAG TATTTATGGT AACTGCCCAC |
| 2641 | TTGGCAGTAC ATCAAGTGTA TCATATGCCA AGTATGCCCC CTATTGATGT CAATGATGGT |
| 2701 | AAATGGCCTG CCTGGCATTA TGCCCAGTAC ATGACCTTAT GGGACTTTCC TACTTGGCAG |
| 2761 | TACATCTATG TATTAGTCAT TGCTATTACC ATGGGAATTC ACTAGTGGAG AAGAGCATGC |
| 2821 | TTGAGGGCTG AGTGCCCCTC AGTGGGCAGA GAGCACATGG CCCACAGTCC CTGAGAAGTT |
| 2881 | GGGGGGAGGG GTGGGCAATT GAACTGGTGC CTAGAGAAGG TGGGGCTTGG GTAAACTGGG |
| 2941 | AAAGTGATGT GGTGTACTGG CTCCACCTTT TTCCCCAGGG TGGGGGAGAA CCATATATAA |
| 3001 | GTGCAGTAGT CTCTGTGAAC ATTCAAGCTT CTGCCTTCTC CCTCCTGTGA GTTTGCTAGC |
| 3061 | CACCAATGCA GATTGAGCTG AGCACCTGCT TCTTCCTGTG CCTGCTGAGG TTCTGCTTCT |
| 3121 | CTGCCACCAG GAGATACTAC CTGGGGGCTG TGGAGCTGAG CTGGGACTAC ATGCAGTCTG |
| 3181 | ACCTGGGGGA GCTGCCTGTG GATGCCAGGT TCCCCCCCAG AGTGCCCAAG AGCTTCCCCT |
| 3241 | TCAACACCTC TGTGGTGTAC AAGAAGACCC TGTTTGTGGA GTTCACTGAC CACCTGTTCA |
| 3301 | ACATTGCCAA GCCCAGGCCC CCCTGGATGG GCCTGCTGGG CCCCACCATC CAGGCTGAGG |
| 3361 | TGTATGACAC TGTGGTGATC ACCCTGAAGA ACATGGCCAG CCACCCTGTG AGCCTGCATG |
| 3421 | CTGTGGGGGT GAGCTACTGG AAGGCCTCTG AGGGGGCTGA GTATGATGAC CAGACCAGCC |
| 3481 | AGAGGGAGAA GGAGGATGAC AAGGTGTTCC CTGGGGGCAG CCACACCTAT GTGTGGCAGG |
| 3541 | TGCTGAAGGA GAATGGCCCC ATGGCCTCTG ACCCCCTGTG CCTGACCTAC AGCTACCTGA |
| 3601 | GCCATGTGGA CCTGGTGAAG GACCTGAACT CTGGCCTGAT TGGGGCCCTG CTGGTGTGCA |
| 3661 | GGGAGGGCAG CCTGGCCAAG GAGAAGACCC AGACCCTGCA CAAGTTCATC CTGCTGTTTG |
| 3721 | CTGTGTTTGA TGAGGGCAAG AGCTGGCACT CTGAAACCAA GAACAGCCTG ATGCAGGACA |
| 3781 | GGGATGCTGC CTCTGCCAGG GCCTGGCCCA AGATGCACAC TGTGAATGGC TATGTGAACA |
| 3841 | GGAGCCTGCC TGGCCTGATT GGCTGCCACA GGAAGTCTGT GTACTGGCAT GTGATTGGCA |
| 3901 | TGGGCACCAC CCCTGAGGTG CACAGCATCT TCCTGGAGGG CCACACCTTC CTGGTCAGGA |
| 3961 | ACCACAGGCA GGCCAGCCTG GAGATCAGCC CCATCACCTT CCTGACTGCC CAGACCCTGC |
| 4021 | TGATGGACCT GGGCCAGTTC CTGCTGTTCT GCCACATCAG CAGCCACCAG CATGATGGCA |
| 4081 | TGGAGGCCTA TGTGAAGGTG GACAGCTGCC CTGAGGAGCC CCAGCTGAGG ATGAAGAACA |
| 4141 | ATGAGGAGGC TGAGGACTAT GATGATGACC TGACTGACTC TGAGATGGAT GTGGTGAGGT |
| 4201 | TTGATGATGA CAACAGCCCC AGCTTCATCC AGATCAGGTC TGTGGCCAAG AAGCACCCCA |
| 4261 | AGACCTGGGT GCACTACATT GCTGCTGAGG AGGAGGACTG GGACTATGCC CCCCTGGTGC |
| 4321 | TGGCCCCTGA TGACAGGAGC TACAAGAGCC AGTACCTGAA CAATGGCCCC CAGAGGATTG |
| 4381 | GCAGGAAGTA CAAGAAGGTC AGGTTCATGG CCTACACTGA TGAAACCTTC AAGACCAGGG |
| 4441 | AGGCCATCCA GCATGAGTCT GGCATCCTGG GCCCCCTGCT GTATGGGGAG GTGGGGGACA |
| 4501 | CCCTGCTGAT CATCTTCAAG AACCAGGCCA GCAGGCCCTA CAACATCTAC CCCCATGGCA |
| 4561 | TCACTGATGT GAGGCCCCTG TACAGCAGGA GGCTGCCCAA GGGGGTGAAG CACCTGAAGG |
| 4621 | ACTTCCCCAT CCTGCCTGGG GAGATCTTCA AGTACAAGTG GACTGTGACT GTGGAGGATG |
| 4681 | GCCCCACCAA GTCTGACCCC AGGTGCCTGA CCAGATACTA CAGCAGCTTT GTGAACATGG |
| 4741 | AGAGGGACCT GGCCTCTGGC CTGATTGGCC CCCTGCTGAT CTGCTACAAG GAGTCTGTGG |
| 4801 | ACCAGAGGGG CAACCAGATC ATGTCTGACA AGAGGAATGT GATCCTGTTC TCTGTGTTTG |
| 4861 | ATGAGAACAG GAGCTGGTAC CTGACTGAGA ACATCCAGAG GTTCCTGCCC AACCCTGCTG |
| 4921 | GGGTGCAGCT GGAGGACCCT GAGTTCCAGG CCAGCAACAT CATGCACAGC ATCAATGGCT |
| 4981 | ATGTGTTTGA CAGCCTGCAG CTGTCTGTGT GCCTGCATGA GGTGGCCTAC TGGTACATCC |
| 5041 | TGAGCATTGG GGCCCAGACT GACTTCCTGT CTGTGTTCTT CTCTGGCTAC ACCTTCAAGC |
| 5101 | ACAAGATGGT GTATGAGGAC ACCCTGACCC TGTTCCCCTT CTCTGGGGAG ACTGTGTTCA |
| 5161 | TGAGCATGGA GAACCCTGGC CTGTGGATTC TGGGCTGCCA CAACTCTGAC TTCAGGAACA |
| 5221 | GGGGCATGAC TGCCCTGCTG AAAGTCTCCA GCTGTGACAA GAACACTGGG GACTACTATG |
| 5281 | AGGACAGCTA TGAGGACATC TCTGCCTACC TGCTGAGCAA GAACAATGCC ATTGAGCCCA |
| 5341 | GGAGCTTCAG CCAGAATGCC ACTAATGTGT CTAACAACAG CAACACCAGC AATGACAGCA |
| 5401 | ATGTGTCTCC CCCAGTGCTG AAGAGGCACC AGAGGGAGAT CACCAGGACC ACCCTGCAGT |
| 5461 | CTGACCAGGA GGAGATTGAC TATGATGACA CCATCTCTGT GGAGATGAAG AAGGAGGACT |
| 5521 | TTGACATCTA CGACGAGGAC GAGAACCAGA GCCCCAGGAG CTTCCAGAAG AAGACCAGGC |
| 5581 | ACTACTTCAT TGCTGCTGTG GAGAGGCTGT GGGACTATGG CATGAGCAGC AGCCCCCATG |
| 5641 | TGCTGAGGAA CAGGGCCCAG TCTGGCTCTG TGCCCCAGTT CAAGAAGGTG GTGTTCCAGG |
| 5701 | AGTTCACTGA TGGCAGCTTC ACCCAGCCCC TGTACAGAGG GGAGCTGAAT GAGCACCTGG |
| 5761 | GCCTGCTGGG CCCCTACATC AGGGCTGAGG TGGAGGACAA CATCATGGTG ACCTTCAGGA |
| 5821 | ACCAGGCCAG CAGGCCCTAC AGCTTCTACA GCAGCCTGAT CAGCTATGAG GAGGACCAGA |
| 5881 | GGCAGGGGGC TGAGCCCAGG AAGAACTTTG TGAAGCCCAA TGAAACCAAG ACCTACTTCT |
| 5941 | GGAAGGTGCA GCACCACATG GCCCCCACCA AGGATGAGTT TGACTGCAAG GCCTGGGCCT |
| 6001 | ACTTCTCTGA TGTGGACCTG GAGAAGGATG TGCACTCTGG CCTGATTGGC CCCCTGCTGG |
| 6061 | TGTGCCACAC CAACACCCTG AACCCTGCCC ATGGCAGGCA GGTGACTGTG CAGGAGTTTG |
| 6121 | CCCTGTTCTT CACCATCTTT GATGAAACCA AGAGCTGGTA CTTCACTGAG AACATGGAGA |
| 6181 | GGAACTGCAG GGCCCCCTGC AACATCCAGA TGGAGGACCC CACCTTCAAG GAGAACTACA |
| 6241 | GGTTCCATGC CATCAATGGC TACATCATGG ACACCCTGCC TGGCCTGGTG ATGGCCCAGG |
| 6301 | ACCAGAGGAT CAGGTGGTAC CTGCTGAGCA TGGGCAGCAA TGAGAACATC CACAGCATCC |
| 6361 | ACTTCTCTGG CCATGTGTTC ACTGTGAGGA AGAAGGAGGA GTACAAGATG GCCCTGTACA |
| 6421 | ACCTGTACCC TGGGGTGTTT GAGACTGTGG AGATGCTGCC CAGCAAGGCT GGCATCTGGA |
| 6481 | GGGTGGAGTG CCTGATTGGG GAGCACCTGC ATGCTGGCAT GAGCACCCTG TTCCTGGTGT |
| 6541 | ACAGCAACAA GTGCCAGACC CCCCTGGGCA TGGCCTCTGG CCACATCAGG GACTTCCAGA |
| 6601 | TCACTGCCTC TGGCCAGTAT GGCCAGTGGG CCCCCAAGCT GGCCAGGCTG CACTACTCTG |
| 6661 | GCAGCATCAA TGCCTGGAGC ACCAAGGAGC CCTTCAGCTG GATCAAGGTG GACCTGCTGG |
| 6721 | CCCCCATGAT CATCCATGGC ATCAAGACCC AGGGGGCCAG GCAGAAGTTC AGCAGCCTGT |
| 6781 | ACATCAGCCA GTTCATCATC ATGTACAGCC TGGATGGCAA GAAGTGGCAG ACCTACAGGG |
| 6841 | GCAACAGCAC TGGCACCCTG ATGGTGTTCT TTGGCAATGT GGACAGCTCT GGCATCAAGC |
| 6901 | ACAACATCTT CAACCCCCCC ATCATTGCCA GATACATCAG GCTGCACCCC ACCCACTACA |
| 6961 | GCATCAGGAG CACCCTGAGG ATGGAGCTGA TGGGCTGTGA CCTGAACAGC TGCAGCATGC |
| 7021 | CCCTGGGCAT GGAGAGCAAG GCCATCTCTG ATGCCCAGAT CACTGCCAGC AGCTACTTCA |
| 7081 | CCAACATGTT TGCCACCTGG AGCCCCAGCA AGGCCAGGCT GCACCTGCAG GGCAGGAGCA |
| 7141 | ATGCCTGGAG GCCCCAGGTC AACAACCCCA AGGAGTGGCT GCAGGTGGAC TTCCAGAAGA |
| 7201 | CCATGAAGGT GACTGGGGTG ACCACCCAGG GGGTGAAGAG CCTGCTGACC AGCATGTATG |
| 7261 | TGAAGGAGTT CCTGATCAGC AGCAGCCAGG ATGGCCACCA GTGGACCCTG TTCTTCCAGA |
| 7321 | ATGGCAAGGT GAAGGTGTTC CAGGGCAACC AGGACAGCTT CACCCCTGTG GTGAACAGCC |
| 7381 | TGGACCCCCC CCTGCTGACC AGATACCTGA GGATTCACCC CCAGAGCTGG GTGCACCAGA |
| 7441 | TTGCCCTGAG GATGGAGGTG CTGGGCTGTG AGGCCCAGGA CCTGTACTGA GCGGCCGCGG |
| 7501 | GCCCAATCAA CCTCTGGATT ACAAAATTTG TGAAAGATTG ACTGGTATTC TTAACTATGT |
| 7561 | TGCTCCTTTT ACGCTATGTG GATACGCTGC TTTAATGCCT TTGTATCATG CTATTGCTTC |
| 7621 | CCGTATGGCT TTCATTTTCT CCTCCTTGTA TAAATCCTGG TTGCTGTCTC TTTATGAGGA |
| 7681 | GTTGTGGCCC GTTGTCAGGC AACGTGGCGT GGTGTGCACT GTGTTTGCTG ACGCAACCCC |
| 7741 | CACTGGTTGG GGCATTGCCA CCACCTGTCA GCTCCTTTCC GGGACTTTCG CTTTCCCCCT |
| 7801 | CCCTATTGCC ACGGCGGAAC TCATCGCCGC CTGCCTTGCC CGCTGCTGGA CAGGGGCTCG |
| 7861 | GCTGTTGGGC ACTGACAATT CCGTGGTGTT GTCGGGGAAA TCATCGTCCT TTCCTTGGCT |
| 7921 | GCTCGCCTGT GTTGCCACCT GGATTCTGCG CGGGACGTCC TTCTGCTACG TCCCTTCGGC |
| 7981 | CCTCAATCCA GCGGACCTTC CTTCCCGCGG CCTGCTGCCG GCTCTGCGGC CTCTTCCGCG |
| 8041 | TCTTCGCCTT CGCCCTCAGA CGAGTCGGAT CTCCCTTTGG GCCGCCTCCC CGCAAGCTTC |
| 8101 | GCACTTTTTA AAAGAAAAGG GAGGACTGGA TGGGATTTAT TACTCCGATA GGACGCTGGC |
| 8161 | TTGTAACTCA GTCTCTTACT AGGAGACCAG CTTGAGCCTG GGTGTTCGCT GGTTAGCCTA |
| 8221 | ACCTGGTTGG CCACCAGGGG TAAGGACTCC TTGGCTTAGA AAGCTAATAA ACTTGCCTGC |
| 8281 | ATTAGAGCTC TTACGCGTCC CGGGCTCGAG ATCCGCATCT CAATTAGTCA GCAACCATAG |
| 8341 | TCCCGCCCCT AACTCCGCCC ATCCCGCCCC TAACTCCGCC CAGTTCCGCC CATTCTCCGC |
| 8401 | CCCATGGCTG ACTAATTTTT TTTATTTATG CAGAGGCCGA GGCCGCCTCG GCCTCTGAGC |
| 8461 | TATTCCAGAA GTAGTGAGGA GGCTTTTTTG GAGGCCTAGG CTTTTGCAAA AAGCTAACTT |
| 8521 | GTTTATTGCA GCTTATAATG GTTACAAATA AAGCAATAGC ATCACAAATT TCACAAATAA |
| 8581 | AGCATTTTTT TCACTGCATT CTAGTTGTGG TTTGTCCAAA CTCATCAATG TATCTTATCA |
| 8641 | TGTCTGTCCG CTTCCTCGCT CACTGACTCG CTGCGCTCGG TCGTTCGGCT GCGGCGAGCG |
| 8701 | GTATCAGCTC ACTCAAAGGC GGTAATACGG TTATCCACAG AATCAGGGGA TAACGCAGGA |
| 8761 | AAGAACATGT GAGCAAAAGG CCAGCAAAAG GCCAGGAACC GTAAAAAGGC CGCGTTGCTG |
| 8821 | GCGTTTTTCC ATAGGCTCCG CCCCCCTGAC GAGCATCACA AAAATCGACG CTCAAGTCAG |
| 8881 | AGGTGGCGAA ACCCGACAGG ACTATAAAGA TACCAGGCGT TTCCCCCTGG AAGCTCCCTC |
| 8941 | GTGCGCTCTC CTGTTCCGAC CCTGCCGCTT ACCGGATACC TGTCCGCCTT TCTCCCTTCG |
| 9001 | GGAAGCGTGG CGCTTTCTCA TAGCTCACGC TGTAGGTATC TCAGTTCGGT GTAGGTCGTT |
| 9061 | CGCTCCAAGC TGGGCTGTGT GCACGAACCC CCCGTTCAGC CCGACCGCTG CGCCTTATCC |
| 9121 | GGTAACTATC GTCTTGAGTC CAACCCGGTA AGACACGACT TATCGCCACT GGCAGCAGCC |
| 9181 | ACTGGTAACA GGATTAGCAG AGCGAGGTAT GTAGGCGGTG CTACAGAGTT CTTGAAGTGG |
| 9241 | TGGCCTAACT ACGGCTACAC TAGAAGAACA GTATTTGGTA TCTGCGCTCT GCTGAAGCCA |
| 9301 | GTTACCTTCG GAAAAAGAGT TGGTAGCTCT TGATCCGGCA AACAAACCAC CGCTGGTAGC |
| 9361 | GGTGGTTTTT TTGTTTGCAA GCAGCAGATT ACGCGCAGAA AAAAAGGATC TCAAGAAGAT |
| 9421 | CCTTTGATCT TTTCTACGGG GTCTGACGCT CAGTGGAACG AAAACTCACG TTAAGGGATT |
| 9481 | TTGGTCATGA GATTATCAAA AAGGATCTTC ACCTAGATCC TTTTAAATTA AAAATGAAGT |
| 9541 | TTTAAATCAA TCTAAAGTAT ATATGAGTAA ACTTGGTCTG ACAGTTAGAA AAACTCATCG |
| 9601 | AGCATCAAAT GAAACTGCAA TTTATTCATA TCAGGATTAT CAATACCATA TTTTTGAAAA |
| 9661 | AGCCGTTTCT GTAATGAAGG AGAAAACTCA CCGAGGCAGT TCCATAGGAT GGCAAGATCC |
| 9721 | TGGTATCGGT CTGCGATTCC GACTCGTCCA ACATCAATAC AACCTATTAA TTTCCCCTCG |
| 9781 | TCAAAAATAA GGTTATCAAG TGAGAAATCA CCATGAGTGA CGACTGAATC CGGTGAGAAT |
| 9841 | GGCAACAGCT TATGCATTTC TTTCCAGACT TGTTCAACAG GCCAGCCATT ACGCTCGTCA |
| 9901 | TCAAAATCAC TCGCATCAAC CAAACCGTTA TTCATTCGTG ATTGCGCCTG AGCGAGACGA |
| 9961 | AATACGCGAT CGCTGTTAAA AGGACAATTA CAAACAGGAA TCGAATGCAA CCGGCGCAGG |
| 10021 | AACACTGCCA GCGCATCAAC AATATTTTCA CCTGAATCAG GATATTCTTC TAATACCTGG |
| 10081 | AATGCTGTTT TTCCGGGGAT CGCAGTGGTG AGTAACCATG CATCATCAGG AGTACGGATA |
| 10141 | AAATGCTTGA TGGTCGGAAG AGGCATAAAT TCCGTCAGCC AGTTTAGTCT GACCATCTCA |
| 10201 | TCTGTAACAT CATTGGCAAC GCTACCTTTG CCATGTTTCA GAAACAACTC TGGCGCATCG |
| 10261 | GGCTTCCCAT ACAATCGATA GATTGTCGCA CCTGATTGCC CGACATTATC GCGAGCCCAT |
| 10321 | TTATACCCAT ATAAATCAGC ATCCATGTTG GAATTTAATC GCGGCCTAGA GCAAGACGTT |
| 10381 | TCCCGTTGAA TATGGCTCAT AACACCCCTT GTATTACTGT TTATGTAAGC AGACAGTTTT |
| 10441 | ATTGTTCATG ATGATATATT TTTATCTTGT GCAATGTAAC ATCAGAGATT TTGAGACACA |
| 10501 | ACAATTGGTC GACGGATCC |
| F/HN-SIV-CMV-HFVIII-N6-co plasmid as defined in FIG. 4C (pDNA1 pGM412) | |
| Length: 11400; Molecule Type: DNA; Features Location/Qualifiers: source, | |
| 1..11400; mol_type, other DNA; note, pGM412; organism, synthetic construct | |
| SEQ ID NO: 27 | |
| 1 | GGTACCTCAA TATTGGCCAT TAGCCATATT ATTCATTGGT TATATAGCAT AAATCAATAT |
| 61 | TGGCTATTGG CCATTGCATA CGTTGTATCT ATATCATAAT ATGTACATTT ATATTGGCTC |
| 121 | ATGTCCAATA TGACCGCCAT GTTGGCATTG ATTATTGACT AGTTATTAAT AGTAATCAAT |
| 181 | TACGGGGTCA TTAGTTCATA GCCCATATAT GGAGTTCCGC GTTACATAAC TTACGGTAAA |
| 241 | TGGCCCGCCT GGCTGACCGC CCAACGACCC CCGCCCATTG ACGTCAATAA TGACGTATGT |
| 301 | TCCCATAGTA ACGCCAATAG GGACTTTCCA TTGACGTCAA TGGGTGGAGT ATTTACGGTA |
| 361 | AACTGCCCAC TTGGCAGTAC ATCAAGTGTA TCATATGCCA AGTCCGCCCC CTATTGACGT |
| 421 | CAATGACGGT AAATGGCCCG CCTGGCATTA TGCCCAGTAC ATGACCTTAC GGGACTTTCC |
| 481 | TACTTGGCAG TACATCTACG TATTAGTCAT CGCTATTACC ATGGTGATGC GGTTTTGGCA |
| 541 | GTACACCAAT GGGCGTGGAT AGCGGTTTGA CTCACGGGGA TTTCCAAGTC TCCACCCCAT |
| 601 | TGACGTCAAT GGGAGTTTGT TTTGGCACCA AAATCAACGG GACTTTCCAA AATGTCGTAA |
| 661 | CAACTGCGAT CGCCCGCCCC GTTGACGCAA ATGGGCGGTA GGCGTGTACG GTGGGAGGTC |
| 721 | TATATAAGCA GAGCTCGCTG GCTTGTAACT CAGTCTCTTA CTAGGAGACC AGCTTGAGCC |
| 781 | TGGGTGTTCG CTGGTTAGCC TAACCTGGTT GGCCACCAGG GGTAAGGACT CCTTGGCTTA |
| 841 | GAAAGCTAAT AAACTTGCCT GCATTAGAGC TTATCTGAGT CAAGTGTCCT CATTGACGCC |
| 901 | TCACTCTCTT GAACGGGAAT CTTCCTTACT GGGTTCTCTC TCTGACCCAG GCGAGAGAAA |
| 961 | CTCCAGCAGT GGCGCCCGAA CAGGGACTTG AGTGAGAGTG TAGGCACGTA CAGCTGAGAA |
| 1021 | GGCGTCGGAC GCGAAGGAAG CGCGGGGTGC GACGCGACCA AGAAGGAGAC TTGGTGAGTA |
| 1081 | GGCTTCTCGA GTGCCGGGAA AAAGCTCGAG CCTAGTTAGA GGACTAGGAG AGGCCGTAGC |
| 1141 | CGTAACTACT CTGGGCAAGT AGGGCAGGCG GTGGGTACGC AATGGGGGCG GCTACCTCAG |
| 1201 | CACTAAATAG GAGACAATTA GACCAATTTG AGAAAATACG ACTTCGCCCG AACGGAAAGA |
| 1261 | AAAAGTACCA AATTAAACAT TTAATATGGG CAGGCAAGGA GATGGAGCGC TTCGGCCTCC |
| 1321 | ATGAGAGGTT GTTGGAGACA GAGGAGGGGT GTAAAAGAAT CATAGAAGTC CTCTACCCCC |
| 1381 | TAGAACCAAC AGGATCGGAG GGCTTAAAAA GTCTGTTCAA TCTTGTGTGC GTGCTATATT |
| 1441 | GCTTGCACAA GGAACAGAAA GTGAAAGACA CAGAGGAAGC AGTAGCAACA GTAAGACAAC |
| 1501 | ACTGCCATCT AGTGGAAAAA GAAAAAAGTG CAACAGAGAC ATCTAGTGGA CAAAAGAAAA |
| 1561 | ATGACAAGGG AATAGCAGCG CCACCTGGTG GCAGTCAGAA TTTTCCAGCG CAACAACAAG |
| 1621 | GAAATGCCTG GGTACATGTA CCCTTGTCAC CGCGCACCTT AAATGCGTGG GTAAAAGCAG |
| 1681 | TAGAGGAGAA AAAATTTGGA GCAGAAATAG TACCCATGTT TCAAGCCCTA TCGAATTCCC |
| 1741 | GTTTGTGCTA GGGTTCTTAG GCTTCTTGGG GGCTGCTGGA ACTGCAATGG GAGCAGCGGC |
| 1801 | GACAGCCCTG ACGGTCCAGT CTCAGCATTT GCTTGCTGGG ATACTGCAGC AGCAGAAGAA |
| 1861 | TCTGCTGGCG GCTGTGGAGG CTCAACAGCA GATGTTGAAG CTGACCATTT GGGGTGTTAA |
| 1921 | AAACCTCAAT GCCCGCGTCA CAGCCCTTGA GAAGTACCTA GAGGATCAGG CACGACTAAA |
| 1981 | CTCCTGGGGG TGCGCATGGA AACAAGTATG TCATACCACA GTGGAGTGGC CCTGGACAAA |
| 2041 | TCGGACTCCG GATTGGCAAA ATATGACTTG GTTGGAGTGG GAAAGACAAA TAGCTGATTT |
| 2101 | GGAAAGCAAC ATTACGAGAC AATTAGTGAA GGCTAGAGAA CAAGAGGAAA AGAATCTAGA |
| 2161 | TGCCTATCAG AAGTTAACTA GTTGGTCAGA TTTCTGGTCT TGGTTCGATT TCTCAAAATG |
| 2221 | GCTTAACATT TTAAAAATGG GATTTTTAGT AATAGTAGGA ATAATAGGGT TAAGATTACT |
| 2281 | TTACACAGTA TATGGATGTA TAGTGAGGGT TAGGCAGGGA TATGTTCCTC TATCTCCACA |
| 2341 | GATCCATATC CGCGGCAATT TTAAAAGAAA GGGAGGAATA GGGGGACAGA CTTCAGCAGA |
| 2401 | GAGACTAATT AATATAATAA CAACACAATT AGAAATACAA CATTTACAAA CCAAAATTCA |
| 2461 | AAAAATTTTA AATTTTAGAG CCGCGGAGAT CTCAATATTG GCCATTAGCC ATATTATTCA |
| 2521 | TTGGTTATAT AGCATAAATC AATATTGGCT ATTGGCCATT GCATACGTTG TATCTATATC |
| 2581 | ATAATATGTA CATTTATATT GGCTCATGTC CAATATGACC GCCATGTTGG CATTGATTAT |
| 2641 | TGACTAGTTA TTAATAGTAA TCAATTACGG GGTCATTAGT TCATAGCCCA TATATGGAGT |
| 2701 | TCCGCGTTAC ATAACTTACG GTAAATGGCC CGCCTGGCTG ACCGCCCAAC GACCCCCGCC |
| 2761 | CATTGACGTC AATAATGACG TATGTTCCCA TAGTAACGCC AATAGGGACT TTCCATTGAC |
| 2821 | GTCAATGGGT GGAGTATTTA CGGTAAACTG CCCACTTGGC AGTACATCAA GTGTATCATA |
| 2881 | TGCCAAGTCC GCCCCCTATT GACGTCAATG ACGGTAAATG GCCCGCCTGG CATTATGCCC |
| 2941 | AGTACATGAC CTTACGGGAC TTTCCTACTT GGCAGTACAT CTACGTATTA GTCATCGCTA |
| 3001 | TTACCATGGT GATGCGGTTT TGGCAGTACA CCAATGGGCG TGGATAGCGG TTTGACTCAC |
| 3061 | GGGGATTTCC AAGTCTCCAC CCCATTGACG TCAATGGGAG TTTGTTTTGG CACCAAAATC |
| 3121 | AACGGGACTT TCCAAAATGT CGTAATAACC CCGCCCCGTT GACGCAAATG GGCGGTAGGC |
| 3181 | GTGTACGGTG GGAGGTCTAT ATAAGCAGAG CTCGTTTAGT GAACCGTCAG ATCACTAGAA |
| 3241 | GCTTTATTGC GGTAGTTTAT CACAGTTAAA TTGCTAACGC AGTCAGTGCT TCTGACACAA |
| 3301 | CAGTCTCGAA CTTAAGCTGC AGAAGTTGGT CGTGAGGCAC TGGGCAGGCT AGCCACCAAT |
| 3361 | GCAGATTGAG CTGAGCACCT GCTTCTTCCT GTGCCTGCTG AGGTTCTGCT TCTCTGCCAC |
| 3421 | CAGGAGATAC TACCTGGGGG CTGTGGAGCT GAGCTGGGAC TACATGCAGT CTGACCTGGG |
| 3481 | GGAGCTGCCT GTGGATGCCA GGTTCCCCCC CAGAGTGCCC AAGAGCTTCC CCTTCAACAC |
| 3541 | CTCTGTGGTG TACAAGAAGA CCCTGTTTGT GGAGTTCACT GACCACCTGT TCAACATTGC |
| 3601 | CAAGCCCAGG CCCCCCTGGA TGGGCCTGCT GGGCCCCACC ATCCAGGCTG AGGTGTATGA |
| 3661 | CACTGTGGTG ATCACCCTGA AGAACATGGC CAGCCACCCT GTGAGCCTGC ATGCTGTGGG |
| 3721 | GGTGAGCTAC TGGAAGGCCT CTGAGGGGGC TGAGTATGAT GACCAGACCA GCCAGAGGGA |
| 3781 | GAAGGAGGAT GACAAGGTGT TCCCTGGGGG CAGCCACACC TATGTGTGGC AGGTGCTGAA |
| 3841 | GGAGAATGGC CCCATGGCCT CTGACCCCCT GTGCCTGACC TACAGCTACC TGAGCCATGT |
| 3901 | GGACCTGGTG AAGGACCTGA ACTCTGGCCT GATTGGGGCC CTGCTGGTGT GCAGGGAGGG |
| 3961 | CAGCCTGGCC AAGGAGAAGA CCCAGACCCT GCACAAGTTC ATCCTGCTGT TTGCTGTGTT |
| 4021 | TGATGAGGGC AAGAGCTGGC ACTCTGAAAC CAAGAACAGC CTGATGCAGG ACAGGGATGC |
| 4081 | TGCCTCTGCC AGGGCCTGGC CCAAGATGCA CACTGTGAAT GGCTATGTGA ACAGGAGCCT |
| 4141 | GCCTGGCCTG ATTGGCTGCC ACAGGAAGTC TGTGTACTGG CATGTGATTG GCATGGGCAC |
| 4201 | CACCCCTGAG GTGCACAGCA TCTTCCTGGA GGGCCACACC TTCCTGGTCA GGAACCACAG |
| 4261 | GCAGGCCAGC CTGGAGATCA GCCCCATCAC CTTCCTGACT GCCCAGACCC TGCTGATGGA |
| 4321 | CCTGGGCCAG TTCCTGCTGT TCTGCCACAT CAGCAGCCAC CAGCATGATG GCATGGAGGC |
| 4381 | CTATGTGAAG GTGGACAGCT GCCCTGAGGA GCCCCAGCTG AGGATGAAGA ACAATGAGGA |
| 4441 | GGCTGAGGAC TATGATGATG ACCTGACTGA CTCTGAGATG GATGTGGTGA GGTTTGATGA |
| 4501 | TGACAACAGC CCCAGCTTCA TCCAGATCAG GTCTGTGGCC AAGAAGCACC CCAAGACCTG |
| 4561 | GGTGCACTAC ATTGCTGCTG AGGAGGAGGA CTGGGACTAT GCCCCCCTGG TGCTGGCCCC |
| 4621 | TGATGACAGG AGCTACAAGA GCCAGTACCT GAACAATGGC CCCCAGAGGA TTGGCAGGAA |
| 4681 | GTACAAGAAG GTCAGGTTCA TGGCCTACAC TGATGAAACC TTCAAGACCA GGGAGGCCAT |
| 4741 | CCAGCATGAG TCTGGCATCC TGGGCCCCCT GCTGTATGGG GAGGTGGGGG ACACCCTGCT |
| 4801 | GATCATCTTC AAGAACCAGG CCAGCAGGCC CTACAACATC TACCCCCATG GCATCACTGA |
| 4861 | TGTGAGGCCC CTGTACAGCA GGAGGCTGCC CAAGGGGGTG AAGCACCTGA AGGACTTCCC |
| 4921 | CATCCTGCCT GGGGAGATCT TCAAGTACAA GTGGACTGTG ACTGTGGAGG ATGGCCCCAC |
| 4981 | CAAGTCTGAC CCCAGGTGCC TGACCAGATA CTACAGCAGC TTTGTGAACA TGGAGAGGGA |
| 5041 | CCTGGCCTCT GGCCTGATTG GCCCCCTGCT GATCTGCTAC AAGGAGTCTG TGGACCAGAG |
| 5101 | GGGCAACCAG ATCATGTCTG ACAAGAGGAA TGTGATCCTG TTCTCTGTGT TTGATGAGAA |
| 5161 | CAGGAGCTGG TACCTGACTG AGAACATCCA GAGGTTCCTG CCCAACCCTG CTGGGGTGCA |
| 5221 | GCTGGAGGAC CCTGAGTTCC AGGCCAGCAA CATCATGCAC AGCATCAATG GCTATGTGTT |
| 5281 | TGACAGCCTG CAGCTGTCTG TGTGCCTGCA TGAGGTGGCC TACTGGTACA TCCTGAGCAT |
| 5341 | TGGGGCCCAG ACTGACTTCC TGTCTGTGTT CTTCTCTGGC TACACCTTCA AGCACAAGAT |
| 5401 | GGTGTATGAG GACACCCTGA CCCTGTTCCC CTTCTCTGGG GAGACTGTGT TCATGAGCAT |
| 5461 | GGAGAACCCT GGCCTGTGGA TTCTGGGCTG CCACAACTCT GACTTCAGGA ACAGGGGCAT |
| 5521 | GACTGCCCTG CTGAAAGTCT CCAGCTGTGA CAAGAACACT GGGGACTACT ATGAGGACAG |
| 5581 | CTATGAGGAC ATCTCTGCCT ACCTGCTGAG CAAGAACAAT GCCATTGAGC CCAGGAGCTT |
| 5641 | CAGCCAGAAC AGCAGGCACC CCAGCACCAG GCAGAAGCAG TTCAATGCCA CCACCATCCC |
| 5701 | TGAGAATGAC ATAGAGAAGA CAGACCCATG GTTTGCCCAC CGGACCCCCA TGCCCAAGAT |
| 5761 | CCAGAATGTG AGCAGCTCTG ACCTGCTGAT GCTGCTGAGG CAGAGCCCCA CCCCCCATGG |
| 5821 | CCTGAGCCTG TCTGACCTGC AGGAGGCCAA GTATGAAACC TTCTCTGATG ACCCCAGCCC |
| 5881 | TGGGGCCATT GACAGCAACA ACAGCCTGTC TGAGATGACC CACTTCAGGC CCCAGCTGCA |
| 5941 | CCACTCTGGG GACATGGTGT TCACCCCTGA GTCTGGCCTG CAGCTGAGGC TGAATGAGAA |
| 6001 | GCTGGGCACC ACTGCTGCCA CTGAGCTGAA GAAGCTGGAC TTCAAAGTCT CCAGCACCAG |
| 6061 | CAACAACCTG ATCAGCACCA TCCCCTCTGA CAACCTGGCT GCTGGCACTG ACAACACCAG |
| 6121 | CAGCCTGGGC CCCCCCAGCA TGCCTGTGCA CTATGACAGC CAGCTGGACA CCACCCTGTT |
| 6181 | TGGCAAGAAG AGCAGCCCCC TGACTGAGTC TGGGGGCCCC CTGAGCCTGT CTGAGGAGAA |
| 6241 | CAATGACAGC AAGCTGCTGG AGTCTGGCCT GATGAACAGC CAGGAGAGCA GCTGGGGCAA |
| 6301 | GAATGTGAGC AGCAGGGAGA TCACCAGGAC CACCCTGCAG TCTGACCAGG AGGAGATTGA |
| 6361 | CTATGATGAC ACCATCTCTG TGGAGATGAA GAAGGAGGAC TTTGACATCT ACGACGAGGA |
| 6421 | CGAGAACCAG AGCCCCAGGA GCTTCCAGAA GAAGACCAGG CACTACTTCA TTGCTGCTGT |
| 6481 | GGAGAGGCTG TGGGACTATG GCATGAGCAG CAGCCCCCAT GTGCTGAGGA ACAGGGCCCA |
| 6541 | GTCTGGCTCT GTGCCCCAGT TCAAGAAGGT GGTGTTCCAG GAGTTCACTG ATGGCAGCTT |
| 6601 | CACCCAGCCC CTGTACAGAG GGGAGCTGAA TGAGCACCTG GGCCTGCTGG GCCCCTACAT |
| 6661 | CAGGGCTGAG GTGGAGGACA ACATCATGGT GACCTTCAGG AACCAGGCCA GCAGGCCCTA |
| 6721 | CAGCTTCTAC AGCAGCCTGA TCAGCTATGA GGAGGACCAG AGGCAGGGGG CTGAGCCCAG |
| 6781 | GAAGAACTTT GTGAAGCCCA ATGAAACCAA GACCTACTTC TGGAAGGTGC AGCACCACAT |
| 6841 | GGCCCCCACC AAGGATGAGT TTGACTGCAA GGCCTGGGCC TACTTCTCTG ATGTGGACCT |
| 6901 | GGAGAAGGAT GTGCACTCTG GCCTGATTGG CCCCCTGCTG GTGTGCCACA CCAACACCCT |
| 6961 | GAACCCTGCC CATGGCAGGC AGGTGACTGT GCAGGAGTTT GCCCTGTTCT TCACCATCTT |
| 7021 | TGATGAAACC AAGAGCTGGT ACTTCACTGA GAACATGGAG AGGAACTGCA GGGCCCCCTG |
| 7081 | CAACATCCAG ATGGAGGACC CCACCTTCAA GGAGAACTAC AGGTTCCATG CCATCAATGG |
| 7141 | CTACATCATG GACACCCTGC CTGGCCTGGT GATGGCCCAG GACCAGAGGA TCAGGTGGTA |
| 7201 | CCTGCTGAGC ATGGGCAGCA ATGAGAACAT CCACAGCATC CACTTCTCTG GCCATGTGTT |
| 7261 | CACTGTGAGG AAGAAGGAGG AGTACAAGAT GGCCCTGTAC AACCTGTACC CTGGGGTGTT |
| 7321 | TGAGACTGTG GAGATGCTGC CCAGCAAGGC TGGCATCTGG AGGGTGGAGT GCCTGATTGG |
| 7381 | GGAGCACCTG CATGCTGGCA TGAGCACCCT GTTCCTGGTG TACAGCAACA AGTGCCAGAC |
| 7441 | CCCCCTGGGC ATGGCCTCTG GCCACATCAG GGACTTCCAG ATCACTGCCT CTGGCCAGTA |
| 7501 | TGGCCAGTGG GCCCCCAAGC TGGCCAGGCT GCACTACTCT GGCAGCATCA ATGCCTGGAG |
| 7561 | CACCAAGGAG CCCTTCAGCT GGATCAAGGT GGACCTGCTG GCCCCCATGA TCATCCATGG |
| 7621 | CATCAAGACC CAGGGGGCCA GGCAGAAGTT CAGCAGCCTG TACATCAGCC AGTTCATCAT |
| 7681 | CATGTACAGC CTGGATGGCA AGAAGTGGCA GACCTACAGG GGCAACAGCA CTGGCACCCT |
| 7741 | GATGGTGTTC TTTGGCAATG TGGACAGCTC TGGCATCAAG CACAACATCT TCAACCCCCC |
| 7801 | CATCATTGCC AGATACATCA GGCTGCACCC CACCCACTAC AGCATCAGGA GCACCCTGAG |
| 7861 | GATGGAGCTG ATGGGCTGTG ACCTGAACAG CTGCAGCATG CCCCTGGGCA TGGAGAGCAA |
| 7921 | GGCCATCTCT GATGCCCAGA TCACTGCCAG CAGCTACTTC ACCAACATGT TTGCCACCTG |
| 7981 | GAGCCCCAGC AAGGCCAGGC TGCACCTGCA GGGCAGGAGC AATGCCTGGA GGCCCCAGGT |
| 8041 | CAACAACCCC AAGGAGTGGC TGCAGGTGGA CTTCCAGAAG ACCATGAAGG TGACTGGGGT |
| 8101 | GACCACCCAG GGGGTGAAGA GCCTGCTGAC CAGCATGTAT GTGAAGGAGT TCCTGATCAG |
| 8161 | CAGCAGCCAG GATGGCCACC AGTGGACCCT GTTCTTCCAG AATGGCAAGG TGAAGGTGTT |
| 8221 | CCAGGGCAAC CAGGACAGCT TCACCCCTGT GGTGAACAGC CTGGACCCCC CCCTGCTGAC |
| 8281 | CAGATACCTG AGGATTCACC CCCAGAGCTG GGTGCACCAG ATTGCCCTGA GGATGGAGGT |
| 8341 | GCTGGGCTGT GAGGCCCAGG ACCTGTACTG AGCGGCCGCG GGCCCAATCA ACCTCTGGAT |
| 8401 | TACAAAATTT GTGAAAGATT GACTGGTATT CTTAACTATG TTGCTCCTTT TACGCTATGT |
| 8461 | GGATACGCTG CTTTAATGCC TTTGTATCAT GCTATTGCTT CCCGTATGGC TTTCATTTTC |
| 8521 | TCCTCCTTGT ATAAATCCTG GTTGCTGTCT CTTTATGAGG AGTTGTGGCC CGTTGTCAGG |
| 8581 | CAACGTGGCG TGGTGTGCAC TGTGTTTGCT GACGCAACCC CCACTGGTTG GGGCATTGCC |
| 8641 | ACCACCTGTC AGCTCCTTTC CGGGACTTTC GCTTTCCCCC TCCCTATTGC CACGGCGGAA |
| 8701 | CTCATCGCCG CCTGCCTTGC CCGCTGCTGG ACAGGGGCTC GGCTGTTGGG CACTGACAAT |
| 8761 | TCCGTGGTGT TGTCGGGGAA ATCATCGTCC TTTCCTTGGC TGCTCGCCTG TGTTGCCACC |
| 8821 | TGGATTCTGC GCGGGACGTC CTTCTGCTAC GTCCCTTCGG CCCTCAATCC AGCGGACCTT |
| 8881 | CCTTCCCGCG GCCTGCTGCC GGCTCTGCGG CCTCTTCCGC GTCTTCGCCT TCGCCCTCAG |
| 8941 | ACGAGTCGGA TCTCCCTTTG GGCCGCCTCC CCGCAAGCTT CGCACTTTTT AAAAGAAAAG |
| 9001 | GGAGGACTGG ATGGGATTTA TTACTCCGAT AGGACGCTGG CTTGTAACTC AGTCTCTTAC |
| 9061 | TAGGAGACCA GCTTGAGCCT GGGTGTTCGC TGGTTAGCCT AACCTGGTTG GCCACCAGGG |
| 9121 | GTAAGGACTC CTTGGCTTAG AAAGCTAATA AACTTGCCTG CATTAGAGCT CTTACGCGTC |
| 9181 | CCGGGCTCGA GATCCGCATC TCAATTAGTC AGCAACCATA GTCCCGCCCC TAACTCCGCC |
| 9241 | CATCCCGCCC CTAACTCCGC CCAGTTCCGC CCATTCTCCG CCCCATGGCT GACTAATTTT |
| 9301 | TTTTATTTAT GCAGAGGCCG AGGCCGCCTC GGCCTCTGAG CTATTCCAGA AGTAGTGAGG |
| 9361 | AGGCTTTTTT GGAGGCCTAG GCTTTTGCAA AAAGCTAACT TGTTTATTGC AGCTTATAAT |
| 9421 | GGTTACAAAT AAAGCAATAG CATCACAAAT TTCACAAATA AAGCATTTTT TTCACTGCAT |
| 9481 | TCTAGTTGTG GTTTGTCCAA ACTCATCAAT GTATCTTATC ATGTCTGTCC GCTTCCTCGC |
| 9541 | TCACTGACTC GCTGCGCTCG GTCGTTCGGC TGCGGCGAGC GGTATCAGCT CACTCAAAGG |
| 9601 | CGGTAATACG GTTATCCACA GAATCAGGGG ATAACGCAGG AAAGAACATG TGAGCAAAAG |
| 9661 | GCCAGCAAAA GGCCAGGAAC CGTAAAAAGG CCGCGTTGCT GGCGTTTTTC CATAGGCTCC |
| 9721 | GCCCCCCTGA CGAGCATCAC AAAAATCGAC GCTCAAGTCA GAGGTGGCGA AACCCGACAG |
| 9781 | GACTATAAAG ATACCAGGCG TTTCCCCCTG GAAGCTCCCT CGTGCGCTCT CCTGTTCCGA |
| 9841 | CCCTGCCGCT TACCGGATAC CTGTCCGCCT TTCTCCCTTC GGGAAGCGTG GCGCTTTCTC |
| 9901 | ATAGCTCACG CTGTAGGTAT CTCAGTTCGG TGTAGGTCGT TCGCTCCAAG CTGGGCTGTG |
| 9961 | TGCACGAACC CCCCGTTCAG CCCGACCGCT GCGCCTTATC CGGTAACTAT CGTCTTGAGT |
| 10021 | CCAACCCGGT AAGACACGAC TTATCGCCAC TGGCAGCAGC CACTGGTAAC AGGATTAGCA |
| 10081 | GAGCGAGGTA TGTAGGCGGT GCTACAGAGT TCTTGAAGTG GTGGCCTAAC TACGGCTACA |
| 10141 | CTAGAAGAAC AGTATTTGGT ATCTGCGCTC TGCTGAAGCC AGTTACCTTC GGAAAAAGAG |
| 10201 | TTGGTAGCTC TTGATCCGGC AAACAAACCA CCGCTGGTAG CGGTGGTTTT TTTGTTTGCA |
| 10261 | AGCAGCAGAT TACGCGCAGA AAAAAAGGAT CTCAAGAAGA TCCTTTGATC TTTTCTACGG |
| 10321 | GGTCTGACGC TCAGTGGAAC GAAAACTCAC GTTAAGGGAT TTTGGTCATG AGATTATCAA |
| 10381 | AAAGGATCTT CACCTAGATC CTTTTAAATT AAAAATGAAG TTTTAAATCA ATCTAAAGTA |
| 10441 | TATATGAGTA AACTTGGTCT GACAGTTAGA AAAACTCATC GAGCATCAAA TGAAACTGCA |
| 10501 | ATTTATTCAT ATCAGGATTA TCAATACCAT ATTTTTGAAA AAGCCGTTTC TGTAATGAAG |
| 10561 | GAGAAAACTC ACCGAGGCAG TTCCATAGGA TGGCAAGATC CTGGTATCGG TCTGCGATTC |
| 10621 | CGACTCGTCC AACATCAATA CAACCTATTA ATTTCCCCTC GTCAAAAATA AGGTTATCAA |
| 10681 | GTGAGAAATC ACCATGAGTG ACGACTGAAT CCGGTGAGAA TGGCAACAGC TTATGCATTT |
| 10741 | CTTTCCAGAC TTGTTCAACA GGCCAGCCAT TACGCTCGTC ATCAAAATCA CTCGCATCAA |
| 10801 | CCAAACCGTT ATTCATTCGT GATTGCGCCT GAGCGAGACG AAATACGCGA TCGCTGTTAA |
| 10861 | AAGGACAATT ACAAACAGGA ATCGAATGCA ACCGGCGCAG GAACACTGCC AGCGCATCAA |
| 10921 | CAATATTTTC ACCTGAATCA GGATATTCTT CTAATACCTG GAATGCTGTT TTTCCGGGGA |
| 10981 | TCGCAGTGGT GAGTAACCAT GCATCATCAG GAGTACGGAT AAAATGCTTG ATGGTCGGAA |
| 11041 | GAGGCATAAA TTCCGTCAGC CAGTTTAGTC TGACCATCTC ATCTGTAACA TCATTGGCAA |
| 11101 | CGCTACCTTT GCCATGTTTC AGAAACAACT CTGGCGCATC GGGCTTCCCA TACAATCGAT |
| 11161 | AGATTGTCGC ACCTGATTGC CCGACATTAT CGCGAGCCCA TTTATACCCA TATAAATCAG |
| 11221 | CATCCATGTT GGAATTTAAT CGCGGCCTAG AGCAAGACGT TTCCCGTTGA ATATGGCTCA |
| 11281 | TAACACCCCT TGTATTACTG TTTATGTAAG CAGACAGTTT TATTGTTCAT GATGATATAT |
| 11341 | TTTTATCTTG TGCAATGTAA CATCAGAGAT TTTGAGACAC AACAATTGGT CGACGGATCC |
| F/HN-SIV-hCEF-HFVIII-N6-co plasmid as defined in FIG. 4D (pDNA1 pGM414) | |
| Length: 11108; Molecule Type: DNA; Features Location/Qualifiers: source, | |
| 1..11108; mol_type, other DNA; note, pGM414; organism, synthetic construct | |
| SEQ ID NO: 28 | |
| 1 | GGTACCTCAA TATTGGCCAT TAGCCATATT ATTCATTGGT TATATAGCAT AAATCAATAT |
| 61 | TGGCTATTGG CCATTGCATA CGTTGTATCT ATATCATAAT ATGTACATTT ATATTGGCTC |
| 121 | ATGTCCAATA TGACCGCCAT GTTGGCATTG ATTATTGACT AGTTATTAAT AGTAATCAAT |
| 181 | TACGGGGTCA TTAGTTCATA GCCCATATAT GGAGTTCCGC GTTACATAAC TTACGGTAAA |
| 241 | TGGCCCGCCT GGCTGACCGC CCAACGACCC CCGCCCATTG ACGTCAATAA TGACGTATGT |
| 301 | TCCCATAGTA ACGCCAATAG GGACTTTCCA TTGACGTCAA TGGGTGGAGT ATTTACGGTA |
| 361 | AACTGCCCAC TTGGCAGTAC ATCAAGTGTA TCATATGCCA AGTCCGCCCC CTATTGACGT |
| 421 | CAATGACGGT AAATGGCCCG CCTGGCATTA TGCCCAGTAC ATGACCTTAC GGGACTTTCC |
| 481 | TACTTGGCAG TACATCTACG TATTAGTCAT CGCTATTACC ATGGTGATGC GGTTTTGGCA |
| 541 | GTACACCAAT GGGCGTGGAT AGCGGTTTGA CTCACGGGGA TTTCCAAGTC TCCACCCCAT |
| 601 | TGACGTCAAT GGGAGTTTGT TTTGGCACCA AAATCAACGG GACTTTCCAA AATGTCGTAA |
| 661 | CAACTGCGAT CGCCCGCCCC GTTGACGCAA ATGGGCGGTA GGCGTGTACG GTGGGAGGTC |
| 721 | TATATAAGCA GAGCTCGCTG GCTTGTAACT CAGTCTCTTA CTAGGAGACC AGCTTGAGCC |
| 781 | TGGGTGTTCG CTGGTTAGCC TAACCTGGTT GGCCACCAGG GGTAAGGACT CCTTGGCTTA |
| 841 | GAAAGCTAAT AAACTTGCCT GCATTAGAGC TTATCTGAGT CAAGTGTCCT CATTGACGCC |
| 901 | TCACTCTCTT GAACGGGAAT CTTCCTTACT GGGTTCTCTC TCTGACCCAG GCGAGAGAAA |
| 961 | CTCCAGCAGT GGCGCCCGAA CAGGGACTTG AGTGAGAGTG TAGGCACGTA CAGCTGAGAA |
| 1021 | GGCGTCGGAC GCGAAGGAAG CGCGGGGTGC GACGCGACCA AGAAGGAGAC TTGGTGAGTA |
| 1081 | GGCTTCTCGA GTGCCGGGAA AAAGCTCGAG CCTAGTTAGA GGACTAGGAG AGGCCGTAGC |
| 1141 | CGTAACTACT CTTGGGCAAG TAGGGCAGGC GGTGGGTACG CAATGGGGGC GGCTACCTCA |
| 1201 | GCACTAAATA GGAGACAATT AGACCAATTT GAGAAAATAC GACTTCGCCC GAACGGAAAG |
| 1261 | AAAAAGTACC AAATTAAACA TTTAATATGG GCAGGCAAGG AGATGGAGCG CTTCGGCCTC |
| 1321 | CATGAGAGGT TGTTGGAGAC AGAGGAGGGG TGTAAAAGAA TCATAGAAGT CCTCTACCCC |
| 1381 | CTAGAACCAA CAGGATCGGA GGGCTTAAAA AGTCTGTTCA ATCTTGTGTG CGTGCTATAT |
| 1441 | TGCTTGCACA AGGAACAGAA AGTGAAAGAC ACAGAGGAAG CAGTAGCAAC AGTAAGACAA |
| 1501 | CACTGCCATC TAGTGGAAAA AGAAAAAAGT GCAACAGAGA CATCTAGTGG ACAAAAGAAA |
| 1561 | AATGACAAGG GAATAGCAGC GCCACCTGGT GGCAGTCAGA ATTTTCCAGC GCAACAACAA |
| 1621 | GGAAATGCCT GGGTACATGT ACCCTTGTCA CCGCGCACCT TAAATGCGTG GGTAAAAGCA |
| 1681 | GTAGAGGAGA AAAAATTTGG AGCAGAAATA GTACCCATGT TTCAAGCCCT ATCGAATTCC |
| 1741 | CGTTTGTGCT AGGGTTCTTA GGCTTCTTGG GGGCTGCTGG AACTGCAATG GGAGCAGCGG |
| 1801 | CGACAGCCCT GACGGTCCAG TCTCAGCATT TGCTTGCTGG GATACTGCAG CAGCAGAAGA |
| 1861 | ATCTGCTGGC GGCTGTGGAG GCTCAACAGC AGATGTTGAA GCTGACCATT TGGGGTGTTA |
| 1921 | AAAACCTCAA TGCCCGCGTC ACAGCCCTTG AGAAGTACCT AGAGGATCAG GCACGACTAA |
| 1981 | ACTCCTGGGG GTGCGCATGG AAACAAGTAT GTCATACCAC AGTGGAGTGG CCCTGGACAA |
| 2041 | ATCGGACTCC GGATTGGCAA AATATGACTT GGTTGGAGTG GGAAAGACAA ATAGCTGATT |
| 2101 | TGGAAAGCAA CATTACGAGA CAATTAGTGA AGGCTAGAGA ACAAGAGGAA AAGAATCTAG |
| 2161 | ATGCCTATCA GAAGTTAACT AGTTGGTCAG ATTTCTGGTC TTGGTTCGAT TTCTCAAAAT |
| 2221 | GGCTTAACAT TTTAAAAATG GGATTTTTAG TAATAGTAGG AATAATAGGG TTAAGATTAC |
| 2281 | TTTACACAGT ATATGGATGT ATAGTGAGGG TTAGGCAGGG ATATGTTCCT CTATCTCCAC |
| 2341 | AGATCCATAT CCGCGGCAAT TTTAAAAGAA AGGGAGGAAT AGGGGGACAG ACTTCAGCAG |
| 2401 | AGAGACTAAT TAATATAATA ACAACACAAT TAGAAATACA ACATTTACAA ACCAAAATTC |
| 2461 | AAAAAATTTT AAATTTTAGA GCCGCGGAGA TCTGTTACAT AACTTATGGT AAATGGCCTG |
| 2521 | CCTGGCTGAC TGCCCAATGA CCCCTGCCCA ATGATGTCAA TAATGATGTA TGTTCCCATG |
| 2581 | TAATGCCAAT AGGGACTTTC CATTGATGTC AATGGGTGGA GTATTTATGG TAACTGCCCA |
| 2641 | CTTGGCAGTA CATCAAGTGT ATCATATGCC AAGTATGCCC CCTATTGATG TCAATGATGG |
| 2701 | TAAATGGCCT GCCTGGCATT ATGCCCAGTA CATGACCTTA TGGGACTTTC CTACTTGGCA |
| 2761 | GTACATCTAT GTATTAGTCA TTGCTATTAC CATGGGAATT CACTAGTGGA GAAGAGCATG |
| 2821 | CTTGAGGGCT GAGTGCCCCT CAGTGGGCAG AGAGCACATG GCCCACAGTC CCTGAGAAGT |
| 2881 | TGGGGGGAGG GGTGGGCAAT TGAACTGGTG CCTAGAGAAG GTGGGGCTTG GGTAAACTGG |
| 2941 | GAAAGTGATG TGGTGTACTG GCTCCACCTT TTTCCCCAGG GTGGGGGAGA ACCATATATA |
| 3001 | AGTGCAGTAG TCTCTGTGAA CATTCAAGCT TCTGCCTTCT CCCTCCTGTG AGTTTGCTAG |
| 3061 | CCACCAATGC AGATTGAGCT GAGCACCTGC TTCTTCCTGT GCCTGCTGAG GTTCTGCTTC |
| 3121 | TCTGCCACCA GGAGATACTA CCTGGGGGCT GTGGAGCTGA GCTGGGACTA CATGCAGTCT |
| 3181 | GACCTGGGGG AGCTGCCTGT GGATGCCAGG TTCCCCCCCA GAGTGCCCAA GAGCTTCCCC |
| 3241 | TTCAACACCT CTGTGGTGTA CAAGAAGACC CTGTTTGTGG AGTTCACTGA CCACCTGTTC |
| 3301 | AACATTGCCA AGCCCAGGCC CCCCTGGATG GGCCTGCTGG GCCCCACCAT CCAGGCTGAG |
| 3361 | GTGTATGACA CTGTGGTGAT CACCCTGAAG AACATGGCCA GCCACCCTGT GAGCCTGCAT |
| 3421 | GCTGTGGGGG TGAGCTACTG GAAGGCCTCT GAGGGGGCTG AGTATGATGA CCAGACCAGC |
| 3481 | CAGAGGGAGA AGGAGGATGA CAAGGTGTTC CCTGGGGGCA GCCACACCTA TGTGTGGCAG |
| 3541 | GTGCTGAAGG AGAATGGCCC CATGGCCTCT GACCCCCTGT GCCTGACCTA CAGCTACCTG |
| 3601 | AGCCATGTGG ACCTGGTGAA GGACCTGAAC TCTGGCCTGA TTGGGGCCCT GCTGGTGTGC |
| 3661 | AGGGAGGGCA GCCTGGCCAA GGAGAAGACC CAGACCCTGC ACAAGTTCAT CCTGCTGTTT |
| 3721 | GCTGTGTTTG ATGAGGGCAA GAGCTGGCAC TCTGAAACCA AGAACAGCCT GATGCAGGAC |
| 3781 | AGGGATGCTG CCTCTGCCAG GGCCTGGCCC AAGATGCACA CTGTGAATGG CTATGTGAAC |
| 3841 | AGGAGCCTGC CTGGCCTGAT TGGCTGCCAC AGGAAGTCTG TGTACTGGCA TGTGATTGGC |
| 3901 | ATGGGCACCA CCCCTGAGGT GCACAGCATC TTCCTGGAGG GCCACACCTT CCTGGTCAGG |
| 3961 | AACCACAGGC AGGCCAGCCT GGAGATCAGC CCCATCACCT TCCTGACTGC CCAGACCCTG |
| 4021 | CTGATGGACC TGGGCCAGTT CCTGCTGTTC TGCCACATCA GCAGCCACCA GCATGATGGC |
| 4081 | ATGGAGGCCT ATGTGAAGGT GGACAGCTGC CCTGAGGAGC CCCAGCTGAG GATGAAGAAC |
| 4141 | AATGAGGAGG CTGAGGACTA TGATGATGAC CTGACTGACT CTGAGATGGA TGTGGTGAGG |
| 4201 | TTTGATGATG ACAACAGCCC CAGCTTCATC CAGATCAGGT CTGTGGCCAA GAAGCACCCC |
| 4261 | AAGACCTGGG TGCACTACAT TGCTGCTGAG GAGGAGGACT GGGACTATGC CCCCCTGGTG |
| 4321 | CTGGCCCCTG ATGACAGGAG CTACAAGAGC CAGTACCTGA ACAATGGCCC CCAGAGGATT |
| 4381 | GGCAGGAAGT ACAAGAAGGT CAGGTTCATG GCCTACACTG ATGAAACCTT CAAGACCAGG |
| 4441 | GAGGCCATCC AGCATGAGTC TGGCATCCTG GGCCCCCTGC TGTATGGGGA GGTGGGGGAC |
| 4501 | ACCCTGCTGA TCATCTTCAA GAACCAGGCC AGCAGGCCCT ACAACATCTA CCCCCATGGC |
| 4561 | ATCACTGATG TGAGGCCCCT GTACAGCAGG AGGCTGCCCA AGGGGGTGAA GCACCTGAAG |
| 4621 | GACTTCCCCA TCCTGCCTGG GGAGATCTTC AAGTACAAGT GGACTGTGAC TGTGGAGGAT |
| 4681 | GGCCCCACCA AGTCTGACCC CAGGTGCCTG ACCAGATACT ACAGCAGCTT TGTGAACATG |
| 4741 | GAGAGGGACC TGGCCTCTGG CCTGATTGGC CCCCTGCTGA TCTGCTACAA GGAGTCTGTG |
| 4801 | GACCAGAGGG GCAACCAGAT CATGTCTGAC AAGAGGAATG TGATCCTGTT CTCTGTGTTT |
| 4861 | GATGAGAACA GGAGCTGGTA CCTGACTGAG AACATCCAGA GGTTCCTGCC CAACCCTGCT |
| 4921 | GGGGTGCAGC TGGAGGACCC TGAGTTCCAG GCCAGCAACA TCATGCACAG CATCAATGGC |
| 4981 | TATGTGTTTG ACAGCCTGCA GCTGTCTGTG TGCCTGCATG AGGTGGCCTA CTGGTACATC |
| 5041 | CTGAGCATTG GGGCCCAGAC TGACTTCCTG TCTGTGTTCT TCTCTGGCTA CACCTTCAAG |
| 5101 | CACAAGATGG TGTATGAGGA CACCCTGACC CTGTTCCCCT TCTCTGGGGA GACTGTGTTC |
| 5161 | ATGAGCATGG AGAACCCTGG CCTGTGGATT CTGGGCTGCC ACAACTCTGA CTTCAGGAAC |
| 5221 | AGGGGCATGA CTGCCCTGCT GAAAGTCTCC AGCTGTGACA AGAACACTGG GGACTACTAT |
| 5281 | GAGGACAGCT ATGAGGACAT CTCTGCCTAC CTGCTGAGCA AGAACAATGC CATTGAGCCC |
| 5341 | AGGAGCTTCA GCCAGAACAG CAGGCACCCC AGCACCAGGC AGAAGCAGTT CAATGCCACC |
| 5401 | ACCATCCCTG AGAATGACAT AGAGAAGACA GACCCATGGT TTGCCCACCG GACCCCCATG |
| 5461 | CCCAAGATCC AGAATGTGAG CAGCTCTGAC CTGCTGATGC TGCTGAGGCA GAGCCCCACC |
| 5521 | CCCCATGGCC TGAGCCTGTC TGACCTGCAG GAGGCCAAGT ATGAAACCTT CTCTGATGAC |
| 5581 | CCCAGCCCTG GGGCCATTGA CAGCAACAAC AGCCTGTCTG AGATGACCCA CTTCAGGCCC |
| 5641 | CAGCTGCACC ACTCTGGGGA CATGGTGTTC ACCCCTGAGT CTGGCCTGCA GCTGAGGCTG |
| 5701 | AATGAGAAGC TGGGCACCAC TGCTGCCACT GAGCTGAAGA AGCTGGACTT CAAAGTCTCC |
| 5761 | AGCACCAGCA ACAACCTGAT CAGCACCATC CCCTCTGACA ACCTGGCTGC TGGCACTGAC |
| 5821 | AACACCAGCA GCCTGGGCCC CCCCAGCATG CCTGTGCACT ATGACAGCCA GCTGGACACC |
| 5881 | ACCCTGTTTG GCAAGAAGAG CAGCCCCCTG ACTGAGTCTG GGGGCCCCCT GAGCCTGTCT |
| 5941 | GAGGAGAACA ATGACAGCAA GCTGCTGGAG TCTGGCCTGA TGAACAGCCA GGAGAGCAGC |
| 6001 | TGGGGCAAGA ATGTGAGCAG CAGGGAGATC ACCAGGACCA CCCTGCAGTC TGACCAGGAG |
| 6061 | GAGATTGACT ATGATGACAC CATCTCTGTG GAGATGAAGA AGGAGGACTT TGACATCTAC |
| 6121 | GACGAGGACG AGAACCAGAG CCCCAGGAGC TTCCAGAAGA AGACCAGGCA CTACTTCATT |
| 6181 | GCTGCTGTGG AGAGGCTGTG GGACTATGGC ATGAGCAGCA GCCCCCATGT GCTGAGGAAC |
| 6241 | AGGGCCCAGT CTGGCTCTGT GCCCCAGTTC AAGAAGGTGG TGTTCCAGGA GTTCACTGAT |
| 6301 | GGCAGCTTCA CCCAGCCCCT GTACAGAGGG GAGCTGAATG AGCACCTGGG CCTGCTGGGC |
| 6361 | CCCTACATCA GGGCTGAGGT GGAGGACAAC ATCATGGTGA CCTTCAGGAA CCAGGCCAGC |
| 6421 | AGGCCCTACA GCTTCTACAG CAGCCTGATC AGCTATGAGG AGGACCAGAG GCAGGGGGCT |
| 6481 | GAGCCCAGGA AGAACTTTGT GAAGCCCAAT GAAACCAAGA CCTACTTCTG GAAGGTGCAG |
| 6541 | CACCACATGG CCCCCACCAA GGATGAGTTT GACTGCAAGG CCTGGGCCTA CTTCTCTGAT |
| 6601 | GTGGACCTGG AGAAGGATGT GCACTCTGGC CTGATTGGCC CCCTGCTGGT GTGCCACACC |
| 6661 | AACACCCTGA ACCCTGCCCA TGGCAGGCAG GTGACTGTGC AGGAGTTTGC CCTGTTCTTC |
| 6721 | ACCATCTTTG ATGAAACCAA GAGCTGGTAC TTCACTGAGA ACATGGAGAG GAACTGCAGG |
| 6781 | GCCCCCTGCA ACATCCAGAT GGAGGACCCC ACCTTCAAGG AGAACTACAG GTTCCATGCC |
| 6841 | ATCAATGGCT ACATCATGGA CACCCTGCCT GGCCTGGTGA TGGCCCAGGA CCAGAGGATC |
| 6901 | AGGTGGTACC TGCTGAGCAT GGGCAGCAAT GAGAACATCC ACAGCATCCA CTTCTCTGGC |
| 6961 | CATGTGTTCA CTGTGAGGAA GAAGGAGGAG TACAAGATGG CCCTGTACAA CCTGTACCCT |
| 7021 | GGGGTGTTTG AGACTGTGGA GATGCTGCCC AGCAAGGCTG GCATCTGGAG GGTGGAGTGC |
| 7081 | CTGATTGGGG AGCACCTGCA TGCTGGCATG AGCACCCTGT TCCTGGTGTA CAGCAACAAG |
| 7141 | TGCCAGACCC CCCTGGGCAT GGCCTCTGGC CACATCAGGG ACTTCCAGAT CACTGCCTCT |
| 7201 | GGCCAGTATG GCCAGTGGGC CCCCAAGCTG GCCAGGCTGC ACTACTCTGG CAGCATCAAT |
| 7261 | GCCTGGAGCA CCAAGGAGCC CTTCAGCTGG ATCAAGGTGG ACCTGCTGGC CCCCATGATC |
| 7321 | ATCCATGGCA TCAAGACCCA GGGGGCCAGG CAGAAGTTCA GCAGCCTGTA CATCAGCCAG |
| 7381 | TTCATCATCA TGTACAGCCT GGATGGCAAG AAGTGGCAGA CCTACAGGGG CAACAGCACT |
| 7441 | GGCACCCTGA TGGTGTTCTT TGGCAATGTG GACAGCTCTG GCATCAAGCA CAACATCTTC |
| 7501 | AACCCCCCCA TCATTGCCAG ATACATCAGG CTGCACCCCA CCCACTACAG CATCAGGAGC |
| 7561 | ACCCTGAGGA TGGAGCTGAT GGGCTGTGAC CTGAACAGCT GCAGCATGCC CCTGGGCATG |
| 7621 | GAGAGCAAGG CCATCTCTGA TGCCCAGATC ACTGCCAGCA GCTACTTCAC CAACATGTTT |
| 7681 | GCCACCTGGA GCCCCAGCAA GGCCAGGCTG CACCTGCAGG GCAGGAGCAA TGCCTGGAGG |
| 7741 | CCCCAGGTCA ACAACCCCAA GGAGTGGCTG CAGGTGGACT TCCAGAAGAC CATGAAGGTG |
| 7801 | ACTGGGGTGA CCACCCAGGG GGTGAAGAGC CTGCTGACCA GCATGTATGT GAAGGAGTTC |
| 7861 | CTGATCAGCA GCAGCCAGGA TGGCCACCAG TGGACCCTGT TCTTCCAGAA TGGCAAGGTG |
| 7921 | AAGGTGTTCC AGGGCAACCA GGACAGCTTC ACCCCTGTGG TGAACAGCCT GGACCCCCCC |
| 7981 | CTGCTGACCA GATACCTGAG GATTCACCCC CAGAGCTGGG TGCACCAGAT TGCCCTGAGG |
| 8041 | ATGGAGGTGC TGGGCTGTGA GGCCCAGGAC CTGTACTGAG CGGCCGCGGG CCCAATCAAC |
| 8101 | CTCTGGATTA CAAAATTTGT GAAAGATTGA CTGGTATTCT TAACTATGTT GCTCCTTTTA |
| 8161 | CGCTATGTGG ATACGCTGCT TTAATGCCTT TGTATCATGC TATTGCTTCC CGTATGGCTT |
| 8221 | TCATTTTCTC CTCCTTGTAT AAATCCTGGT TGCTGTCTCT TTATGAGGAG TTGTGGCCCG |
| 8281 | TTGTCAGGCA ACGTGGCGTG GTGTGCACTG TGTTTGCTGA CGCAACCCCC ACTGGTTGGG |
| 8341 | GCATTGCCAC CACCTGTCAG CTCCTTTCCG GGACTTTCGC TTTCCCCCTC CCTATTGCCA |
| 8401 | CGGCGGAACT CATCGCCGCC TGCCTTGCCC GCTGCTGGAC AGGGGCTCGG CTGTTGGGCA |
| 8461 | CTGACAATTC CGTGGTGTTG TCGGGGAAAT CATCGTCCTT TCCTTGGCTG CTCGCCTGTG |
| 8521 | TTGCCACCTG GATTCTGCGC GGGACGTCCT TCTGCTACGT CCCTTCGGCC CTCAATCCAG |
| 8581 | CGGACCTTCC TTCCCGCGGC CTGCTGCCGG CTCTGCGGCC TCTTCCGCGT CTTCGCCTTC |
| 8641 | GCCCTCAGAC GAGTCGGATC TCCCTTTGGG CCGCCTCCCC GCAAGCTTCG CACTTTTTAA |
| 8701 | AAGAAAAGGG AGGACTGGAT GGGATTTATT ACTCCGATAG GACGCTGGCT TGTAACTCAG |
| 8761 | TCTCTTACTA GGAGACCAGC TTGAGCCTGG GTGTTCGCTG GTTAGCCTAA CCTGGTTGGC |
| 8821 | CACCAGGGGT AAGGACTCCT TGGCTTAGAA AGCTAATAAA CTTGCCTGCA TTAGAGCTCT |
| 8881 | TACGCGTCCC GGGCTCGAGA TCCGCATCTC AATTAGTCAG CAACCATAGT CCCGCCCCTA |
| 8941 | ACTCCGCCCA TCCCGCCCCT AACTCCGCCC AGTTCCGCCC ATTCTCCGCC CCATGGCTGA |
| 9001 | CTAATTTTTT TTATTTATGC AGAGGCCGAG GCCGCCTCGG CCTCTGAGCT ATTCCAGAAG |
| 9061 | TAGTGAGGAG GCTTTTTTGG AGGCCTAGGC TTTTGCAAAA AGCTAACTTG TTTATTGCAG |
| 9121 | CTTATAATGG TTACAAATAA AGCAATAGCA TCACAAATTT CACAAATAAA GCATTTTTTT |
| 9181 | CACTGCATTC TAGTTGTGGT TTGTCCAAAC TCATCAATGT ATCTTATCAT GTCTGTCCGC |
| 9241 | TTCCTCGCTC ACTGACTCGC TGCGCTCGGT CGTTCGGCTG CGGCGAGCGG TATCAGCTCA |
| 9301 | CTCAAAGGCG GTAATACGGT TATCCACAGA ATCAGGGGAT AACGCAGGAA AGAACATGTG |
| 9361 | AGCAAAAGGC CAGCAAAAGG CCAGGAACCG TAAAAAGGCC GCGTTGCTGG CGTTTTTCCA |
| 9421 | TAGGCTCCGC CCCCCTGACG AGCATCACAA AAATCGACGC TCAAGTCAGA GGTGGCGAAA |
| 9481 | CCCGACAGGA CTATAAAGAT ACCAGGCGTT TCCCCCTGGA AGCTCCCTCG TGCGCTCTCC |
| 9541 | TGTTCCGACC CTGCCGCTTA CCGGATACCT GTCCGCCTTT CTCCCTTCGG GAAGCGTGGC |
| 9601 | GCTTTCTCAT AGCTCACGCT GTAGGTATCT CAGTTCGGTG TAGGTCGTTC GCTCCAAGCT |
| 9661 | GGGCTGTGTG CACGAACCCC CCGTTCAGCC CGACCGCTGC GCCTTATCCG GTAACTATCG |
| 9721 | TCTTGAGTCC AACCCGGTAA GACACGACTT ATCGCCACTG GCAGCAGCCA CTGGTAACAG |
| 9781 | GATTAGCAGA GCGAGGTATG TAGGCGGTGC TACAGAGTTC TTGAAGTGGT GGCCTAACTA |
| 9841 | CGGCTACACT AGAAGAACAG TATTTGGTAT CTGCGCTCTG CTGAAGCCAG TTACCTTCGG |
| 9901 | AAAAAGAGTT GGTAGCTCTT GATCCGGCAA ACAAACCACC GCTGGTAGCG GTGGTTTTTT |
| 9961 | TGTTTGCAAG CAGCAGATTA CGCGCAGAAA AAAAGGATCT CAAGAAGATC CTTTGATCTT |
| 10021 | TTCTACGGGG TCTGACGCTC AGTGGAACGA AAACTCACGT TAAGGGATTT TGGTCATGAG |
| 10081 | ATTATCAAAA AGGATCTTCA CCTAGATCCT TTTAAATTAA AAATGAAGTT TTAAATCAAT |
| 10141 | CTAAAGTATA TATGAGTAAA CTTGGTCTGA CAGTTAGAAA AACTCATCGA GCATCAAATG |
| 10201 | AAACTGCAAT TTATTCATAT CAGGATTATC AATACCATAT TTTTGAAAAA GCCGTTTCTG |
| 10261 | TAATGAAGGA GAAAACTCAC CGAGGCAGTT CCATAGGATG GCAAGATCCT GGTATCGGTC |
| 10321 | TGCGATTCCG ACTCGTCCAA CATCAATACA ACCTATTAAT TTCCCCTCGT CAAAAATAAG |
| 10381 | GTTATCAAGT GAGAAATCAC CATGAGTGAC GACTGAATCC GGTGAGAATG GCAACAGCTT |
| 10441 | ATGCATTTCT TTCCAGACTT GTTCAACAGG CCAGCCATTA CGCTCGTCAT CAAAATCACT |
| 10501 | CGCATCAACC AAACCGTTAT TCATTCGTGA TTGCGCCTGA GCGAGACGAA ATACGCGATC |
| 10561 | GCTGTTAAAA GGACAATTAC AAACAGGAAT CGAATGCAAC CGGCGCAGGA ACACTGCCAG |
| 10621 | CGCATCAACA ATATTTTCAC CTGAATCAGG ATATTCTTCT AATACCTGGA ATGCTGTTTT |
| 10681 | TCCGGGGATC GCAGTGGTGA GTAACCATGC ATCATCAGGA GTACGGATAA AATGCTTGAT |
| 10741 | GGTCGGAAGA GGCATAAATT CCGTCAGCCA GTTTAGTCTG ACCATCTCAT CTGTAACATC |
| 10801 | ATTGGCAACG CTACCTTTGC CATGTTTCAG AAACAACTCT GGCGCATCGG GCTTCCCATA |
| 10861 | CAATCGATAG ATTGTCGCAC CTGATTGCCC GACATTATCG CGAGCCCATT TATACCCATA |
| 10921 | TAAATCAGCA TCCATGTTGG AATTTAATCG CGGCCTAGAG CAAGACGTTT CCCGTTGAAT |
| 10981 | ATGGCTCATA ACACCCCTTG TATTACTGTT TATGTAAGCA GACAGTTTTA TTGTTCATGA |
| 11041 | TGATATATTT TTATCTTGTG CAATGTAACA TCAGAGATTT TGAGACACAA CAATTGGTCG |
| 11101 | ACGGATCC |

Exemplary CAG promoter
Length: 1738; Molecule Type: DNA; Features Location/Qualifiers: source,
1..1738; mol_type, other DNA; note, CAG promoter; organism, synthetic
construct
SEQ ID NO: 29
ATTGATTATTGACTAGTTATTAATAGTAATCAATTACGGGGTCATTAGTTCATAGCCCATATATGGAGTTCCGCG
TTACATAACTTACGGTAAATGGCCCGCCTGGCTGACCGCCCAACGACCCCCGCCCATTGACGTCAATAATGACGT
ATGTTCCCATAGTAACGCCAATAGGGACTTTCCATTGACGTCAATGGGTGGAGTATTTACGGTAAACTGCCCACT
TGGCAGTACATCAAGTGTATCATATGCCAAGTACGCCCCCTATTGACGTCAATGACGGTAAATGGCCCGCCTGGC
ATTATGCCCAGTACATGACCTTATGGGACTTTCCTACTTGGCAGTACATCTACGTATTAGTCATCGCTATTACCA
TGGTCGAGGTGAGCCCCACGTTCTGCTTCACTCTCCCCATCTCCCCCCCCTCCCCACCCCCAATTTTGTATTTAT
TTATTTTTTAATTATTTTGTGCAGCGATGGGGGCGGGGGGGGGGGGGGGGCGCGCGCCAGGCGGGGCGGGGCGGG
GCGAGGGGCGGGGCGGGGCGAGGCGGAGAGGTGCGGCGGCAGCCAATCAGAGCGGCGCGCTCCGAAAGTTTCCTT
TTATGGCGAGGCGGCGGCGGCGGCGGCCCTATAAAAAGCGAAGCGCGCGGCGGGCGGGAGTCGCTGCGCGCTGCC
TTCGCCCCGTGCCCCGCTCCGCCGCCGCCTCGCGCCGCCCGCCCCGGCTCTGACTGACCGCGTTACTCCCACAGG
TGAGCGGGCGGGACGGCCCTTCTCCTCCGGGCTGTAATTAGCGCTTGGTTTAATGACGGCTTGTTTCTTTTCTGT
GGCTGCGTGAAAGCCTTGAGGGGCTCCGGGAGGGCCCTTTGTGCGGGGGGAGCGGCTCGGGGGGTGCGTGCGTGT
GTGTGTGCGTGGGGAGCGCCGCGTGCGGCTCCGCGCTGCCCGGCGGCTGTGAGCGCTGCGGGCGCGGCGCGGGGC
TTTGTGCGCTCCGCAGTGTGCGCGAGGGGAGCGCGGCCGGGGGCGGTGCCCCGCGGTGCGGGGGGGGCTGCGAGG
GGAACAAAGGCTGCGTGCGGGGTGTGTGCGTGGGGGGGTGAGCAGGGGGTGTGGGCGCGTCGGTCGGGCTGCAAC
CCCCCCTGCACCCCCCTCCCCGAGTTGCTGAGCACGGCCCGGCTTCGGGTGCGGGGCTCCGTACGGGGCGTGGCG
CGGGGCTCGCCGTGCCGGGCGGGGGGTGGCGGCAGGTGGGGGTGCCGGGCGGGGCGGGGCCGCCTCGGGCCGGGG
AGGGCTCGGGGGAGGGGCGCGGCGGCCCCCGGAGCGCCGGCGGCTGTCGAGGCGCGGCGAGCCGCAGCCATTGCC
TTTTATGGTAATCGTGCGAGAGGGCGCAGGGACTTCCTTTGTCCCAAATCTGTGCGGAGCCGAAATCTGGGAGGC
GCCGCCGCACCCCCTCTAGCGGGCGCGGGGCGAAGCGGTGCGGCGCCGGCAGGAAGGAAATGGGCGGGGAGGGCC
TTCGTGCGTCGCCGCGCCGCCGTCCCCTTCTCCCTCTCCAGCCTCGGGGCTGTCCGCGGGGGGACGGCTGCCTTC
GGGGGGGACGGGGCAGGGCGGGGTTCGGCTTCTGGCGTGTGACCGGCGGCTCTAGAGCCTCTGCTAACCATGTTC
ATGCCTTCTTCTTTTTCCTACAGCTCCTGGGCAACGTGCTGGTTATTGTGCTGTCTCATCATTTTGGCAAAGAAT
TGCTCGAGCCACC

1.-33. (canceled)

34. A retroviral vector pseudotyped with hemagglutinin-neuraminidase (HN) and fusion (F) proteins from a respiratory paramyxovirus, which is obtainable by a method comprising obtaining codon-optimised gag-pol genes, and transfecting cells with one or more plasmids encoding the retroviral vector and the codon-optimised gag-pol genes, wherein the codon-optimised gag-pol genes comprise a nucleic acid sequence having at least 80% sequence identity to SEQ ID NO:1, wherein the retroviral vector comprises a promoter and a transgene.

35. The retroviral vector according to claim 34, wherein the retroviral vector is a lentiviral vector.

36. The retroviral vector according to claim 35, wherein the lentiviral vector is an SIV vector.

37. The retroviral vector according to claim 34, wherein the codon-optimised gag-pol genes are SIV gag-pol genes.

38. The retroviral vector according to claim 34, wherein the codon-optimised gag-pol genes comprise a nucleic acid sequence having at least 85%, at least 90%, at least 95%, at least 96%, at least 97%, at least 98%, at least 99% or more sequence identity to SEQ ID NO:1.

39. The retroviral vector according to claim 34, wherein the codon-optimised gag-pol genes comprise the nucleic acid sequence of SEQ ID NO:1.

40. The retroviral vector according to claim 34, wherein the codon-optimised gag-pol genes consist of a nucleic acid sequence having at least 80% sequence identity to SEQ ID NO:1.

41. The retroviral vector according to claim 34, wherein the codon-optimised gag-pol genes consist of a nucleic acid sequence having at least 85%, at least 90%, at least 95%, at least 96%, at least 97%, at least 98%, at least 99% or more sequence identity to SEQ ID NO:1.

42. The retroviral vector according to claim 34, wherein the codon-optimised gag-pol genes consist of the nucleic acid sequence of SEQ ID NO:1.

43. The retroviral vector according to claim 34, wherein the codon-optimised gag-pol genes are comprised in a plasmid that comprises a nucleic acid sequence having at least 80% sequence identity to SEQ ID NO:5.

44. The retroviral vector according to claim 43, wherein the codon-optimised gag-pol genes are comprised in a plasmid that comprises a nucleic acid sequence having at least 85%, at least 90%, at least 95%, at least 96%, at least 97%, at least 98%, at least 99% or more sequence identity to SEQ ID NO:5.

45. The retroviral vector according to claim 43, wherein the codon-optimised gag-pol genes are comprised in a plasmid that comprises the nucleic acid sequence of SEQ ID NO:5.

46. The retroviral vector according to claim 43, wherein the codon-optimised gag-pol genes are comprised in a plasmid that consists of a nucleic acid sequence having at least 80% sequence identity to SEQ ID NO:5.

47. The retroviral vector according to claim 43, wherein the codon-optimised gag-pol genes are comprised in a plasmid that consists of a nucleic acid sequence having at least 85%, at least 90%, at least 95%, at least 96%, at least 97%, at least 98%, at least 99% or more sequence identity to SEQ ID NO:5.

48. The retroviral vector according to claim 43, wherein the codon-optimised gag-pol genes are comprised in a plasmid that consists of the nucleic acid sequence of SEQ ID NO:5.

49. The retroviral vector according to claim 34, wherein the respiratory paramyxovirus is a Sendai virus.

50. The retroviral vector according to claim 34, wherein the titre of retroviral vector produced is:
a. equivalent to the titre of retroviral vector produced by an otherwise identical method which does not use codon-optimised gag-pol genes; or
b. increased compared with the titre of retroviral vector produced by an otherwise identical method which does not use codon-optimised gag-pol genes.

51. The retroviral vector according to claim 50, wherein the titre of retroviral vector is at least 2-fold, or at least 2.5-fold greater than the titre of retroviral vector produced by an otherwise identical method which does not use codon-optimised gag-pol genes.

52. The retroviral vector according to claim 34, wherein the vector comprises a hybrid human CMV enhancer/EF1a (hCEF) promoter.

53. The retroviral vector according to claim 34, wherein the transgene encodes:
a. CFTR;
b. A1AT; or
c. FVIII.

54. The retroviral vector according to claim 34, wherein:
a. the promoter is a hCEF promoter and the transgene encodes CFTR;
b. the promoter is a hCEF promoter and the transgene encodes A1AT; or
c. the promoter is a hCEF or CMV promoter and the transgene encodes FVIII.

55. The retroviral vector according to claim 34, wherein said method comprises the following steps:
a. growing cells in suspension;
b. transfecting the cells with one or more plasmids comprising genes for retroviral production and packaging;
c. adding a nuclease to the cell suspension;
d. harvesting the retrovirus from the cell suspension;
e. adding trypsin to the harvested retrovirus; and
f. purifying the retrovirus.

56. The retroviral vector according to claim 55, wherein the one or more plasmids comprise:
a. a vector genome plasmid;
b. a co-gagpol plasmid;
c. a Rev plasmid;
d. a fusion (F) protein plasmid; and/or
e. a hemagglutinin-neuraminidase (HN) plasmid.

57. The retroviral vector according to claim 56, wherein:
a. the vector genome plasmid is selected from pGM830 and pGM326;
b. the co-gagpol plasmid is pGM691;
c. the Rev plasmid is pGM299;
d. the fusion (F) protein plasmid is pGM301; and
e. the hemagglutinin-neuraminidase (HN) plasmid is pGM303.

58. The retroviral vector according to claim 56, wherein the ratio of vector genome plasmid:co-gagpol plasmid:Rev plasmid:F plasmid:HN plasmid is 20:9:6:6:6.

59. The retroviral vector according to claim 55, wherein steps (a)-(f) are carried out sequentially.

60. The retroviral vector according to claim 55, wherein the cells are HEK293T or 293T/17 cells.

61. The retroviral vector according to claim 55, wherein the purification step comprises a chromatography step.

62. The retroviral vector according to claim 56, wherein the vector genome plasmid is modified to reduce the number of retroviral ORFs.


SEQUENCE LISTING (29 sequences)

SEQ ID NO: 1 - DNA, 4391 bp, synthetic construct
  misc_feature 1..4391: note = codon-optimised SIV gal-pol nucleic acid sequence (from pGM691)
  source 1..4391: mol_type = other DNA
atgggagctgccacatctgccctgaatagacggcagctggaccagttcgagaagatcagactgcggcccaacggcaagaagaagtaccagatcaagcacctgatctgggccggcaaagagatggaaagattcggcctgcacgagcggctgctggaaaccgaggaaggctgcaagagaattatcgaggtgctgtaccctctggaacctaccggctctgagggcctgaagtccctgttcaatctcgtgtgcgtgctgtactgcctgcacaaagaacagaaagtgaaggacaccgaagaggccgtggccacagttagacagcactgccacctggtggaaaaagagaagtccgccacagagacaagcagcggccagaagaagaacgacaagggaattgctgcccctcctggcggcagccagaattttcctgctcagcagcagggaaacgcctgggtgcacgttccactgagccctagaacactgaatgcctgggtcaaagccgtggaagagaagaagtttggcgccgagatcgtgcccatgttccaggctctgtctgagggctgcaccccttacgacatcaaccagatgctgaacgtgctgggagatcaccagggcgctctgcagatcgtgaaagagatcatcaacgaagaggctgcccagtgggacgtgacacatccattgcctgctggacctctgccagccggacaactgagagatcctagaggctctgatatcgccggcaccaccagctctgtgcaagagcagctggaatggatctacaccgccaatcctagagtggacgtgggcgccatctacagaagatggatcatcctgggcctgcagaaatgcgtgaagatgtacaaccccgtgtccgtgctggacatcagacagggacccaaagagcccttcaaggactacgtggaccggttctataaggccattagagccgagcaggccagcggcgaagtgaagcagtggatgacagagagcctgctgatccagaacgccaatccagactgcaaagtgatcctgaaaggcctgggcatgcaccccacactggaagagatgctgacagcctgtcaaggcgttggcggcccttcttacaaagccaaagtgatggccgagatgatgcagaccatgcagaaccagaacatggtgcagcaaggcggccctaagagacagaggcctcctctgagatgctacaactgcggcaagttcggccacatgcagagacagtgtcctgagcctaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagaccgtgtacatcgagggcgtgcccatcaaggctctgctggatacaggcgccgacgacaccatcatcaaagagaacgacctgcagctgagcggcccttggaggcctaagatcattggaggaatcggcggaggcctgaacgtcaaagagtacaacgaccgggaagtgaagatcgaggacaagatcctgaggggcacaatcctgctgggcgccacacctatcaacatcatcggcagaaatctgctggcccctgccggcgctagactggttatgggacagctctctgagaagatccccgtgacacccgtgaagctgaaagaaggcgctagaggaccttgtgtgcgacagtggcctctgagcaaagagaagattgaggccctgcaagaaatctgtagccagctggaacaagagggcaagatcagcagagttggcggcgagaacgcctacaatacccctatcttctgcatcaagaaaaaggacaagagccagtggcggatgctggtggactttagagagctgaacaaggctacccaggacttcttcgaggtgcagctgggaattcctcatcctgccggcctgcggaagatgagacagatcacagtgctggatgtgggcgacgcctactacagcatccctctggaccccaacttcagaaagtacaccgccttcacaatccccaccgtgaacaatcaaggccctggcatcagataccagttcaactgcctgcctcaaggctggaagggcagccccaccatttttcagaataccgccgccagcatcctggaagaaatcaagagaaacctgcctgctctgaccatcgtgcagtacatggacgatctgtgggtcggaagccaagagaatgagcacacccacgacaagctggtggaacagctgagaacaaagctgcaggcctggggcctcgaaacccctgagaagaaggtgcagaaagaacctccttacgagtggatgggctacaagctgtggcctcacaagtgggagctgagccggattcagctcgaagagaaggacgagtggaccgtgaacgacatccagaaactcgtgggcaagctgaattgggcagcccagctgtatcccggcctgaggaccaagaacatctgcaagctgatccggggaaagaagaacctgctggaactggtcacatggacacctgaggccgaggccgaatatgccgagaatgccgaaatcctgaaaaccgagcaagaggggacctactacaagcctggcattccaatcagagctgccgtgcagaaactggaaggcggccagtggtcctaccagtttaagcaagaaggccaggtcctgaaagtgggcaagtacaccaagcagaagaacacccacaccaacgagctgaggacactggctggcctggtccagaaaatctgcaaagaggccctggtcatttggggcatcctgcctgttctggaactgcccattgagcgggaagtgtgggaacagtggtgggccgattactggcaagtgtcttggatccccgagtgggacttcgtgtctacccctcctctgctgaaactgtggtacaccctgacaaaagagcccattcctaaagaggacgtctactacgttgacggcgcctgcaaccggaactccaaagaaggcaaggccggctacatcagccagtacggcaagcagagagtggaaaccctggaaaacaccaccaaccagcaggccgagctgaccgccattaagatggccctggaagatagcggccccaatgtgaacatcgtgaccgactctcagtacgccatgggaatcctgacagcccagcctacacagagcgatagccctctggttgagcagatcattgccctgatgattcagaagcagcaaatctacctgcagtgggtgcccgctcacaaaggcatcggcggaaacgaagagatcgataagctggtgtccaagggaatcagacgggtgctgttcctggaaaagattgaagaggcccaagaggaacacgagcgctaccacaacaactggaagaatctggccgacacctacggactgccccagatcgtggccaaagaaatcgtggctatgtgccccaagtgtcagatcaagggcgaacctgtgcacggccaagtggatgcttctcctggcacatggcagatggactgtacccacctggaaggcaaagtggtcatcgtggctgtgcacgtggcctccggctttattgaggccgaagtgatccccagagagacaggcaaagaaaccgccaagttcctgctgaagatcctgtccagatggcccatcacacagctgcacaccgacaacggccctaacttcacatctcaagaggtggccgccatctgttggtggggaaagattgagcacacaaccggcattccctacaatccacagagccagggcagcatcgagtccatgaacaagcagctcaaagagattatcggcaagatccgggacgactgccagtacacagaaacagccgtgctgatggcctgtcacatccacaacttcaagcggaaaggcggcatcggaggacagacatctgccgagagactgatcaatatcatcaccactcagctggaaatccagcacctccagaccaagatccagaagattctgaacttccgggtgtactaccgcgagggcagagatcctgtttggaaaggcccagcacagctgatctggaaaggcgaaggtgccgtggtgctgaaggatggctctgatctgaaggtggtgcccagacggaaggccaagattatcaaggattacgagcccaaacagcgcgtgggcaatgaaggcgacgttgagggcacaagaggcagcgacaattga

SEQ ID NO: 2 - DNA, 4391 bp, Simian immunodeficiency virus
  source 1..4391: mol_type = genomic DNA
atgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtactatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcagaaggctgcacaccctatgacattaatcagatgcttaatgtgctaggagatcatcaaggggcattacaaatagtgaaagagatcattaatgaagaagcagcccagtgggatgtaacacacccactacccgcaggacccctaccagcaggacagctcagggaccctcgcggctcagatatagcagggaccaccagctcagtacaagaacagttagaatggatctatactgctaacccccgggtagatgtaggtgccatctaccggagatggattattctaggacttcaaaagtgtgtcaaaatgtacaacccagtatcagtcctagacattaggcagggacctaaagagcccttcaaggattatgtggacagattttacaaggcaattagagcagaacaagcctcaggggaagtgaaacaatggatgacagaatcattactcattcaaaatgctaatccagattgtaaggtcatcctgaagggcctaggaatgcaccccacccttgaagaaatgttaacggcttgtcagggggtaggaggcccaagctacaaagcaaaagtaatggcagaaatgatgcagaccatgcaaaatcaaaacatggtgcagcagggaggtccaaaaagacaaagacccccactaagatgttataattgtggaaaatttggccatatgcaaagacaatgtccggaaccaaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagacagtgtatatagaaggggtccccattaaggcactgctagacacaggggcagatgacaccataattaaagaaaatgatttacaattatcaggtccatggagacccaaaattatagggggcataggaggaggccttaatgtaaaagaatataacgacagggaagtaaaaatagaagataaaattttgagaggaacaatattgttaggagcaactcccattaatataataggtagaaatttgctggccccggcaggtgcccggttagtaatgggacaattatcagaaaaaattcctgtcacacctgtcaaattgaaggaaggggctcggggaccctgtgtaagacaatggcctctctctaaagagaagattgaagctttacaggaaatatgttcccaattagagcaggaaggaaaaatcagtagagtaggaggagaaaatgcatacaataccccaatattttgcataaagaagaaggacaaatcccagtggaggatgctagtagactttagagagttaaataaggcaacccaagatttctttgaagtgcaattagggataccccacccagcaggattaagaaagatgagacagataacagttttagatgtaggagacgcctattattccataccattggatccaaattttaggaaatatactgcttttactattcccacagtgaataatcagggacccgggattaggtatcaattcaactgtctcccgcaagggtggaaaggatctcctacaatcttccaaaatacagcagcatccattttggaggagataaaaagaaacttgccagcactaaccattgtacaatacatggatgatttatgggtaggttctcaagaaaatgaacacacccatgacaaattagtagaacagttaagaacaaaattacaagcctggggcttagaaaccccagaaaagaaggtgcaaaaagaaccaccttatgagtggatgggatacaaactttggcctcacaaatgggaactaagcagaatacaactggaggaaaaagatgaatggactgtcaatgacatccagaagttagttgggaaactaaattgggcagcacaattgtatccaggtcttaggaccaagaatatatgcaagttaattagaggaaagaaaaatctgttagagctagtgacttggacacctgaggcagaagctgaatatgcagaaaatgcagagattcttaaaacagaacaggaaggaacctattacaaaccaggaatacctattagggcagcagtacagaaattggaaggaggacagtggagttaccaattcaaacaagaaggacaagtcttgaaagtaggaaaatacaccaagcaaaagaacacccatacaaatgaacttcgcacattagctggtttagtgcagaagatttgcaaagaagctctagttatttgggggatattaccagttctagaactcccgatagaaagagaggtatgggaacaatggtgggcggattactggcaggtaagctggattcccgaatgggattttgtcagcaccccacctttgctcaaactatggtacacattaacaaaagaacccatacccaaggaggacgtttactatgtagatggagcatgcaacagaaattcaaaagaaggaaaagcaggatacatctcacaatacggaaaacagagagtagaaacattagaaaacactaccaatcagcaagcagaattaacagctataaaaatggctttggaagacagtgggcctaatgtgaacatagtaacagactctcaatatgcaatgggaattttgacagcacaacccacacaaagtgattcaccattagtagagcaaattatagccttaatgatacaaaagcaacaaatatatttgcagtgggtaccagcacataaaggaataggaggaaatgaggagatagataaattagtgagtaaaggcattagaagagttttattcttagaaaaaatagaagaagctcaagaagagcatgaaagatatcataataattggaaaaacctagcagatacatatgggcttccacaaatagtagcaaaagagatagtggccatgtgtccaaaatgtcagataaagggagaaccagtgcatggacaagtggatgcctcacctggaacatggcagatggattgtactcatctagaaggaaaagtagtcatagttgcggtccatgtagccagtggattcatagaagcagaagtcatacctagggaaacaggaaaagaaacggcaaagtttctattaaaaatactgagtagatggcctataacacagttacacacagacaatgggcctaactttacctcccaagaagtggcagcaatatgttggtggggaaaaattgaacatacaacaggtataccatataacccccaatctcaaggatcaatagaaagcatgaacaaacaattaaaagagataattgggaaaataagagatgattgccaatatacagagacagcagtactgatggcttgccatattcacaattttaaaagaaagggaggaatagggggacagacttcagcagagagactaattaatataataacaacacaattagaaatacaacatttacaaaccaaaattcaaaaaattttaaattttagagtctactacagagaagggagagaccctgtgtggaaaggaccagcacaattaatctggaaaggggaaggagcagtggtcctcaaggacggaagtgacctaaaggttgtaccaagaaggaaagctaaaattattaaggattatgaacccaaacaaagagtgggtaatgagggtgacgtggaaggtaccaggggatctgataactaa

SEQ ID NO: 3 - DNA, 10528 bp, synthetic construct
  misc_feature 1..10528: note = pGM326
  source 1..10528: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccatgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtgagggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 4 - DNA, 10536 bp, synthetic construct
  misc_feature 1..10536: note = pGM830
  source 1..10536: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaattgggggcggctacctcagcactaaataggagacaattagaccaatttgagaaaatacgacttcgcccgaacggaaagaaaaagtaccaaattaaacatttaatattgggcaggcaaggagattggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaattgcctgggtacatgtacccttgtcaccgcgcaccttaaatgcgtgggtaaaagcagtagaggagaaaaaatttggagcagaaatagtacccatgtttcaagccctatcgcctgcaggccgtttgtgctagggttcttaggcttcttgggggctgctggaactgcattgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaataagacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaaagggatttttagtaatagtaggaataatagggttaagattactttacacagtatatggatgtatagtgagggttaggcagggatatgttcctctatctccacagatccatataaagcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccatgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtgagggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 5 - DNA, 9064 bp, synthetic construct
  misc_feature 1..9064: note = pGM691
  source 1..9064: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagccaccatgggagctgccacatctgccctgaatagacggcagctggaccagttcgagaagatcagactgcggcccaacggcaagaagaagtaccagatcaagcacctgatctgggccggcaaagagatggaaagattcggcctgcacgagcggctgctggaaaccgaggaaggctgcaagagaattatcgaggtgctgtaccctctggaacctaccggctctgagggcctgaagtccctgttcaatctcgtgtgcgtgctgtactgcctgcacaaagaacagaaagtgaaggacaccgaagaggccgtggccacagttagacagcactgccacctggtggaaaaagagaagtccgccacagagacaagcagcggccagaagaagaacgacaagggaattgctgcccctcctggcggcagccagaattttcctgctcagcagcagggaaacgcctgggtgcacgttccactgagccctagaacactgaatgcctgggtcaaagccgtggaagagaagaagtttggcgccgagatcgtgcccatgttccaggctctgtctgagggctgcaccccttacgacatcaaccagatgctgaacgtgctgggagatcaccagggcgctctgcagatcgtgaaagagatcatcaacgaagaggctgcccagtgggacgtgacacatccattgcctgctggacctctgccagccggacaactgagagatcctagaggctctgatatcgccggcaccaccagctctgtgcaagagcagctggaatggatctacaccgccaatcctagagtggacgtgggcgccatctacagaagatggatcatcctgggcctgcagaaatgcgtgaagatgtacaaccccgtgtccgtgctggacatcagacagggacccaaagagcccttcaaggactacgtggaccggttctataaggccattagagccgagcaggccagcggcgaagtgaagcagtggatgacagagagcctgctgatccagaacgccaatccagactgcaaagtgatcctgaaaggcctgggcatgcaccccacactggaagagatgctgacagcctgtcaaggcgttggcggcccttcttacaaagccaaagtgatggccgagatgatgcagaccatgcagaaccagaacatggtgcagcaaggcggccctaagagacagaggcctcctctgagatgctacaactgcggcaagttcggccacatgcagagacagtgtcctgagcctaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagaccgtgtacatcgagggcgtgcccatcaaggctctgctggatacaggcgccgacgacaccatcatcaaagagaacgacctgcagctgagcggcccttggaggcctaagatcattggaggaatcggcggaggcctgaacgtcaaagagtacaacgaccgggaagtgaagatcgaggacaagatcctgaggggcacaatcctgctgggcgccacacctatcaacatcatcggcagaaatctgctggcccctgccggcgctagactggttatgggacagctctctgagaagatccccgtgacacccgtgaagctgaaagaaggcgctagaggaccttgtgtgcgacagtggcctctgagcaaagagaagattgaggccctgcaagaaatctgtagccagctggaacaagagggcaagatcagcagagttggcggcgagaacgcctacaatacccctatcttctgcatcaagaaaaaggacaagagccagtggcggatgctggtggactttagagagctgaacaaggctacccaggacttcttcgaggtgcagctgggaattcctcatcctgccggcctgcggaagatgagacagatcacagtgctggatgtgggcgacgcctactacagcatccctctggaccccaacttcagaaagtacaccgccttcacaatccccaccgtgaacaatcaaggccctggcatcagataccagttcaactgcctgcctcaaggctggaagggcagccccaccatttttcagaataccgccgccagcatcctggaagaaatcaagagaaacctgcctgctctgaccatcgtgcagtacatggacgatctgtgggtcggaagccaagagaatgagcacacccacgacaagctggtggaacagctgagaacaaagctgcaggcctggggcctcgaaacccctgagaagaaggtgcagaaagaacctccttacgagtggatgggctacaagctgtggcctcacaagtgggagctgagccggattcagctcgaagagaaggacgagtggaccgtgaacgacatccagaaactcgtgggcaagctgaattgggcagcccagctgtatcccggcctgaggaccaagaacatctgcaagctgatccggggaaagaagaacctgctggaactggtcacatggacacctgaggccgaggccgaatatgccgagaatgccgaaatcctgaaaaccgagcaagaggggacctactacaagcctggcattccaatcagagctgccgtgcagaaactggaaggcggccagtggtcctaccagtttaagcaagaaggccaggtcctgaaagtgggcaagtacaccaagcagaagaacacccacaccaacgagctgaggacactggctggcctggtccagaaaatctgcaaagaggccctggtcatttggggcatcctgcctgttctggaactgcccattgagcgggaagtgtgggaacagtggtgggccgattactggcaagtgtcttggatccccgagtgggacttcgtgtctacccctcctctgctgaaactgtggtacaccctgacaaaagagcccattcctaaagaggacgtctactacgttgacggcgcctgcaaccggaactccaaagaaggcaaggccggctacatcagccagtacggcaagcagagagtggaaaccctggaaaacaccaccaaccagcaggccgagctgaccgccattaagatggccctggaagatagcggccccaatgtgaacatcgtgaccgactctcagtacgccatgggaatcctgacagcccagcctacacagagcgatagccctctggttgagcagatcattgccctgatgattcagaagcagcaaatctacctgcagtgggtgcccgctcacaaaggcatcggcggaaacgaagagatcgataagctggtgtccaagggaatcagacgggtgctgttcctggaaaagattgaagaggcccaagaggaacacgagcgctaccacaacaactggaagaatctggccgacacctacggactgccccagatcgtggccaaagaaatcgtggctatgtgccccaagtgtcagatcaagggcgaacctgtgcacggccaagtggatgcttctcctggcacatggcagatggactgtacccacctggaaggcaaagtggtcatcgtggctgtgcacgtggcctccggctttattgaggccgaagtgatccccagagagacaggcaaagaaaccgccaagttcctgctgaagatcctgtccagatggcccatcacacagctgcacaccgacaacggccctaacttcacatctcaagaggtggccgccatctgttggtggggaaagattgagcacacaaccggcattccctacaatccacagagccagggcagcatcgagtccatgaacaagcagctcaaagagattatcggcaagatccgggacgactgccagtacacagaaacagccgtgctgatggcctgtcacatccacaacttcaagcggaaaggcggcatcggaggacagacatctgccgagagactgatcaatatcatcaccactcagctggaaatccagcacctccagaccaagatccagaagattctgaacttccgggtgtactaccgcgagggcagagatcctgtttggaaaggcccagcacagctgatctggaaaggcgaaggtgccgtggtgctgaaggatggctctgatctgaaggtggtgcccagacggaaggccaagattatcaaggattacgagcccaaacagcgcgtgggcaatgaaggcgacgttgagggcacaagaggcagcgacaattgaaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 6 - DNA, 3384 bp, synthetic construct
  misc_feature 1..3384: note = pGM299
  source 1..3384: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgattatgcccctaggaccagaagaaagaagattgcttcgcttgatttggctcctttacagcaccaatccatatccaccaagtggggaagggacggccagacaacgccgacgagccaggagaaggtggagacaacagcaggatcaaattagagtcttggtagaaagactccaagagcaggtgtatgcagttgaccgcctggctgacgaggctcaacacttggctatacaacagttgcctgaccctcctcattcagcttagaatcactagtgaattcacgcgtggtacctctagagtcgacccgggcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgtcgaccaattgttgtgtctcaaaatctctgatgttacattgcacaagataaaaatatatcatcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagctgttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 7 - DNA, 6264 bp, synthetic construct
  misc_feature 1..6264: note = pGM301
  source 1..6264: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgattgccatggcaacatatatccagagagtacagtgcatctcaacatcactactggttgttctcaccacattggtctcgtgtcagattcccagggataggctctctaacataggggtcatagtcgatgaagggaaatcactgaagatagctggatcccacgaatcgaggtacatagtactgagtctagttccgggggtagactttgagaatgggtgcggaacagcccaggttatccagtacaagagcctactgaacaggctgttaatcccattgagggatgccttagatcttcaggaggctctgataactgtcaccaatgatacgacacaaaatgccggtgctccccagtcgagattcttcggtgctgtgattggtactatcgcacttggagtggcgacatcagcacaaatcaccgcagggattgcactagccgaagcgagggaggccaaaagagacatagcgctcatcaaagaatcgatgacaaaaacacacaagtctatagaactgctgcaaaacgctgtgggggaacaaattcttgctctaaagacactccaggatttcgtgaatgatgagatcaaacccgcaataagcgaattaggctgtgagactgctgccttaagactgggtataaaattgacacagcattactccgagctgttaactgcgttcggctcgaatttcggaaccatcggagagaagagcctcacgctgcaggcgctgtcttcactttactctgctaacattactgagattatgaccacaatcaggacagggcagtctaacatctatgatgtcatttatacagaacagatcaaaggaacggtgatagatgtggatctagagagatacatggtcaccctgtctgtgaagatccctattctttctgaagtcccaggtgtgctcatacacaaggcatcatctatttcttacaacatagacggggaggaatggtatgtgactgtccccagccatatactcagtcgtgcttctttcttagggggtgcagacataaccgattgtgttgagtccagattgacctatatatgccccagggatcccgcacaactgatacctgacagccagcaaaagtgtatcctgggggacacaacaaggtgtcctgtcacaaaagttgtggacagccttatccccaagtttgcttttgtgaatgggggcgttgttgctaactgcatagcatccacatgtacctgcgggacaggccgaagaccaatcagtcaggatcgctctaaaggtgtagtattcctaacccatgacaactgtggtcttataggtgtcaatggggtagaattgtatgctaaccggagagggcacgatgccacttggggggtccagaacttgacagtcggtcctgcaattgctatcagacccgttgatatttctctcaaccttgctgatgctacgaatttcttgcaagactctaaggctgagcttgagaaagcacggaaaatcctctcggaggtaggtagatggtacaactcaagagagactgtgattacgatcatagtagttatggtcgtaatattggtggtcattatagtgatcatcatcgtgctttatagactcagaaggtgaaatcactagtgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 8 - DNA, 6522 bp, synthetic construct
  misc_feature 1..6522: note = pGM303
  source 1..6522: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcctcgagcatgtggtctgagttaaaaatcaggagcaacgacggaggtgaaggaccagaggacgccaacgacccccggggaaagggggtgcaacacatccatatccagccatctctacctgtttatggacagagggttagggatggtgataggggcaaacgtgactcgtactggtctacttctcctagtggtagcaccacaaaaccagcatcaggttgggagaggtcaagtaaagccgacacatggttgctgattctctcattcacccagtgggctttgtcaattgccacagtgatcatctgtatcataatttctgctagacaagggtatagtatgaaagagtactcaatgactgtagaggcattgaacatgagcagcagggaggtgaaagagtcacttaccagtctaataaggcaagaggttatagcaagggctgtcaacattcagagctctgtgcaaaccggaatcccagtcttgttgaacaaaaacagcagggatgtcatccagatgattgataagtcgtgcagcagacaagagctcactcagcactgtgagagtacgatcgcagtccaccatgccgatggaattgccccacttgagccacatagtttctggagatgccctgtcggagaaccgtatcttagctcagatcctgaaatctcattgctgcctggtccgagcttgttatctggttctacaacgatctctggatgtgttaggctcccttcactctcaattggcgaggcaatctatgcctattcatcaaatctcattacacaaggttgtgctgacatagggaaatcatatcaggtcctgcagctagggtacatatcactcaattcagatatgttccctgatcttaaccccgtagtgtcccacacttatgacatcaacgacaatcggaaatcatgctctgtggtggcaaccgggactaggggttatcagctttgctccatgccgactgtagacgaaagaaccgactactctagtgatggtattgaggatctggtccttgatgtcctggatctcaaagggagaactaagtctcaccggtatcgcaacagcgaggtagatcttgatcacccgttctctgcactataccccagtgtaggcaacggcattgcaacagaaggctcattgatatttcttgggtatggtggactaaccacccctctgcagggtgatacaaaatgtaggacccaaggatgccaacaggtgtcgcaagacacatgcaatgaggctctgaaaattacatggctaggagggaaacaggtggtcagcgtgatcatccaggtcaatgactatctctcagagaggccaaagataagagtcacaaccattccaatcactcaaaactatctcggggcggaaggtagattattaaaattgggtgatcgggtgtacatctatacaagatcatcaggctggcactctcaactgcagataggagtacttgatgtcagccaccctttgactatcaactggacacctcatgaagccttgtctagaccaggaaataaagagtgcaattggtacaataagtgtccgaaggaatgcatatcaggcgtatacactgatgcttatccattgtcccctgatgcagctaacgtcgctaccgtcacgctatatgccaatacatcgcgtgtcaacccaacaatcatgtattctaacactactaacattataaatatgttaaggataaaggatgttcaattagaggctgcatataccacgacatcgtgtatcacgcattttggtaaaggctactgctttcacatcatcgagatcaatcagaagagcctgaataccttacagccgatgctctttaagactagcatccctaaattatgcaaggccgagtcttaagcggccgcgcatgcgaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtctattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 9 - DNA, 9886 bp, synthetic construct
  misc_feature 1..9886: note = pGM297
  source 1..9886: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagactagtgacttggtgagtaggcttcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtactatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcagaaggctgcacaccctatgacattaatcagatgcttaatgtgctaggagatcatcaaggggcattacaaatagtgaaagagatcattaatgaagaagcagcccagtgggatgtaacacacccactacccgcaggacccctaccagcaggacagctcagggaccctcgcggctcagatatagcagggaccaccagctcagtacaagaacagttagaatggatctatactgctaacccccgggtagatgtaggtgccatctaccggagatggattattctaggacttcaaaagtgtgtcaaaatgtacaacccagtatcagtcctagacattaggcagggacctaaagagcccttcaaggattatgtggacagattttacaaggcaattagagcagaacaagcctcaggggaagtgaaacaatggatgacagaatcattactcattcaaaatgctaatccagattgtaaggtcatcctgaagggcctaggaatgcaccccacccttgaagaaatgttaacggcttgtcagggggtaggaggcccaagctacaaagcaaaagtaatggcagaaatgatgcagaccatgcaaaatcaaaacatggtgcagcagggaggtccaaaaagacaaagacccccactaagatgttataattgtggaaaatttggccatatgcaaagacaatgtccggaaccaaggaaaacaaaatgtctaaagtgtggaaaattgggacacctagcaaaagactgcaggggacaggtgaattttttagggtatggacggtggatgggggcaaaaccgagaaattttcccgccgctactcttggagcggaaccgagtgcgcctcctccaccgagcggcaccaccccatacgacccagcaaagaagctcctgcagcaatatgcagagaaagggaaacaactgagggagcaaaagaggaatccaccggcaatgaatccggattggaccgagggatattctttgaactccctctttggagaagaccaataaagacagtgtatatagaaggggtccccattaaggcactgctagacacaggggcagatgacaccataattaaagaaaatgatttacaattatcaggtccatggagacccaaaattatagggggcataggaggaggccttaatgtaaaagaatataacgacagggaagtaaaaatagaagataaaattttgagaggaacaatattgttaggagcaactcccattaatataataggtagaaatttgctggccccggcaggtgcccggttagtaatgggacaattatcagaaaaaattcctgtcacacctgtcaaattgaaggaaggggctcggggaccctgtgtaagacaatggcctctctctaaagagaagattgaagctttacaggaaatatgttcccaattagagcaggaaggaaaaatcagtagagtaggaggagaaaatgcatacaataccccaatattttgcataaagaagaaggacaaatcccagtggaggatgctagtagactttagagagttaaataaggcaacccaagatttctttgaagtgcaattagggataccccacccagcaggattaagaaagatgagacagataacagttttagatgtaggagacgcctattattccataccattggatccaaattttaggaaatatactgcttttactattcccacagtgaataatcagggacccgggattaggtatcaattcaactgtctcccgcaagggtggaaaggatctcctacaatcttccaaaatacagcagcatccattttggaggagataaaaagaaacttgccagcactaaccattgtacaatacatggatgatttatgggtaggttctcaagaaaatgaacacacccatgacaaattagtagaacagttaagaacaaaattacaagcctggggcttagaaaccccagaaaagaaggtgcaaaaagaaccaccttatgagtggatgggatacaaactttggcctcacaaatgggaactaagcagaatacaactggaggaaaaagatgaatggactgtcaatgacatccagaagttagttgggaaactaaattgggcagcacaattgtatccaggtcttaggaccaagaatatatgcaagttaattagaggaaagaaaaatctgttagagctagtgacttggacacctgaggcagaagctgaatatgcagaaaatgcagagattcttaaaacagaacaggaaggaacctattacaaaccaggaatacctattagggcagcagtacagaaattggaaggaggacagtggagttaccaattcaaacaagaaggacaagtcttgaaagtaggaaaatacaccaagcaaaagaacacccatacaaatgaacttcgcacattagctggtttagtgcagaagatttgcaaagaagctctagttatttgggggatattaccagttctagaactcccgatagaaagagaggtatgggaacaatggtgggcggattactggcaggtaagctggattcccgaatgggattttgtcagcaccccacctttgctcaaactatggtacacattaacaaaagaacccatacccaaggaggacgtttactatgtagatggagcatgcaacagaaattcaaaagaaggaaaagcaggatacatctcacaatacggaaaacagagagtagaaacattagaaaacactaccaatcagcaagcagaattaacagctataaaaatggctttggaagacagtgggcctaatgtgaacatagtaacagactctcaatatgcaatgggaattttgacagcacaacccacacaaagtgattcaccattagtagagcaaattatagccttaatgatacaaaagcaacaaatatatttgcagtgggtaccagcacataaaggaataggaggaaatgaggagatagataaattagtgagtaaaggcattagaagagttttattcttagaaaaaatagaagaagctcaagaagagcatgaaagatatcataataattggaaaaacctagcagatacatatgggcttccacaaatagtagcaaaagagatagtggccatgtgtccaaaatgtcagataaagggagaaccagtgcatggacaagtggatgcctcacctggaacatggcagatggattgtactcatctagaaggaaaagtagtcatagttgcggtccatgtagccagtggattcatagaagcagaagtcatacctagggaaacaggaaaagaaacggcaaagtttctattaaaaatactgagtagatggcctataacacagttacacacagacaatgggcctaactttacctcccaagaagtggcagcaatatgttggtggggaaaaattgaacatacaacaggtataccatataacccccaatctcaaggatcaatagaaagcatgaacaaacaattaaaagagataattgggaaaataagagatgattgccaatatacagagacagcagtactgatggcttgccatattcacaattttaaaagaaagggaggaatagggggacagacttcagcagagagactaattaatataataacaacacaattagaaatacaacatttacaaaccaaaattcaaaaaattttaaattttagagtctactacagagaagggagagaccctgtgtggaaaggaccagcacaattaatctggaaaggggaaggagcagtggtcctcaaggacggaagtgacctaaaggttgtaccaagaaggaaagctaaaattattaaggattatgaacccaaacaaagagtgggtaatgagggtgacgtggaaggtaccaggggatctgataactaaatggcagggaatagtcagatattggatgagacaaagaaatttgaaatggaactattatatgcatcagctggcggccgcgaattcactagtgattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccaatcgaattcccgcggccgcaattcactcctcaggtgcaggctgcctatcagaaggtggtggctggtgtggccaatgccctggctcacaaataccactgagatctttttccctctgccaaaaattatggggacatcatgaagccccttgagcatctgacttctggctaataaaggaaatttattttcattgcaatagtgtgttggaattttttgtgtctctcactcggaaggacatatgggagggcaaatcatttaaaacatcagaatgagtatttggtttagagtttggcaacatatgcccatatgctggctgccatgaacaaaggttggctataaagaggtcatcagtatatgaaacagccccctgctgtccattccttattccatagaaaagccttgacttgaggttagattttttttatattttgttttgtgttatttttttctttaacatccctaaaattttccttacatgttttactagccagatttttcctcctctcctgactactcccagtcatagctgtccctcttctcttatggagatccctcgacctgcagcccaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagcggatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgac

SEQ ID NO: 10 - DNA, 574 bp, synthetic construct
  misc_feature 1..574: note = hCEF promoter
  source 1..574: mol_type = other DNA
agatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagc

SEQ ID NO: 11 - DNA, 873 bp, Human cytomegalovirus
  source 1..873: mol_type = genomic DNA
ccgcggagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggctagc

SEQ ID NO: 12 - DNA, 395 bp, Homo sapiens
  source 1..395: mol_type = genomic DNA
agatccatatccgcggcaattttaaaagaaagggaggaatagggggacagacttcagcagagagactaattaatataataacaacacaattagaaatacaacatttacaaaccaaaattcaaaaaattttaaattttagagccgcggagatcccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggctagc

SEQ ID NO: 13 - DNA, 4459 bp, synthetic construct
  misc_feature 1..4459: note = soCFTR2
  source 1..4459: mol_type = other DNA
gctagccaccatgcagagaagccctctggagaaggcctctgtggtgagcaagctgttcttcagctggaccaggcccatcctgaggaagggctacaggcagagactggagctgtctgacatctaccagatcccctctgtggactctgctgacaacctgtctgagaagctggagagggagtgggatagagagctggccagcaagaagaaccccaagctgatcaatgccctgaggagatgcttcttctggagattcatgttctatggcatcttcctgtacctgggggaagtgaccaaggctgtgcagcctctgctgctgggcagaatcattgccagctatgaccctgacaacaaggaggagaggagcattgccatctacctgggcattggcctgtgcctgctgttcattgtgaggaccctgctgctgcaccctgccatctttggcctgcaccacattggcatgcagatgaggattgccatgttcagcctgatctacaagaaaaccctgaagctgtccagcagagtgctggacaagatcagcattggccagctggtgagcctgctgagcaacaacctgaacaagtttgatgagggcctggccctggcccactttgtgtggattgcccctctgcaggtggccctgctgatgggcctgatttgggagctgctgcaggcctctgccttttgtggcctgggcttcctgattgtgctggccctgtttcaggctggcctgggcaggatgatgatgaagtacagggaccagagggcaggcaagatcagtgagaggctggtgatcacctctgagatgattgagaacatccagtctgtgaaggcctactgttgggaggaagctatggagaagatgattgaaaacctgaggcagacagagctgaagctgaccaggaaggctgcctatgtgagatacttcaacagctctgccttcttcttctctggcttctttgtggtgttcctgtctgtgctgccctatgccctgatcaaggggatcatcctgagaaagattttcaccaccatcagcttctgcattgtgctgaggatggctgtgaccagacagttcccctgggctgtgcagacctggtatgacagcctgggggccatcaacaagatccaggacttcctgcagaagcaggagtacaagaccctggagtacaacctgaccaccacagaagtggtgatggagaatgtgacagccttctgggaggagggctttggggagctgtttgagaaggccaagcagaacaacaacaacagaaagaccagcaatggggatgactccctgttcttctccaacttctccctgctgggcacacctgtgctgaaggacatcaacttcaagattgagagggggcagctgctggctgtggctggatctacaggggctggcaagaccagcctgctgatgatgatcatgggggagctggagccttctgagggcaagatcaagcactctggcaggatcagcttttgcagccagttcagctggatcatgcctggcaccatcaaggagaacatcatctttggagtgagctatgatgagtacagatacaggagtgtgatcaaggcctgccagctggaggaggacatcagcaagtttgctgagaaggacaacattgtgctgggggagggaggcattacactgtctgggggccagagagccagaatcagcctggccagggctgtgtacaaggatgctgacctgtacctgctggactccccctttggctacctggatgtgctgacagagaaggagatttttgagagctgtgtgtgcaagctgatggccaacaagaccagaatcctggtgaccagcaagatggagcacctgaagaaggctgacaagatcctgatcctgcatgagggcagcagctacttctatgggaccttctctgagctgcagaacctgcagcctgacttcagctctaagctgatgggctgtgacagctttgaccagttctctgctgagaggaggaacagcatcctgacagagaccctgcacagattcagcctggagggagatgcccctgtgagctggacagagaccaagaagcagagcttcaagcagacaggggagtttggggagaagaggaagaactccatcctgaaccccatcaacagcatcaggaagttcagcattgtgcagaaaacccccctgcagatgaatggcattgaggaagattctgatgagcccctggagaggagactgagcctggtgcctgattctgagcagggagaggccatcctgcctaggatctctgtgatcagcacaggccctacactgcaggccagaaggaggcagtctgtgctgaacctgatgacccactctgtgaaccagggccagaacatccacaggaaaaccacagcctccaccaggaaagtgagcctggcccctcaggccaatctgacagagctggacatctacagcaggaggctgtctcaggagacaggcctggagatttctgaggagatcaatgaggaggacctgaaagagtgcttctttgatgacatggagagcatccctgctgtgaccacctggaacacctacctgagatacatcacagtgcacaagagcctgatctttgtgctgatctggtgcctggtgatcttcctggctgaagtggctgcctctctggtggtgctgtggctgctgggaaacaccccactgcaggacaagggcaacagcacccacagcaggaacaacagctatgctgtgatcatcacctccacctccagctactatgtgttctacatctatgtgggagtggctgataccctgctggctatgggcttctttagaggcctgcccctggtgcacacactgatcacagtgagcaagatcctccaccacaagatgctgcactctgtgctgcaggctcctatgagcaccctgaataccctgaaggctgggggcatcctgaacagattctccaaggatattgccatcctggatgacctgctgcctctcaccatctttgacttcatccagctgctgctgattgtgattggggccattgctgtggtggcagtgctgcagccctacatctttgtggccacagtgcctgtgattgtggccttcatcatgctgagggcctactttctgcagacctcccagcagctgaagcagctggagtctgagggcagaagccccatcttcacccacctggtgacaagcctgaagggcctgtggaccctgagagcctttggcaggcagccctactttgagaccctgttccacaaggccctgaacctgcacacagccaactggttcctctacctgtccaccctgagatggttccagatgagaattgagatgatctttgtcatcttcttcattgctgtgaccttcatcagcattctgaccacaggagagggagagggcagagtgggcattatcctgaccctggccatgaacatcatgagcacactgcagtgggcagtgaacagcagcattgatgtggacagcctgatgaggagtgtgagcagagtgttcaagttcattgatatgcccacagagggcaagcctaccaagagcaccaagccctacaagaatggccagctgagcaaagtgatgatcattgagaacagccatgtgaagaaggatgatatctggcccagtggaggccagatgacagtgaaggacctgacagccaagtacacagaggggggcaatgctatcctggagaacatctccttcagcatctcccctggccagagagtgggactgctgggaagaacaggctctggcaagtctaccctgctgtctgccttcctgaggctgctgaacacagagggagagatccagattgatggagtgtcctgggacagcatcacactgcagcagtggaggaaggcctttggtgtgatcccccagaaagtgttcatcttcagtggcaccttcaggaagaacctggacccctatgagcagtggtctgaccaggagatttggaaagtggctgatgaagtgggcctgagaagtgtgattgagcagttccctggcaagctggactttgtcctggtggatgggggctgtgtgctgagccatggccacaagcagctgatgtgcctggccagatcagtgctgagcaaggccaagatcctgctgctggatgagccttctgcccacctggatcctgtgacctaccagatcatcaggaggaccctcaagcaggcctttgctgactgcacagtcatcctgtgtgagcacaggattgaggccatgctggagtgccagcagttcctggtgattgaggagaacaaagtgaggcagtatgacagcatccagaagctgctgaatgagaggagcctgttcaggcaggccatcagcccctctgatagagtgaagctgttcccccacaggaacagctccaagtgcaagagcaagccccagattgctgccctgaaggaggagacagaggaggaagtgcaggacaccaggctgtgagggccc

SEQ ID NO: 14 - DNA, 1257 bp, synthetic construct
  misc_feature 1..1257: note = sohAAT
  source 1..1257: mol_type = other DNA
atgcccagctctgtgtcctggggcattctgctgctggctggcctgtgctgtctggtgcctgtgtccctggctgaggaccctcagggggatgctgcccagaaaacagacacctcccaccatgaccaggaccaccccaccttcaacaagatcacccccaacctggcagagtttgccttcagcctgtacagacagctggcccaccagagcaacagcaccaacatctttttcagccctgtgtccattgccacagcctttgccatgctgagcctgggcaccaaggctgacacccatgatgagatcctggaaggcctgaacttcaacctgacagagatccctgaggcccagatccatgagggcttccaggaactgctgagaaccctgaaccagccagacagccagctgcagctgacaacaggcaatgggctgttcctgtctgagggcctgaagctggtggacaagtttctggaagatgtgaagaagctgtaccactctgaggccttcacagtgaactttggggacacagaagaggccaagaaacagatcaatgactatgtggaaaagggcacccagggcaagattgtggaccttgtgaaagagctggacagggacactgtgtttgcccttgtgaactacatcttcttcaagggcaagtgggagaggccctttgaagtgaaggacactgaggaagaggacttccatgtggaccaagtgaccacagtgaaggtgccaatgatgaagagactggggatgttcaatatccagcactgcaagaaactgagcagctgggtgctgctgatgaagtacctgggcaatgctacagccatattctttctgcctgatgagggcaagctgcagcacctggaaaatgagctgacccatgacatcatcaccaaatttctggaaaatgaggacagaagatctgccagcctgcatctgcccaagctgagcatcacaggcacatatgacctgaagtctgtgctgggacagctgggaatcaccaaggtgttcagcaatggggcagacctgagtggagtgacagaggaagcccctctgaagctgtccaaggctgtgcacaaggcagtgctgaccattgatgagaagggcacagaggctgctggggccatgtttctggaagccatccccatgtccatccccccagaagtgaagttcaacaagccctttgtgttcctgatgattgagcagaacaccaagagccccctgttcatgggcaaggttgtgaaccccacccagaaatga

SEQ ID NO: 15 - DNA, 1257 bp, synthetic construct
  misc_feature 1..1257: note = sohAAT completmentary strand
  source 1..1257: mol_type = other DNA
tacgggtcgagacacaggaccccgtaagacgacgaccgaccggacacgacagaccacggacacagggaccgactcctgggagtccccctacgacgggtcttttgtctgtggagggtggtactggtcctggtggggtggaagttgttctagtgggggttggaccgtctcaaacggaagtcggacatgtctgtcgaccgggtggtctcgttgtcgtggttgtagaaaaagtcgggacacaggtaacggtgtcggaaacggtacgactcggacccgtggttccgactgtgggtactactctaggaccttccggacttgaagttggactgtctctagggactccgggtctaggtactcccgaaggtccttgacgactcttgggacttggtcggtctgtcggtcgacgtcgactgttgtccgttacccgacaaggacagactcccggacttcgaccacctgttcaaagaccttctacacttcttcgacatggtgagactccggaagtgtcacttgaaacccctgtgtcttctccggttctttgtctagttactgatacaccttttcccgtgggtcccgttctaacacctggaacactttctcgacctgtccctgtgacacaaacgggaacacttgatgtagaagaagttcccgttcaccctctccgggaaacttcacttcctgtgactccttctcctgaaggtacacctggttcactggtgtcacttccacggttactacttctctgacccctacaagttataggtcgtgacgttctttgactcgtcgacccacgacgactacttcatggacccgttacgatgtcggtataagaaagacggactactcccgttcgacgtcgtggaccttttactcgactgggtactgtagtagtggtttaaagaccttttactcctgtcttctagacggtcggacgtagacgggttcgactcgtagtgtccgtgtatactggacttcagacacgaccctgtcgacccttagtggttccacaagtcgttaccccgtctggactcacctcactgtctccttcggggagacttcgacaggttccgacacgtgttccgtcacgactggtaactactcttcccgtgtctccgacgaccccggtacaaagaccttcggtaggggtacaggtaggggggtcttcacttcaagttgttcgggaaacacaaggactactaactcgtcttgtggttctcgggggacaagtacccgttccaacacttggggtgggtctttact

SEQ ID NO: 16 - protein, 419 aa, Homo sapiens
AEDPQGDAAQKTDTSHHDQDHPTFAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 17 - DNA, 5013 bp, synthetic construct
  misc_feature 1..5013: note = codon-optimised FVIII transgene (N6)
  source 1..5013: mol_type = other DNA
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaacagcaggcaccccagcaccaggcagaagcagttcaatgccaccaccatccctgagaatgacatagagaagacagacccatggtttgcccaccggacccccatgcccaagatccagaatgtgagcagctctgacctgctgatgctgctgaggcagagccccaccccccatggcctgagcctgtctgacctgcaggaggccaagtatgaaaccttctctgatgaccccagccctggggccattgacagcaacaacagcctgtctgagatgacccacttcaggccccagctgcaccactctggggacatggtgttcacccctgagtctggcctgcagctgaggctgaatgagaagctgggcaccactgctgccactgagctgaagaagctggacttcaaagtctccagcaccagcaacaacctgatcagcaccatcccctctgacaacctggctgctggcactgacaacaccagcagcctgggcccccccagcatgcctgtgcactatgacagccagctggacaccaccctgtttggcaagaagagcagccccctgactgagtctgggggccccctgagcctgtctgaggagaacaatgacagcaagctgctggagtctggcctgatgaacagccaggagagcagctggggcaagaatgtgagcagcagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 18 - DNA, 4425 bp, synthetic construct
  misc_feature 1..4425: note = codon-optimised FVIII transgene (V3)
  source 1..4425: mol_type = other DNA
atgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactga

SEQ ID NO: 19 - DNA, 5013 bp, synthetic construct
  misc_feature 1..5013: note = codon-optimised FVIII transgene (N6) complementary strand
  source 1..5013: mol_type = other DNA
tacgtctaactcgactcgtggacgaagaaggacacggacgactccaagacgaagagacggtggtcctctatgatggacccccgacacctcgactcgaccctgatgtacgtcagactggaccccctcgacggacacctacggtccaagggggggtctcacgggttctcgaaggggaagttgtggagacaccacatgttcttctgggacaaacacctcaagtgactggtggacaagttgtaacggttcgggtccggggggacctacccggacgacccggggtggtaggtccgactccacatactgtgacaccactagtgggacttcttgtaccggtcggtgggacactcggacgtacgacacccccactcgatgaccttccggagactcccccgactcatactactggtctggtcggtctccctcttcctcctactgttccacaagggacccccgtcggtgtggatacacaccgtccacgacttcctcttaccggggtaccggagactgggggacacggactggatgtcgatggactcggtacacctggaccacttcctggacttgagaccggactaaccccgggacgaccacacgtccctcccgtcggaccggttcctcttctgggtctgggacgtgttcaagtaggacgacaaacgacacaaactactcccgttctcgaccgtgagactttggttcttgtcggactacgtcctgtccctacgacggagacggtcccggaccgggttctacgtgtgacacttaccgatacacttgtcctcggacggaccggactaaccgacggtgtccttcagacacatgaccgtacactaaccgtacccgtggtggggactccacgtgtcgtagaaggacctcccggtgtggaaggaccagtccttggtgtccgtccggtcggacctctagtcggggtagtggaaggactgacgggtctgggacgactacctggacccggtcaaggacgacaagacggtgtagtcgtcggtggtcgtactaccgtacctccggatacacttccacctgtcgacgggactcctcggggtcgactcctacttcttgttactcctccgactcctgatactactactggactgactgagactctacctacaccactccaaactactactgttgtcggggtcgaagtaggtctagtccagacaccggttcttcgtggggttctggacccacgtgatgtaacgacgactcctcctcctgaccctgatacggggggaccacgaccggggactactgtcctcgatgttctcggtcatggacttgttaccgggggtctcctaaccgtccttcatgttcttccagtccaagtaccggatgtgactactttggaagttctggtccctccggtaggtcgtactcagaccgtaggacccgggggacgacatacccctccaccccctgtgggacgactagtagaagttcttggtccggtcgtccgggatgttgtagatgggggtaccgtagtgactacactccggggacatgtcgtcctccgacgggttcccccacttcgtggacttcctgaaggggtaggacggacccctctagaagttcatgttcacctgacactgacacctcctaccggggtggttcagactggggtccacggactggtctatgatgtcgtcgaaacacttgtacctctccctggaccggagaccggactaaccgggggacgactagacgatgttcctcagacacctggtctccccgttggtctagtacagactgttctccttacactaggacaagagacacaaactactcttgtcctcgaccatggactgactcttgtaggtctccaaggacgggttgggacgaccccacgtcgacctcctgggactcaaggtccggtcgttgtagtacgtgtcgtagttaccgatacacaaactgtcggacgtcgacagacacacggacgtactccaccggatgaccatgtaggactcgtaaccccgggtctgactgaaggacagacacaagaagagaccgatgtggaagttcgtgttctaccacatactcctgtgggactgggacaaggggaagagacccctctgacacaagtactcgtacctcttgggaccggacacctaagacccgacggtgttgagactgaagtccttgtccccgtactgacgggacgactttcagaggtcgacactgttcttgtgacccctgatgatactcctgtcgatactcctgtagagacggatggacgactcgttcttgttacggtaactcgggtcctcgaagtcggtcttgtcgtccgtggggtcgtggtccgtcttcgtcaagttacggtggtggtagggactcttactgtatctcttctgtctgggtaccaaacgggtggcctgggggtacgggttctaggtcttacactcgtcgagactggacgactacgacgactccgtctcggggtggggggtaccggactcggacagactggacgtcctccggttcatactttggaagagactactggggtcgggaccccggtaactgtcgttgttgtcggacagactctactgggtgaagtccggggtcgacgtggtgagacccctgtaccacaagtggggactcagaccggacgtcgactccgacttactcttcgacccgtggtgacgacggtgactcgacttcttcgacctgaagtttcagaggtcgtggtcgttgttggactagtcgtggtaggggagactgttggaccgacgaccgtgactgttgtggtcgtcggacccgggggggtcgtacggacacgtgatactgtcggtcgacctgtggtgggacaaaccgttcttctcgtcgggggactgactcagacccccgggggactcggacagactcctcttgttactgtcgttcgacgacctcagaccggactacttgtcggtcctctcgtcgaccccgttcttacactcgtcgtccctctagtggtcctggtgggacgtcagactggtcctcctctaactgatactactgtggtagagacacctctacttcttcctcctgaaactgtagatgctgctcctgctcttggtctcggggtcctcgaaggtcttcttctggtccgtgatgaagtaacgacgacacctctccgacaccctgataccgtactcgtcgtcgggggtacacgactccttgtcccgggtcagaccgagacacggggtcaagttcttccaccacaaggtcctcaagtgactaccgtcgaagtgggtcggggacatgtctcccctcgacttactcgtggacccggacgacccggggatgtagtcccgactccacctcctgttgtagtaccactggaagtccttggtccggtcgtccgggatgtcgaagatgtcgtcggactagtcgatactcctcctggtctccgtcccccgactcgggtccttcttgaaacacttcgggttactttggttctggatgaagaccttccacgtcgtggtgtaccgggggtggttcctactcaaactgacgttccggacccggatgaagagactacacctggacctcttcctacacgtgagaccggactaaccgggggacgaccacacggtgtggttgtgggacttgggacgggtaccgtccgtccactgacacgtcctcaaacgggacaagaagtggtagaaactactttggttctcgaccatgaagtgactcttgtacctctccttgacgtcccgggggacgttgtaggtctacctcctggggtggaagttcctcttgatgtccaaggtacggtagttaccgatgtagtacctgtgggacggaccggaccactaccgggtcctggtctcctagtccaccatggacgactcgtacccgtcgttactcttgtaggtgtcgtaggtgaagagaccggtacacaagtgacactccttcttcctcctcatgttctaccgggacatgttggacatgggaccccacaaactctgacacctctacgacgggtcgttccgaccgtagacctcccacctcacggactaacccctcgtggacgtacgaccgtactcgtgggacaaggaccacatgtcgttgttcacggtctggggggacccgtaccggagaccggtgtagtccctgaaggtctagtgacggagaccggtcataccggtcacccgggggttcgaccggtccgacgtgatgagaccgtcgtagttacggacctcgtggttcctcgggaagtcgacctagttccacctggacgaccgggggtactagtaggtaccgtagttctgggtcccccggtccgtcttcaagtcgtcggacatgtagtcggtcaagtagtagtacatgtcggacctaccgttcttcaccgtctggatgtccccgttgtcgtgaccgtgggactaccacaagaaaccgttacacctgtcgagaccgtagttcgtgttgtagaagttgggggggtagtaacggtctatgtagtccgacgtggggtgggtgatgtcgtagtcctcgtgggactcctacctcgactacccgacactggacttgtcgacgtcgtacggggacccgtacctctcgttccggtagagactacgggtctagtgacggtcgtcgatgaagtggttgtacaaacggtggacctcggggtcgttccggtccgacgtggacgtcccgtcctcgttacggacctccggggtccagttgttggggttcctcaccgacgtccacctgaaggtcttctggtacttccactgaccccactggtgggtcccccacttctcggacgactggtcgtacatacacttcctcaaggactagtcgtcgtcggtcctaccggtggtcacctgggacaagaaggtcttaccgttccacttccacaaggtcccgttggtcctgtcgaagtggggacaccacttgtcggacctggggggggacgactggtctatggactcctaagtgggggtctcgacccacgtggtctaacgggactcctacctccacgacccgacactccgggtcctggacatgact

SEQ ID NO: 20 - DNA, 4425 bp, synthetic construct
  misc_feature 1..4425: note = codon-optimised FVIII transgene (V3) complementary strand
  source 1..4425: mol_type = other DNA
tacgtctaactcgactcgtggacgaagaaggacacggacgactccaagacgaagagacggtggtcctctatgatggacccccgacacctcgactcgaccctgatgtacgtcagactggaccccctcgacggacacctacggtccaagggggggtctcacgggttctcgaaggggaagttgtggagacaccacatgttcttctgggacaaacacctcaagtgactggtggacaagttgtaacggttcgggtccggggggacctacccggacgacccggggtggtaggtccgactccacatactgtgacaccactagtgggacttcttgtaccggtcggtgggacactcggacgtacgacacccccactcgatgaccttccggagactcccccgactcatactactggtctggtcggtctccctcttcctcctactgttccacaagggacccccgtcggtgtggatacacaccgtccacgacttcctcttaccggggtaccggagactgggggacacggactggatgtcgatggactcggtacacctggaccacttcctggacttgagaccggactaaccccgggacgaccacacgtccctcccgtcggaccggttcctcttctgggtctgggacgtgttcaagtaggacgacaaacgacacaaactactcccgttctcgaccgtgagactttggttcttgtcggactacgtcctgtccctacgacggagacggtcccggaccgggttctacgtgtgacacttaccgatacacttgtcctcggacggaccggactaaccgacggtgtccttcagacacatgaccgtacactaaccgtacccgtggtggggactccacgtgtcgtagaaggacctcccggtgtggaaggaccagtccttggtgtccgtccggtcggacctctagtcggggtagtggaaggactgacgggtctgggacgactacctggacccggtcaaggacgacaagacggtgtagtcgtcggtggtcgtactaccgtacctccggatacacttccacctgtcgacgggactcctcggggtcgactcctacttcttgttactcctccgactcctgatactactactggactgactgagactctacctacaccactccaaactactactgttgtcggggtcgaagtaggtctagtccagacaccggttcttcgtggggttctggacccacgtgatgtaacgacgactcctcctcctgaccctgatacggggggaccacgaccggggactactgtcctcgatgttctcggtcatggacttgttaccgggggtctcctaaccgtccttcatgttcttccagtccaagtaccggatgtgactactttggaagttctggtccctccggtaggtcgtactcagaccgtaggacccgggggacgacatacccctccaccccctgtgggacgactagtagaagttcttggtccggtcgtccgggatgttgtagatgggggtaccgtagtgactacactccggggacatgtcgtcctccgacgggttcccccacttcgtggacttcctgaaggggtaggacggacccctctagaagttcatgttcacctgacactgacacctcctaccggggtggttcagactggggtccacggactggtctatgatgtcgtcgaaacacttgtacctctccctggaccggagaccggactaaccgggggacgactagacgatgttcctcagacacctggtctccccgttggtctagtacagactgttctccttacactaggacaagagacacaaactactcttgtcctcgaccatggactgactcttgtaggtctccaaggacgggttgggacgaccccacgtcgacctcctgggactcaaggtccggtcgttgtagtacgtgtcgtagttaccgatacacaaactgtcggacgtcgacagacacacggacgtactccaccggatgaccatgtaggactcgtaaccccgggtctgactgaaggacagacacaagaagagaccgatgtggaagttcgtgttctaccacatactcctgtgggactgggacaaggggaagagacccctctgacacaagtactcgtacctcttgggaccggacacctaagacccgacggtgttgagactgaagtccttgtccccgtactgacgggacgactttcagaggtcgacactgttcttgtgacccctgatgatactcctgtcgatactcctgtagagacggatggacgactcgttcttgttacggtaactcgggtcctcgaagtcggtcttacggtgattacacagattgttgtcgttgtggtcgttactgtcgttacacagagggggtcacgacttctccgtggtctccctctagtggtcctggtgggacgtcagactggtcctcctctaactgatactactgtggtagagacacctctacttcttcctcctgaaactgtagatgctgctcctgctcttggtctcggggtcctcgaaggtcttcttctggtccgtgatgaagtaacgacgacacctctccgacaccctgataccgtactcgtcgtcgggggtacacgactccttgtcccgggtcagaccgagacacggggtcaagttcttccaccacaaggtcctcaagtgactaccgtcgaagtgggtcggggacatgtctcccctcgacttactcgtggacccggacgacccggggatgtagtcccgactccacctcctgttgtagtaccactggaagtccttggtccggtcgtccgggatgtcgaagatgtcgtcggactagtcgatactcctcctggtctccgtcccccgactcgggtccttcttgaaacacttcgggttactttggttctggatgaagaccttccacgtcgtggtgtaccgggggtggttcctactcaaactgacgttccggacccggatgaagagactacacctggacctcttcctacacgtgagaccggactaaccgggggacgaccacacggtgtggttgtgggacttgggacgggtaccgtccgtccactgacacgtcctcaaacgggacaagaagtggtagaaactactttggttctcgaccatgaagtgactcttgtacctctccttgacgtcccgggggacgttgtaggtctacctcctggggtggaagttcctcttgatgtccaaggtacggtagttaccgatgtagtacctgtgggacggaccggaccactaccgggtcctggtctcctagtccaccatggacgactcgtacccgtcgttactcttgtaggtgtcgtaggtgaagagaccggtacacaagtgacactccttcttcctcctcatgttctaccgggacatgttggacatgggaccccacaaactctgacacctctacgacgggtcgttccgaccgtagacctcccacctcacggactaacccctcgtggacgtacgaccgtactcgtgggacaaggaccacatgtcgttgttcacggtctggggggacccgtaccggagaccggtgtagtccctgaaggtctagtgacggagaccggtcataccggtcacccgggggttcgaccggtccgacgtgatgagaccgtcgtagttacggacctcgtggttcctcgggaagtcgacctagttccacctggacgaccgggggtactagtaggtaccgtagttctgggtcccccggtccgtcttcaagtcgtcggacatgtagtcggtcaagtagtagtacatgtcggacctaccgttcttcaccgtctggatgtccccgttgtcgtgaccgtgggactaccacaagaaaccgttacacctgtcgagaccgtagttcgtgttgtagaagttgggggggtagtaacggtctatgtagtccgacgtggggtgggtgatgtcgtagtcctcgtgggactcctacctcgactacccgacactggacttgtcgacgtcgtacggggacccgtacctctcgttccggtagagactacgggtctagtgacggtcgtcgatgaagtggttgtacaaacggtggacctcggggtcgttccggtccgacgtggacgtcccgtcctcgttacggacctccggggtccagttgttggggttcctcaccgacgtccacctgaaggtcttctggtacttccactgaccccactggtgggtcccccacttctcggacgactggtcgtacatacacttcctcaaggactagtcgtcgtcggtcctaccggtggtcacctgggacaagaaggtcttaccgttccacttccacaaggtcccgttggtcctgtcgaagtggggacaccacttgtcggacctggggggggacgactggtctatggactcctaagtgggggtctcgacccacgtggtctaacgggactcctacctccacgacccgacactccgggtcctggacatgact

SEQ ID NO: 21 - protein, 1670 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 22 - protein, 1474 aa, Homo sapiens [sequence omitted]

SEQ ID NO: 23 - DNA, 600 bp, Woodchuck hepatitis virus
  source 1..600: mol_type = genomic DNA
gggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagct

SEQ ID NO: 24 - DNA, 7349 bp, synthetic construct
  misc_feature 1..7349: note = pGM407
  source 1..7349: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactcttgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatttttttgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccatgcccagctctgtgtcctggggcattctgctgctggctggcctgtgctgtctggtgcctgtgtccctggctgaggaccctcagggggatgctgcccagaaaacagacacctcccaccatgaccaggaccaccccaccttcaacaagatcacccccaacctggcagagtttgccttcagcctgtacagacagctggcccaccagagcaacagcaccaacatctttttcagccctgtgtccattgccacagcctttgccatgctgagcctgggcaccaaggctgacacccatgatgagatcctggaaggcctgaacttcaacctgacagagatccctgaggcccagatccatgagggcttccaggaactgctgagaaccctgaaccagccagacagccagctgcagctgacaacaggcaatgggctgttcctgtctgagggcctgaagctggtggacaagtttctggaagatgtgaagaagctgtaccactctgaggccttcacagtgaactttggggacacagaagaggccaagaaacagatcaatgactatgtggaaaagggcacccagggcaagattgtggaccttgtgaaagagctggacagggacactgtgtttgcccttgtgaactacatcttcttcaagggcaagtgggagaggccctttgaagtgaaggacactgaggaagaggacttccatgtggaccaagtgaccacagtgaaggtgccaatgatgaagagactggggatgttcaatatccagcactgcaagaaactgagcagctgggtgctgctgatgaagtacctgggcaatgctacagccatattctttctgcctgatgagggcaagctgcagcacctggaaaatgagctgacccatgacatcatcaccaaatttctggaaaatgaggacagaagatctgccagcctgcatctgcccaagctgagcatcacaggcacatatgacctgaagtctgtgctgggacagctgggaatcaccaaggtgttcagcaatggggcagacctgagtggagtgacagaggaagcccctctgaagctgtccaaggctgtgcacaaggcagtgctgaccattgatgagaagggcacagaggctgctggggccatgtttctggaagccatccccatgtccatccccccagaagtgaagttcaacaagccctttgtgttcctgatgattgagcagaacaccaagagccccctgttcatgggcaaggttgtgaaccccacccagaaatgagggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 25 - DNA, 10812 bp, synthetic construct
  misc_feature 1..10812: note = pGM411
  source 1..10812: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggctagccaccaatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgagcggccgcgggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 26 - DNA, 10519 bp, synthetic construct
  misc_feature 1..10519: note = pGM413
  source 1..10519: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccaatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaatgccactaatgtgtctaacaacagcaacaccagcaatgacagcaatgtgtctcccccagtgctgaagaggcaccagagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgagcggccgcgggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 27 - DNA, 11400 bp, synthetic construct
  misc_feature 1..11400: note = pGM412
  source 1..11400: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactctgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggctagccaccaatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaacagcaggcaccccagcaccaggcagaagcagttcaatgccaccaccatccctgagaatgacatagagaagacagacccatggtttgcccaccggacccccatgcccaagatccagaatgtgagcagctctgacctgctgatgctgctgaggcagagccccaccccccatggcctgagcctgtctgacctgcaggaggccaagtatgaaaccttctctgatgaccccagccctggggccattgacagcaacaacagcctgtctgagatgacccacttcaggccccagctgcaccactctggggacatggtgttcacccctgagtctggcctgcagctgaggctgaatgagaagctgggcaccactgctgccactgagctgaagaagctggacttcaaagtctccagcaccagcaacaacctgatcagcaccatcccctctgacaacctggctgctggcactgacaacaccagcagcctgggcccccccagcatgcctgtgcactatgacagccagctggacaccaccctgtttggcaagaagagcagccccctgactgagtctgggggccccctgagcctgtctgaggagaacaatgacagcaagctgctggagtctggcctgatgaacagccaggagagcagctggggcaagaatgtgagcagcagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgagcggccgcgggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 28 - DNA, 11108 bp, synthetic construct
  misc_feature 1..11108: note = pGM414
  source 1..11108: mol_type = other DNA
ggtacctcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaacaactgcgatcgcccgccccgttgacgcaaatgggcggtaggcgtgtacggtgggaggtctatataagcagagctcgctggcttgtaactcagtctcttactaggagaccagcttgagcctgggtgttcgctggttagcctaacctggttggccaccaggggtaaggactccttggcttagaaagctaataaacttgcctgcattagagcttatctgagtcaagtgtcctcattgacgcctcactctcttgaacgggaatcttccttactgggttctctctctgacccaggcgagagaaactccagcagtggcgcccgaacagggacttgagtgagagtgtaggcacgtacagctgagaaggcgtcggacgcgaaggaagcgcggggtgcgacgcgaccaagaaggagacttggtgagtaggcttctcgagtgccgggaaaaagctcgagcctagttagaggactaggagaggccgtagccgtaactactcttgggcaagtagggcaggcggtgggtacgcaatgggggcggctacctcagcactaaataggagacaattagaccaatttgagaaaatacgacttcgcccgaacggaaagaaaaagtaccaaattaaacatttaatatgggcaggcaaggagatggagcgcttcggcctccatgagaggttgttggagacagaggaggggtgtaaaagaatcatagaagtcctctaccccctagaaccaacaggatcggagggcttaaaaagtctgttcaatcttgtgtgcgtgctatattgcttgcacaaggaacagaaagtgaaagacacagaggaagcagtagcaacagtaagacaacactgccatctagtggaaaaagaaaaaagtgcaacagagacatctagtggacaaaagaaaaatgacaagggaatagcagcgccacctggtggcagtcagaattttccagcgcaacaacaaggaaatgcctgggtacatgtacccttgtcaccgcgcaccttaaatgcgtgggtaaaagcagtagaggagaaaaaatttggagcagaaatagtacccatgtttcaagccctatcgaattcccgtttgtgctagggttcttaggcttcttgggggctgctggaactgcaatgggagcagcggcgacagccctgacggtccagtctcagcatttgcttgctgggatactgcagcagcagaagaatctgctggcggctgtggaggctcaacagcagatgttgaagctgaccatttggggtgttaaaaacctcaatgcccgcgtcacagcccttgagaagtacctagaggatcaggcacgactaaactcctgggggtgcgcatggaaacaagtatgtcataccacagtggagtggccctggacaaatcggactccggattggcaaaatatgacttggttggagtgggaaagacaaatagctgatttggaaagcaacattacgagacaattagtgaaggctagagaacaagaggaaaagaatctagatgcctatcagaagttaactagttggtcagatttctggtcttggttcgatttctcaaaatggcttaacattttaaaaatgggatttttagtaatagtaggaataatagggttaagattactttacacagtatatggatgtatagtgagggttaggcagggatatgttcctctatctccacagatccatatccgcggcaattttaaaagaaagggaggaatagggggacagacttcagcagagagactaattaatataataacaacacaattagaaatacaacatttacaaaccaaaattcaaaaaattttaaattttagagccgcggagatctgttacataacttatggtaaatggcctgcctggctgactgcccaatgacccctgcccaatgatgtcaataatgatgtatgttcccatgtaatgccaatagggactttccattgatgtcaatgggtggagtatttatggtaactgcccacttggcagtacatcaagtgtatcatatgccaagtatgccccctattgatgtcaatgatggtaaatggcctgcctggcattatgcccagtacatgaccttatgggactttcctacttggcagtacatctatgtattagtcattgctattaccatgggaattcactagtggagaagagcatgcttgagggctgagtgcccctcagtgggcagagagcacatggcccacagtccctgagaagttggggggaggggtgggcaattgaactggtgcctagagaaggtggggcttgggtaaactgggaaagtgatgtggtgtactggctccacctttttccccagggtgggggagaaccatatataagtgcagtagtctctgtgaacattcaagcttctgccttctccctcctgtgagtttgctagccaccaatgcagattgagctgagcacctgcttcttcctgtgcctgctgaggttctgcttctctgccaccaggagatactacctgggggctgtggagctgagctgggactacatgcagtctgacctgggggagctgcctgtggatgccaggttcccccccagagtgcccaagagcttccccttcaacacctctgtggtgtacaagaagaccctgtttgtggagttcactgaccacctgttcaacattgccaagcccaggcccccctggatgggcctgctgggccccaccatccaggctgaggtgtatgacactgtggtgatcaccctgaagaacatggccagccaccctgtgagcctgcatgctgtgggggtgagctactggaaggcctctgagggggctgagtatgatgaccagaccagccagagggagaaggaggatgacaaggtgttccctgggggcagccacacctatgtgtggcaggtgctgaaggagaatggccccatggcctctgaccccctgtgcctgacctacagctacctgagccatgtggacctggtgaaggacctgaactctggcctgattggggccctgctggtgtgcagggagggcagcctggccaaggagaagacccagaccctgcacaagttcatcctgctgtttgctgtgtttgatgagggcaagagctggcactctgaaaccaagaacagcctgatgcaggacagggatgctgcctctgccagggcctggcccaagatgcacactgtgaatggctatgtgaacaggagcctgcctggcctgattggctgccacaggaagtctgtgtactggcatgtgattggcatgggcaccacccctgaggtgcacagcatcttcctggagggccacaccttcctggtcaggaaccacaggcaggccagcctggagatcagccccatcaccttcctgactgcccagaccctgctgatggacctgggccagttcctgctgttctgccacatcagcagccaccagcatgatggcatggaggcctatgtgaaggtggacagctgccctgaggagccccagctgaggatgaagaacaatgaggaggctgaggactatgatgatgacctgactgactctgagatggatgtggtgaggtttgatgatgacaacagccccagcttcatccagatcaggtctgtggccaagaagcaccccaagacctgggtgcactacattgctgctgaggaggaggactgggactatgcccccctggtgctggcccctgatgacaggagctacaagagccagtacctgaacaatggcccccagaggattggcaggaagtacaagaaggtcaggttcatggcctacactgatgaaaccttcaagaccagggaggccatccagcatgagtctggcatcctgggccccctgctgtatggggaggtgggggacaccctgctgatcatcttcaagaaccaggccagcaggccctacaacatctacccccatggcatcactgatgtgaggcccctgtacagcaggaggctgcccaagggggtgaagcacctgaaggacttccccatcctgcctggggagatcttcaagtacaagtggactgtgactgtggaggatggccccaccaagtctgaccccaggtgcctgaccagatactacagcagctttgtgaacatggagagggacctggcctctggcctgattggccccctgctgatctgctacaaggagtctgtggaccagaggggcaaccagatcatgtctgacaagaggaatgtgatcctgttctctgtgtttgatgagaacaggagctggtacctgactgagaacatccagaggttcctgcccaaccctgctggggtgcagctggaggaccctgagttccaggccagcaacatcatgcacagcatcaatggctatgtgtttgacagcctgcagctgtctgtgtgcctgcatgaggtggcctactggtacatcctgagcattggggcccagactgacttcctgtctgtgttcttctctggctacaccttcaagcacaagatggtgtatgaggacaccctgaccctgttccccttctctggggagactgtgttcatgagcatggagaaccctggcctgtggattctgggctgccacaactctgacttcaggaacaggggcatgactgccctgctgaaagtctccagctgtgacaagaacactggggactactatgaggacagctatgaggacatctctgcctacctgctgagcaagaacaatgccattgagcccaggagcttcagccagaacagcaggcaccccagcaccaggcagaagcagttcaatgccaccaccatccctgagaatgacatagagaagacagacccatggtttgcccaccggacccccatgcccaagatccagaatgtgagcagctctgacctgctgatgctgctgaggcagagccccaccccccatggcctgagcctgtctgacctgcaggaggccaagtatgaaaccttctctgatgaccccagccctggggccattgacagcaacaacagcctgtctgagatgacccacttcaggccccagctgcaccactctggggacatggtgttcacccctgagtctggcctgcagctgaggctgaatgagaagctgggcaccactgctgccactgagctgaagaagctggacttcaaagtctccagcaccagcaacaacctgatcagcaccatcccctctgacaacctggctgctggcactgacaacaccagcagcctgggcccccccagcatgcctgtgcactatgacagccagctggacaccaccctgtttggcaagaagagcagccccctgactgagtctgggggccccctgagcctgtctgaggagaacaatgacagcaagctgctggagtctggcctgatgaacagccaggagagcagctggggcaagaatgtgagcagcagggagatcaccaggaccaccctgcagtctgaccaggaggagattgactatgatgacaccatctctgtggagatgaagaaggaggactttgacatctacgacgaggacgagaaccagagccccaggagcttccagaagaagaccaggcactacttcattgctgctgtggagaggctgtgggactatggcatgagcagcagcccccatgtgctgaggaacagggcccagtctggctctgtgccccagttcaagaaggtggtgttccaggagttcactgatggcagcttcacccagcccctgtacagaggggagctgaatgagcacctgggcctgctgggcccctacatcagggctgaggtggaggacaacatcatggtgaccttcaggaaccaggccagcaggccctacagcttctacagcagcctgatcagctatgaggaggaccagaggcagggggctgagcccaggaagaactttgtgaagcccaatgaaaccaagacctacttctggaaggtgcagcaccacatggcccccaccaaggatgagtttgactgcaaggcctgggcctacttctctgatgtggacctggagaaggatgtgcactctggcctgattggccccctgctggtgtgccacaccaacaccctgaaccctgcccatggcaggcaggtgactgtgcaggagtttgccctgttcttcaccatctttgatgaaaccaagagctggtacttcactgagaacatggagaggaactgcagggccccctgcaacatccagatggaggaccccaccttcaaggagaactacaggttccatgccatcaatggctacatcatggacaccctgcctggcctggtgatggcccaggaccagaggatcaggtggtacctgctgagcatgggcagcaatgagaacatccacagcatccacttctctggccatgtgttcactgtgaggaagaaggaggagtacaagatggccctgtacaacctgtaccctggggtgtttgagactgtggagatgctgcccagcaaggctggcatctggagggtggagtgcctgattggggagcacctgcatgctggcatgagcaccctgttcctggtgtacagcaacaagtgccagacccccctgggcatggcctctggccacatcagggacttccagatcactgcctctggccagtatggccagtgggcccccaagctggccaggctgcactactctggcagcatcaatgcctggagcaccaaggagcccttcagctggatcaaggtggacctgctggcccccatgatcatccatggcatcaagacccagggggccaggcagaagttcagcagcctgtacatcagccagttcatcatcatgtacagcctggatggcaagaagtggcagacctacaggggcaacagcactggcaccctgatggtgttctttggcaatgtggacagctctggcatcaagcacaacatcttcaacccccccatcattgccagatacatcaggctgcaccccacccactacagcatcaggagcaccctgaggatggagctgatgggctgtgacctgaacagctgcagcatgcccctgggcatggagagcaaggccatctctgatgcccagatcactgccagcagctacttcaccaacatgtttgccacctggagccccagcaaggccaggctgcacctgcagggcaggagcaatgcctggaggccccaggtcaacaaccccaaggagtggctgcaggtggacttccagaagaccatgaaggtgactggggtgaccacccagggggtgaagagcctgctgaccagcatgtatgtgaaggagttcctgatcagcagcagccaggatggccaccagtggaccctgttcttccagaatggcaaggtgaaggtgttccagggcaaccaggacagcttcacccctgtggtgaacagcctggacccccccctgctgaccagatacctgaggattcacccccagagctgggtgcaccagattgccctgaggatggaggtgctgggctgtgaggcccaggacctgtactgagcggccgcgggcccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcaagcttcgcactttttaaaagaaaagggaggactggatgggatttattactccgataggacgctggcttgtaactcagtctcttactaggagaccagcttgagcctgggtgttcgctggttagcctaacctggttggccaccaggggtaaggactccttggcttagaaagctaataaacttgcctgcattagagctcttacgcgtcccgggctcgagatccgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctaacttgtttattgcagcttataatggttacaaataaagcaatagcatcacaaatttcacaaataaagcatttttttcactgcattctagttgtggtttgtccaaactcatcaatgtatcttatcatgtctgtccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaacagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcataacaccccttgtattactgtttatgtaagcagacagttttattgttcatgatgatatatttttatcttgtgcaatgtaacatcagagattttgagacacaacaattggtcgacggatcc

SEQ ID NO: 29 - DNA, 1738 bp, synthetic construct
  misc_feature 1..1738: note = CAG promoter
  source 1..1738: mol_type = other DNA
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgctcgagccacc